(19) United States
(12) Patent Application Publication
(10) Pub. No.: US 2026/0297083 A1
(43) Pub. Date: Oct. 1, 2026

(54) SUBSTITUTED ACETYLENE COMPOUND DERIVATIVES AND THEIR PHARMACEUTICAL USE

(71) Applicant: AVELOS THERAPEUTICS INC., Seoul (KR)

(72) Inventors: Jung Hun LEE, Seoul (KR); Ja Heouk KHOO, Seoul (KR); Jung Hwan KIM, Seoul (KR); Soongyu CHOI, Seoul (KR); Young Whan PARK, Seoul (KR); Minyoung IM, Seoul (KR)

(73) Assignee: AVELOS THERAPEUTICS INC., Seoul (KR)

(21) Appl. No.: 19/475,365

(22) PCT Filed: Apr. 16, 2024
(86) PCT No.: PCT/KR2024/005100

§ 371 Date: Oct. 14, 2025

(30) Foreign Application Priority Data
Apr. 17, 2023 (KR) 10-2023-0050316

(51) Int. Cl. C07D 417/14; A61K 31/4439; A61K 31/496; A61K 31/497; A61K 31/501; A61K 31/502; A61K 31/506; A61K 31/53; A61K 31/5355; C07D 403/14; C07D 413/14; C07D 487/04

(52) U.S. Cl. CPC C07D 417/14; A61K 31/4439; A61K 31/496; A61K 31/497; A61K 31/501; A61K 31/502; A61K 31/506; A61K 31/53; A61K 31/5355; C07D 403/14; C07D 413/14; C07D 487/04

(57) ABSTRACT

Provided herein are novel acetylene compound derivatives, compositions containing the compounds, and preparation methods and uses thereof. The acetylene compound derivatives are compounds represented by Formula (I), or tautomers, stereoisomers, prodrugs, crystal forms, isotopically labeled forms, pharmaceutically acceptable salts, hydrates or solvates thereof. The compounds and compositions of the present disclosure can be used to treat diseases or disorders mediated by polθ, in particular, cancer.

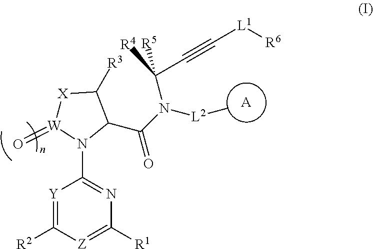

### TECHNICAL FIELD

The present disclosure belongs to a technical field of medicine, and particularly relates to acetylene derivates with inhibitory effects on polymerase theta, pharmaceutical compositions containing the same, and preparation methods and uses thereof.

### BACKGROUND ART

DNA double strand breaks (DSBs) are detrimental to cells as they contribute to genome instability, which can induce cell death. To repair DSBs, three pathways are performed in cells: non-homologous end joining (NHEJ), homologous recombination (HR), and theta-mediated end-joining (TMEJ). The NHEJ pathway joins the broken ends of DNA strands together without using a homologous template. The HR and TMEJ pathways have commonality in that they both use a homologous sequence. However, HR is a high-fidelity DNA repair pathway, and TMEJ is an error-prone DNA repair pathway that uses microhomologies.

The TMEJ pathway involves poly-(ADP-ribose) polymerase PARP1, DNA ligase III, and Polymerase theta. Polymerase theta (Polθ, encoded by POLQ) is a 290 kDa polymerase A family enzyme, and is known to mediate the TMEJ pathway (Feng et al., 2019, Nature Communication, 10:4286). Polθ possesses a N-terminal helicase-like domain and a C-terminal DNA polymerase domain separated by a non-structured central amino acid sequence. In the TMEJ pathway, the helicase domain of Polθ acts to displace Replication Protein A (RPA) bound to a single strand DNA overhang and facilitate annealing of micro-homologous sequences that flank a DSB. Then, the polymerase domain of Polθ acts to initiate DNA synthesis to fill in gaps (Zatreanu et al., 2021, Nature Communication, 12:3636).

The TMEJ pathway is also known as alternative NHEJ (alt-NHEJ) pathway or microhomology-mediated end joining (MMEJ) pathway (Zatreanu et al., 2021, Nature Communication, 12:3636). The TMEJ pathway serves as an essential backup pathway in the event that the NHEJ or HR pathway is compromised (Higgins, G. S. et al., 2018. Science, 359(6381), 1217-1218). Accordingly, DNA-repair deficient cancers are dependent on other compensatory repair pathway, and HR- or NHEJ-deficient cancers are dependent on the TMEJ backup pathway. Based on the above mechanism, PARP inhibitors (PARPi) were developed, and HR-deficient tumors have been successfully treated by way of applying PARPi. However, PARPi often face the issue of developing resistance (Drzewiecka et al., 2022, Genes, 13:1101; and Higgins, G. S. et al., 2018. Science, 359(6381), 1217-1218).

Currently, polθ has been identified as another promising therapeutic target for cancers. While polθ is overexpressed in various cancers, it is little expressed (largely absent) in normal cells (Higgins, G. S. et al., 2018. Science, 359(6381), 1217-1218; and Ceccaldi et al., 2015, Nature, 518:258). Indeed, human neoplasms including those of the lung, stomach, small intestine, rectum, and colon overexpress polθ, and the expression level of pol is particularly high in lung cancer, breast cancer, ovarian cancer, colorectal cancer and gastric cancer, etc. (Drzewiecka et al., 2022, Genes, 13:1101; and Higgins, G. S. et al., 2018. Science, 359(6381), 1217-1218). Furthermore, in vivo tests demonstrated HR-deficient tumors are hypersensitive to inhibition of Polθ-mediated DNA repair, leading to synthetic lethality (Ceccaldi et al., 2015, Nature, 518:258; and Drzewiecka et al., 2022, Genes, 13:1101). Polθ depletion causes both HR-proficient and HR-deficient tumor cells to become more sensitive to other treatments such as radiation or chemotherapy (Drzewiecka et al., 2022, Genes, 13:1101, and Goullet de Rugy T et al., 2016, Biology open, 5(10), 1485-1492).

Therefore, there is a need to develop new polθ inhibitors that effectively inhibit the TMEJ pathway and can be used for treating cancer.

### DISCLOSURE OF INVENTION

### Solution to Problem

The present disclosure provides novel acetylene derivatives, compositions comprising the same, and preparation methods and uses thereof. The acetylene derivatives have an inhibitory activity for polymerase theta, and can be effectively used for treating various types of cancers.

In one aspect, the present disclosure relates to a compound of the following Formula (I):

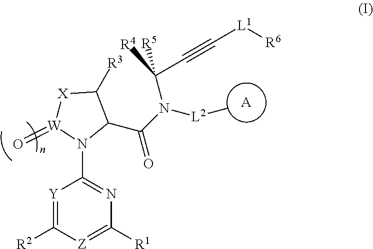

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;

L¹ is selected from the group consisting of absent, —(C₁-C₆)alkylene-, and —(C₁-C₆)alkylene-NH—;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

In another aspect, the present disclosure provides a pharmaceutical composition for treating or preventing diseases or disorders, such as diseases or disorders mediated by polymerase theta, which comprises one or more of the compounds disclosed herein, or a tautomer, stereoisomer, prodrug, crystal form, isotopically labeled form, pharmaceutically acceptable salt, hydrate, or solvate thereof, and a pharmaceutically acceptable carrier(s) or excipient(s). In a specific embodiment, the composition comprises one or more of the compounds in a therapeutically effective amount. In a specific embodiment, the composition comprises one or more of the compounds in a prophylactically effective amount.

In another aspect, the present disclosure provides the use of the compound disclosed herein, or a tautomer, stereoisomer, prodrug, crystal form, isotopically labeled form, pharmaceutically acceptable salt, hydrate, or solvate thereof, or a pharmaceutical composition disclosed herein in the manufacture of a medicament for the treatment or prevention of diseases or disorders mediated by polymerase theta.

In another aspect, the present disclosure provides a method of treating or preventing diseases or disorders, such as diseases or disorders mediated by polymerase theta, in a subject, comprising administering to the subject at least one compound disclosed herein, or a tautomer, stereoisomer, prodrug, crystal form, isotopically labeled form, pharmaceutically acceptable salt, hydrate, or solvate thereof, or a pharmaceutical composition disclosed herein.

In another aspect, the present disclosure provides the compound disclosed herein, or a tautomer, stereoisomer, prodrug, crystal form, isotopically labeled form, pharmaceutically acceptable salt, hydrate, or solvate thereof, or a pharmaceutical composition disclosed herein, for use in treating or preventing diseases or disorders, such as diseases or disorders mediated by polymerase theta.

Other objects and advantages of the present disclosure will be apparent to those skilled in the art from the following specific embodiments, examples, and claims.

### Definitions

### Chemical Terms

The definitions of specific functional groups and chemical terms are described in more detail below.

When a range of values is listed, it is intended to encompass each value and any sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein (unless otherwise specified), the term “C₁₋₆ alkyl” refers to a saturated hydrocarbon group which is straight-chained or branched, and has 1 to 6 carbon atoms. It is also referred to herein as a “lower alkyl” group. In some embodiments, the alkyl group may have 1 to 4 carbon atoms (C₁₋₄ alkyl) or 3 to 6 carbon atoms (C₃₋₆ alkyl). Examples of C₁₋₆ alkyl group include, but are not limited to methyl, ethyl, n-propyl, iso-propyl, n-butyl, tert-butyl, sec-butyl, iso-butyl, n-pentyl, 3-pentyl, 2-pentyl, neo-pentyl, 3-methyl-2-butyl, tert-pentyl, n-hexyl, 2-hexyl, 3-hexyl, and the like.

As used herein (unless otherwise specified), the term “C₂₋₆ alkenyl” refers to a hydrocarbon group which is straight-chained or branched, and has 2-6 carbon atoms and one or more carbon-carbon double bonds (e.g., 1, 2, or 3 carbon-carbon double bonds). One or more carbon-carbon double bonds can be internal (e.g., in 2-butenyl) or terminal (e.g., in 1-butenyl). In some embodiments, the alkenyl group may have 2 to 4 carbon atoms. Examples of C₂₋₆ alkenyl group include, but are not limited to, vinyl, 1-propenyl, 2-propenyl, 1-butenyl, 2-butenyl, butadienyl, pentenyl, pentadienyl, hexenyl, etc.

As used herein (unless otherwise specified), the term “C₂₋₆ alkynyl” refers to a hydrocarbon group which is straight-chained or branched, and has 2-6 carbon atoms, one or more carbon-carbon triple bonds (e.g., 1, 2 or 3 carbon-carbon triple bonds) and optionally one or more carbon-carbon double bonds (e.g., 1, 2 or 3 carbon-carbon double bonds). In some embodiments, the alkynyl group may have 2 to 4 carbon atoms. In some embodiments, the alkynyl group does not contain any double bond. One or more carbon-carbon triple bonds can be internal (e.g., in 2-butynyl) or terminal (e.g., in 1-butynyl). Examples of C₂₋₆ alkynyl group include, but are not limited to, ethynyl, 1-propynyl, 2-propynyl, 1-butynyl, 2-butynyl, pentynyl, hexynyl, etc.

As used herein (unless otherwise specified), the term “C₁₋₆ alkoxy” refers to a —OR group, wherein R is substituted or unsubstituted C₁₋₆ alkyl. In some embodiments, the alkoxy group may have 1 to 4 carbon atoms. Specifically, C₁₋₆ alkoxyl includes, but is not limited to, methoxy, ethoxy, n-propoxy, iso-propoxy, n-butoxy, t-butoxy, sec-butoxy, n-pentyloxy, n-hexyloxy and 1,2-dimethylbutoxy.

As used herein (unless otherwise specified), the terms “halo” or “halogen” refers to fluoro (F), chloro (Cl), bromo (Br) and iodo (I). In some embodiments, the halo group is F, Cl or Br. In some embodiments, the halo group is F or Cl. In some embodiments, the halo group is F.

As used herein (unless otherwise specified), the terms “C₁₋₆haloalkyl” and “C₁₋₆ haloalkoxyl” refer to the above “C₁₋₆ alkyl” and “C₁₋₆ alkoxy” substituted with one or more halo groups. Examples of the haloalkyl group include, but are not limited to, —CF₃, —CH₂F, —CHF₂, —CHFCH₂F, —CH₂CHF₂, —CF₂CF₃, —CCl₃, —CH₂Cl, —CHCl₂, 2,2,2-trifluoro-1,1-dimethyl-ethyl, etc. Examples of the haloalkoxyl group include, but are not limited to, —OCH₂F, —OCHF₂, —OCF₃, etc.

As used herein (unless otherwise specified), the terms “C₃₋₁₀cycloalkyl” or “3-10 membered cycloalkyl” refers to a cyclic hydrocarbon group which is non-aromatic and has 3-10 ring carbon atoms and zero heteroatoms. In some embodiments, the cycloalkyl group may have 3 to 8, 3 to 7, 3 to 6, 3 to 5, 3 to 4, 4 to 8, 4 to 7, 4 to 6, 5 to 8, 5 to 7, or 5 to 6 ring carbon atoms. The cycloalkyl also includes a ring system in which the above cycloalkyl ring is fused with one or more aryl or heteroaryl groups, wherein the point of attachment is on the cycloalkyl ring. Examples of the cycloalkyl group include, but are not limited to, cyclopropyl, cyclopropenyl, cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptyl, bicyclo[2.2.2]octyl, cyclononyl, cyclononenyl, cyclodecyl, cyclodecenyl, octahydro-1H-indenyl, decahydronaphthyl, spiro[4.5]decyl, and the like.

As used herein (unless otherwise specified), the term “3-10 membered heterocyclic group”, or the term “heterocyclyl” refers to a radical of a 3- to 10-membered ring system which is aromatic or non-aromatic and has ring carbon atoms and at least one ring heteroatoms, wherein each heteroatom is independently selected from nitrogen, oxygen, sulfur and phosphorus. Unless stated otherwise specifically in the specification, the heterocyclic group is a monocyclic, bicyclic, tricyclic, or tetracyclic ring system, and includes a fused, spiro, or bridged ring system. In some embodiments, the heterocyclyl group may have 2 to 7, 3 to 8, 3 to 7, 3 to 6, 3 to 5, 3 to 4, 4 to 8, 4 to 7, 4 to 6, 5 to 8, 5 to 7, or 5 to 6 ring carbon atoms, and 1 to 4 heteroatoms. In one embodiment, the heterocyclic group may be 7-11 (e.g., 9) membered bicyclic spiro heterocyclic group. In another embodiment, the heterocyclic group may be 9-10 membered bicyclic fused heterocyclic group. In another embodiment, the heterocyclic group may be 8-10 membered bicyclic bridged heterocyclic group.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, pyridinonyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl) include, without limitation, indolinyl, isoindolinyl, dihydrobenzofuranyl, dihydrobenzothienyl, benzoxazolinonyl, and the like. Exemplary 6-membered heterocyclyl groups fused to a C₆ aryl ring (also referred to herein as a 6,6-bicyclic heterocyclyl) include, without limitation, tetrahydroquinolinyl, tetrahydroisoquinolinyl, and the like. Exemplary 6-membered heterocyclyl groups with substituted with oxo include, without limitation, pyridinonyl.

In one embodiment, the heterocyclic group includes a saturated ring radical that comprises carbon atoms and from heteroatoms selected from nitrogen, oxygen, sulfur and phosphorus. In an embodiment, the saturated heterocyclic group may have a 3-10 membered heterocyclic group. In an embodiment, the saturated heterocyclic group may have 8-11 (e.g., 9) membered bicyclic spiro heterocyclic group. In some embodiments, the saturated heterocyclic group may have 2 to 7, 3 to 8, 3 to 7, 3 to 6, 3 to 5, 3 to 4, 4 to 8, 4 to 7, 4 to 6, 5 to 8, 5 to 7, or 5 to 6 ring carbon atoms, and 1 to 4 heteroatoms. Examples of such saturated heterocyclic group include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Examples of saturated spiro heterocyclic group includes 2-oxa-7-azaspiro[3.5]nonan-7-yl.

As used herein (unless otherwise specified), the term “7-11 membered polycylic heterocyclic group” refers to a monovalent heterocyclic group, which is polycyclic (where there are two or more rings) (e.g, bicyclic) ring system having 7-11 ring atoms, including carbon and one or more heteroatom (e.g., nitrogen, oxygen and sulfur). A polycyclic ring system may be a fused ring ring system, a bridged ring system and a spiro ring system. Which system depends on the bridgehead carbon, which is defined as a carbon atom which is shared by at least two rings. A fused ring ring system is a system in which the two or more rings share a covalent bond and have two bridgehead carbons. A bridged ring system is a system in which there is a carbon that is part of two or more rings and the two or more rings are connected by a bridge containing two bridegehead carbons and there are one or more carbons between the two bridegehead carbons. A spiro ring system is a system in which the two or more rings are joined with a single bridgehead carbon. Examples of polycyclic heterocyclic group includes

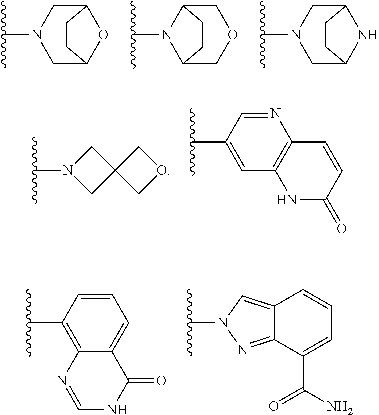

As used herein (unless otherwise specified), the term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

As used herein (unless otherwise specified), the term “C₆₋₁₄aryl” refers to a radical of a carbocyclic aromatic group, whether or not fused to one or more groups, having 6 to 14 ring carbon atoms and zero heteroatoms. In one embodiment, the aryl may have 6-10 membered ring carbon atoms. The aryl group may be monocylic or polycylic (e.g., bicyclic or tricyclic). Examples of the aryl group include, but are not limited to, phenyl, naphtyl, anthracyl, and the like. The aryl group also includes ring systems wherein the aryl ring, as defined herein, is fused with one or more cycloalkyl or heterocyclyl groups wherein the point of attachment is on the aryl ring.

As used herein (unless otherwise specified), the term “5- to 12-membered heteroaryl” refers to any monocyclic or polycyclic (e.g., bi-, or tricyclic) aromatic ring system which has ring carbon atoms and at least one heteroatoms (e.g., nitrogen, oxygen, and sulfur). The heteroaryl group also includes ring systems wherein the heteroaryl ring, as defined herein, is fused with one or more cycloalkyl, heterocyclyl or aryl groups wherein the point of attachment is on the heteroaryl ring. In some embodiments, the heteroaryl group may have 3 to 8, 3 to 7, 4 to 7, 5 to 10, 5 to 7, or 5 to 6 ring carbon atoms or heteroatoms.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl (e.g., 1,2,4-triazinyl, 1,3,5-triazinyl), and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein (unless otherwise specified), the terms “deuterated”, “deuteration”, or “D” means that one or more hydrogens in a compound or group are replaced by deuterium. Deuteration can be mono-, di-, tri-, poly-, or fully-substituted. The term “substituted with one or more deuteriums” can be used interchangeably with “deuterated one or more times”.

As used herein (unless otherwise specified), the term “non-deuterated compound” refers to a compound whose content of deuterium atoms is not higher than the natural content (0.015%) of deuterium isotope.

The content of deuterium isotope at a deuterated position is at least greater than the natural content of deuterium isotope (0.015%), alternatively greater than 30%, yet alternatively greater than 50%, yet alternatively greater than 75%, yet alternatively greater than 95%, or yet alternatively greater than 99%.

As used herein (unless otherwise specified), the term “C₁₋₆ alkylene” refers to a divalent alkyl linking group, which is a linear or branched, saturated hydrocarbon group having 1 to 6 carbon atoms. An alkylene group formally corresponds to an alkane with two C—H bonds replaced by points of attachment of the alkylene group to the remainder of the compound. In some embodiments, the alkylene group may have 1 to 4 carbon atoms (C₁₋₄ alkylene) or 1 to 2 carbon atoms (C₂ alkylene). Examples of the alkylene group include, but are not limited to, methylene, ethylene, propan-1,3-diyl, propan-1,2-diyl, butan-1,4-diyl, butan-1,3-diyl, butan-1,2-diyl, 2-methyl-propan-1,3-diyl and the like.

As used herein (unless otherwise specified), the term “carbamoyl” refers to the group —C(═O)—NR′R″, where R′ and R″ independently represent a hydrogen or C₁₋₆ alkyl group.

As used herein, the terms “optional” or “optionally” mean that the subsequently described event or circumstance may occur or may not occur, and that the description includes instances where the event or circumstance occurs as well as instances in which it does not. For example, “optionally substituted” refers to the event or circumstance that a chemical group (for example, the groups defined herein) may be substituted as well as the event or circumstance where a chemical group is not substituted.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. Exemplary substituents on carbon atoms include, but are not limited to, C₁-C₆ alkyl, halogen, cyano, deuterium, C₂-C₆ alkenyl, C₁-C₆ alkoxy, —(C₁-C₆ alkylene)-OH, —(C₁-C₆ alkylene)-O—(C₁-C₆ alkyl), 3-10 membered (e.g., 3-8 membered, 5-6 membered, 3-5 membered) cycloalkyl, hydroxy, amino, —NH₂, —NH(C₁-C₆ alkyl), —N(C₁-C₆ alkyl)(C₁-C₆ alkyl), mercapto, —S(C₁-C₆ alkyl), —SO₂(C₁-C₆ alkyl), carbamoyl, oxo, a 3-8 membered heterocyclic or heteroaryl group, —COR^{z1} (wherein R^{z1} is selected from the group consisting of hydrogen, C₁-C₆ alkyl, 3-8 membered cycloalkyl, and halogen), and —CON(R^{z2})(R^{z3}) (wherein each of R^{z2} and R^{z3} is independently selected from the group consisting of hydrogen, C₁-C₆ alkyl, or halogen, wherein said alkyl is optionally substituted with one or more selected from C₁-C₆ alkyl and halogen; or R^{z2} and R^{z3}, taken together with the nitrogen atom to which they are attached, form a nitrogen-containing 3-8 membered heterocyclic group, wherein said heterocyclic group is optionally substituted with one or more selected from C₁-C₆ alkyl and halogen), wherein each of said alkyl, alkenyl, alkoxy, alkylene, cycloalkyl, heterocyclic or heteroaryl is independently optionally substituted with one or more selected from C₁-C₆ alkyl and halogen. The number of substituents may be any number as long as valence of this substituted atom and the substituent permit, for example 1 to 5, 1 to 4, 1 to 3, 1 to 2, 2 to 5, 2 to 4, 2 to 3, 3 to 5, 3 to 4, and the like.

### General Terms

The term “about”, when used with a corresponding numeric value, is meant to encompass variations within ±20% of the numeric value, typically ±10% of the numeric value, often ±5% of the numeric value, and most often ±2% of the numeric value. In some embodiments, the term “about” can mean the numeric value itself.

Unless particularly stated otherwise, the concept of any expression in singular form should be considered to encompass the concept of the expression in plural form. Therefore, unless particularly stated otherwise, the concept of any article that expresses the concept of singular (for example, “a”, “an”, “the”, and the like in the case of English language) should be considered to encompass the concept of plural.

Unless particularly stated otherwise, any term used in the present description should be considered as having the conventional meaning for the relevant technical field. Therefore, unless defined otherwise, all the scientific terms and other technical terms used in the present description have the meaning that is generally understood by those skilled in the art to which the present invention pertains. If there is any conflict in meaning, the present description (including the definitions) takes priority.

### BEST MODE FOR CARRYING OUT THE INVENTION

### Compounds

According to an aspect of the present disclosure, provided is a compound of Formula (I) (including subsets of each formula), or a tautomer, stereoisomer, prodrug, crystal form, isotopically labeled form, pharmaceutically acceptable salt, hydrate, or solvate thereof.

As used herein, “compound of the present disclosure” refers to the following compound of Formula (I) (including subsets of each formula), or a tautomer, stereoisomer, prodrug, crystal form, isotopically labeled form, pharmaceutically acceptable salt, hydrate, or solvate thereof.

In one embodiment, the present disclosure relates to a compound of Formula (I):

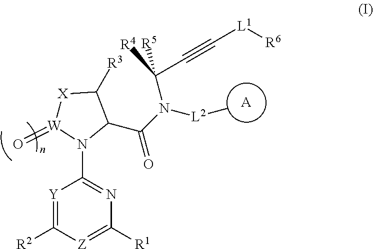

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L¹ is selected from the group consisting of absent, —(C₁-C₆)alkylene-, and —(C₁-C₆)alkylene-NH—;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR R, —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted one or more selected from with C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,

or a tautomer, stereoisomer, prodrug, crystal form, isotopically labeled form, pharmaceutically acceptable salt, hydrate, or solvate thereof.

### Ring A

In one embodiment, A is selected from the group consisting of 5-12 membered monocyclic or bicyclic aryl or heteroaryl, preferably, A is selected from the group consisting of 6-10 membered monocyclic or bicyclic aryl or heteroaryl, more preferably, A is selected from the group consisting of 6-membered monocyclic aryl or heteroaryl and 10-membered bicyclic aryl or heteroaryl, still more preferably, A is 6-membered monocyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; and R¹-R⁶, L¹, L², X, Y, Z, W and n are as defined above.

In one embodiment, A is selected from phenyl, pyridinyl, pyrrolyl, pyrazoly, pyridazinyl, pyrimidinyl, pyrazinyl, 1,2,4-triazinyl, 1,3,5-triazinyl, quinolinyl, isoquinolinyl, 4H-quinolizyinyl, quinoxalinyl, pthalazinyl, quinazolinyl, cinnolinyl, and naphthyl, wherein each of said phenyl, pyridinyl, pyrrolyl, pyrazoly, pyridazinyl, pyrimidinyl, pyrazinyl, 1,2,4-triazinyl, 1,3,5-triazinyl, quinolinyl, isoquinolinyl, 4H-quinolizyinyl, quinoxalinyl, pthalazinyl, quinazolinyl, cinnolinyl, and naphthyl is independently optionally substituted with one or more selected from C₁-C₆ alkyl, halogen, cyano, deuterium, C₂-C₆ alkenyl, hydroxy, C₁-C₆ alkoxy, 3-10 membered cycloalkyl, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; and R¹-R⁶, L¹, L², X, Y, Z, W and n are as defined above.

In one embodiment, A is phenyl, wherein said phenyl is optionally substituted with one or more selected from C₁-C₆ alkyl, halogen, cyano, deuterium, C₂-C₆ alkenyl, hydroxy, C₁-C₆ alkoxy, 3-10 membered cycloalkyl, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; and R¹-R⁶, L¹, L², X, Y, Z, W and n are as defined above.

In one embodiment, A is selected from the group consisting of 5-12 membered monocyclic or bicyclic aryl or heteroaryl, preferably, A is selected from the group consisting of 6-10 membered monocyclic or bicyclic aryl or heteroaryl, more preferably, A is selected from the group consisting of 6-membered monocyclic aryl or heteroaryl and 10-membered bicyclic aryl or heteroaryl, still more preferably, A is 6-membered monocyclic aryl or heteroaryl, wherein each of said aryl and heteroaryl is independently optionally substituted with one or more selected from an unsubstituted C₁-C₆ alkyl, a substituted C₁-C₆ alkyl with halogen, and halogen; more preferably, each of said aryl and heteroaryl is independently optionally substituted with one or more selected from methyl, ethyl, n-propyl, isopropyl, fluoromethyl, difluoromethyl, trifluoromethyl, fluoro, chloro, bromo, and iodo; more preferably, each of said aryl and heteroaryl is independently optionally substituted with one or more selected from methyl, fluoro, and chloro; and R¹-R⁶, L¹, L², X, Y, Z, W and n are as defined above.

In one embodiment, A is selected from a group consisting of phenyl, pyridinyl, pyrrolyl, pyrazoly, pyridazinyl, pyrimidinyl, pyrazinyl, 1,2,4-triazinyl, 1,3,5-triazinyl, quinolinyl, isoquinolinyl, 4H-quinolizyinyl, quinoxalinyl, pthalazinyl, quinazolinyl, cinnolinyl, and naphthyl, wherein each of said phenyl, pyridinyl, pyrrolyl, pyrazoly, pyridazinyl, pyrimidinyl, pyrazinyl, 1,2,4-triazinyl, 1,3,5-triazinyl, quinolinyl, isoquinolinyl, 4H-quinolizyinyl, quinoxalinyl, pthalazinyl, quinazolinyl, cinnolinyl, and naphthyl is independently optionally substituted with one or more selected from an unsubstituted C₁-C₆ alkyl, and a substituted C₁-C₆ alkyl with halogen, halogen, cyano, or deuterium; preferably, each of said aryl and heteroaryl is independently optionally substituted with one or more selected from an unsubstituted C₁-C₆ alkyl, and a substituted C₁-C₆ alkyl with halogen, or halogen; more preferably, each of said aryl and heteroaryl is independently optionally substituted by one or more selected from methyl, ethyl, n-propyl, isopropyl, fluoromethyl, difluoromethyl, trifluoromethyl, fluoro, chloro, bromo, and iodo; more preferably, each of said aryl and heteroaryl is independently optionally substituted with one or more selected from methyl, fluoro, and chloro; and R¹-R⁶, L¹, L², X, Y, Z, W and n are as defined above.

In one embodiment, A is phenyl, wherein said phenyl is optionally substituted with one or more selected from an unsubstituted C₁-C₆ alkyl, a substituted C₁-C₆ alkyl with halogen, halogen, cyano, and deuterium; preferably, said phenyl is optionally substituted with one or more selected from an unsubstituted C₁-C₆ alkyl, a substituted C₁-C₆ alkyl with halogen, and halogen; more preferably, said phenyl is optionally substituted with one or more selected from methyl, ethyl, n-propyl, isopropyl, fluoromethyl, difluoromethyl, trifluoromethyl, fluoro, chloro, bromo, and iodo; more preferably, said phenyl is optionally substituted with one or more selected from methyl, fluoro, and chloro; and R¹-R⁶, L¹, L², X, Y, Z, W and n are as defined above.

In one embodiment, A is

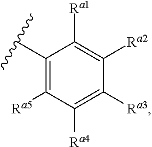

wherein:
R^{a1} is selected from the group consisting of hydrogen and fluoride;
R^{a2} is selected from the group consisting of methyl and chloride;
R^{a3} is selected from the group consisting of hydrogen, and fluoride;
R^{a4} is selected from the group consisting of hydrogen, and methyl;
R^{a5} is selected from the group consisting of hydrogen, and fluoride; and
R¹-R⁶, L¹, L², X, Y, Z, W and n are as defined above.

### X

In one embodiment,
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl,
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl, and
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen;
preferably,
X is selected from the group consisting of methylene, ethylene, n-propylene, —NH—, —NR^{z1}—, and —CR^{z2}R^{z3}—,
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl, and
R^{z2} and R^{z3}, taken together with the carbon atom to which they are attached, form a 3-5 membered cycloalkane ring, wherein said cycloalkane is optionally substituted with one or more selected from C₁-C₆ alkyl and halogen, and
R¹-R⁶, ring A, L¹, L², Y, Z, W and n are as defined above.

In one embodiment, X is selected from the group consisting of methylene, ethylene, n-propylene, —NH—, —NR^{z1}—, and —CR^{z2}R^{z3}—,
R^{z1} is C₁-C₆ alkyl, wherein said alkyl is optionally substituted with one or more selected from C₁-C₆ alkyl, halogen, cyano, and deuterium, and
R^{z2} and R^{z3}, taken together with the carbon atom to which they are attached, form cyclopropane, cyclobutane, or cyclopentane, which is optionally substituted with one or more selected from methyl, ethyl, n-propyl, isopropyl, fluoro, chloro, bromo, and iodo,
preferably,
X is selected from the group consisting of methylene, ethylene, n-propylene, —NH—, and —NR^{z1}—, and —CR^{z2}R^{z3}—,
R^{z1} is C₁-C₆ alkyl, wherein said alkyl is optionally substituted with C₁-C₆ alkyl,
R^{z2} and R^{z3}, taken together with the carbon atom to which they are attached, form cyclopropane, cyclobutane, or cyclopentane, which is optionally substituted with one or more selected from methyl, ethyl, n-propyl, isopropyl, fluoro, chloro, bromo, and iodo, and
R¹-R⁶, ring A, L¹, L², Y, Z, W and n are as defined above.

In one embodiment, X is selected from the group consisting of methylene, —NR^{z1}—, and —CR^{z2}R^{z3}—,
R^{z1} is selected from the group consisting of methyl, ethyl, n-propyl, and n-butyl, which is optionally substituted with C₁-C₆ alkyl, and
R^{z2} and R^{z3}, taken together with the carbon atom to which they are attached, form cyclopropane, or cyclobutane, which is optionally substituted with one or more selected from methyl, isopropyl, fluoro, and chloro,
preferably,
X is selected from the group consisting of methylene, —NR^{z1}—, and —CR^{z2}R^{z3}—,
R^{z1} is methyl, or ethyl, which is optionally substituted with C₁-C₆ alkyl, and
R^{z2} and R^{z3}, taken together with the carbon atom to which they are attached, form cyclopropane, which is optionally substituted with one or more selected from methyl, isopropyl, fluoro, and chloro;
more preferably,
X is selected from the group consisting of methylene, —NR^{z1}—, and —CR^{z2}R^{z3}—,
R^{z1} is methyl, or ethyl, which is optionally substituted with methyl, or ethyl, and
R^{z2} and R^{z3}, taken together with the carbon atom to which they are attached, form cyclopropane;
still more preferably,
X is selected from the group consisting of methylene, —NR^{z1}—, and —CR^{z2}R^{z3}—,
R^{z1} is methyl, or ethyl, which is optionally substituted with methyl,
R^{z2} and R^{z3}, taken together with the carbon atom to which they are attached, form cyclopropane;
further still more preferably,
X is selected from the group consisting of methylene, —NR^{z1}—, and —CR^{z2}R^{z3}—,
R^{z1} is methyl or isopropyl,
R^{z2} and R^{z3}, taken together with the carbon atom to which they are attached, form cyclopropane, and
R¹-R⁶, ring A, L¹, L², Y, Z, W and n are as defined above.

### Y

In one embodiment, Y is selected from the group consisting of —N— and —CR^{w}—; preferably, Y is —CR^{w}—, wherein R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl; more preferably, Y is —CR^{w}—, wherein R^{w} is selected from the group consisting of hydrogen, C₁-C₆ alkyl, halogen, cyano, and deuterium; still more preferably, Y is —CH—; and R¹-R⁶, ring A, L¹, L², X, Z, W and n are as defined above.

### Z

In one embodiment, Z is selected from the group consisting of —N— and —CR^{w}—, wherein R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl; preferably, Z is selected from the group consisting of —N— and —CR^{w}—, wherein R^{w} is selected from the group consisting of hydrogen, C₁-C₆ alkyl, halogen, cyano, and deuterium; more preferably, Z is —CH—; and R¹-R⁶, ring A, L¹, L², X, Y, W and n are as defined above.

### W, n

In one embodiment, W is selected from the group consisting of carbon and sulfur; and n is 1 or 2; and R¹-R⁶, ring A, L¹, L², X, Y, and Z are as defined above. When W is carbon, n is 1; and R¹-R⁶, ring A, L¹, L², X, Y, and Z are as defined above. When W is sulfur, n is 2; and R¹-R⁶, ring A, L¹, L², X, Y, and Z are as defined above.

### L

In one embodiment, L¹ is selected from the group consisting of absent, —(C₁-C₆)alkylene-, and —(C₁-C₆)alkylene-NH—; preferably, L¹ is selected from the group consisting of absent, methylene, ethylene, n-proplyene, iso-propylene, n-buthylene, -methylene-NH—, -ethylene-NH—, -n-proplyene-NH—, -iso-propylene-NH—, and -n-buthylene-NH—; more preferably, L¹ is selected from the group consisting of absent, and methylene; and R¹-R⁶, ring A, X, L², Y, Z, W and n are as defined above.

### L²

In one embodiment, L² is selected from the group consisting of absent, and —(C₁-C₆)alkylene-, wherein said alkylene is optionally substituted with one or more selected from C₁-C₆ alkyl, and halogen; preferably, L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and

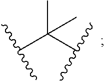 [SMILES: *C(*)(C)C]

more preferably, L² is selected from the group consisting of absent, methylene, and

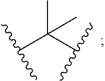 [SMILES: *C(*)(C)C]

and R¹-R⁶, ring A, X, L¹, Y, Z, W and n are as defined above.

### R¹

In one embodiment, R¹ is selected from the group consisting of hydrogen, C₁-C₆ alkyl, C₂-C₆ alkenyl, C₁-C₆ alkoxy, halogen, 3-10 membered cycloalkyl, cyano, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; preferably, R¹ is selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; more preferably, R¹ is selected from the group consisting of hydrogen and C₁-C₆ alkyl, wherein said alkyl is optionally substituted with one or more selected from C₁-C₆ alkyl, halogen, cyano, and deuterium; still more preferably, R¹ is selected from the group consisting of hydrogen and C₁-C₆ alkyl, wherein said alkyl is optionally substituted with halogen; and R²-R⁶, ring A, L¹, L², X, Y, Z, W and n are as defined above.

In one embodiment, R¹ is selected from the group consisting of methyl, ethyl, n-propyl, and isopropyl, which is optionally substituted with halogen; preferably, R¹ is selected from the group consisting of methyl, and trifluoromethyl; more preferably, R¹ is methyl; and R²-R⁶, ring A, L¹, L², X, Y, Z, W and n are as defined above.

### R²

In one embodiment, R² is selected from the group consisting of hydrogen, C₁-C₆ alkyl, C₂-C₆ alkenyl, C₁-C₆ alkoxy, halogen, 3-10 membered cycloalkyl, cyano, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; preferably, R² is selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; more preferably, R² is selected from the group consisting of hydrogen and C₁-C₆ alkyl, wherein said alkyl is optionally substituted with one or more selected from C₁-C₆ alkyl, halogen, cyano, and deuterium; still more preferably, R² is C₁-C₆ alkyl, wherein said alkyl is independently optionally substituted with one or more selected from C₁-C₆ alkyl, halogen, cyano, and deuterium; and R¹, R³-R⁶, ring A, L¹, L², X, Y, Z, W and n are as defined above.

In one embodiment, R² is C₁-C₆ alkyl, wherein said alkyl is independently optionally substituted with halogen; preferably, R² is selected from the group consisting of methyl, ethyl, and propyl, which is independently optionally substituted with halogen; more preferably, R² is trifluoromethyl; and R¹, R³-R⁶, ring A, L¹, L², X, Y, Z, W and n are as defined above.

### R³

In one embodiment, R³ is selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; preferably, R³ is selected from the group consisting of hydrogen, halogen, hydroxy, and C₁-C₆ alkyl, wherein said alkyl is optionally substituted with one or more selected from C₁-C₆ alkyl, halogen, cyano, deuterium, hydroxy, amino, mercapto, and carbamoyl; more preferably, R³ is selected from the group consisting of hydrogen, halogen, hydroxy, and C₁-C₆ alkyl, wherein said alkyl is optionally substituted with one or more selected from C₁-C₆ alkyl, halogen, cyano, and deuterium; still more preferably, R³ is selected from the group consisting of hydrogen, fluoride, chloride, bromide, iodide, hydroxy, and C₁-C₆ alkyl; further still more preferably, R³ is selected from the group consisting of hydrogen, fluoride, and hydroxy; and R¹, R², R⁴-R⁶, ring A, L¹, L², X, Y, Z, W and n are as defined above.

### R⁴

In one embodiment, R⁴ is selected from the group consisting of hydrogen, C₁-C₆ alkyl, C₂-C₆ alkenyl, C₁-C₆ alkoxy, halogen, 3-10 membered cycloalkyl, cyano, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; preferably, R⁴ is selected from the group consisting of hydrogen, and C₁-C₆ alkyl, wherein said alkyl is optionally substituted with one or more selected from C₁-C₆ alkyl, halogen, cyano, deuterium, hydroxy, amino, mercapto, and carbamoyl; more preferably, R⁴ is selected from the group consisting of hydrogen, and C₁-C₆ alkyl, wherein said alkyl is optionally substituted with one or more selected from C₁-C₆ alkyl, halogen, cyano, and deuterium; still more preferably, R⁴ is selected from the group consisting of hydrogen, and C₁-C₆ alkyl; further still more preferably, R⁴ is hydrogen; and R¹-R³, Rs, R⁶, ring A, L¹, L², X, Y, Z, W and n are as defined above.

### R⁵

In one embodiment, R⁵ is selected from the group consisting of hydrogen, C₁-C₆ alkyl, C₂-C₆ alkenyl, C₁-C₆ alkoxy, halogen, 3-10 membered cycloalkyl, cyano, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl; preferably, R⁵ is selected from the group consisting of hydrogen, and C₁-C₆ alkyl, wherein said alkyl is optionally substituted with one or more selected from C₁-C₆ alkyl, halogen, cyano, deuterium, hydroxy, amino, mercapto, and carbamoyl; more preferably, R⁵ is selected from the group consisting of hydrogen, and C₁-C₆ alkyl, wherein said alkyl is optionally substituted with one or more selected from C₁-C₆ alkyl, halogen, cyano, and deuterium; still more preferably, R⁵ is selected from the group consisting of hydrogen, and C₁-C₆ alkyl; further still more preferably, R⁵ is hydrogen; and R¹-R⁴, R⁶, ring A, L¹, L², X, Y, Z, W and n are as defined above.

### R⁶

In one embodiment, R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂ NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and R¹-R⁵, ring A, L¹, L², X, Y, Z, W and n are as defined above.

In one embodiment, R⁶ is a 3-10 membered heterocyclyl group, wherein said heterocyclyl group is optionally substituted with one or more selected from C₁-C₆ alkyl, halogen, cyano, oxo, carbamoyl, C₁-C₆ alkoxy, amino, —COOH, —S(O)₂NH₂—NR¹R², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl;
R⁶⁶ is a 6-10 membered heterocyclyl group, wherein said heterocyclyl group is optionally substituted with one or more selected from oxo, and carbamoyl; and
R¹-R⁵, ring A, L¹, L², X, Y, Z, W and n are as defined above.

In one embodiment, R⁶ is a 5-6 membered monocyclic heterocyclyl group, or a 9-10 membered bicyclic heterocyclic group, wherein said heterocyclyl group is optionally substituted with one or more selected from C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl;
R⁶⁶ is a 6-10 membered heterocyclyl group, wherein said heterocyclyl group is optionally substituted with one or more selected from oxo, and carbamoyl; and
R¹-R⁵, ring A, L¹, L², X, Y, Z, W and n are as defined above.

In one embodiment, R⁶ is selected from the group consisting of:

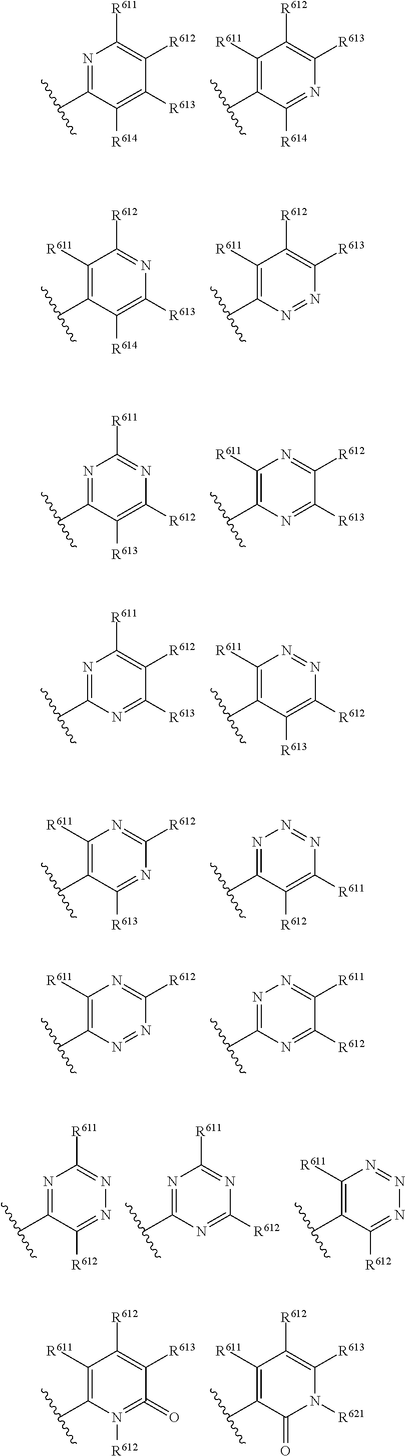

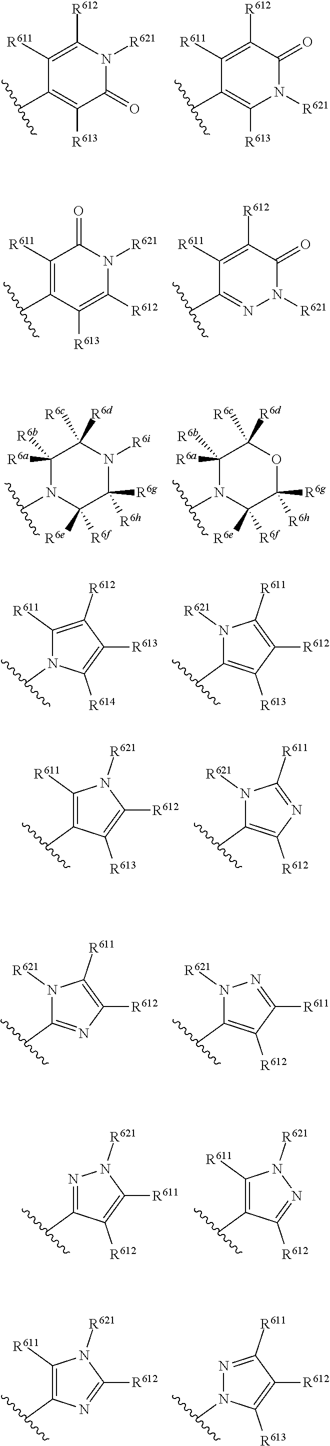

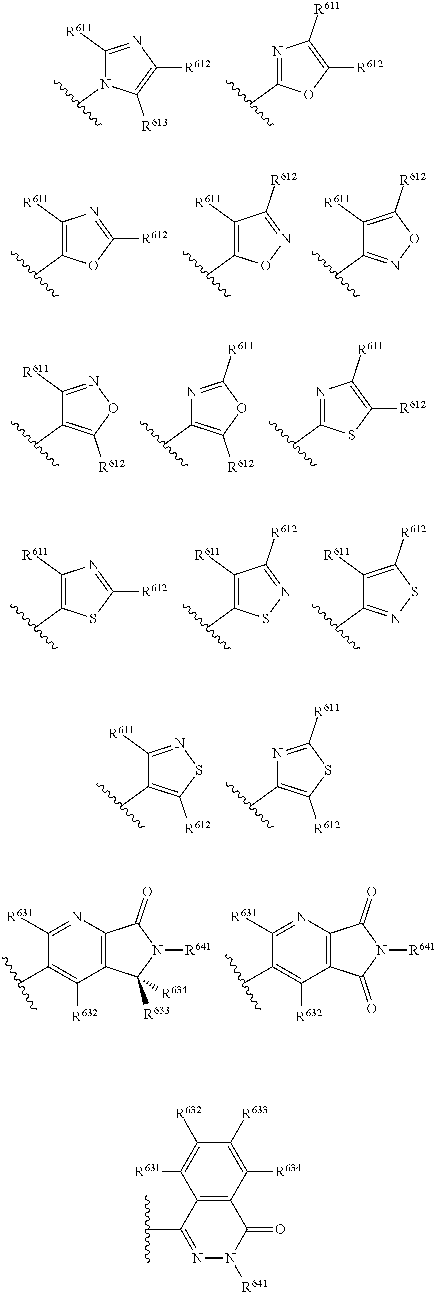

wherein:
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶¹ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl;
R⁶⁶ is a 6-10 membered heterocyclyl group, wherein said heterocyclyl group is optionally substituted with one or more selected from oxo, and carbamoyl; and
R¹-R⁵, ring A, L¹, L², X, Y, Z, W and n are as defined above.

In one embodiment, R⁶ is selected from the group consisting of:

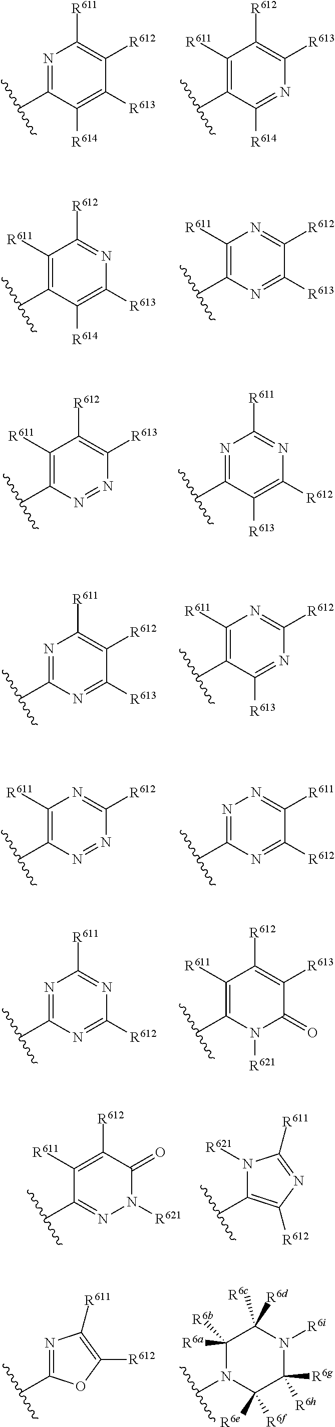

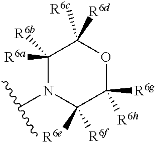

wherein:
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C, —C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl;
R⁶⁶ is a 6-10 membered heterocyclyl group, wherein said heterocyclyl group is optionally substituted with one or more selected from oxo, and carbamoyl; and
R¹-R⁵, ring A, L¹, L², X, Y, Z, W and n are as defined above.

In one embodiment, R⁶ is selected from the group consisting of:

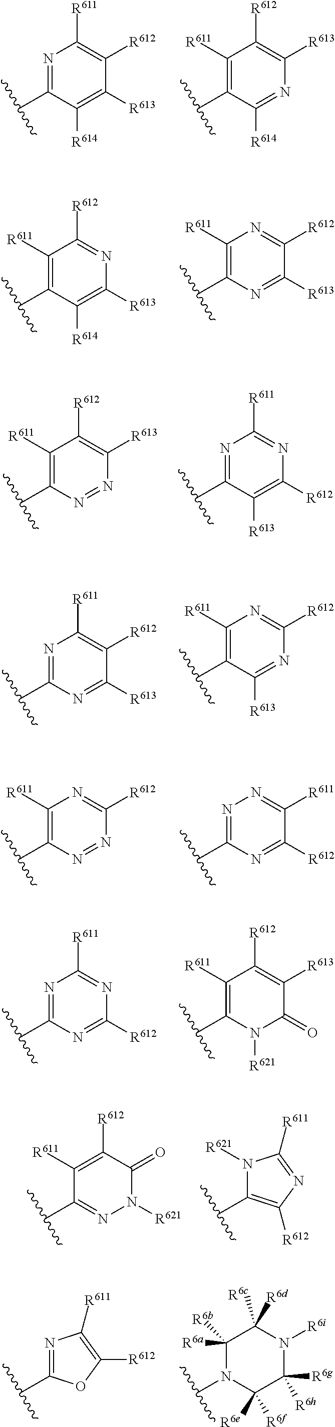

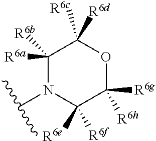

wherein:
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, n-propyl, isopropyl, trifluoromethyl, fluoro, chloro, bromo, iodo, cyano, oxo, carbamoyl, methoxy, fluoromethoxy, difluoromethoxy, trifluoromethoxy, n-propoxy, isopropoxy, amino, —COOH, —S(O)₂NH₂, —NR⁶¹R⁶², —NHC(O)R⁶³, —C(O)NR⁶⁴R⁶⁵, pyrazinyl, pyrimidinyl, triazolyl, oxadiazolyl, and —CH₂—R⁶⁶;
each of R⁶¹ and R⁶² is selected from the group consisting of hydrogen, methyl, ethyl, n-propyl, and isopropyl, or
R⁶¹ and R⁶², taken together with the nitrogen atom to which they are attached, form a nitrogen-containing 6-8 membered heterocyclic group, which is:

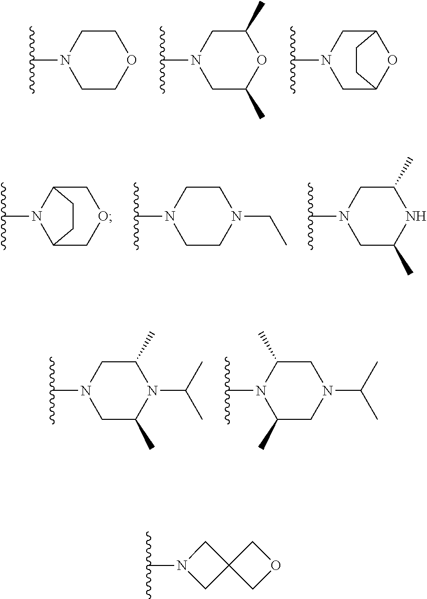

R⁶³ is selected from the group consisting of methyl, ethyl, n-propyl, isopropyl, trifluoromethyl, 1-fluoroethyl, 1,1-difluoroethyl, NH₂, ethenyl, 1-fluoroethenyl, and 1,2-difluoroethenyl;
each of R⁶⁴ and R⁶⁵ is selected from the group consisting of hydrogen, methyl, ethyl, n-propyl, and isopropyl, or
R⁶⁴ and R⁶⁵, taken together with the nitrogen atom to which they are attached, form a nitrogen-containing 6-8 membered heterocyclic group, which is:

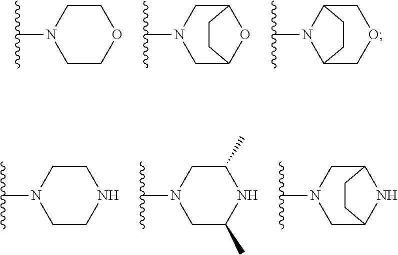

R⁶⁶ is a 6-10 membered heterocyclyl group, which is:

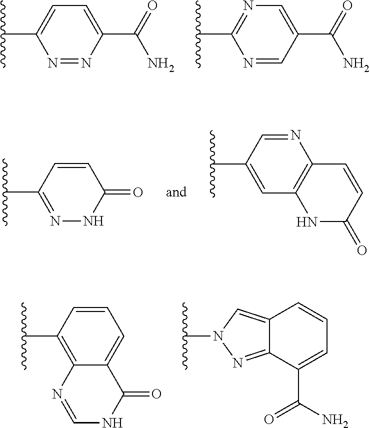 [SMILES: *c1ccc(C(N)=O)nn1 ; *c1ncc(C(N)=O)cn1 ; *c1ccc(=O)[nH]n1 ; *c1cnc2ccc(=O)[nH]c2c1 ; *c1cccc2c(=O)[nH]cnc12 ; *n1cc2cccc(C(N)=O)c2n1]

R¹-R⁵, ring A, L¹, L², X, Y, Z, W and n are as defined above.

In one embodiment, R⁶ is selected from the group consisting of:

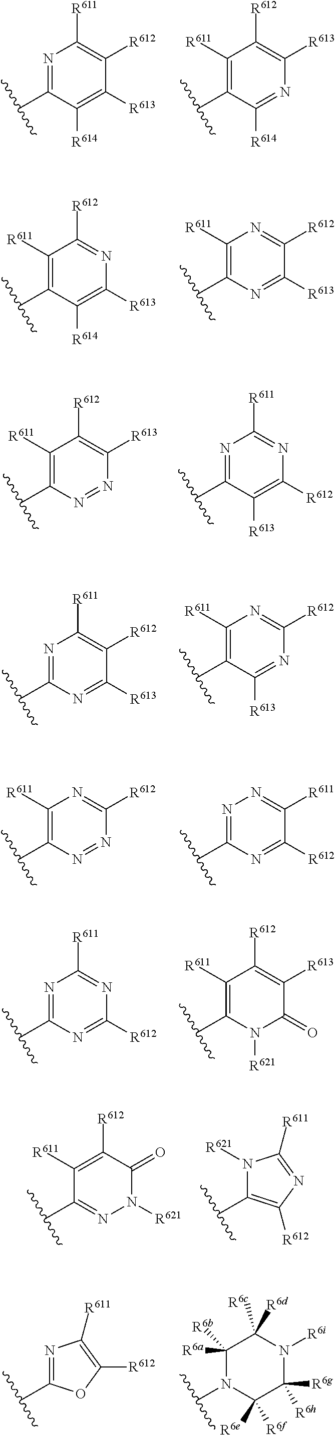

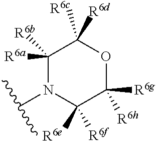

wherein:
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, C(O)NR⁶⁴R⁶⁵, —CH₂—R⁶⁶,

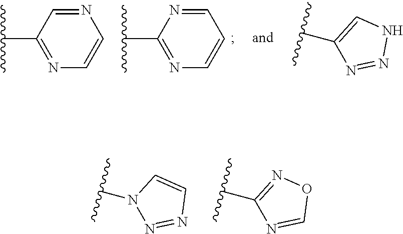

each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

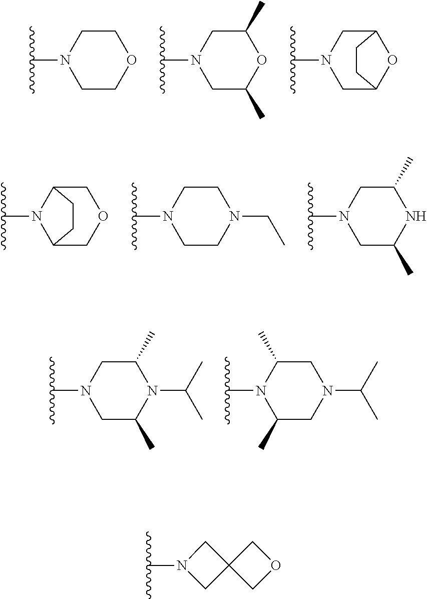

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

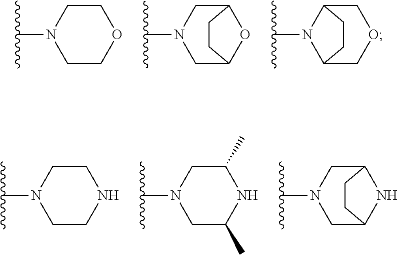

R⁶⁶ is a 6-10 membered heterocyclyl group, which is:

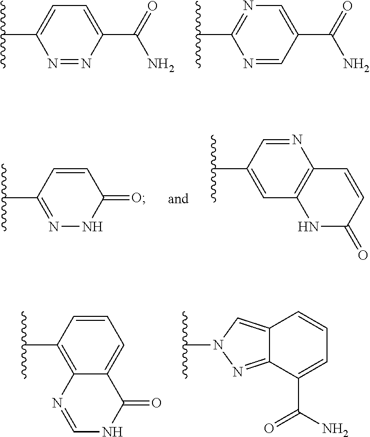 [SMILES: *c1ccc(C(N)=O)nn1 ; *c1ncc(C(N)=O)cn1 ; *c1ccc(=O)[nH]n1 ; *c1cnc2ccc(=O)[nH]c2c1 ; *c1cccc2c(=O)[nH]cnc12 ; *n1cc2cccc(C(N)=O)c2n1]

R¹-R⁵, ring A, L¹, L², X, Y, Z, W and n are as defined above.

In one embodiment, R⁶ is phenyl, wherein said phenyl is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium; and R¹-R⁵, R⁶¹-R⁶⁶, ring A, L¹, L², X, Y, Z, W and n are as defined above.

In one embodiment, R⁶ is phenyl, wherein said phenyl is optionally with one or more from C₁-C₆ alkyl, halogen, cyano, deuterium, oxo, carbamoyl, hydroxy, C₁-C₆ alkoxy, mercapto, amino, —COOH, —S(O)₂NH₂, and —C(O)NR⁶⁴R⁶⁵, wherein each of said alkyl, and alkoxy is optionally substituted with one or more selected from C₁-C₆ alkyl, halogen, and deuterium;
each of R⁶⁴ and R⁶⁵ is selected from the group consisting of hydrogen and C₁-C₆ alkyl, wherein said alkyl is optionally substituted with C₁-C₆ alkyl; and
R¹-R⁵, ring A, L¹, L², X, Y, Z, W and n are as defined above.

In one embodiment, R⁶ is phenyl, wherein said phenyl is optionally with one or more from C₁-C₆ alkyl, halogen, cyano, deuterium, oxo, carbamoyl, hydroxy, and —C(O)NR⁶⁴ R⁶¹, wherein said alkyl is optionally substituted with one or more selected from C₁-C₆ alkyl, halogen, and deuterium;
each of R⁶⁴ and R⁶⁵ is selected from the group consisting of hydrogen and C₁-C₆ alkyl, wherein said alkyl is optionally substituted with C₁-C₆ alkyl; and R¹-R⁵, R⁴-R⁶⁵, ring A, L¹, L², X, Y, Z, W and n are as defined above.

In one embodiment, R⁶ is phenyl, wherein said phenyl is optionally with carbamoyl; and R¹-R⁵, R⁶⁴-R⁶⁵, ring A, L¹, L², X, Y, Z, W and n are as defined above.

In one embodiment, R⁶ is

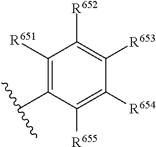

wherein:
each of R⁶⁵¹, R⁶⁵², R⁶⁵³, R⁶⁵⁴, and R⁶⁵⁵ is hydrogen, or carbamoyl; and R¹-R⁵, R⁶⁴-R⁶⁵, ring A, L¹, L², X, Y, Z, W and n are as defined above.

In one embodiment, the present disclosure relates to a compound of Formula (Ia), (Ib), (Ic), (Id), (Ie), (If), (Ig), or (Ih):

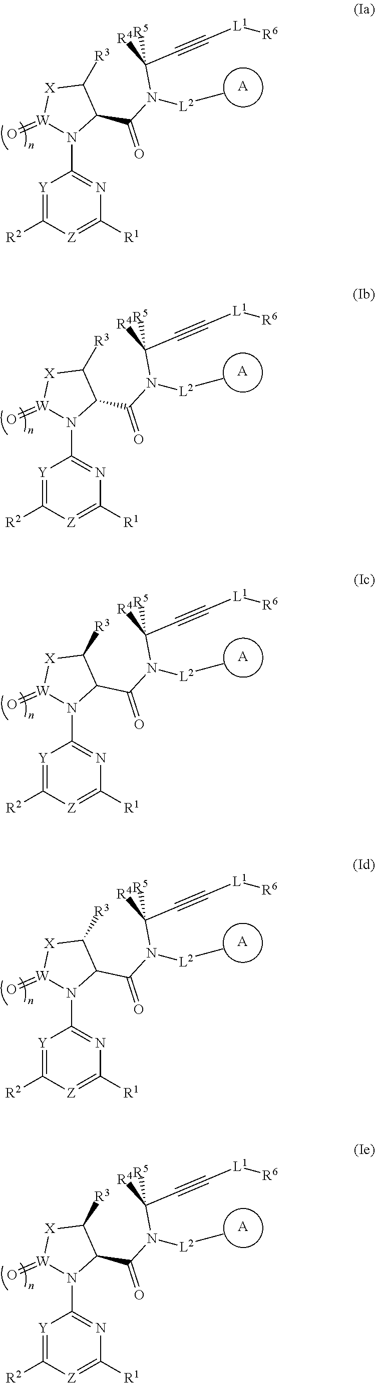

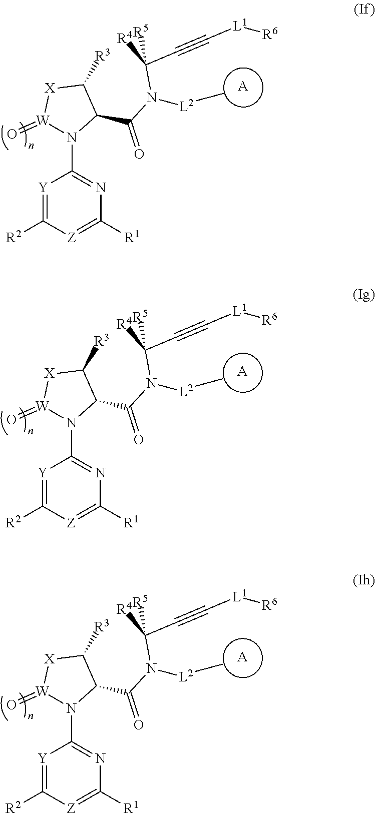

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L¹ is selected from the group consisting of absent, —(C₁-C₆)alkylene-, and —(C₁-C₆)alkylene-NH—;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

### Non-Limiting Exemplary Embodiments

In one embodiment, the present disclosure relates to a compound of Formula (II):

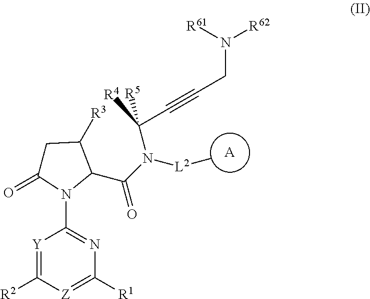

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—,
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl.
or a tautomer, stereoisomer, prodrug, crystal form, isotopically labeled form, pharmaceutically acceptable salt, hydrate, or solvate thereof.

In one embodiment, the present disclosure relates to a compound of Formula (IIa-1):

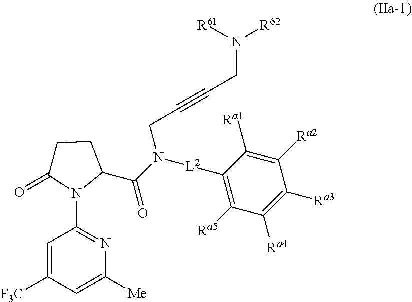

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl,

or a tautomer, stereoisomer, prodrug, crystal form, isotopically labeled form, pharmaceutically acceptable salt, hydrate, or solvate thereof.

In one embodiment of the compound of Formula (IIa-1), each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo;
L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and

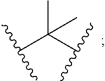 [SMILES: *C(*)(C)C]

and
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen.

In one embodiment of the compound of Formula (IIa-1),
each of R^{a1}, R^{a2}, R^{a3}, R^{a4}, and R^{a5} is selected from the group consisting of hydrogen, methyl, fluoro, and chloro;
L² is selected from the group consisting of absent, methylene, and

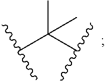 [SMILES: *C(*)(C)C]

and
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

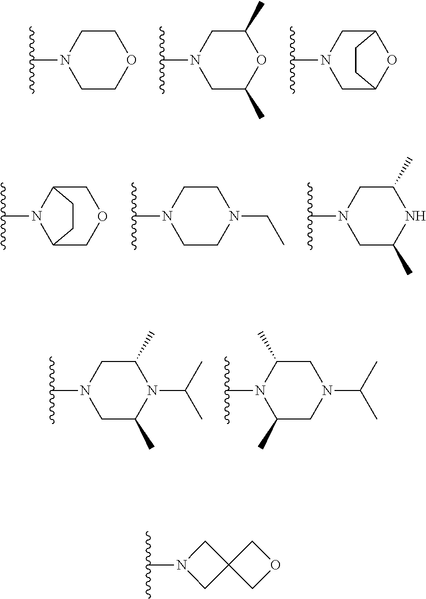

In one embodiment, the present disclosure relates to a compound of Formula (III):

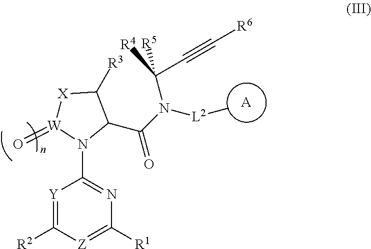

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,

or a tautomer, stereoisomer, prodrug, crystal form, isotopically labeled form, pharmaceutically acceptable salt, hydrate, or solvate thereof.

In one embodiment, the present disclosure relates to a compound of Formula (IIIa):

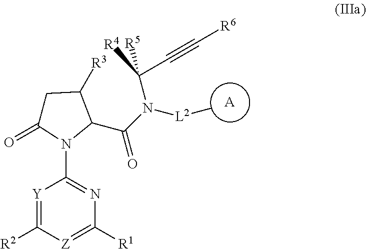

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶¹ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment, the present disclosure relates to a compound of Formula (IIIa-1):

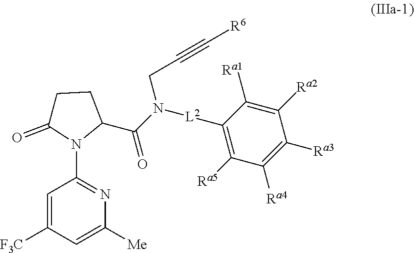

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment of the compound of Formula (IIIa-1), each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo;
L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and

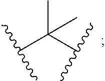 [SMILES: *C(*)(C)C]

R⁶ is selected from the group consisting of:

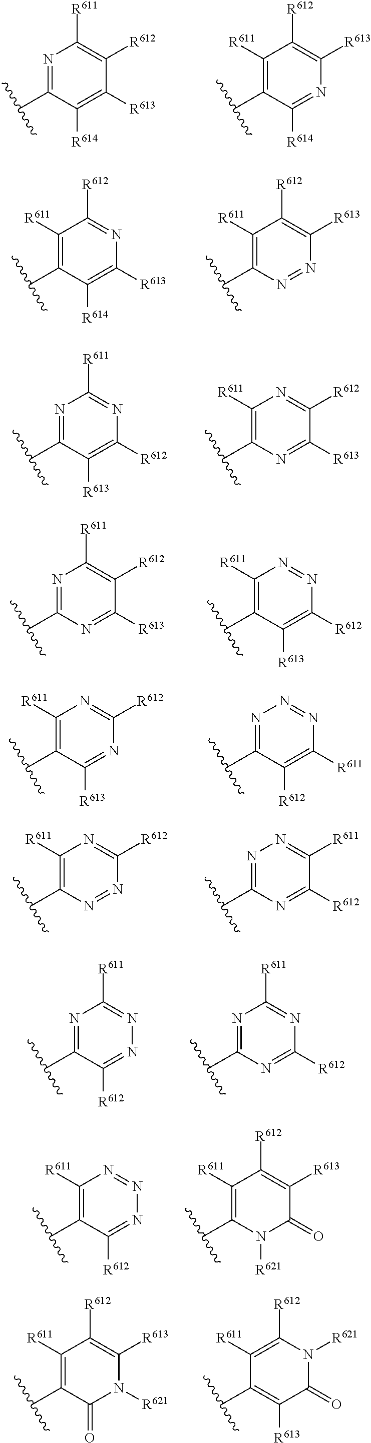

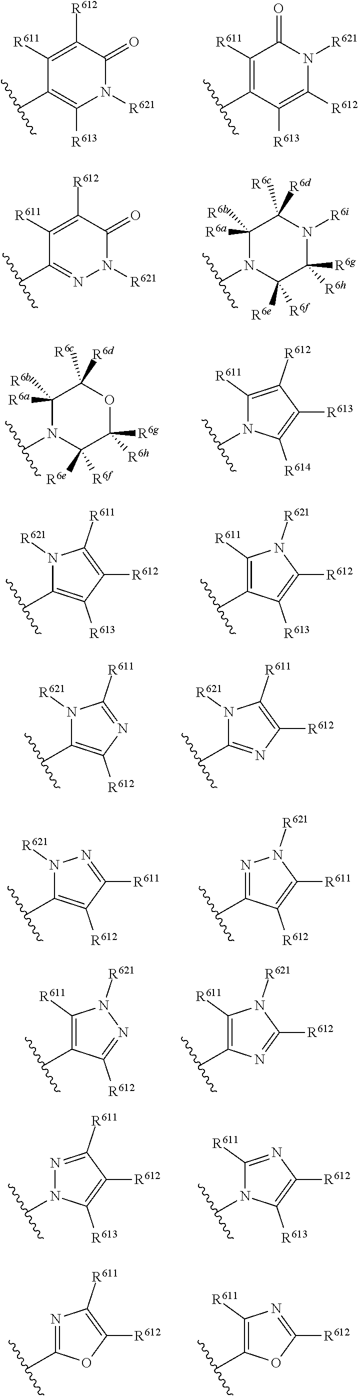

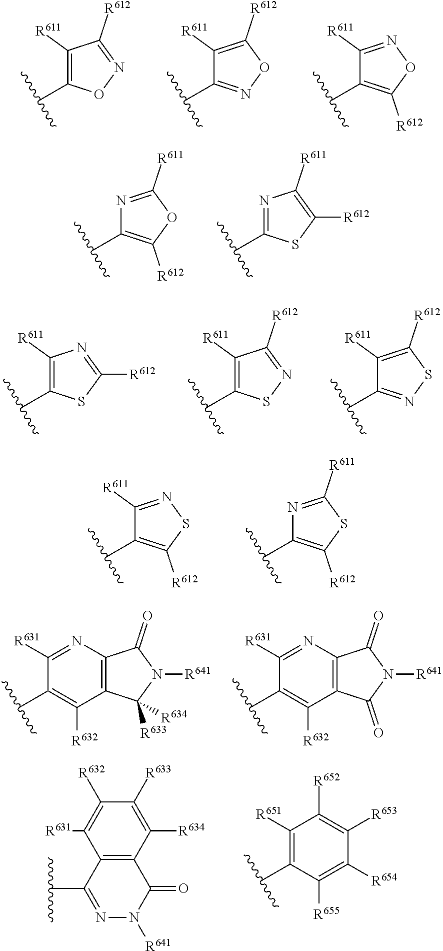

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

In one embodiment of the compound of Formula (IIIa-1),
each of R^{a1}, R^{a2}, R^{a3}, R^{a4}, and R^{a5} is selected from the group consisting of hydrogen, methyl, fluoro, and chloro;
L² is selected from the group consisting of absent, methylene, and

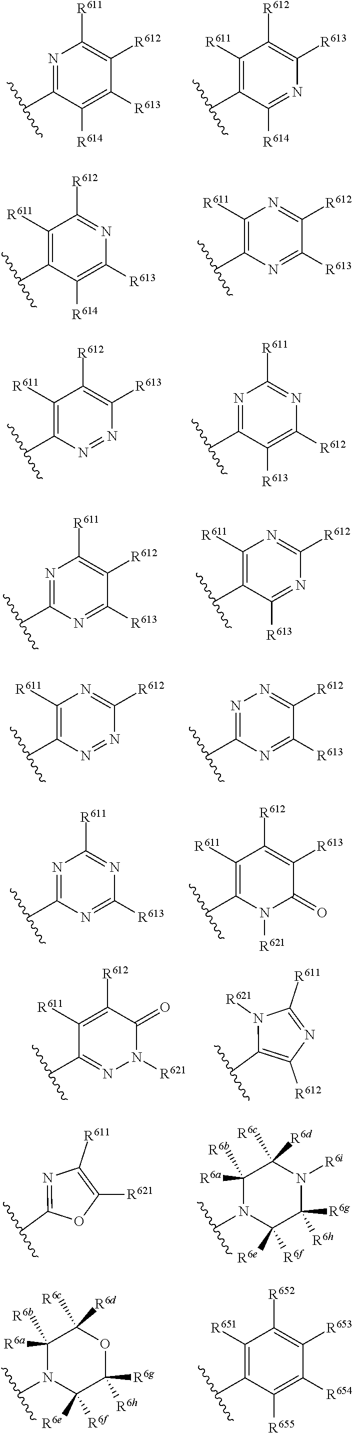

R⁶ is selected from the group consisting of:

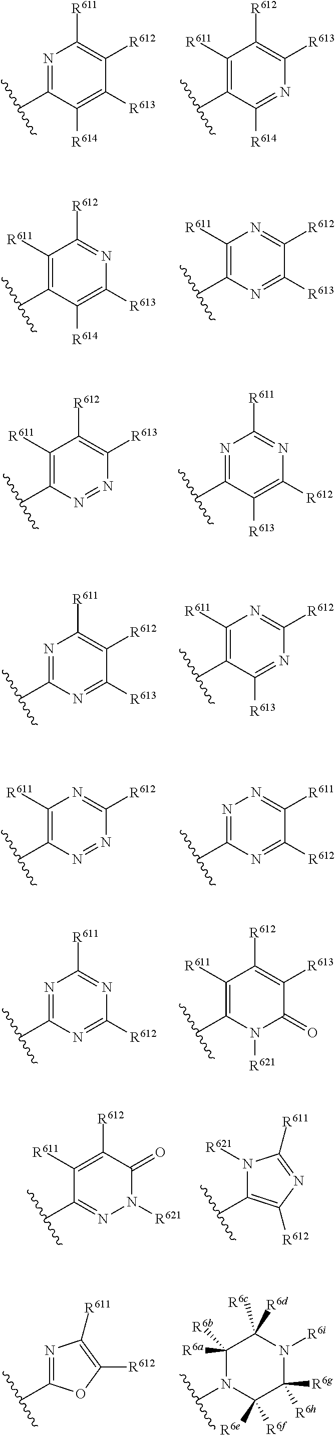

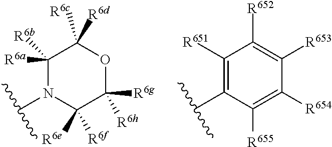

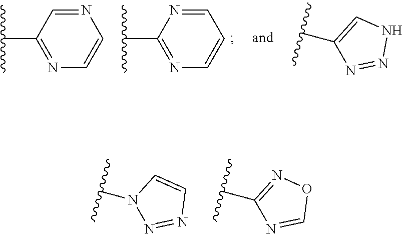

each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

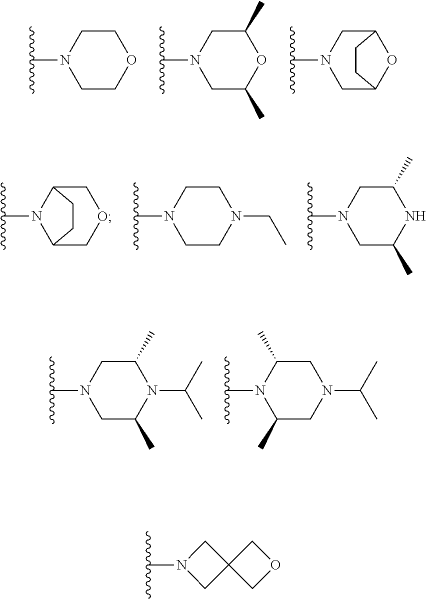

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

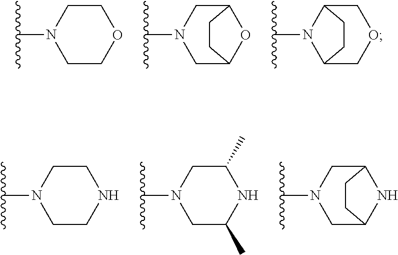

R⁶⁶ is a 6-10 membered heterocyclyl group, which is:

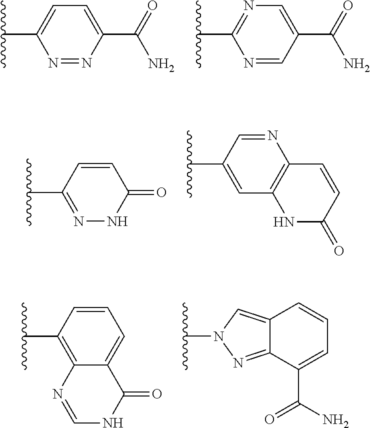

In one embodiment, the present disclosure relates to a compound of Formula (IIIb):

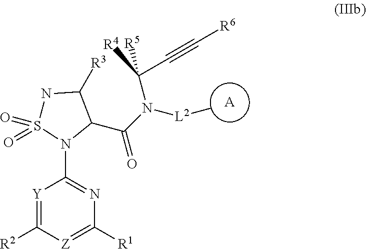

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment, the present disclosure relates to a compound of Formula (IIIb-1):

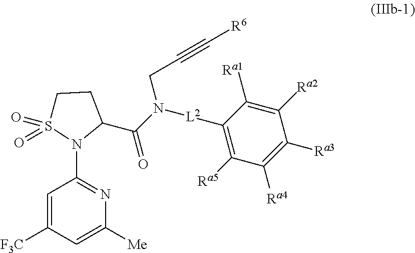

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶¹ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment of the compound of Formula (IIIb-1), each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo;
L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and;

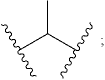 [SMILES: *C(*)C]

R⁶ is selected from the group consisting of:

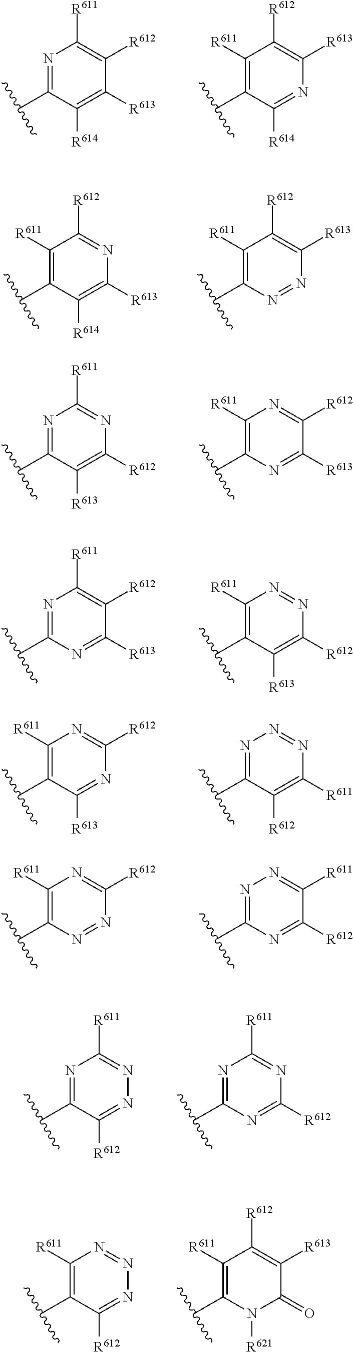

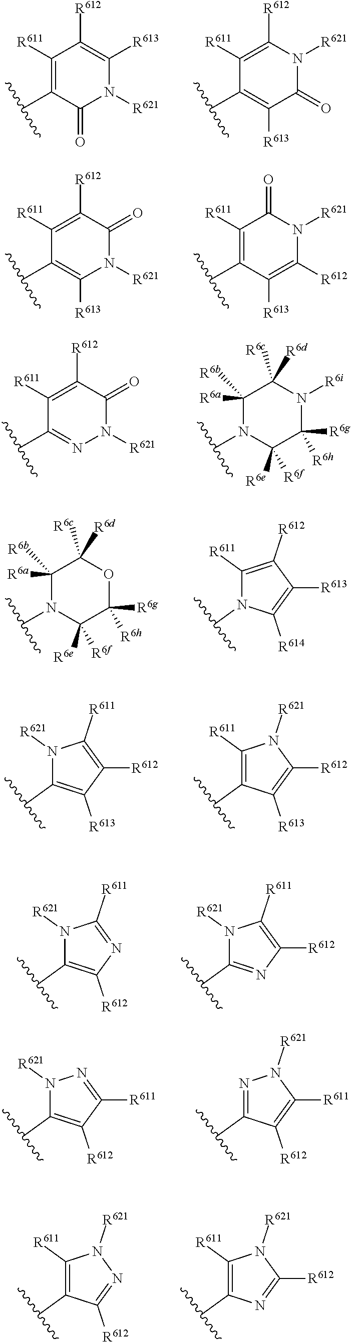

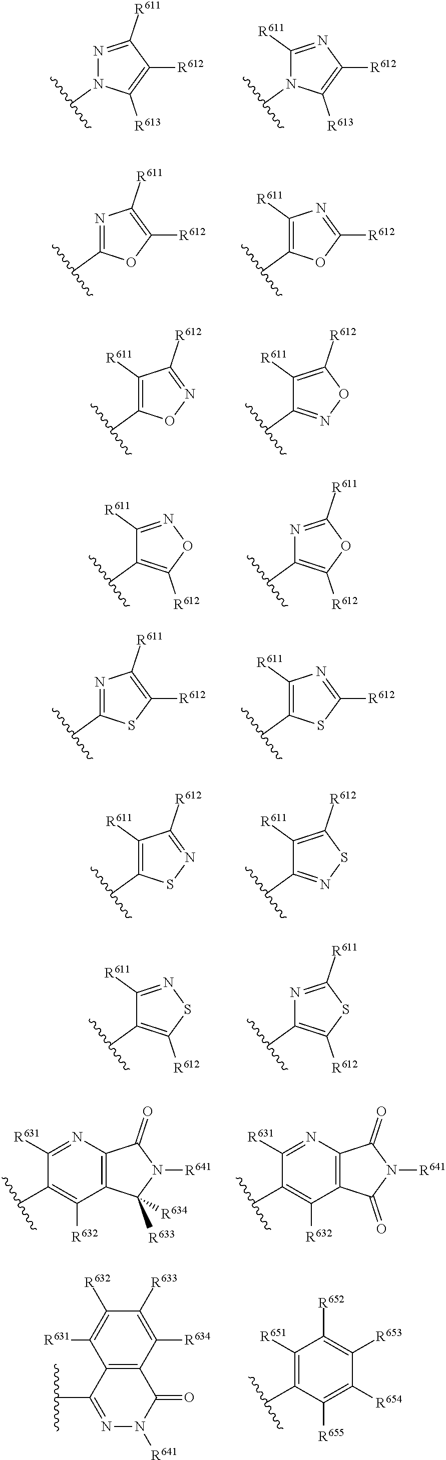

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR R, —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

In one embodiment of the compound of Formula (IIIb-1),
each of R^{a1}, R^{a2}, R^{a3}, R^{a4}, and R^{a5} is selected from the group consisting of hydrogen, methyl, fluoro, and chloro;
L² is selected from the group consisting of absent, methylene, and

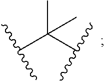 [SMILES: *C(*)(C)C]

R⁶ is selected from the group consisting of:

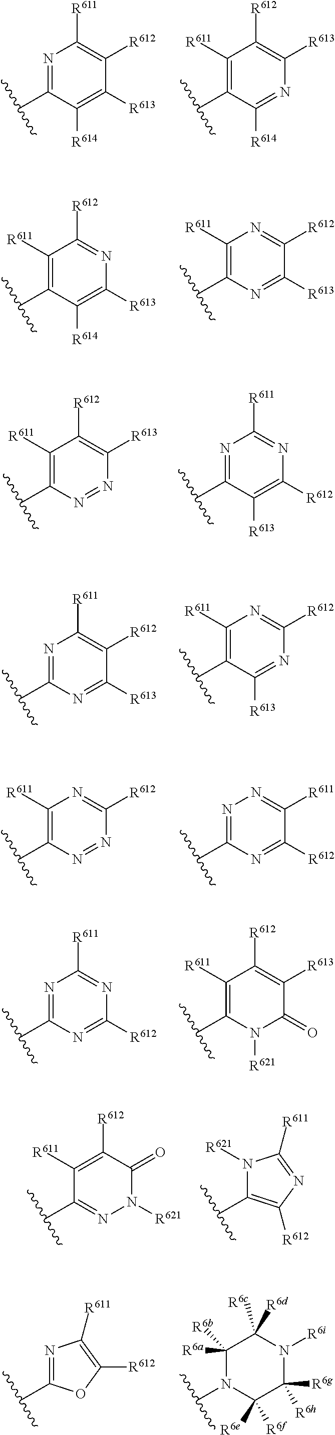

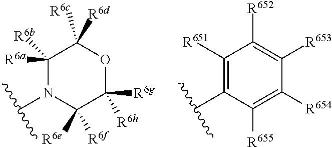

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, R⁶ⁱ, R⁶⁵¹, R⁶⁵², R⁶⁵³, R⁶⁵⁴, and R⁶⁵⁵ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, —C(O)NR⁶⁴R⁶⁵, —CH₂—R⁶⁶,

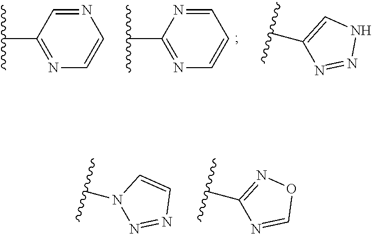

each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

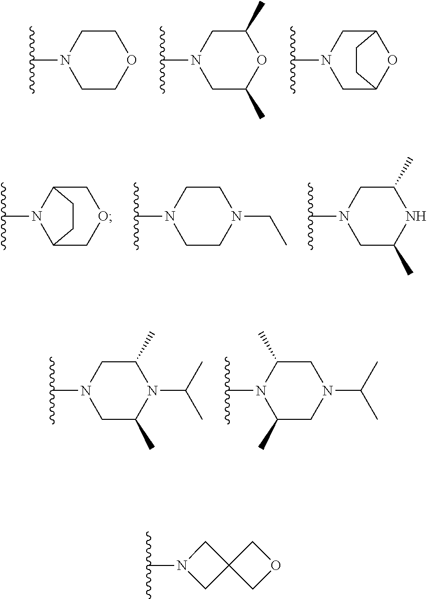

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

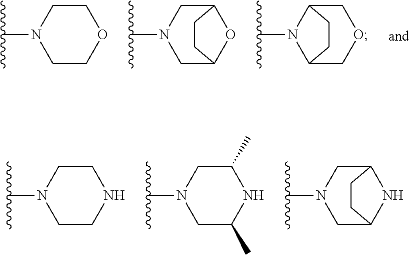

R⁶⁶ is a 6-10 membered heterocyclyl group, which is:

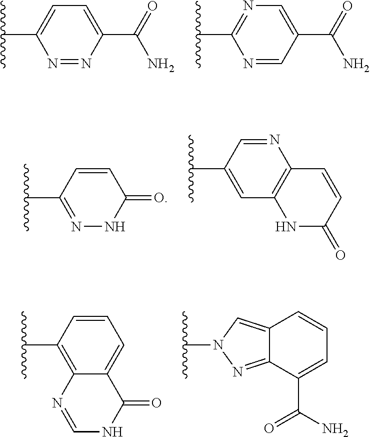

In one embodiment, the present disclosure relates to a compound of Formula (IIIc-1):

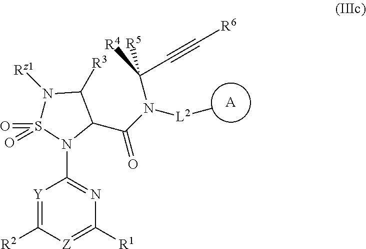

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶¹ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment, the present disclosure relates to a compound of Formula (IIIc-1):

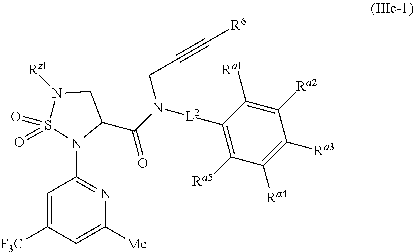

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment of the compound of Formula (IIIc-1), each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo;
R^{z1} is C₁-C₆ alkyl, wherein said alkyl is optionally substituted with C₁-C₆ alkyl;
L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and

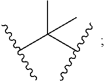 [SMILES: *C(*)(C)C]

R⁶ is selected from the group consisting of:

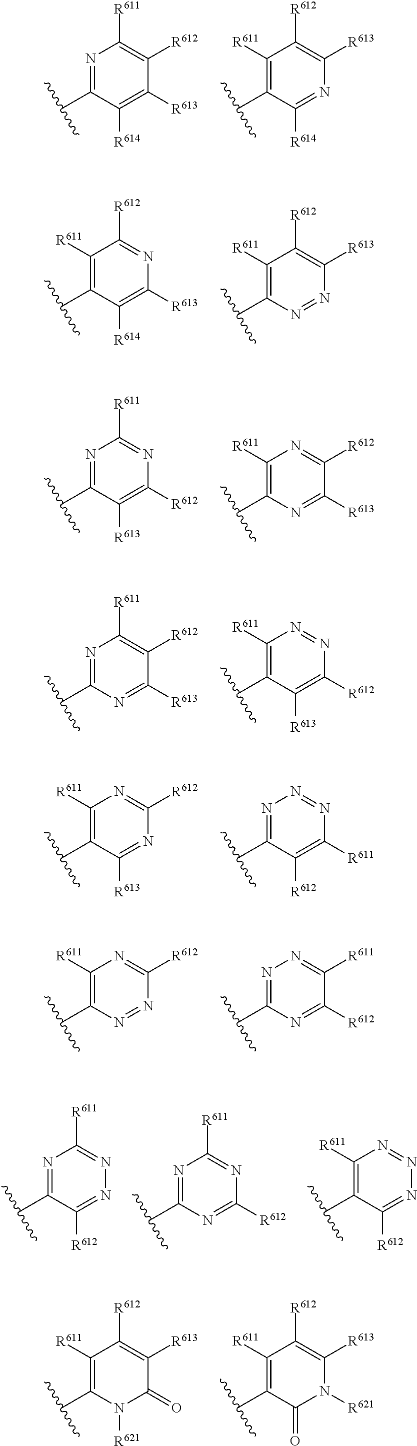

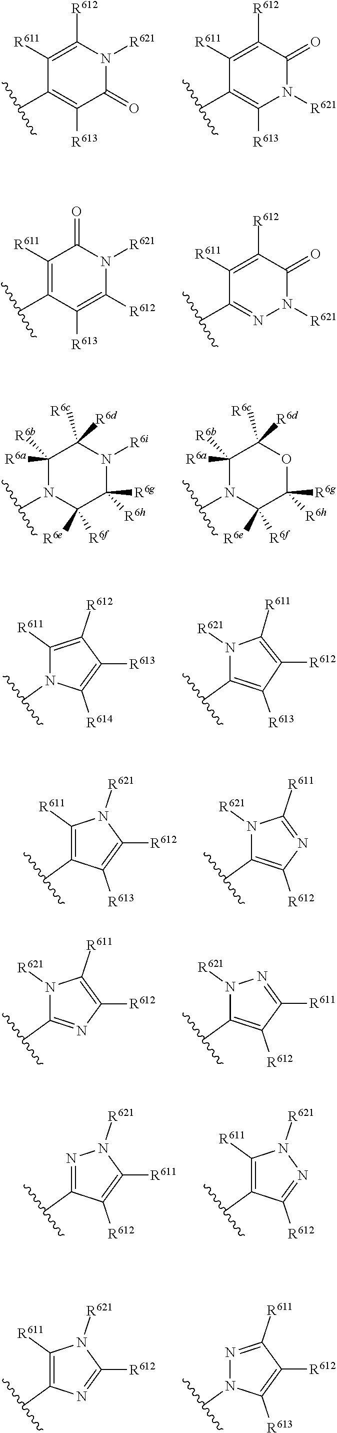

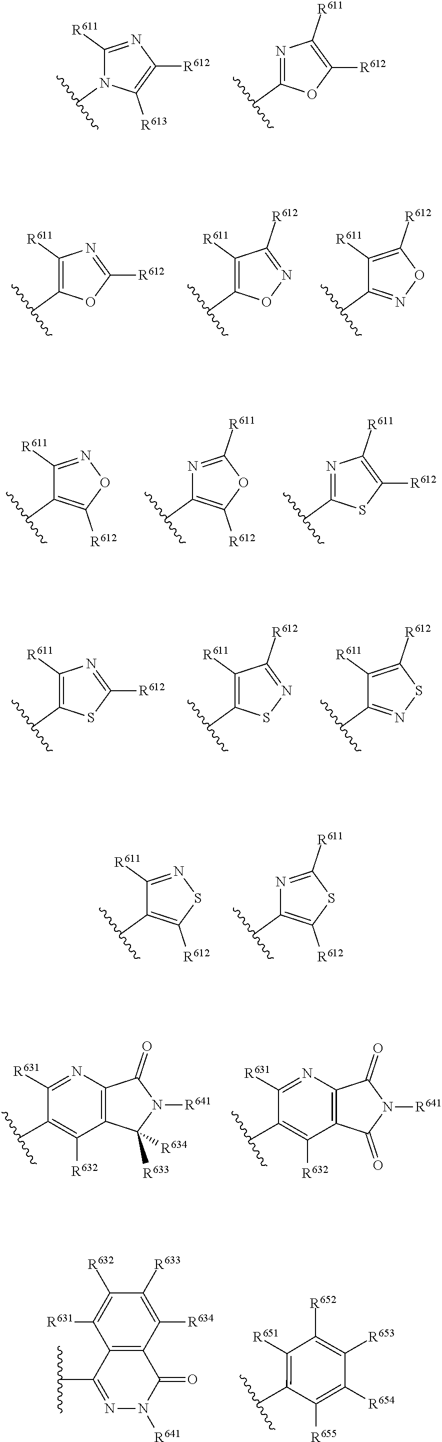

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

In one embodiment of the compound of Formula (IIIc-1),
each of R^{a1}, R^{a2}, R^{a3}, R^{a4}, and R^{a5} is selected from the group consisting of hydrogen, methyl, fluoro, and chloro;
R^{z1} is methyl or isopropyl;
L¹ is selected from the group consisting of absent, and methylene;
L² is selected from the group consisting of absent, methylene, and

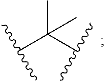 [SMILES: *C(*)(C)C]

R⁶ is selected from the group consisting of:

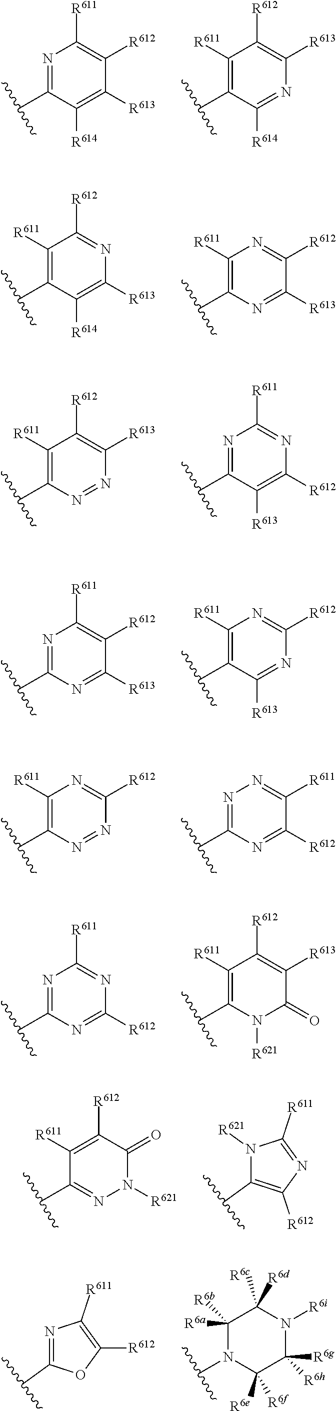

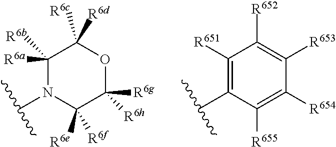

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, —C(O)NR⁶⁴R⁶⁵, —CH₂—R⁶⁶,

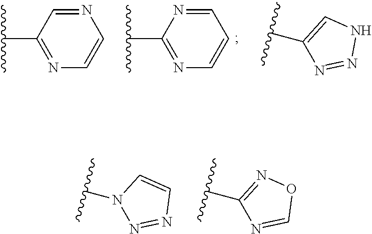

each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

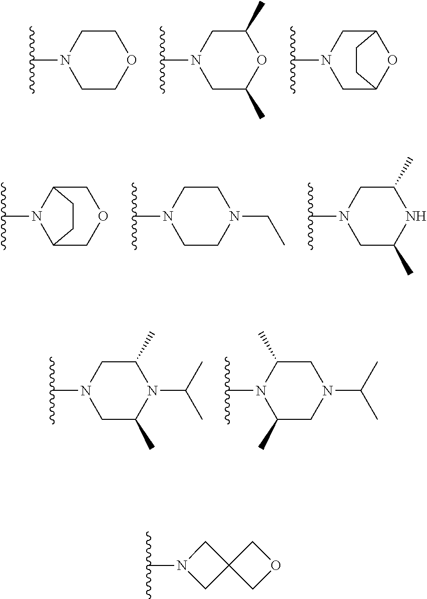

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

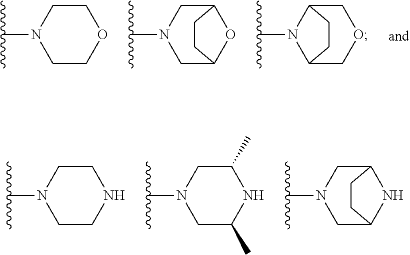

R⁶⁶ is a 6-10 membered heterocyclyl group, which is:

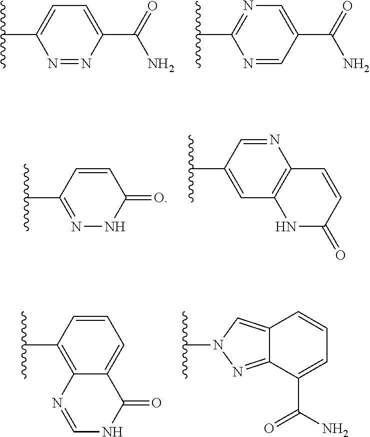

In one embodiment, the present disclosure relates to a compound of Formula (IIId):

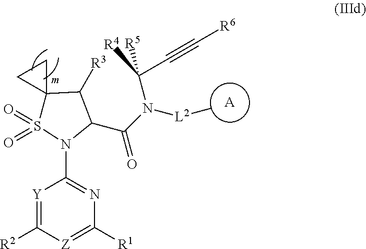

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
m is an integer from 1 to 4;
each of Y and Z is independently selected from the group consisting of —N— and —CR^{w}—;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment, the present disclosure relates to a compound of Formula (IIId-1):

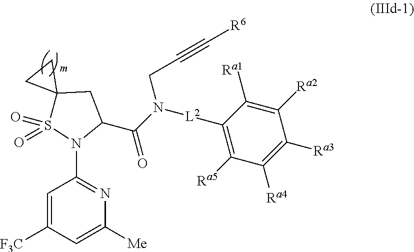

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
m is an integer from 1 to 4;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl,
or a tautomer, stereoisomer, prodrug, crystal form, isotopically labeled form, pharmaceutically acceptable salt, hydrate, or solvate thereof.

In one embodiment of the compound of Formula (IIId-1), each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo;
m is an integer from 1 to 2;
L² is selected from the group consisting of absent, methylene, 1,1-ethylene, 1,2-ethylene, 1,3-proplyene, 1,2-propylene, 1,1-propylene, 1,1-butylene, 1,2-butylene, 2,2-butylene, and

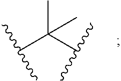 [SMILES: *C(*)(C)C]

R⁶ is selected from the group consisting of:

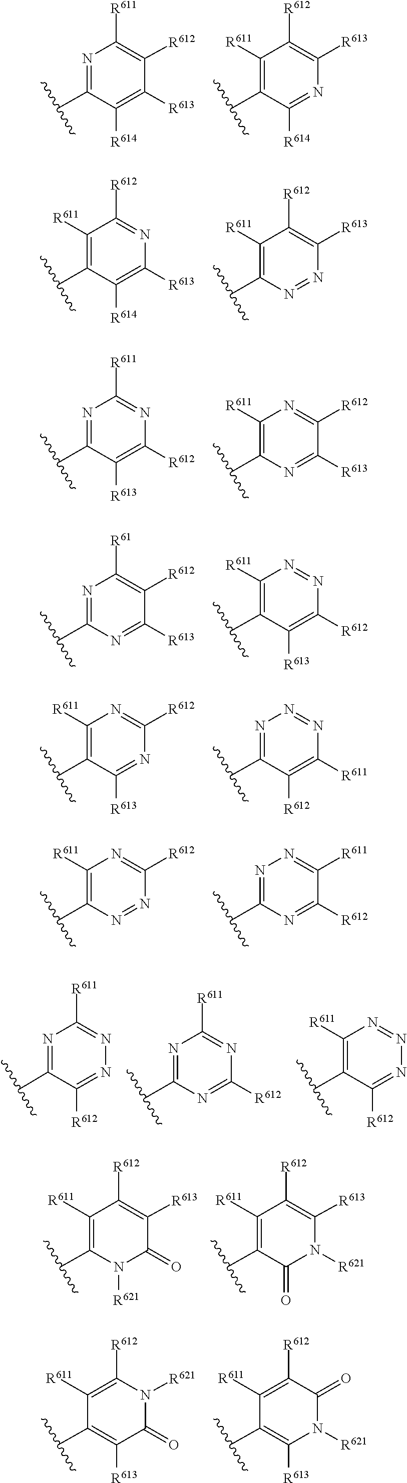

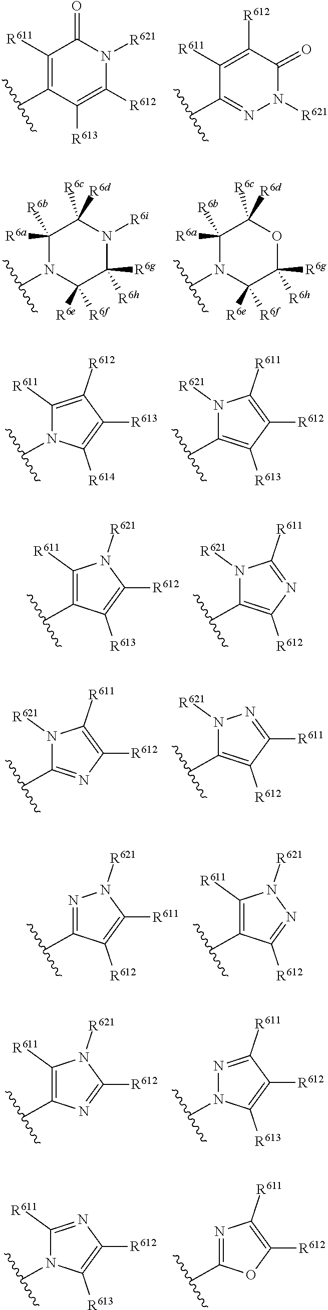

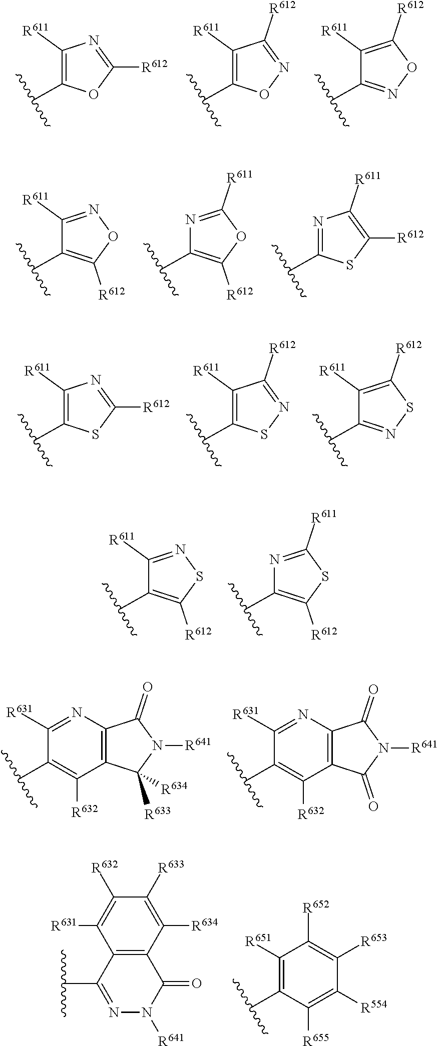

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C, —C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶¹ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

In one embodiment of the compound of Formula (IIId-1),
each of R^{a1}, R^{a2}, R^{a3}, R^{a4}, and R^{a5} is selected from the group consisting of hydrogen, methyl, fluoro, and chloro;
m is 1;
L² is selected from the group consisting of absent, methylene, and

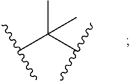 [SMILES: *C(*)(C)C]

R⁶ is selected from the group consisting of:

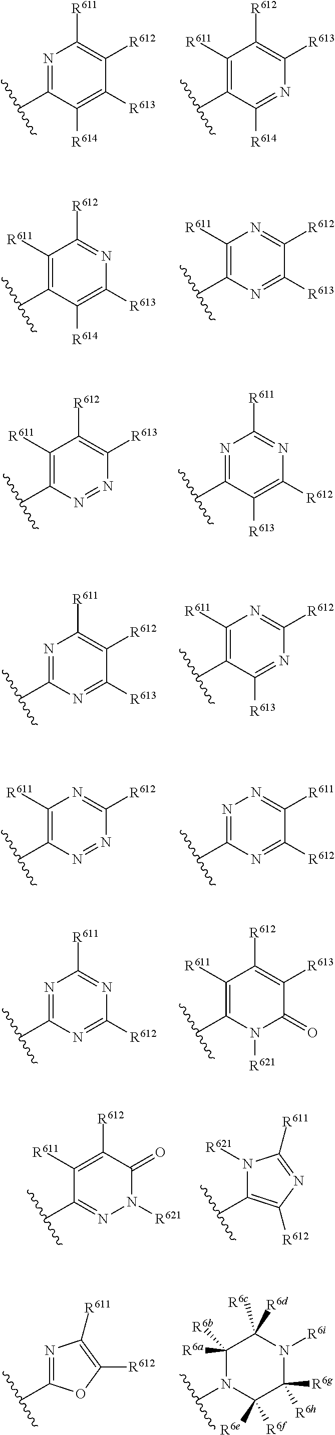

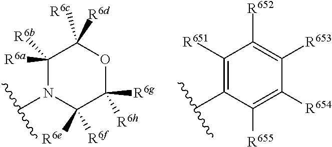

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, —C(O)NR⁶⁴R⁶⁵, —CH₂—R⁶⁶,

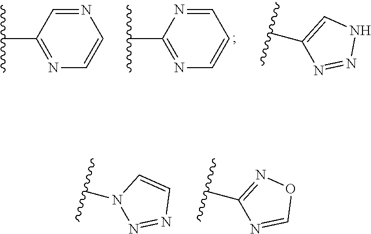

each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

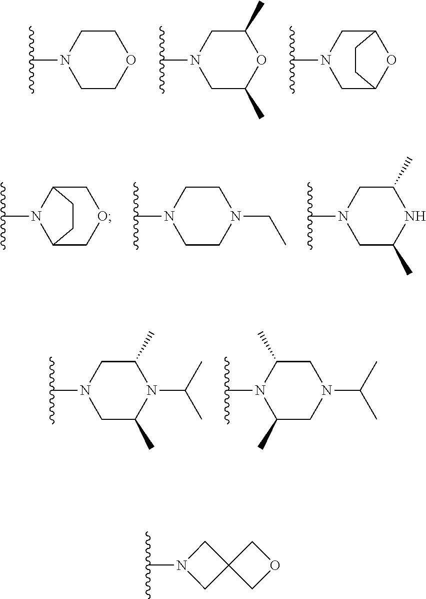

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

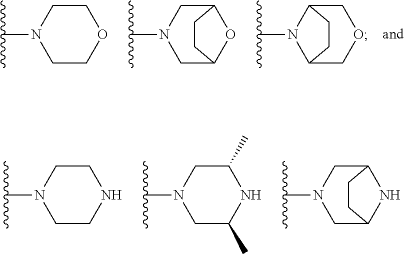

R⁶⁶ is a 6-10 membered heterocyclyl group, which is:

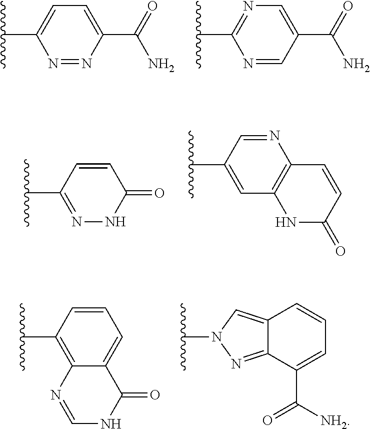

In one embodiment, exemplary compounds of Formula (I) are provided below:

Structure

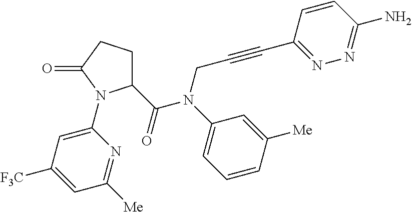 [SMILES: Cc1cccc(N(CC#Cc2ccc(N)nn2)C(=O)C2CCC(=O)N2c2cc(C(F)(F)F)cc(C)n2)c1]

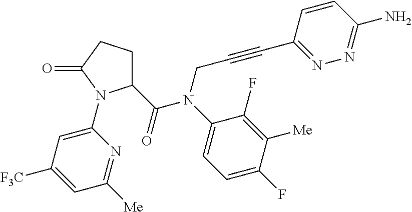 [SMILES: Cc1cc(C(F)(F)F)cc(N2C(=O)CCC2C(=O)N(CC#Cc2ccc(N)nn2)c2ccc(F)c(C)c2F)n1]

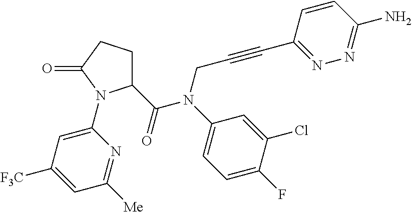 [SMILES: Cc1cc(C(F)(F)F)cc(N2C(=O)CCC2C(=O)N(CC#Cc2ccc(N)nn2)c2ccc(F)c(Cl)c2)n1]

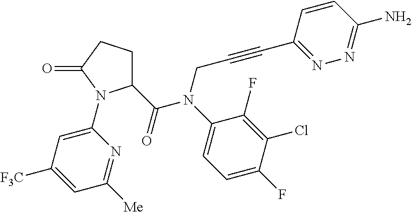 [SMILES: Cc1cc(C(F)(F)F)cc(N2C(=O)CCC2C(=O)N(CC#Cc2ccc(N)nn2)c2ccc(F)c(Cl)c2F)n1]

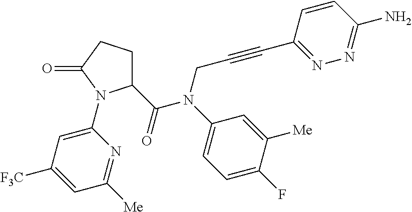 [SMILES: Cc1cc(C(F)(F)F)cc(N2C(=O)CCC2C(=O)N(CC#Cc2ccc(N)nn2)c2ccc(F)c(C)c2)n1]

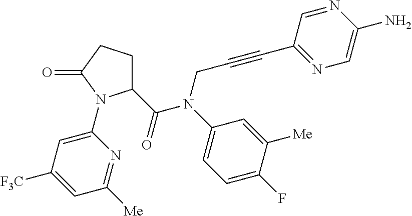 [SMILES: Cc1cc(C(F)(F)F)cc(N2C(=O)CCC2C(=O)N(CC#Cc2cnc(N)cn2)c2ccc(F)c(C)c2)n1]

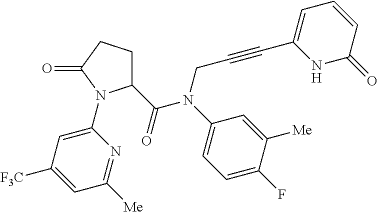 [SMILES: Cc1cc(C(F)(F)F)cc(N2C(=O)CCC2C(=O)N(CC#Cc2cccc(=O)[nH]2)c2ccc(F)c(C)c2)n1]

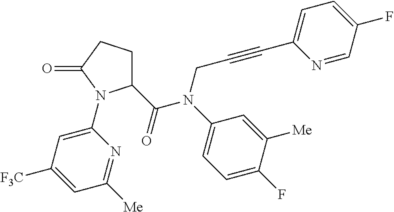 [SMILES: Cc1cc(C(F)(F)F)cc(N2C(=O)CCC2C(=O)N(CC#Cc2ccc(F)cn2)c2ccc(F)c(C)c2)n1]

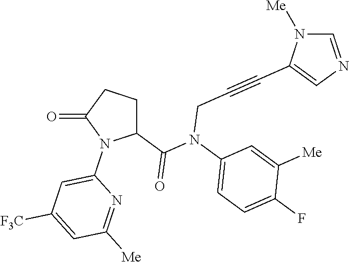 [SMILES: Cc1cc(C(F)(F)F)cc(N2C(=O)CCC2C(=O)N(CC#Cc2cncn2C)c2ccc(F)c(C)c2)n1]

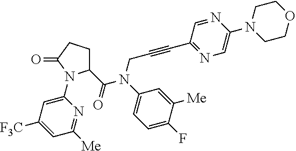 [SMILES: Cc1cc(C(F)(F)F)cc(N2C(=O)CCC2C(=O)N(CC#Cc2cnc(N3CCOCC3)cn2)c2ccc(F)c(C)c2)n1]

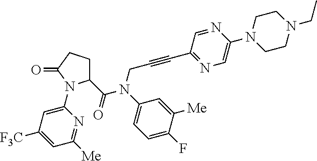 [SMILES: CCN1CCN(c2cnc(C#CCN(C(=O)C3CCC(=O)N3c3cc(C(F)(F)F)cc(C)n3)c3ccc(F)c(C)c3)cn2)CC1]

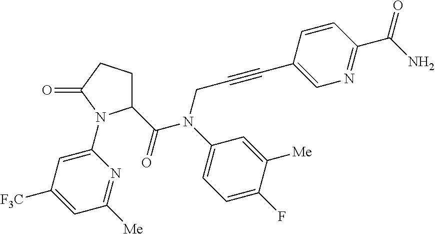 [SMILES: Cc1cc(C(F)(F)F)cc(N2C(=O)CCC2C(=O)N(CC#Cc2ccc(C(N)=O)nc2)c2ccc(F)c(C)c2)n1]

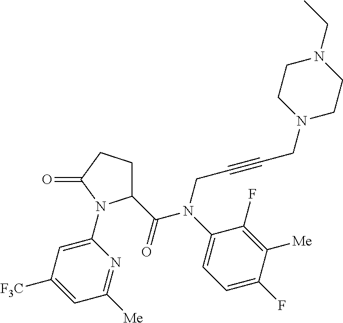 [SMILES: CCN1CCN(CC#CCN(C(=O)C2CCC(=O)N2c2cc(C(F)(F)F)cc(C)n2)c2ccc(F)c(C)c2F)CC1]

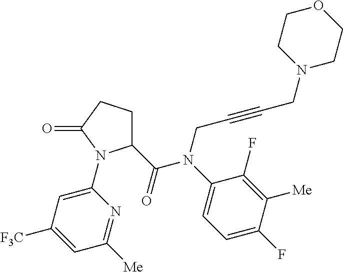 [SMILES: Cc1cc(C(F)(F)F)cc(N2C(=O)CCC2C(=O)N(CC#CCN2CCOCC2)c2ccc(F)c(C)c2F)n1]

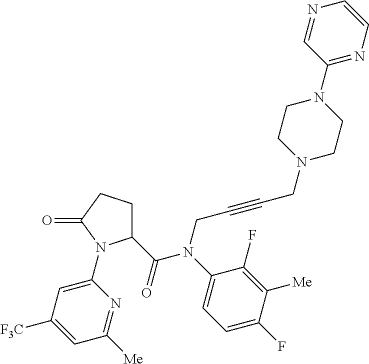 [SMILES: Cc1cc(C(F)(F)F)cc(N2C(=O)CCC2C(=O)N(CC#CCN2CCN(c3cnccn3)CC2)c2ccc(F)c(C)c2F)n1]

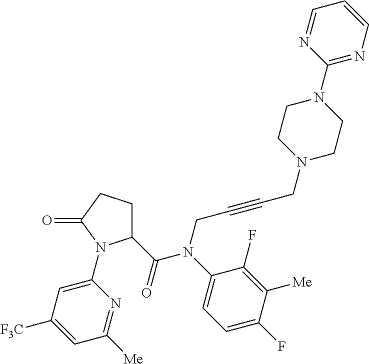 [SMILES: Cc1cc(C(F)(F)F)cc(N2C(=O)CCC2C(=O)N(CC#CCN2CCN(c3ncccn3)CC2)c2ccc(F)c(C)c2F)n1]

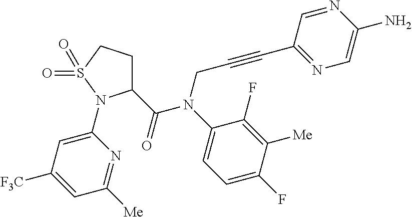 [SMILES: Cc1cc(C(F)(F)F)cc(N2C(C(=O)N(CC#Cc3cnc(N)cn3)c3ccc(F)c(C)c3F)CCS2(=O)=O)n1]

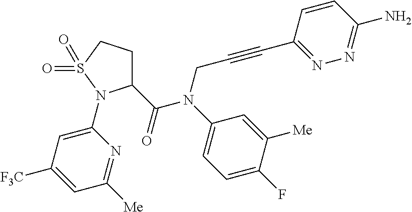 [SMILES: Cc1cc(C(F)(F)F)cc(N2C(C(=O)N(CC#Cc3ccc(N)nn3)c3ccc(F)c(C)c3)CCS2(=O)=O)n1]

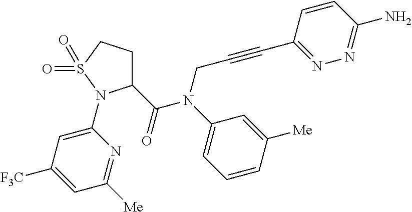 [SMILES: Cc1cccc(N(CC#Cc2ccc(N)nn2)C(=O)C2CCS(=O)(=O)N2c2cc(C(F)(F)F)cc(C)n2)c1]

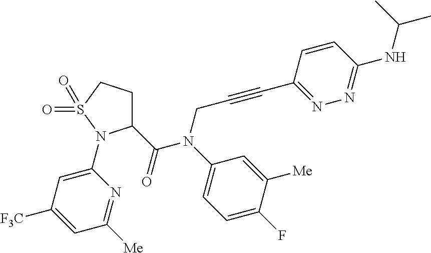 [SMILES: Cc1cc(C(F)(F)F)cc(N2C(C(=O)N(CC#Cc3ccc(NC(C)C)nn3)c3ccc(F)c(C)c3)CCS2(=O)=O)n1]

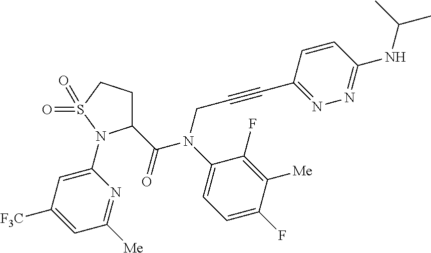 [SMILES: Cc1cc(C(F)(F)F)cc(N2C(C(=O)N(CC#Cc3ccc(NC(C)C)nn3)c3ccc(F)c(C)c3F)CCS2(=O)=O)n1]

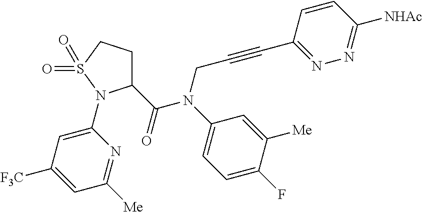 [SMILES: CC(=O)Nc1ccc(C#CCN(C(=O)C2CCS(=O)(=O)N2c2cc(C(F)(F)F)cc(C)n2)c2ccc(F)c(C)c2)nn1]

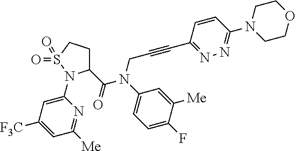 [SMILES: Cc1cc(C(F)(F)F)cc(N2C(C(=O)N(CC#Cc3ccc(N4CCOCC4)nn3)c3ccc(F)c(C)c3)CCS2(=O)=O)n1]

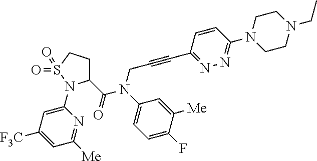 [SMILES: CCN1CCN(c2ccc(C#CCN(C(=O)C3CCS(=O)(=O)N3c3cc(C(F)(F)F)cc(C)n3)c3ccc(F)c(C)c3)nn2)CC1]

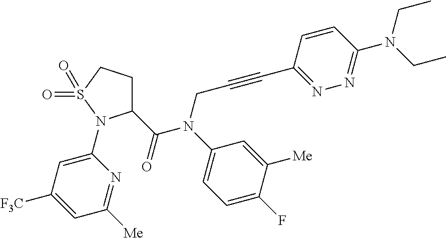 [SMILES: CCN(CC)c1ccc(C#CCN(C(=O)C2CCS(=O)(=O)N2c2cc(C(F)(F)F)cc(C)n2)c2ccc(F)c(C)c2)nn1]

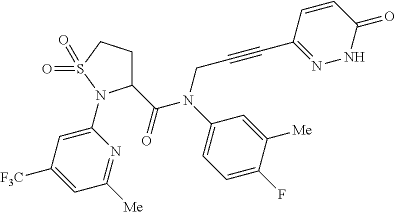 [SMILES: Cc1cc(C(F)(F)F)cc(N2C(C(=O)N(CC#Cc3ccc(=O)[nH]n3)c3ccc(F)c(C)c3)CCS2(=O)=O)n1]

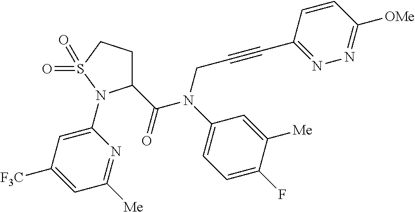 [SMILES: COc1ccc(C#CCN(C(=O)C2CCS(=O)(=O)N2c2cc(C(F)(F)F)cc(C)n2)c2ccc(F)c(C)c2)nn1]

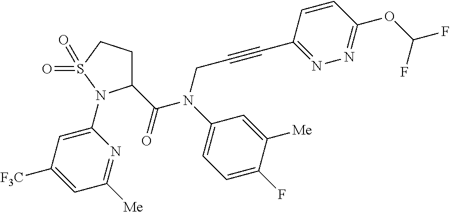 [SMILES: Cc1cc(C(F)(F)F)cc(N2C(C(=O)N(CC#Cc3ccc(OC(F)F)nn3)c3ccc(F)c(C)c3)CCS2(=O)=O)n1]

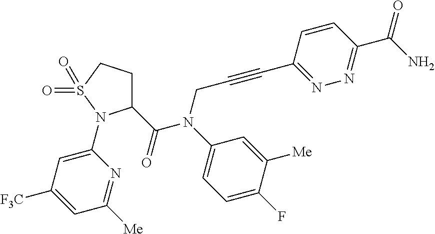 [SMILES: Cc1cc(C(F)(F)F)cc(N2C(C(=O)N(CC#Cc3ccc(C(N)=O)nn3)c3ccc(F)c(C)c3)CCS2(=O)=O)n1]

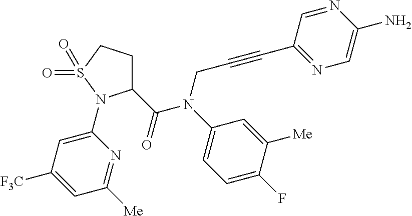 [SMILES: Cc1cc(C(F)(F)F)cc(N2C(C(=O)N(CC#Cc3cnc(N)cn3)c3ccc(F)c(C)c3)CCS2(=O)=O)n1]

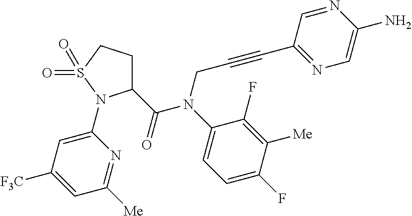 [SMILES: Cc1cc(C(F)(F)F)cc(N2C(C(=O)N(CC#Cc3cnc(N)cn3)c3ccc(F)c(C)c3F)CCS2(=O)=O)n1]

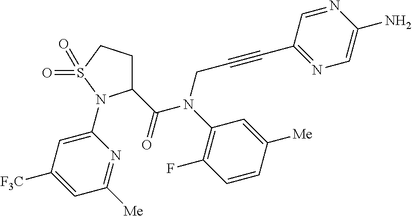 [SMILES: Cc1ccc(F)c(N(CC#Cc2cnc(N)cn2)C(=O)C2CCS(=O)(=O)N2c2cc(C(F)(F)F)cc(C)n2)c1]

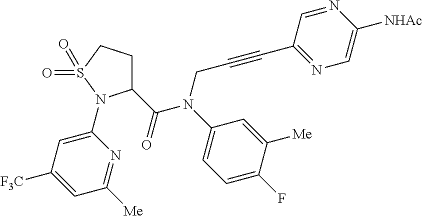 [SMILES: CC(=O)Nc1cnc(C#CCN(C(=O)C2CCS(=O)(=O)N2c2cc(C(F)(F)F)cc(C)n2)c2ccc(F)c(C)c2)cn1]

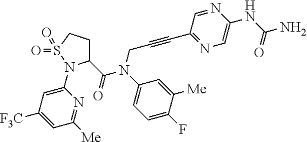 [SMILES: Cc1cc(C(F)(F)F)cc(N2C(C(=O)N(CC#Cc3cnc(NC(N)=O)cn3)c3ccc(F)c(C)c3)CCS2(=O)=O)n1]

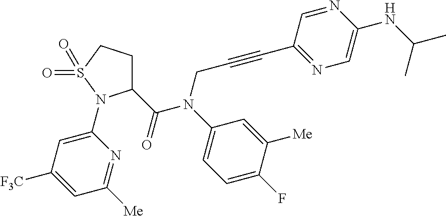 [SMILES: Cc1cc(C(F)(F)F)cc(N2C(C(=O)N(CC#Cc3cnc(NC(C)C)cn3)c3ccc(F)c(C)c3)CCS2(=O)=O)n1]

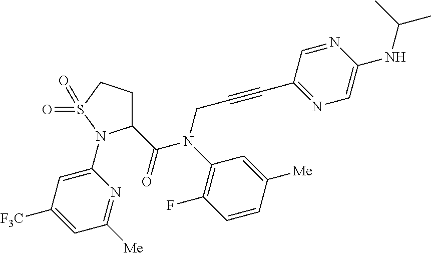 [SMILES: Cc1ccc(F)c(N(CC#Cc2cnc(NC(C)C)cn2)C(=O)C2CCS(=O)(=O)N2c2cc(C(F)(F)F)cc(C)n2)c1]

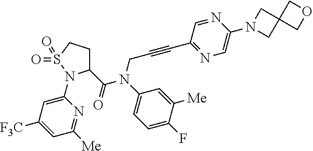 [SMILES: Cc1cc(C(F)(F)F)cc(N2C(C(=O)N(CC#Cc3cnc(N4CC5(COC5)C4)cn3)c3ccc(F)c(C)c3)CCS2(=O)=O)n1]

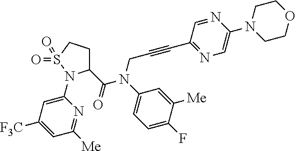 [SMILES: Cc1cc(C(F)(F)F)cc(N2C(C(=O)N(CC#Cc3cnc(N4CCOCC4)cn3)c3ccc(F)c(C)c3)CCS2(=O)=O)n1]

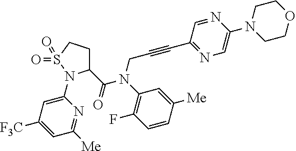 [SMILES: Cc1ccc(F)c(N(CC#Cc2cnc(N3CCOCC3)cn2)C(=O)C2CCS(=O)(=O)N2c2cc(C(F)(F)F)cc(C)n2)c1]

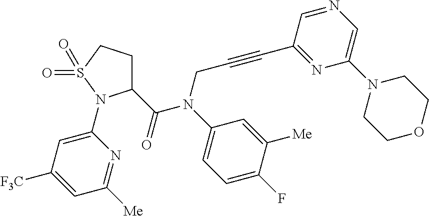 [SMILES: Cc1cc(C(F)(F)F)cc(N2C(C(=O)N(CC#Cc3cncc(N4CCOCC4)n3)c3ccc(F)c(C)c3)CCS2(=O)=O)n1]

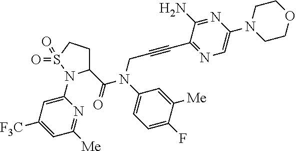 [SMILES: Cc1cc(C(F)(F)F)cc(N2C(C(=O)N(CC#Cc3ncc(N4CCOCC4)nc3N)c3ccc(F)c(C)c3)CCS2(=O)=O)n1]

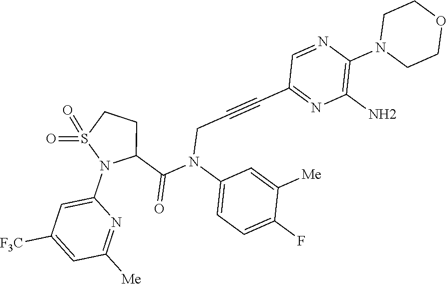 [SMILES: Cc1cc(C(F)(F)F)cc(N2C(C(=O)N(CC#Cc3cnc(N4CCOCC4)c(N)n3)c3ccc(F)c(C)c3)CCS2(=O)=O)n1]

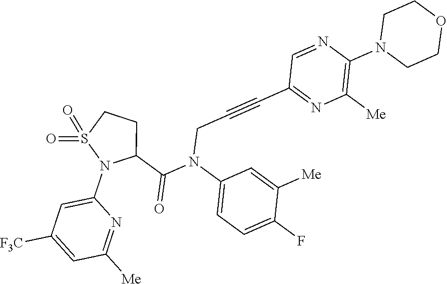 [SMILES: Cc1cc(C(F)(F)F)cc(N2C(C(=O)N(CC#Cc3cnc(N4CCOCC4)c(C)n3)c3ccc(F)c(C)c3)CCS2(=O)=O)n1]

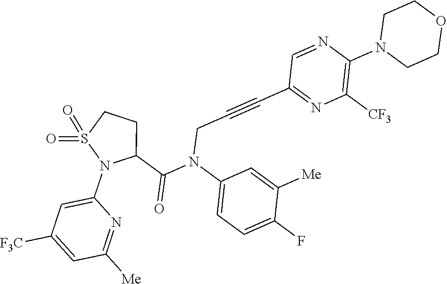 [SMILES: Cc1cc(C(F)(F)F)cc(N2C(C(=O)N(CC#Cc3cnc(N4CCOCC4)c(C(F)(F)F)n3)c3ccc(F)c(C)c3)CCS2(=O)=O)n1]

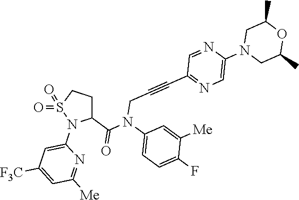 [SMILES: Cc1cc(C(F)(F)F)cc(N2C(C(=O)N(CC#Cc3cnc(N4C[C@@H](C)O[C@@H](C)C4)cn3)c3ccc(F)c(C)c3)CCS2(=O)=O)n1]

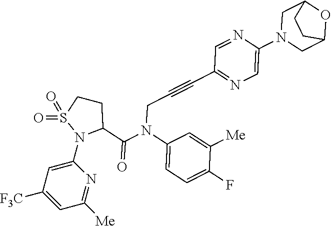 [SMILES: Cc1cc(C(F)(F)F)cc(N2C(C(=O)N(CC#Cc3cnc(N4CC5CCC(C4)O5)cn3)c3ccc(F)c(C)c3)CCS2(=O)=O)n1]

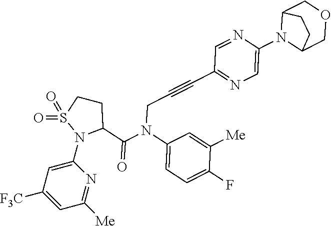 [SMILES: Cc1cc(C(F)(F)F)cc(N2C(C(=O)N(CC#Cc3cnc(N4C5CCC4COC5)cn3)c3ccc(F)c(C)c3)CCS2(=O)=O)n1]

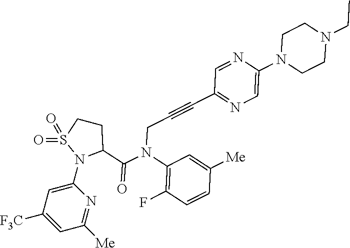 [SMILES: CCN1CCN(c2cnc(C#CCN(C(=O)C3CCS(=O)(=O)N3c3cc(C(F)(F)F)cc(C)n3)c3cc(C)ccc3F)cn2)CC1]

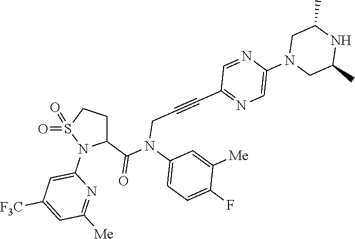 [SMILES: Cc1cc(C(F)(F)F)cc(N2C(C(=O)N(CC#Cc3cnc(N4C[C@H](C)N[C@@H](C)C4)cn3)c3ccc(F)c(C)c3)CCS2(=O)=O)n1]

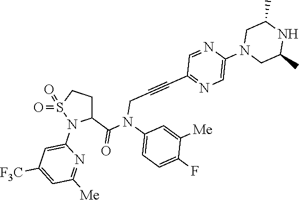 [SMILES: Cc1cc(C(F)(F)F)cc(N2C(C(=O)N(CC#Cc3cnc(N4C[C@H](C)N[C@@H](C)C4)cn3)c3ccc(F)c(C)c3)CCS2(=O)=O)n1]

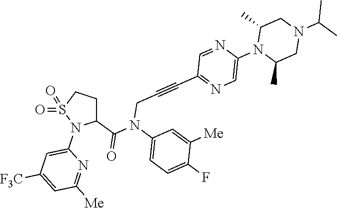 [SMILES: Cc1cc(C(F)(F)F)cc(N2C(C(=O)N(CC#Cc3cnc(N4[C@H](C)CN(C(C)C)C[C@H]4C)cn3)c3ccc(F)c(C)c3)CCS2(=O)=O)n1]

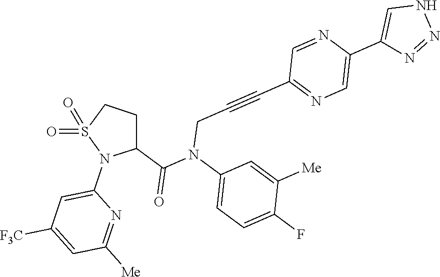 [SMILES: Cc1cc(C(F)(F)F)cc(N2C(C(=O)N(CC#Cc3cnc(-c4c[nH]nn4)cn3)c3ccc(F)c(C)c3)CCS2(=O)=O)n1]

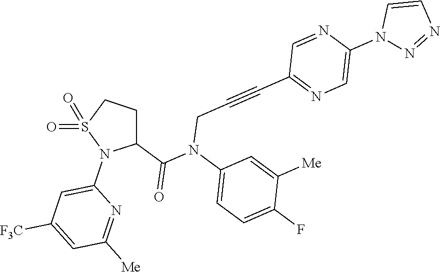 [SMILES: Cc1cc(C(F)(F)F)cc(N2C(C(=O)N(CC#Cc3cnc(-n4ccnn4)cn3)c3ccc(F)c(C)c3)CCS2(=O)=O)n1]

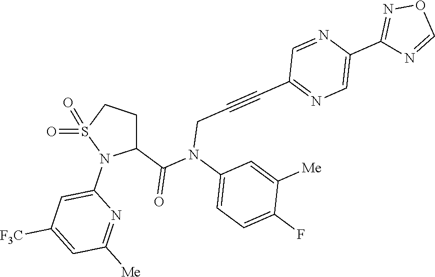 [SMILES: Cc1cc(C(F)(F)F)cc(N2C(C(=O)N(CC#Cc3cnc(-c4ncon4)cn3)c3ccc(F)c(C)c3)CCS2(=O)=O)n1]

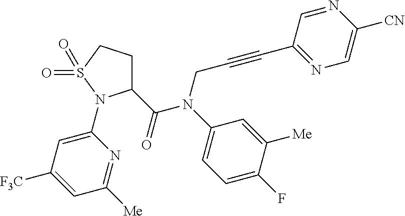 [SMILES: Cc1cc(C(F)(F)F)cc(N2C(C(=O)N(CC#Cc3cnc(C#N)cn3)c3ccc(F)c(C)c3)CCS2(=O)=O)n1]

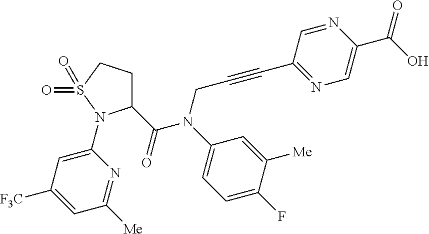 [SMILES: Cc1cc(C(F)(F)F)cc(N2C(C(=O)N(CC#Cc3cnc(C(=O)O)cn3)c3ccc(F)c(C)c3)CCS2(=O)=O)n1]

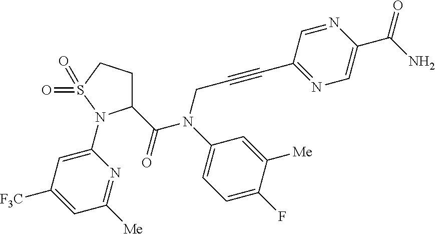 [SMILES: Cc1cc(C(F)(F)F)cc(N2C(C(=O)N(CC#Cc3cnc(C(N)=O)cn3)c3ccc(F)c(C)c3)CCS2(=O)=O)n1]

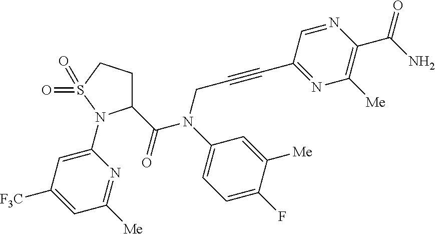 [SMILES: Cc1cc(C(F)(F)F)cc(N2C(C(=O)N(CC#Cc3cnc(C(N)=O)c(C)n3)c3ccc(F)c(C)c3)CCS2(=O)=O)n1]

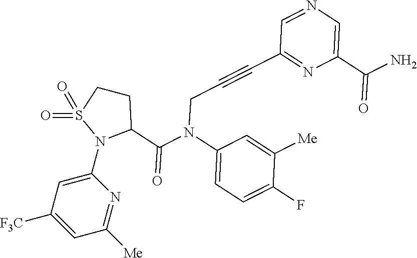 [SMILES: Cc1cc(C(F)(F)F)cc(N2C(C(=O)N(CC#Cc3cncc(C(N)=O)n3)c3ccc(F)c(C)c3)CCS2(=O)=O)n1]

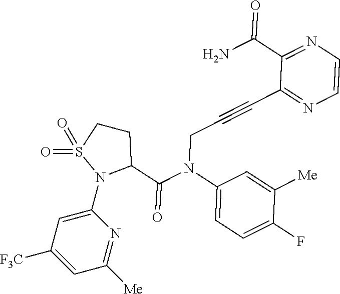 [SMILES: Cc1cc(C(F)(F)F)cc(N2C(C(=O)N(CC#Cc3nccnc3C(N)=O)c3ccc(F)c(C)c3)CCS2(=O)=O)n1]

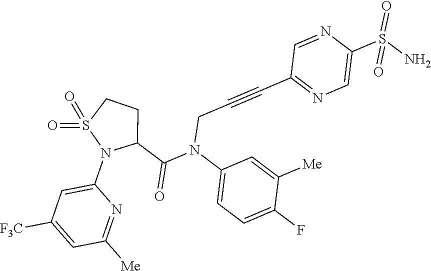 [SMILES: Cc1cc(C(F)(F)F)cc(N2C(C(=O)N(CC#Cc3cnc(S(N)(=O)=O)cn3)c3ccc(F)c(C)c3)CCS2(=O)=O)n1]

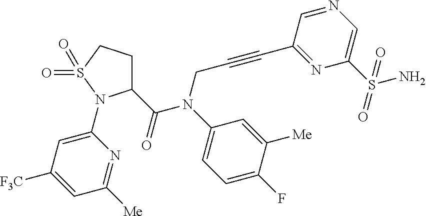 [SMILES: Cc1cc(C(F)(F)F)cc(N2C(C(=O)N(CC#Cc3cncc(S(N)(=O)=O)n3)c3ccc(F)c(C)c3)CCS2(=O)=O)n1]

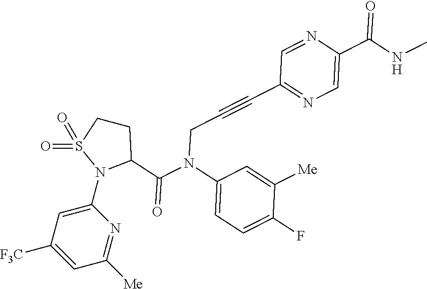 [SMILES: CNC(=O)c1cnc(C#CCN(C(=O)C2CCS(=O)(=O)N2c2cc(C(F)(F)F)cc(C)n2)c2ccc(F)c(C)c2)cn1]

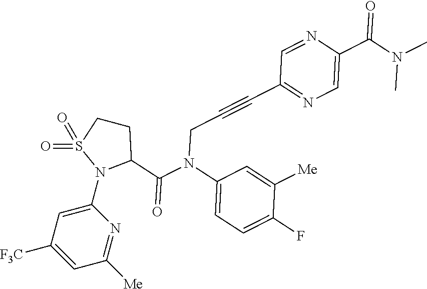 [SMILES: Cc1cc(C(F)(F)F)cc(N2C(C(=O)N(CC#Cc3cnc(C(=O)N(C)C)cn3)c3ccc(F)c(C)c3)CCS2(=O)=O)n1]

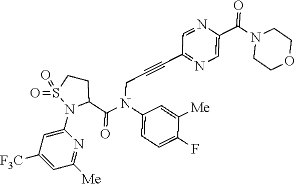 [SMILES: Cc1cc(C(F)(F)F)cc(N2C(C(=O)N(CC#Cc3cnc(C(=O)N4CCOCC4)cn3)c3ccc(F)c(C)c3)CCS2(=O)=O)n1]

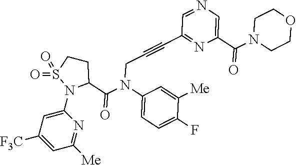 [SMILES: Cc1cc(C(F)(F)F)cc(N2C(C(=O)N(CC#Cc3cncc(C(=O)N4CCOCC4)n3)c3ccc(F)c(C)c3)CCS2(=O)=O)n1]

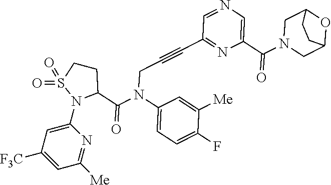 [SMILES: Cc1cc(C(F)(F)F)cc(N2C(C(=O)N(CC#Cc3cncc(C(=O)N4CC5CCC(C4)O5)n3)c3ccc(F)c(C)c3)CCS2(=O)=O)n1]

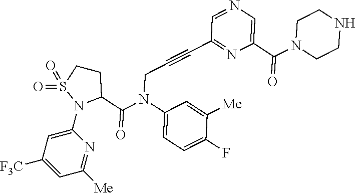 [SMILES: Cc1cc(C(F)(F)F)cc(N2C(C(=O)N(CC#Cc3cncc(C(=O)N4CCNCC4)n3)c3ccc(F)c(C)c3)CCS2(=O)=O)n1]

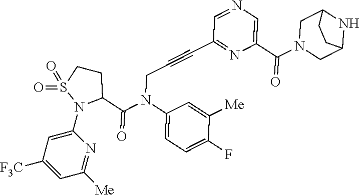 [SMILES: Cc1cc(C(F)(F)F)cc(N2C(C(=O)N(CC#Cc3cncc(C(=O)N4CC5CCC(C4)N5)n3)c3ccc(F)c(C)c3)CCS2(=O)=O)n1]

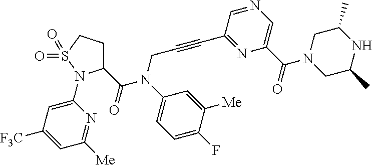 [SMILES: Cc1cc(C(F)(F)F)cc(N2C(C(=O)N(CC#Cc3cncc(C(=O)N4C[C@H](C)N[C@@H](C)C4)n3)c3ccc(F)c(C)c3)CCS2(=O)=O)n1]

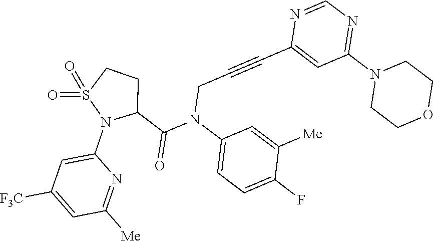 [SMILES: Cc1cc(C(F)(F)F)cc(N2C(C(=O)N(CC#Cc3cc(N4CCOCC4)ncn3)c3ccc(F)c(C)c3)CCS2(=O)=O)n1]

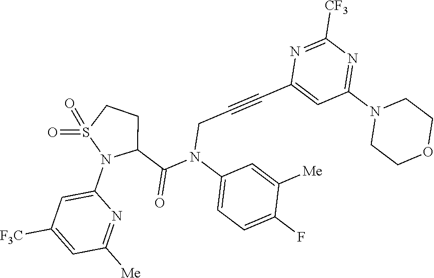 [SMILES: Cc1cc(C(F)(F)F)cc(N2C(C(=O)N(CC#Cc3cc(N4CCOCC4)nc(C(F)(F)F)n3)c3ccc(F)c(C)c3)CCS2(=O)=O)n1]

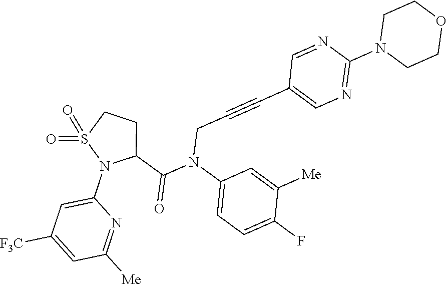 [SMILES: Cc1cc(C(F)(F)F)cc(N2C(C(=O)N(CC#Cc3cnc(N4CCOCC4)nc3)c3ccc(F)c(C)c3)CCS2(=O)=O)n1]

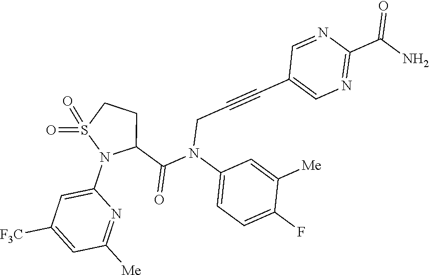 [SMILES: Cc1cc(C(F)(F)F)cc(N2C(C(=O)N(CC#Cc3cnc(C(N)=O)nc3)c3ccc(F)c(C)c3)CCS2(=O)=O)n1]

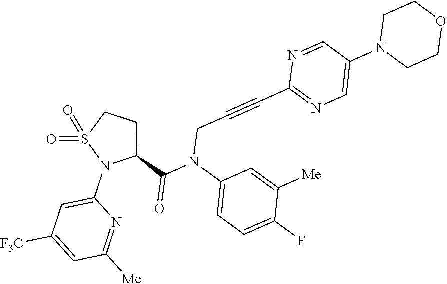 [SMILES: Cc1cc(C(F)(F)F)cc(N2[C@H](C(=O)N(CC#Cc3ncc(N4CCOCC4)cn3)c3ccc(F)c(C)c3)CCS2(=O)=O)n1]

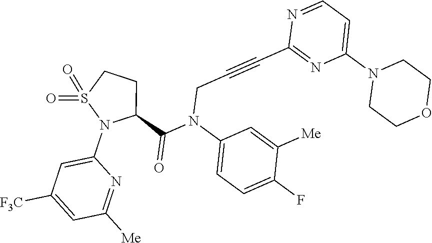 [SMILES: Cc1cc(C(F)(F)F)cc(N2[C@H](C(=O)N(CC#Cc3nccc(N4CCOCC4)n3)c3ccc(F)c(C)c3)CCS2(=O)=O)n1]

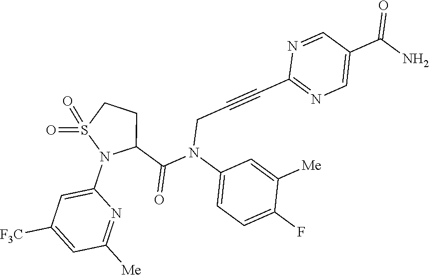 [SMILES: Cc1cc(C(F)(F)F)cc(N2C(C(=O)N(CC#Cc3ncc(C(N)=O)cn3)c3ccc(F)c(C)c3)CCS2(=O)=O)n1]

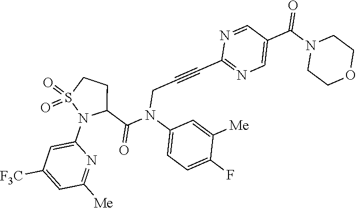 [SMILES: Cc1cc(C(F)(F)F)cc(N2C(C(=O)N(CC#Cc3ncc(C(=O)N4CCOCC4)cn3)c3ccc(F)c(C)c3)CCS2(=O)=O)n1]

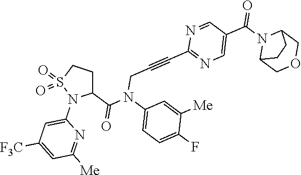 [SMILES: Cc1cc(C(F)(F)F)cc(N2C(C(=O)N(CC#Cc3ncc(C(=O)N4C5CCC4COC5)cn3)c3ccc(F)c(C)c3)CCS2(=O)=O)n1]

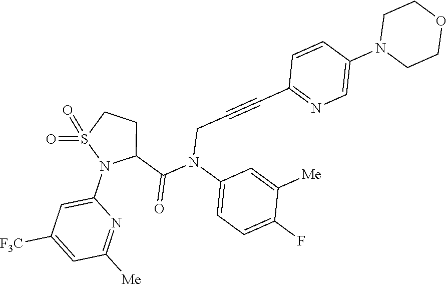 [SMILES: Cc1cc(C(F)(F)F)cc(N2C(C(=O)N(CC#Cc3ccc(N4CCOCC4)cn3)c3ccc(F)c(C)c3)CCS2(=O)=O)n1]

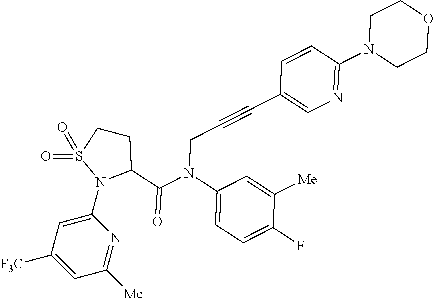 [SMILES: Cc1cc(C(F)(F)F)cc(N2C(C(=O)N(CC#Cc3ccc(N4CCOCC4)nc3)c3ccc(F)c(C)c3)CCS2(=O)=O)n1]

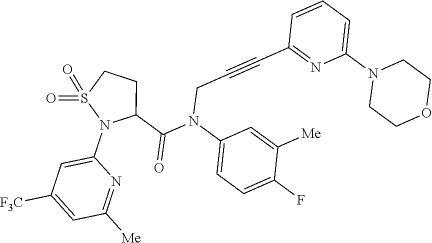 [SMILES: Cc1cc(C(F)(F)F)cc(N2C(C(=O)N(CC#Cc3cccc(N4CCOCC4)n3)c3ccc(F)c(C)c3)CCS2(=O)=O)n1]

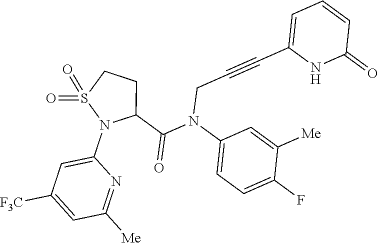 [SMILES: Cc1cc(C(F)(F)F)cc(N2C(C(=O)N(CC#Cc3cccc(=O)[nH]3)c3ccc(F)c(C)c3)CCS2(=O)=O)n1]

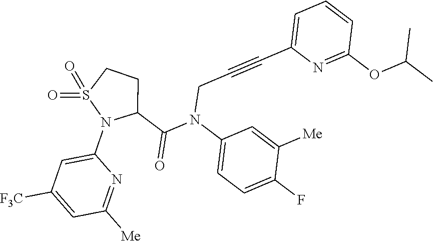 [SMILES: Cc1cc(C(F)(F)F)cc(N2C(C(=O)N(CC#Cc3cccc(OC(C)C)n3)c3ccc(F)c(C)c3)CCS2(=O)=O)n1]

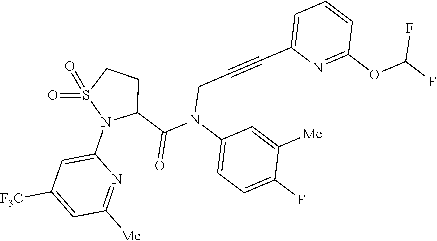 [SMILES: Cc1cc(C(F)(F)F)cc(N2C(C(=O)N(CC#Cc3cccc(OC(F)F)n3)c3ccc(F)c(C)c3)CCS2(=O)=O)n1]

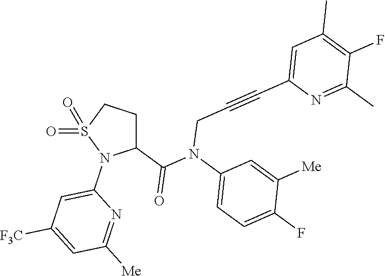 [SMILES: Cc1cc(C(F)(F)F)cc(N2C(C(=O)N(CC#Cc3cc(C)c(F)c(C)n3)c3ccc(F)c(C)c3)CCS2(=O)=O)n1]

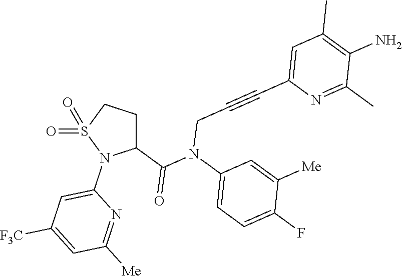 [SMILES: Cc1cc(C(F)(F)F)cc(N2C(C(=O)N(CC#Cc3cc(C)c(N)c(C)n3)c3ccc(F)c(C)c3)CCS2(=O)=O)n1]

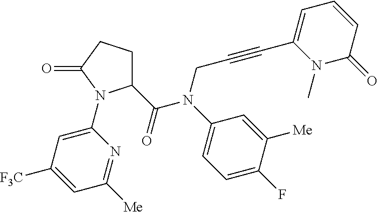 [SMILES: Cc1cc(C(F)(F)F)cc(N2C(=O)CCC2C(=O)N(CC#Cc2cccc(=O)n2C)c2ccc(F)c(C)c2)n1]

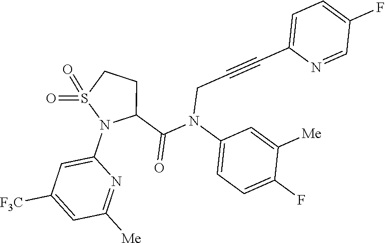 [SMILES: Cc1cc(C(F)(F)F)cc(N2C(C(=O)N(CC#Cc3ccc(F)cn3)c3ccc(F)c(C)c3)CCS2(=O)=O)n1]

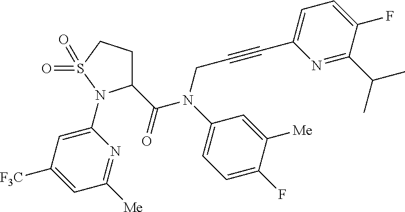 [SMILES: Cc1cc(C(F)(F)F)cc(N2C(C(=O)N(CC#Cc3ccc(F)c(C(C)C)n3)c3ccc(F)c(C)c3)CCS2(=O)=O)n1]

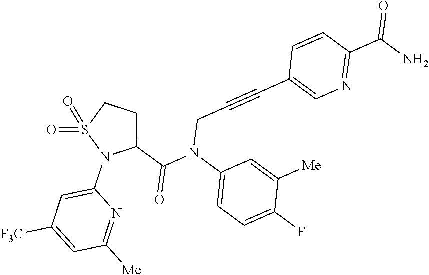 [SMILES: Cc1cc(C(F)(F)F)cc(N2C(C(=O)N(CC#Cc3ccc(C(N)=O)nc3)c3ccc(F)c(C)c3)CCS2(=O)=O)n1]

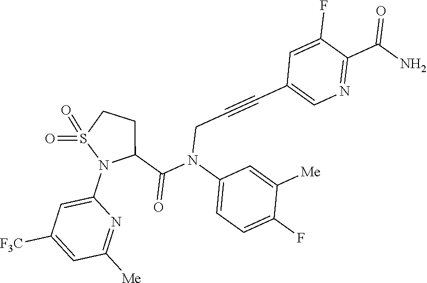 [SMILES: Cc1cc(C(F)(F)F)cc(N2C(C(=O)N(CC#Cc3cnc(C(N)=O)c(F)c3)c3ccc(F)c(C)c3)CCS2(=O)=O)n1]

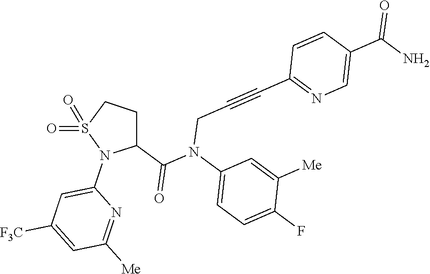 [SMILES: Cc1cc(C(F)(F)F)cc(N2C(C(=O)N(CC#Cc3ccc(C(N)=O)cn3)c3ccc(F)c(C)c3)CCS2(=O)=O)n1]

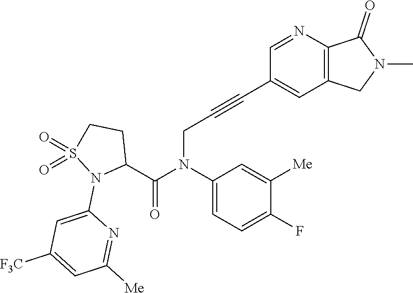 [SMILES: Cc1cc(C(F)(F)F)cc(N2C(C(=O)N(CC#Cc3cnc4c(c3)CN(C)C4=O)c3ccc(F)c(C)c3)CCS2(=O)=O)n1]

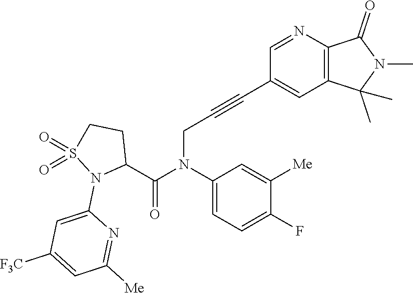 [SMILES: Cc1cc(C(F)(F)F)cc(N2C(C(=O)N(CC#Cc3cnc4c(c3)C(C)(C)N(C)C4=O)c3ccc(F)c(C)c3)CCS2(=O)=O)n1]

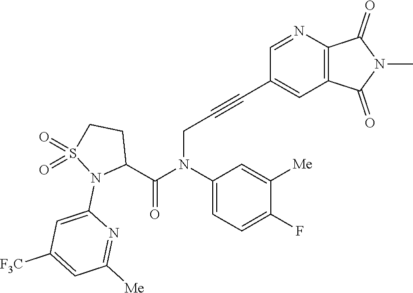 [SMILES: Cc1cc(C(F)(F)F)cc(N2C(C(=O)N(CC#Cc3cnc4c(c3)C(=O)N(C)C4=O)c3ccc(F)c(C)c3)CCS2(=O)=O)n1]

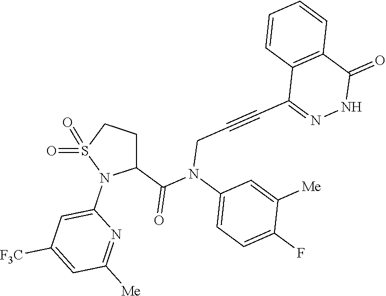 [SMILES: Cc1cc(C(F)(F)F)cc(N2C(C(=O)N(CC#Cc3n[nH]c(=O)c4ccccc34)c3ccc(F)c(C)c3)CCS2(=O)=O)n1]

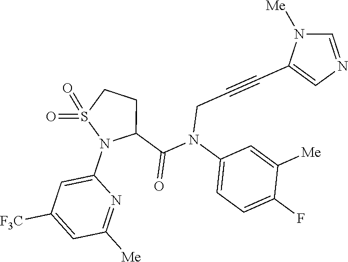 [SMILES: Cc1cc(C(F)(F)F)cc(N2C(C(=O)N(CC#Cc3cncn3C)c3ccc(F)c(C)c3)CCS2(=O)=O)n1]

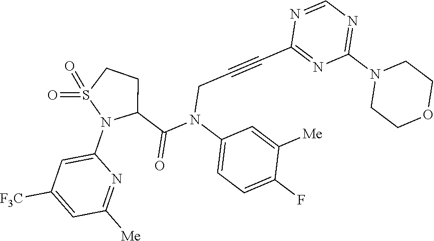 [SMILES: Cc1cc(C(F)(F)F)cc(N2C(C(=O)N(CC#Cc3ncnc(N4CCOCC4)n3)c3ccc(F)c(C)c3)CCS2(=O)=O)n1]

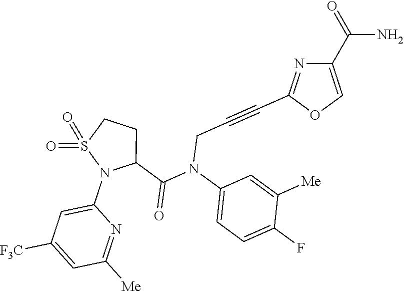 [SMILES: Cc1cc(C(F)(F)F)cc(N2C(C(=O)N(CC#Cc3nc(C(N)=O)co3)c3ccc(F)c(C)c3)CCS2(=O)=O)n1]

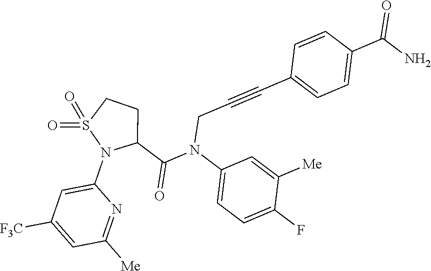 [SMILES: Cc1cc(C(F)(F)F)cc(N2C(C(=O)N(CC#Cc3ccc(C(N)=O)cc3)c3ccc(F)c(C)c3)CCS2(=O)=O)n1]

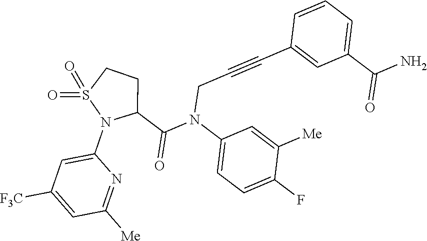 [SMILES: Cc1cc(C(F)(F)F)cc(N2C(C(=O)N(CC#Cc3cccc(C(N)=O)c3)c3ccc(F)c(C)c3)CCS2(=O)=O)n1]

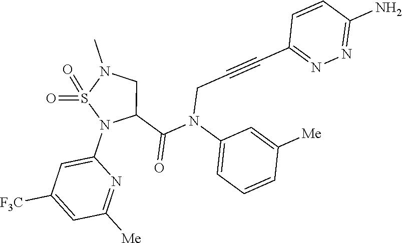 [SMILES: Cc1cccc(N(CC#Cc2ccc(N)nn2)C(=O)C2CN(C)S(=O)(=O)N2c2cc(C(F)(F)F)cc(C)n2)c1]

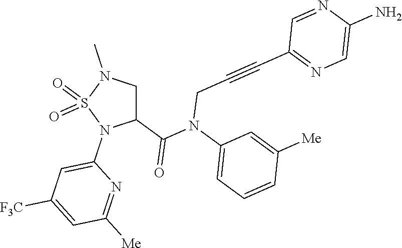 [SMILES: Cc1cccc(N(CC#Cc2cnc(N)cn2)C(=O)C2CN(C)S(=O)(=O)N2c2cc(C(F)(F)F)cc(C)n2)c1]

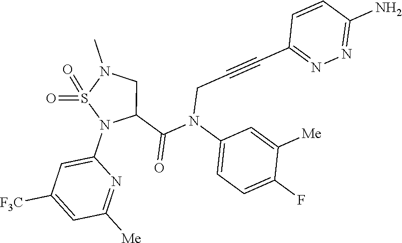 [SMILES: Cc1cc(C(F)(F)F)cc(N2C(C(=O)N(CC#Cc3ccc(N)nn3)c3ccc(F)c(C)c3)CN(C)S2(=O)=O)n1]

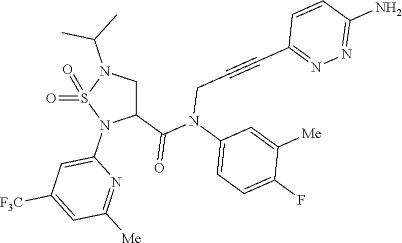 [SMILES: Cc1cc(C(F)(F)F)cc(N2C(C(=O)N(CC#Cc3ccc(N)nn3)c3ccc(F)c(C)c3)CN(C(C)C)S2(=O)=O)n1]

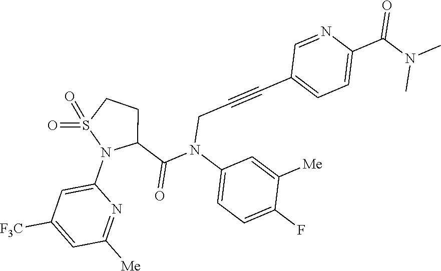 [SMILES: Cc1cc(C(F)(F)F)cc(N2C(C(=O)N(CC#Cc3ccc(C(=O)N(C)C)nc3)c3ccc(F)c(C)c3)CCS2(=O)=O)n1]

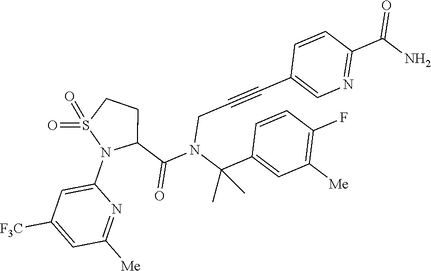 [SMILES: Cc1cc(C(F)(F)F)cc(N2C(C(=O)N(CC#Cc3ccc(C(N)=O)nc3)C(C)(C)c3ccc(F)c(C)c3)CCS2(=O)=O)n1]

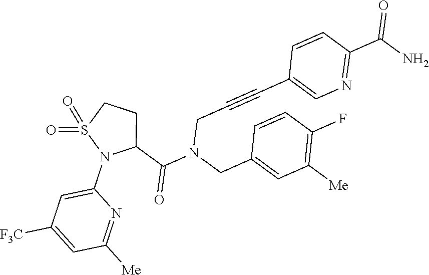 [SMILES: Cc1cc(C(F)(F)F)cc(N2C(C(=O)N(CC#Cc3ccc(C(N)=O)nc3)Cc3ccc(F)c(C)c3)CCS2(=O)=O)n1]

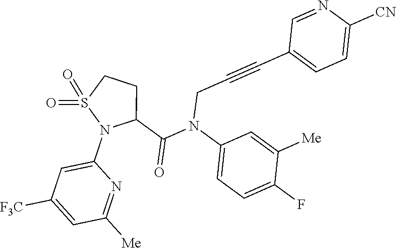 [SMILES: Cc1cc(C(F)(F)F)cc(N2C(C(=O)N(CC#Cc3ccc(C#N)nc3)c3ccc(F)c(C)c3)CCS2(=O)=O)n1]

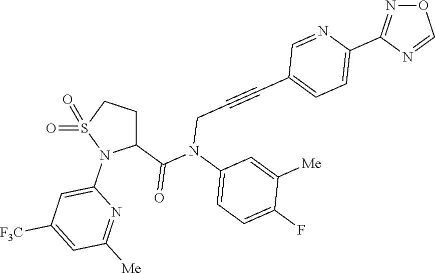 [SMILES: Cc1cc(C(F)(F)F)cc(N2C(C(=O)N(CC#Cc3ccc(-c4ncon4)nc3)c3ccc(F)c(C)c3)CCS2(=O)=O)n1]

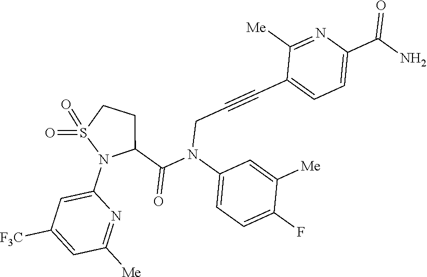 [SMILES: Cc1cc(C(F)(F)F)cc(N2C(C(=O)N(CC#Cc3ccc(C(N)=O)nc3C)c3ccc(F)c(C)c3)CCS2(=O)=O)n1]

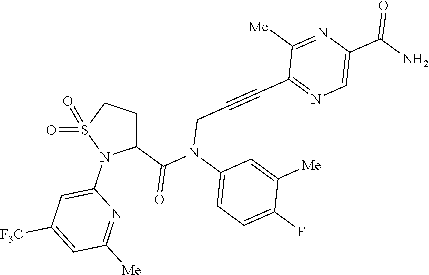 [SMILES: Cc1cc(C(F)(F)F)cc(N2C(C(=O)N(CC#Cc3ncc(C(N)=O)nc3C)c3ccc(F)c(C)c3)CCS2(=O)=O)n1]

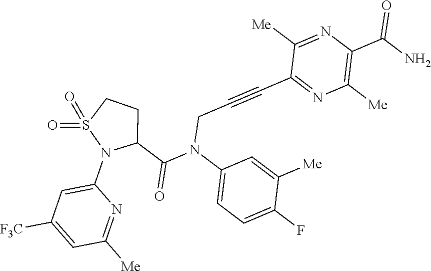 [SMILES: Cc1cc(C(F)(F)F)cc(N2C(C(=O)N(CC#Cc3nc(C)c(C(N)=O)nc3C)c3ccc(F)c(C)c3)CCS2(=O)=O)n1]

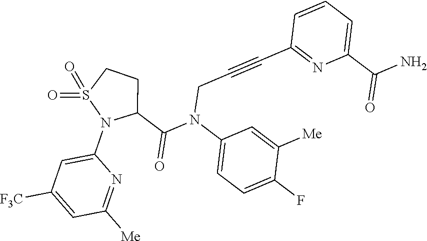 [SMILES: Cc1cc(C(F)(F)F)cc(N2C(C(=O)N(CC#Cc3cccc(C(N)=O)n3)c3ccc(F)c(C)c3)CCS2(=O)=O)n1]

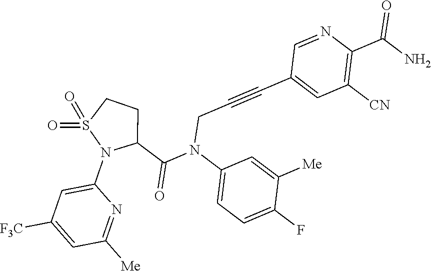 [SMILES: Cc1cc(C(F)(F)F)cc(N2C(C(=O)N(CC#Cc3cnc(C(N)=O)c(C#N)c3)c3ccc(F)c(C)c3)CCS2(=O)=O)n1]

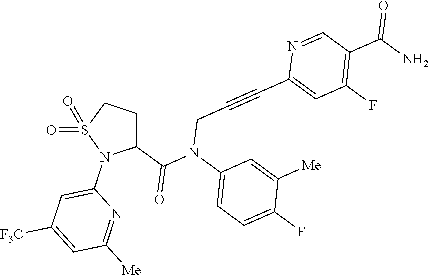 [SMILES: Cc1cc(C(F)(F)F)cc(N2C(C(=O)N(CC#Cc3cc(F)c(C(N)=O)cn3)c3ccc(F)c(C)c3)CCS2(=O)=O)n1]

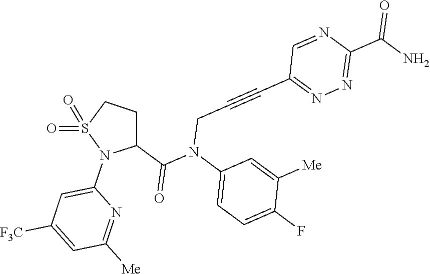 [SMILES: Cc1cc(C(F)(F)F)cc(N2C(C(=O)N(CC#Cc3cnc(C(N)=O)nn3)c3ccc(F)c(C)c3)CCS2(=O)=O)n1]

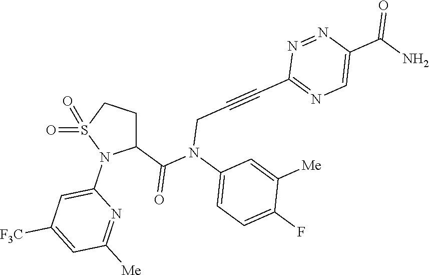 [SMILES: Cc1cc(C(F)(F)F)cc(N2C(C(=O)N(CC#Cc3ncc(C(N)=O)nn3)c3ccc(F)c(C)c3)CCS2(=O)=O)n1]

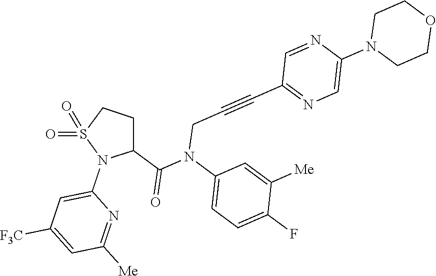 [SMILES: Cc1cc(C(F)(F)F)cc(N2C(C(=O)N(CC#Cc3cnc(N4CCOCC4)cn3)c3ccc(F)c(C)c3)CCS2(=O)=O)n1]

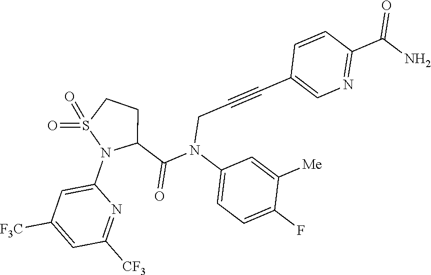 [SMILES: Cc1cc(N(CC#Cc2ccc(C(N)=O)nc2)C(=O)C2CCS(=O)(=O)N2c2cc(C(F)(F)F)cc(C(F)(F)F)n2)ccc1F]

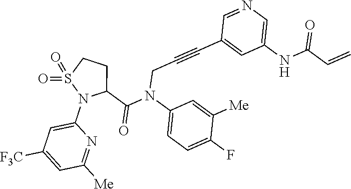 [SMILES: C=CC(=O)Nc1cncc(C#CCN(C(=O)C2CCS(=O)(=O)N2c2cc(C(F)(F)F)cc(C)n2)c2ccc(F)c(C)c2)c1]

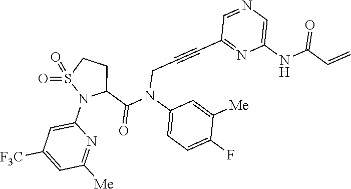 [SMILES: C=CC(=O)Nc1cncc(C#CCN(C(=O)C2CCS(=O)(=O)N2c2cc(C(F)(F)F)cc(C)n2)c2ccc(F)c(C)c2)n1]

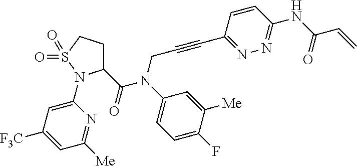 [SMILES: C=CC(=O)Nc1ccc(C#CCN(C(=O)C2CCS(=O)(=O)N2c2cc(C(F)(F)F)cc(C)n2)c2ccc(F)c(C)c2)nn1]

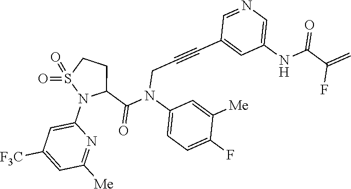 [SMILES: C=C(F)C(=O)Nc1cncc(C#CCN(C(=O)C2CCS(=O)(=O)N2c2cc(C(F)(F)F)cc(C)n2)c2ccc(F)c(C)c2)c1]

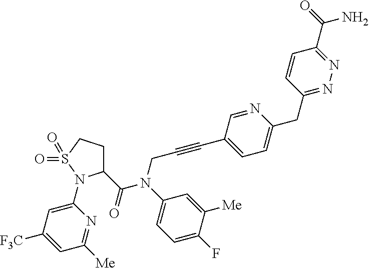 [SMILES: Cc1cc(C(F)(F)F)cc(N2C(C(=O)N(CC#Cc3ccc(Cc4ccc(C(N)=O)nn4)nc3)c3ccc(F)c(C)c3)CCS2(=O)=O)n1]

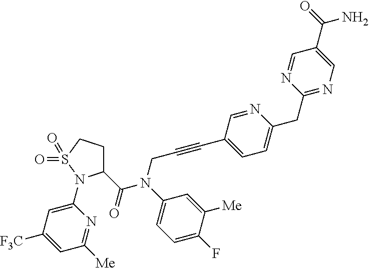 [SMILES: Cc1cc(C(F)(F)F)cc(N2C(C(=O)N(CC#Cc3ccc(Cc4ncc(C(N)=O)cn4)nc3)c3ccc(F)c(C)c3)CCS2(=O)=O)n1]

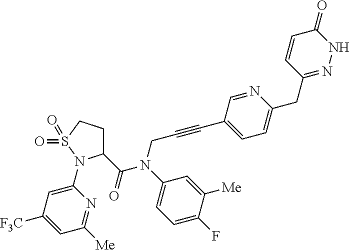 [SMILES: Cc1cc(C(F)(F)F)cc(N2C(C(=O)N(CC#Cc3ccc(Cc4ccc(=O)[nH]n4)nc3)c3ccc(F)c(C)c3)CCS2(=O)=O)n1]

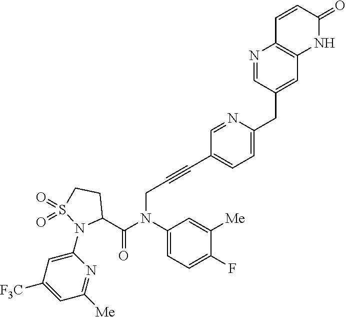 [SMILES: Cc1cc(C(F)(F)F)cc(N2C(C(=O)N(CC#Cc3ccc(Cc4cnc5ccc(=O)[nH]c5c4)nc3)c3ccc(F)c(C)c3)CCS2(=O)=O)n1]

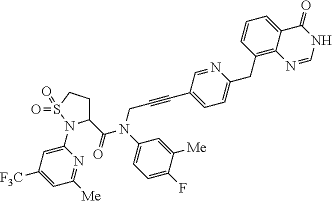 [SMILES: Cc1cc(C(F)(F)F)cc(N2C(C(=O)N(CC#Cc3ccc(Cc4cccc5c(=O)[nH]cnc45)nc3)c3ccc(F)c(C)c3)CCS2(=O)=O)n1]

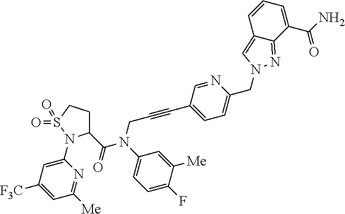 [SMILES: Cc1cc(C(F)(F)F)cc(N2C(C(=O)N(CC#Cc3ccc(Cn4cc5cccc(C(N)=O)c5n4)nc3)c3ccc(F)c(C)c3)CCS2(=O)=O)n1]

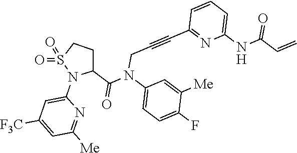 [SMILES: C=CC(=O)Nc1cccc(C#CCN(C(=O)C2CCS(=O)(=O)N2c2cc(C(F)(F)F)cc(C)n2)c2ccc(F)c(C)c2)n1]

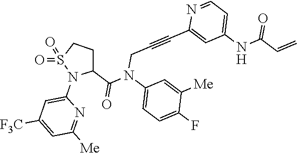 [SMILES: C=CC(=O)Nc1ccnc(C#CCN(C(=O)C2CCS(=O)(=O)N2c2cc(C(F)(F)F)cc(C)n2)c2ccc(F)c(C)c2)c1]

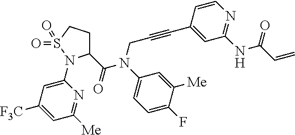 [SMILES: C=CC(=O)Nc1cc(C#CCN(C(=O)C2CCS(=O)(=O)N2c2cc(C(F)(F)F)cc(C)n2)c2ccc(F)c(C)c2)ccn1]

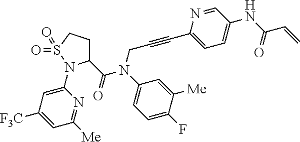 [SMILES: C=CC(=O)Nc1ccc(C#CCN(C(=O)C2CCS(=O)(=O)N2c2cc(C(F)(F)F)cc(C)n2)c2ccc(F)c(C)c2)nc1]

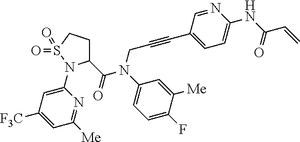 [SMILES: C=CC(=O)Nc1ccc(C#CCN(C(=O)C2CCS(=O)(=O)N2c2cc(C(F)(F)F)cc(C)n2)c2ccc(F)c(C)c2)cn1]

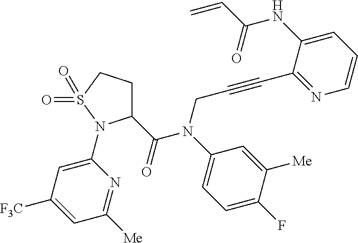 [SMILES: C=CC(=O)Nc1cccnc1C#CCN(C(=O)C1CCS(=O)(=O)N1c1cc(C(F)(F)F)cc(C)n1)c1ccc(F)c(C)c1]

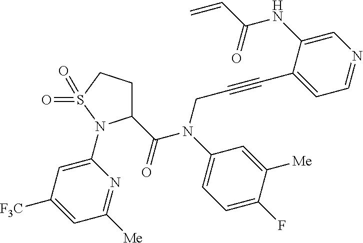 [SMILES: C=CC(=O)Nc1cnccc1C#CCN(C(=O)C1CCS(=O)(=O)N1c1cc(C(F)(F)F)cc(C)n1)c1ccc(F)c(C)c1]

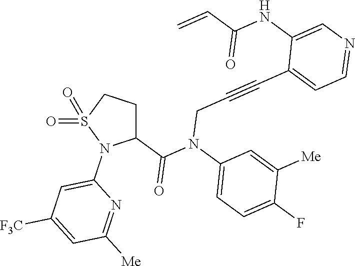 [SMILES: C=CC(=O)Nc1cnccc1C#CCN(C(=O)C1CCS(=O)(=O)N1c1cc(C(F)(F)F)cc(C)n1)c1ccc(F)c(C)c1]

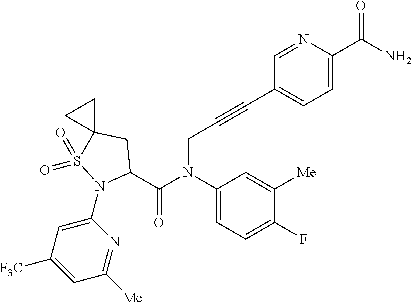 [SMILES: Cc1cc(C(F)(F)F)cc(N2C(C(=O)N(CC#Cc3ccc(C(N)=O)nc3)c3ccc(F)c(C)c3)CC3(CC3)S2(=O)=O)n1]

In another embodiment, exemplary compounds of Formula (I) are provided below:

| NO. | Structure |
|---|---|
| 1 | 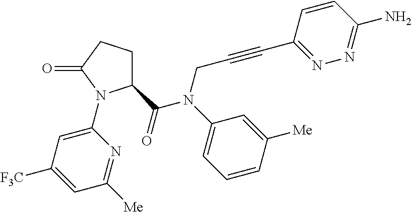 [SMILES: Cc1cccc(N(CC#Cc2ccc(N)nn2)C(=O)[C@@H]2CCC(=O)N2c2cc(C(F)(F)F)cc(C)n2)c1] |
| 2 | 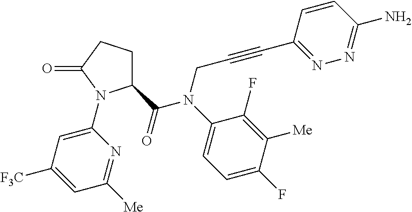 [SMILES: Cc1cc(C(F)(F)F)cc(N2C(=O)CC[C@H]2C(=O)N(CC#Cc2ccc(N)nn2)c2ccc(F)c(C)c2F)n1] |
| 3 | 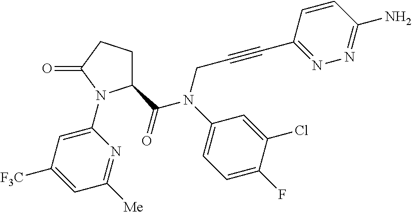 [SMILES: Cc1cc(C(F)(F)F)cc(N2C(=O)CC[C@H]2C(=O)N(CC#Cc2ccc(N)nn2)c2ccc(F)c(Cl)c2)n1] |
| 4 | 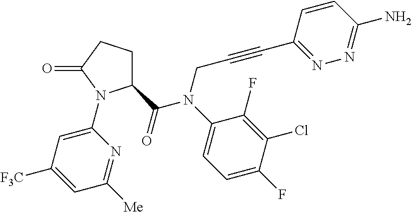 [SMILES: Cc1cc(C(F)(F)F)cc(N2C(=O)CC[C@H]2C(=O)N(CC#Cc2ccc(N)nn2)c2ccc(F)c(Cl)c2F)n1] |
| 5 | 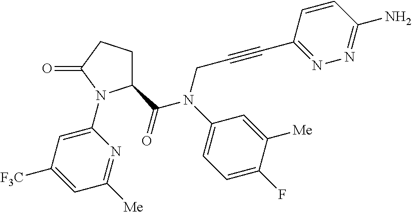 [SMILES: Cc1cc(C(F)(F)F)cc(N2C(=O)CC[C@H]2C(=O)N(CC#Cc2ccc(N)nn2)c2ccc(F)c(C)c2)n1] |
| 6 | 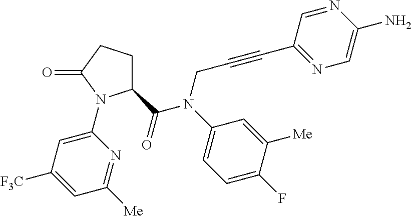 [SMILES: Cc1cc(C(F)(F)F)cc(N2C(=O)CC[C@H]2C(=O)N(CC#Cc2cnc(N)cn2)c2ccc(F)c(C)c2)n1] |
| 7 | 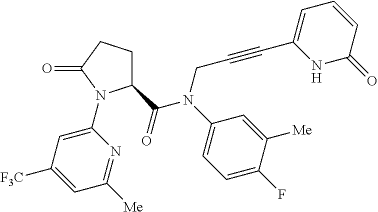 [SMILES: Cc1cc(C(F)(F)F)cc(N2C(=O)CC[C@H]2C(=O)N(CC#Cc2cccc(=O)[nH]2)c2ccc(F)c(C)c2)n1] |
| 8 | 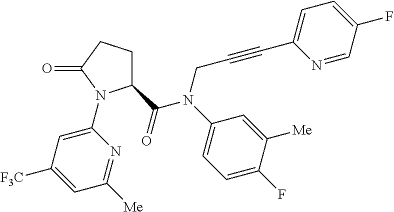 [SMILES: Cc1cc(C(F)(F)F)cc(N2C(=O)CC[C@H]2C(=O)N(CC#Cc2ccc(F)cn2)c2ccc(F)c(C)c2)n1] |
| 9 | 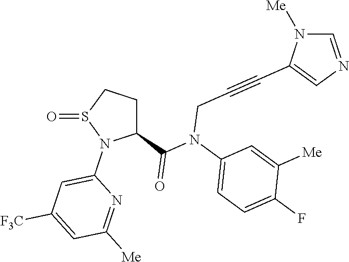 [SMILES: Cc1cc(C(F)(F)F)cc(N2[C@H](C(=O)N(CC#Cc3cncn3C)c3ccc(F)c(C)c3)CCS2=O)n1] |
| 10 | 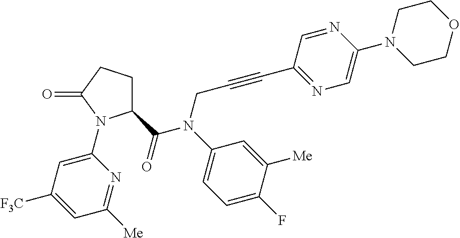 [SMILES: Cc1cc(C(F)(F)F)cc(N2C(=O)CC[C@H]2C(=O)N(CC#Cc2cnc(N3CCOCC3)cn2)c2ccc(F)c(C)c2)n1] |
| 11 | 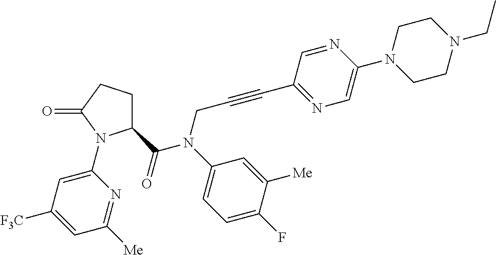 [SMILES: CCN1CCN(c2cnc(C#CCN(C(=O)[C@@H]3CCC(=O)N3c3cc(C(F)(F)F)cc(C)n3)c3ccc(F)c(C)c3)cn2)CC1] |
| 12 | 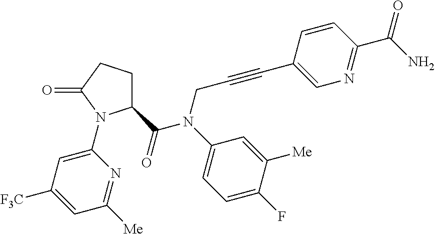 [SMILES: Cc1cc(C(F)(F)F)cc(N2C(=O)CC[C@H]2C(=O)N(CC#Cc2ccc(C(N)=O)nc2)c2ccc(F)c(C)c2)n1] |
| 13 | 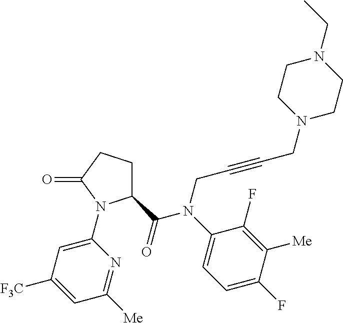 [SMILES: CCN1CCN(CC#CCN(C(=O)[C@@H]2CCC(=O)N2c2cc(C(F)(F)F)cc(C)n2)c2ccc(F)c(C)c2F)CC1] |
| 14 | 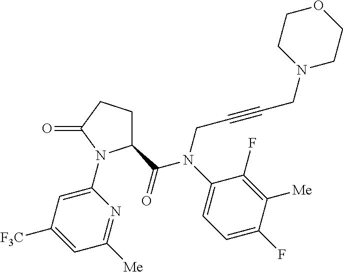 [SMILES: Cc1cc(C(F)(F)F)cc(N2C(=O)CC[C@H]2C(=O)N(CC#CCN2CCOCC2)c2ccc(F)c(C)c2F)n1] |
| 15 | 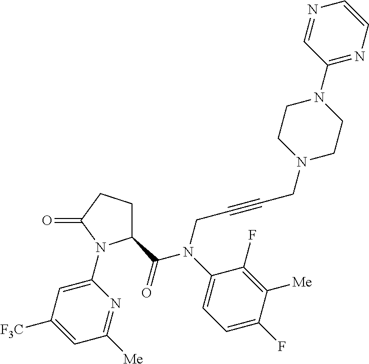 [SMILES: Cc1cc(C(F)(F)F)cc(N2C(=O)CC[C@H]2C(=O)N(CC#CCN2CCN(c3cnccn3)CC2)c2ccc(F)c(C)c2F)n1] |
| 16 | 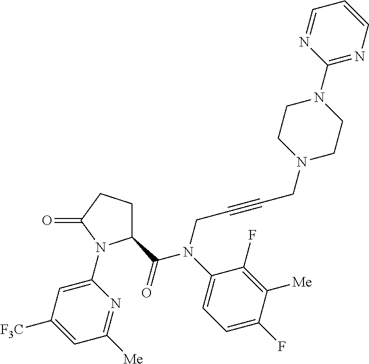 [SMILES: Cc1cc(C(F)(F)F)cc(N2C(=O)CC[C@H]2C(=O)N(CC#CCN2CCN(c3ncccn3)CC2)c2ccc(F)c(C)c2F)n1] |
| 17 | 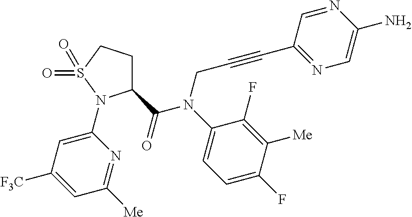 [SMILES: Cc1cc(C(F)(F)F)cc(N2[C@H](C(=O)N(CC#Cc3cnc(N)cn3)c3ccc(F)c(C)c3F)CCS2(=O)=O)n1] |
| 18 | 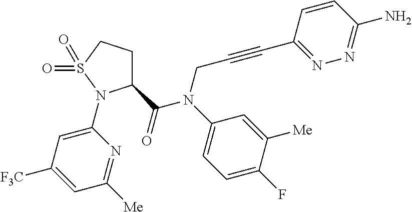 [SMILES: Cc1cc(C(F)(F)F)cc(N2[C@H](C(=O)N(CC#Cc3ccc(N)nn3)c3ccc(F)c(C)c3)CCS2(=O)=O)n1] |
| 19 | 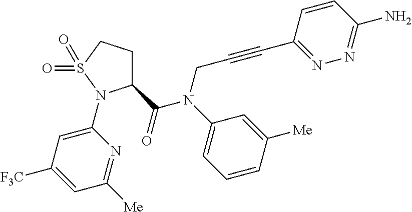 [SMILES: Cc1cccc(N(CC#Cc2ccc(N)nn2)C(=O)[C@@H]2CCS(=O)(=O)N2c2cc(C(F)(F)F)cc(C)n2)c1] |
| 20 | 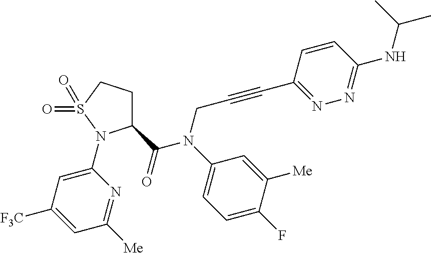 [SMILES: Cc1cc(C(F)(F)F)cc(N2[C@H](C(=O)N(CC#Cc3ccc(NC(C)C)nn3)c3ccc(F)c(C)c3)CCS2(=O)=O)n1] |
| 21 | 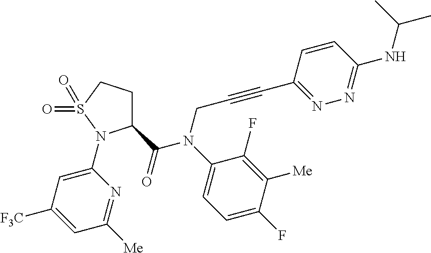 [SMILES: Cc1cc(C(F)(F)F)cc(N2[C@H](C(=O)N(CC#Cc3ccc(NC(C)C)nn3)c3ccc(F)c(C)c3F)CCS2(=O)=O)n1] |
| 22 | 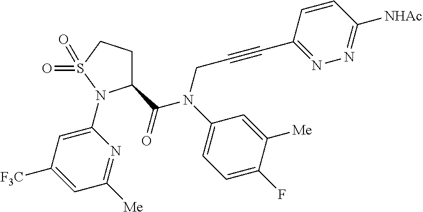 [SMILES: CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 23 | 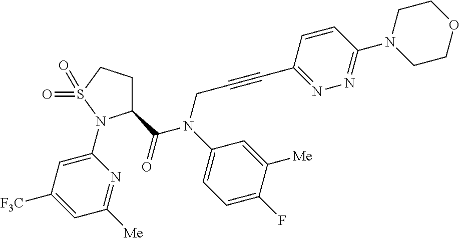 [SMILES: Cc1cc(C(F)(F)F)cc(N2[C@H](C(=O)N(CC#Cc3ccc(N4CCOCC4)nn3)c3ccc(F)c(C)c3)CCS2(=O)=O)n1] |
| 24 | 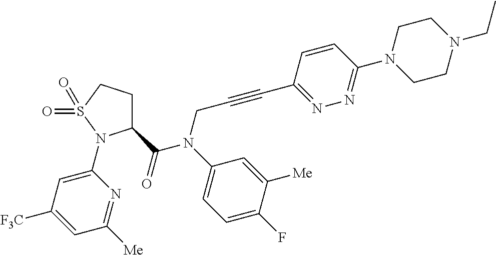 [SMILES: CCN1CCN(c2ccc(C#CCN(C(=O)[C@@H]3CCS(=O)(=O)N3c3cc(C(F)(F)F)cc(C)n3)c3ccc(F)c(C)c3)nn2)CC1] |
| 25 | 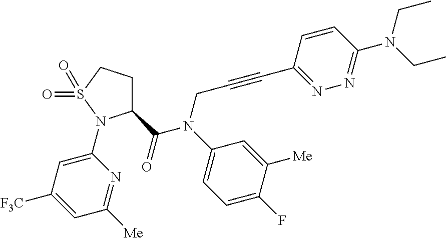 [SMILES: CCN(CC)c1ccc(C#CCN(C(=O)[C@@H]2CCS(=O)(=O)N2c2cc(C(F)(F)F)cc(C)n2)c2ccc(F)c(C)c2)nn1] |
| 26 | 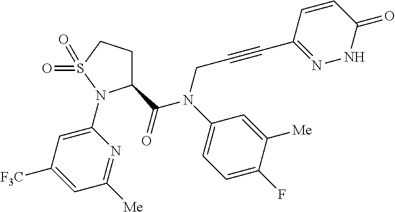 [SMILES: Cc1cc(C(F)(F)F)cc(N2[C@H](C(=O)N(CC#Cc3ccc(=O)[nH]n3)c3ccc(F)c(C)c3)CCS2(=O)=O)n1] |
| 27 | 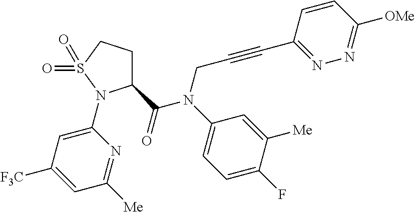 [SMILES: COc1ccc(C#CCN(C(=O)[C@@H]2CCS(=O)(=O)N2c2cc(C(F)(F)F)cc(C)n2)c2ccc(F)c(C)c2)nn1] |
| 28 | 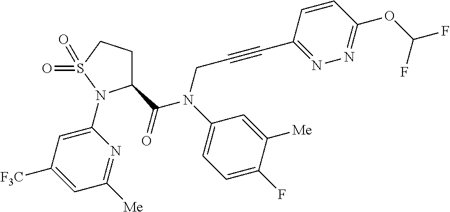 [SMILES: Cc1cc(C(F)(F)F)cc(N2[C@H](C(=O)N(CC#Cc3ccc(OC(F)F)nn3)c3ccc(F)c(C)c3)CCS2(=O)=O)n1] |
| 29 | 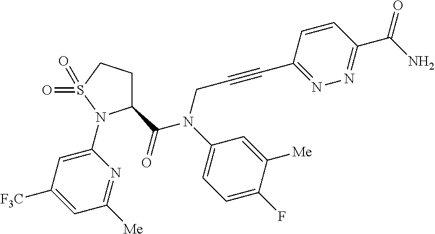 [SMILES: Cc1cc(C(F)(F)F)cc(N2[C@H](C(=O)N(CC#Cc3ccc(C(N)=O)nn3)c3ccc(F)c(C)c3)CCS2(=O)=O)n1] |
| 30 | 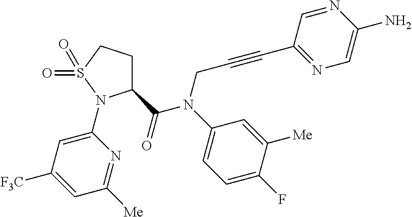 [SMILES: Cc1cc(C(F)(F)F)cc(N2[C@H](C(=O)N(CC#Cc3cnc(N)cn3)c3ccc(F)c(C)c3)CCS2(=O)=O)n1] |
| 31 | 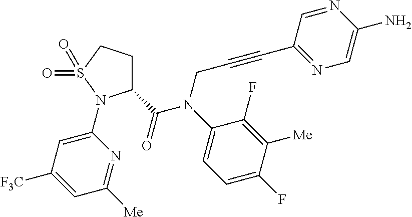 [SMILES: Cc1cc(C(F)(F)F)cc(N2[C@@H](C(=O)N(CC#Cc3cnc(N)cn3)c3ccc(F)c(C)c3F)CCS2(=O)=O)n1] |
| 32 | 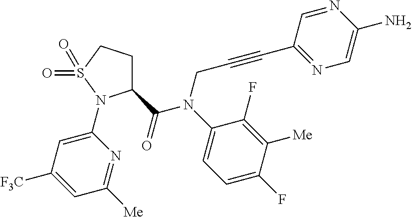 [SMILES: Cc1cc(C(F)(F)F)cc(N2[C@H](C(=O)N(CC#Cc3cnc(N)cn3)c3ccc(F)c(C)c3F)CCS2(=O)=O)n1] |
| 33 | 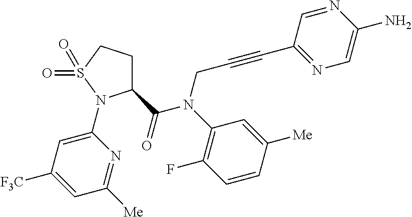 [SMILES: Cc1ccc(F)c(N(CC#Cc2cnc(N)cn2)C(=O)[C@@H]2CCS(=O)(=O)N2c2cc(C(F)(F)F)cc(C)n2)c1] |
| 34 | 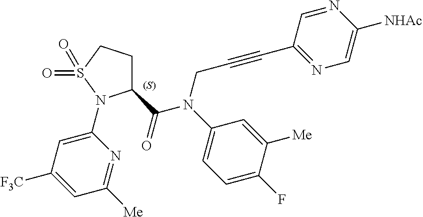 |
| 35 | 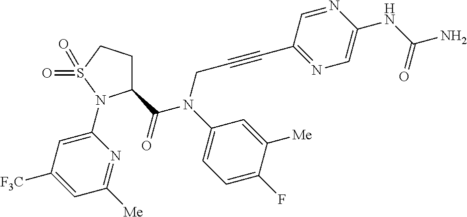 [SMILES: Cc1cc(C(F)(F)F)cc(N2[C@H](C(=O)N(CC#Cc3cnc(NC(N)=O)cn3)c3ccc(F)c(C)c3)CCS2(=O)=O)n1] |
| 36 | 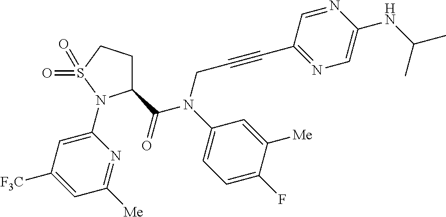 [SMILES: Cc1cc(C(F)(F)F)cc(N2[C@H](C(=O)N(CC#Cc3cnc(NC(C)C)cn3)c3ccc(F)c(C)c3)CCS2(=O)=O)n1] |
| 37 | 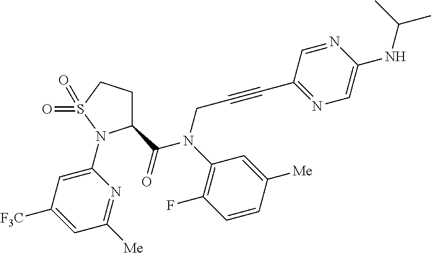 [SMILES: Cc1ccc(F)c(N(CC#Cc2cnc(NC(C)C)cn2)C(=O)[C@@H]2CCS(=O)(=O)N2c2cc(C(F)(F)F)cc(C)n2)c1] |
| 38 | 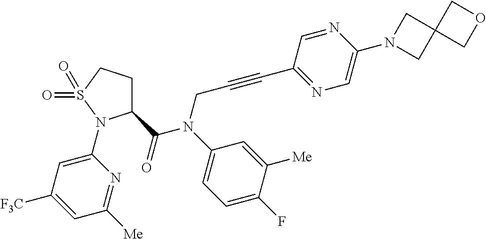 [SMILES: Cc1cc(C(F)(F)F)cc(N2[C@H](C(=O)N(CC#Cc3cnc(N4CC5(COC5)C4)cn3)c3ccc(F)c(C)c3)CCS2(=O)=O)n1] |
| 39 | 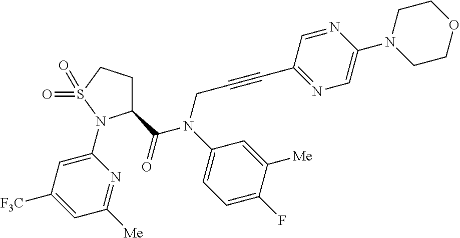 [SMILES: Cc1cc(C(F)(F)F)cc(N2[C@H](C(=O)N(CC#Cc3cnc(N4CCOCC4)cn3)c3ccc(F)c(C)c3)CCS2(=O)=O)n1] |
| 40 | 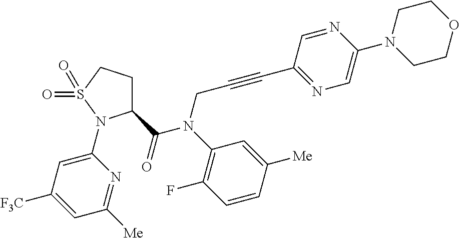 [SMILES: Cc1ccc(F)c(N(CC#Cc2cnc(N3CCOCC3)cn2)C(=O)[C@@H]2CCS(=O)(=O)N2c2cc(C(F)(F)F)cc(C)n2)c1] |
| 41 | 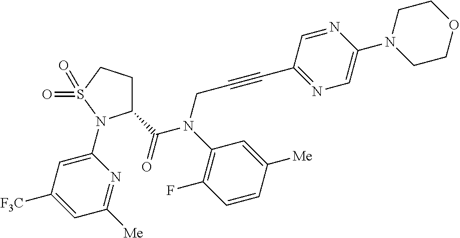 [SMILES: Cc1ccc(F)c(N(CC#Cc2cnc(N3CCOCC3)cn2)C(=O)[C@H]2CCS(=O)(=O)N2c2cc(C(F)(F)F)cc(C)n2)c1] |
| 42 | 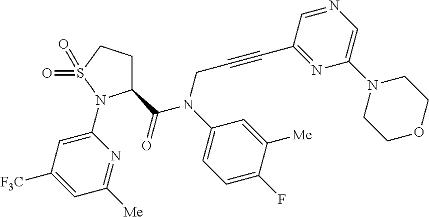 [SMILES: Cc1cc(C(F)(F)F)cc(N2[C@H](C(=O)N(CC#Cc3cncc(N4CCOCC4)n3)c3ccc(F)c(C)c3)CCS2(=O)=O)n1] |
| 43 | 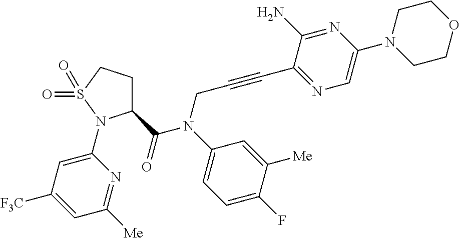 [SMILES: Cc1cc(C(F)(F)F)cc(N2[C@H](C(=O)N(CC#Cc3ncc(N4CCOCC4)nc3N)c3ccc(F)c(C)c3)CCS2(=O)=O)n1] |
| 44 | 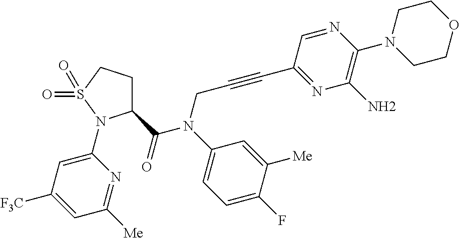 [SMILES: Cc1cc(C(F)(F)F)cc(N2[C@H](C(=O)N(CC#Cc3cnc(N4CCOCC4)c(N)n3)c3ccc(F)c(C)c3)CCS2(=O)=O)n1] |
| 45 | 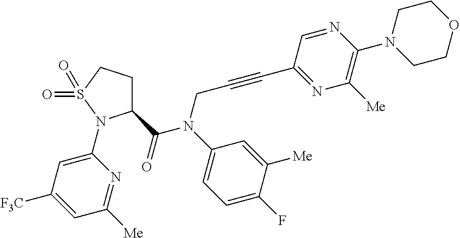 [SMILES: Cc1cc(C(F)(F)F)cc(N2[C@H](C(=O)N(CC#Cc3cnc(N4CCOCC4)c(C)n3)c3ccc(F)c(C)c3)CCS2(=O)=O)n1] |
| 46 | 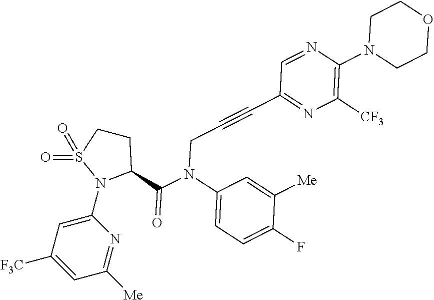 [SMILES: Cc1cc(C(F)(F)F)cc(N2[C@H](C(=O)N(CC#Cc3cnc(N4CCOCC4)c(C(F)(F)F)n3)c3ccc(F)c(C)c3)CCS2(=O)=O)n1] |
| 47 | 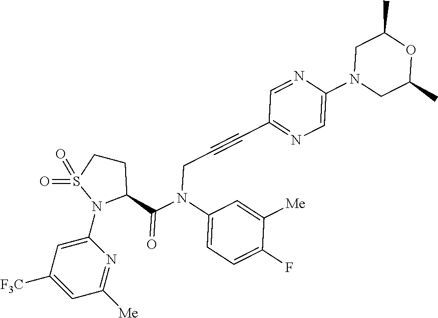 [SMILES: Cc1cc(C(F)(F)F)cc(N2[C@H](C(=O)N(CC#Cc3cnc(N4C[C@@H](C)O[C@@H](C)C4)cn3)c3ccc(F)c(C)c3)CCS2(=O)=O)n1] |
| 48 | 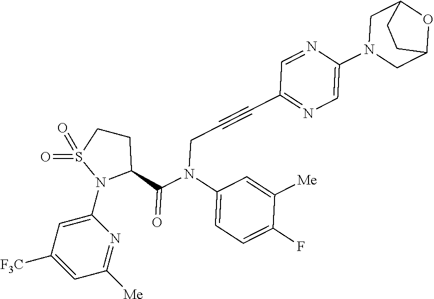 [SMILES: Cc1cc(C(F)(F)F)cc(N2[C@H](C(=O)N(CC#Cc3cnc(N4CC5CCC(C4)O5)cn3)c3ccc(F)c(C)c3)CCS2(=O)=O)n1] |
| 49 | 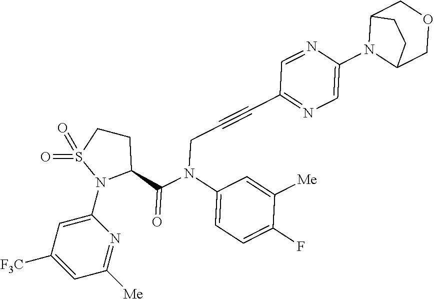 [SMILES: Cc1cc(C(F)(F)F)cc(N2[C@H](C(=O)N(CC#Cc3cnc(N4C5CCC4COC5)cn3)c3ccc(F)c(C)c3)CCS2(=O)=O)n1] |
| 50 | 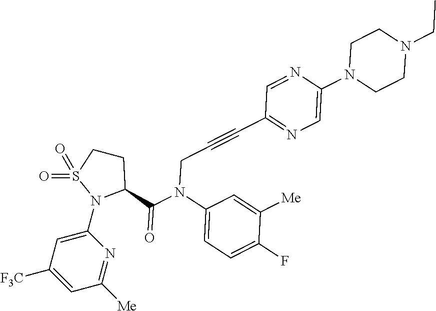 [SMILES: CCN1CCN(c2cnc(C#CCN(C(=O)[C@@H]3CCS(=O)(=O)N3c3cc(C(F)(F)F)cc(C)n3)c3ccc(F)c(C)c3)cn2)CC1] |
| 51 | 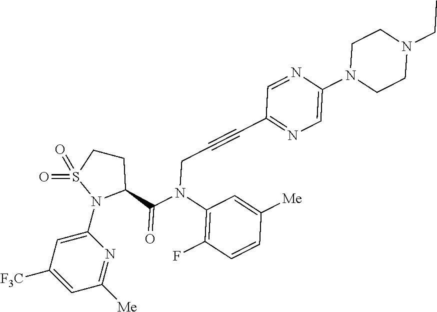 [SMILES: CCN1CCN(c2cnc(C#CCN(C(=O)[C@@H]3CCS(=O)(=O)N3c3cc(C(F)(F)F)cc(C)n3)c3cc(C)ccc3F)cn2)CC1] |
| 52 | 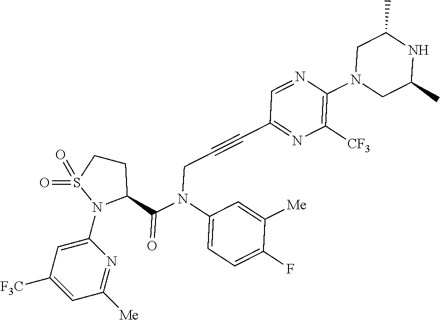 [SMILES: Cc1cc(C(F)(F)F)cc(N2[C@H](C(=O)N(CC#Cc3cnc(N4C[C@H](C)N[C@@H](C)C4)c(C(F)(F)F)n3)c3ccc(F)c(C)c3)CCS2(=O)=O)n1] |
| 53 | 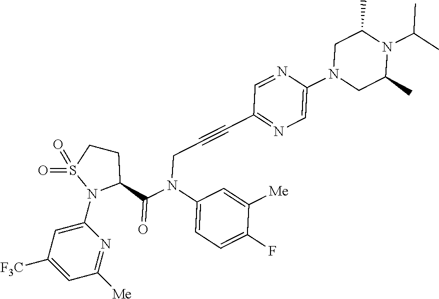 [SMILES: Cc1cc(C(F)(F)F)cc(N2[C@H](C(=O)N(CC#Cc3cnc(N4C[C@H](C)N(C(C)C)[C@@H](C)C4)cn3)c3ccc(F)c(C)c3)CCS2(=O)=O)n1] |
| 54 | 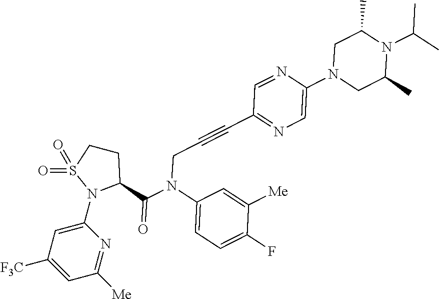 [SMILES: Cc1cc(C(F)(F)F)cc(N2[C@H](C(=O)N(CC#Cc3cnc(N4C[C@H](C)N(C(C)C)[C@@H](C)C4)cn3)c3ccc(F)c(C)c3)CCS2(=O)=O)n1] |
| 55 | 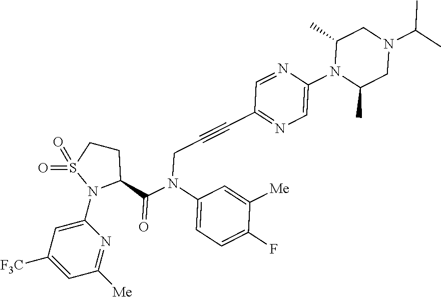 [SMILES: Cc1cc(C(F)(F)F)cc(N2[C@H](C(=O)N(CC#Cc3cnc(N4[C@H](C)CN(C(C)C)C[C@H]4C)cn3)c3ccc(F)c(C)c3)CCS2(=O)=O)n1] |
| 56 | 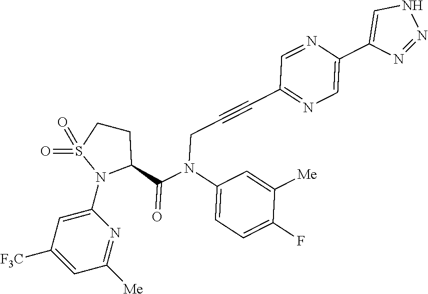 [SMILES: Cc1cc(C(F)(F)F)cc(N2[C@H](C(=O)N(CC#Cc3cnc(-c4c[nH]nn4)cn3)c3ccc(F)c(C)c3)CCS2(=O)=O)n1] |
| 57 | 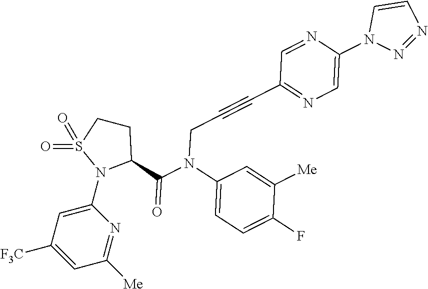 [SMILES: Cc1cc(C(F)(F)F)cc(N2[C@H](C(=O)N(CC#Cc3cnc(-n4ccnn4)cn3)c3ccc(F)c(C)c3)CCS2(=O)=O)n1] |
| 58 | 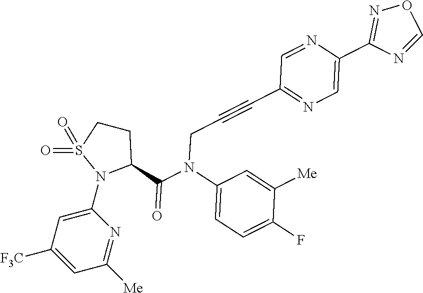 [SMILES: Cc1cc(C(F)(F)F)cc(N2[C@H](C(=O)N(CC#Cc3cnc(-c4ncon4)cn3)c3ccc(F)c(C)c3)CCS2(=O)=O)n1] |
| 59 | 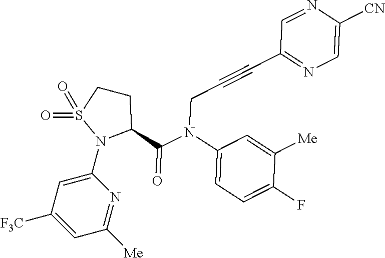 [SMILES: Cc1cc(C(F)(F)F)cc(N2[C@H](C(=O)N(CC#Cc3cnc(C#N)cn3)c3ccc(F)c(C)c3)CCS2(=O)=O)n1] |
| 60 | 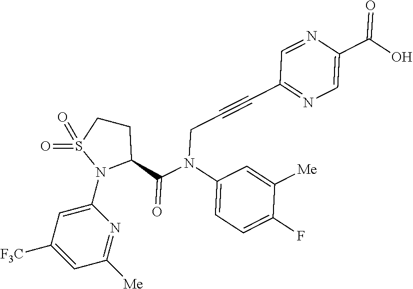 [SMILES: Cc1cc(C(F)(F)F)cc(N2[C@H](C(=O)N(CC#Cc3cnc(C(=O)O)cn3)c3ccc(F)c(C)c3)CCS2(=O)=O)n1] |
| 61 | 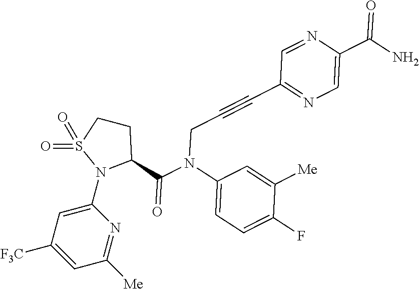 [SMILES: Cc1cc(C(F)(F)F)cc(N2[C@H](C(=O)N(CC#Cc3cnc(C(N)=O)cn3)c3ccc(F)c(C)c3)CCS2(=O)=O)n1] |
| 62 | 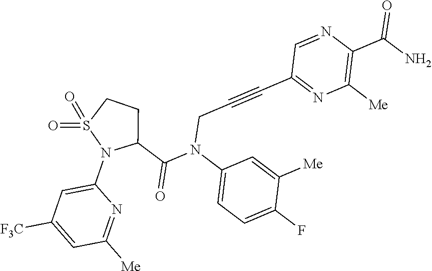 [SMILES: Cc1cc(C(F)(F)F)cc(N2C(C(=O)N(CC#Cc3cnc(C(N)=O)c(C)n3)c3ccc(F)c(C)c3)CCS2(=O)=O)n1] |
| 63 | 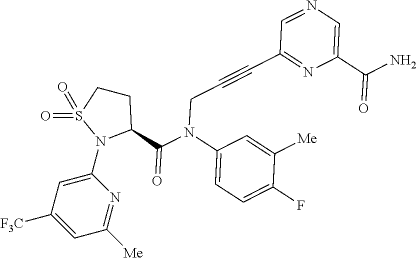 [SMILES: Cc1cc(C(F)(F)F)cc(N2[C@H](C(=O)N(CC#Cc3cncc(C(N)=O)n3)c3ccc(F)c(C)c3)CCS2(=O)=O)n1] |
| 64 | 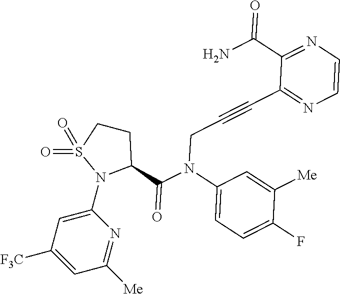 [SMILES: Cc1cc(C(F)(F)F)cc(N2[C@H](C(=O)N(CC#Cc3nccnc3C(N)=O)c3ccc(F)c(C)c3)CCS2(=O)=O)n1] |
| 65 | 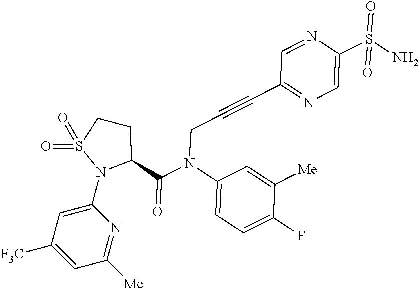 [SMILES: Cc1cc(C(F)(F)F)cc(N2[C@H](C(=O)N(CC#Cc3cnc(S(N)(=O)=O)cn3)c3ccc(F)c(C)c3)CCS2(=O)=O)n1] |
| 66 | 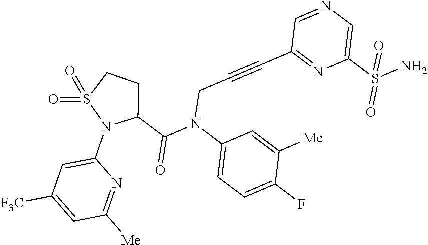 [SMILES: Cc1cc(C(F)(F)F)cc(N2C(C(=O)N(CC#Cc3cncc(S(N)(=O)=O)n3)c3ccc(F)c(C)c3)CCS2(=O)=O)n1] |
| 67 | 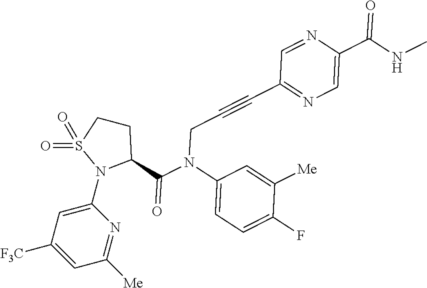 [SMILES: CNC(=O)c1cnc(C#CCN(C(=O)[C@@H]2CCS(=O)(=O)N2c2cc(C(F)(F)F)cc(C)n2)c2ccc(F)c(C)c2)cn1] |
| 68 | 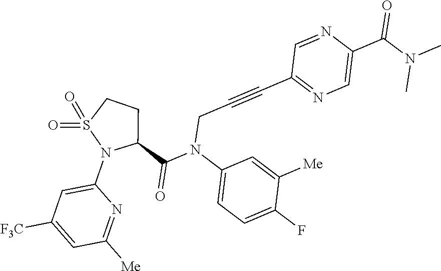 [SMILES: Cc1cc(C(F)(F)F)cc(N2[C@H](C(=O)N(CC#Cc3cnc(C(=O)N(C)C)cn3)c3ccc(F)c(C)c3)CCS2(=O)=O)n1] |
| 69 | 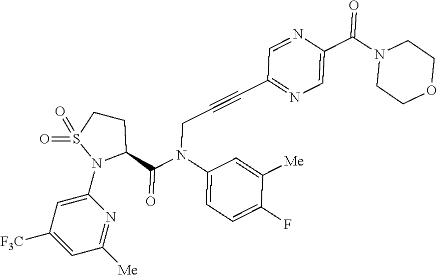 [SMILES: Cc1cc(C(F)(F)F)cc(N2[C@H](C(=O)N(CC#Cc3cnc(C(=O)N4CCOCC4)cn3)c3ccc(F)c(C)c3)CCS2(=O)=O)n1] |
| 70 | 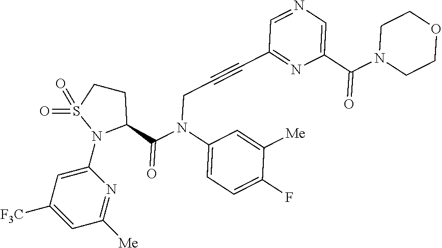 [SMILES: Cc1cc(C(F)(F)F)cc(N2[C@H](C(=O)N(CC#Cc3cncc(C(=O)N4CCOCC4)n3)c3ccc(F)c(C)c3)CCS2(=O)=O)n1] |
| 71 | 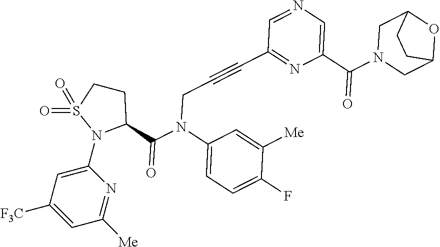 [SMILES: Cc1cc(C(F)(F)F)cc(N2[C@H](C(=O)N(CC#Cc3cncc(C(=O)N4CC5CCC(C4)O5)n3)c3ccc(F)c(C)c3)CCS2(=O)=O)n1] |
| 72 | 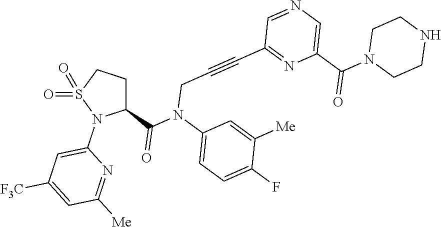 [SMILES: Cc1cc(C(F)(F)F)cc(N2[C@H](C(=O)N(CC#Cc3cncc(C(=O)N4CCNCC4)n3)c3ccc(F)c(C)c3)CCS2(=O)=O)n1] |
| 73 | 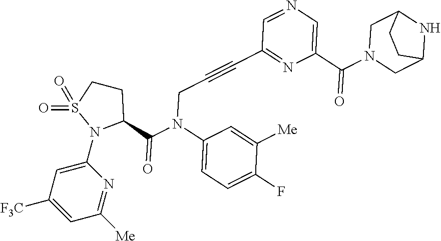 [SMILES: Cc1cc(C(F)(F)F)cc(N2[C@H](C(=O)N(CC#Cc3cncc(C(=O)N4CC5CCC(C4)N5)n3)c3ccc(F)c(C)c3)CCS2(=O)=O)n1] |
| 74 | 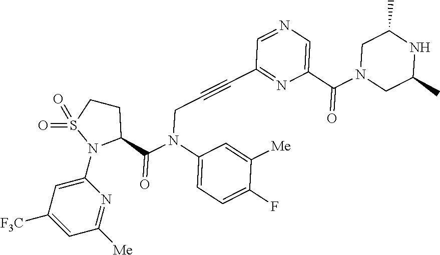 [SMILES: Cc1cc(C(F)(F)F)cc(N2[C@H](C(=O)N(CC#Cc3cncc(C(=O)N4C[C@H](C)N[C@@H](C)C4)n3)c3ccc(F)c(C)c3)CCS2(=O)=O)n1] |
| 75 | 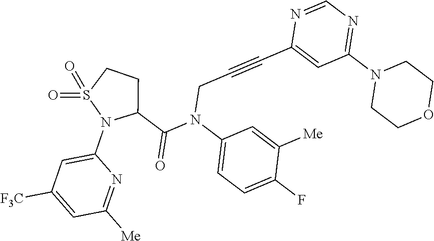 [SMILES: Cc1cc(C(F)(F)F)cc(N2C(C(=O)N(CC#Cc3cc(N4CCOCC4)ncn3)c3ccc(F)c(C)c3)CCS2(=O)=O)n1] |
| 76 | 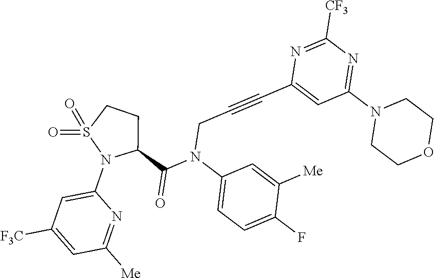 [SMILES: Cc1cc(C(F)(F)F)cc(N2[C@H](C(=O)N(CC#Cc3cc(N4CCOCC4)nc(C(F)(F)F)n3)c3ccc(F)c(C)c3)CCS2(=O)=O)n1] |
| 77 | 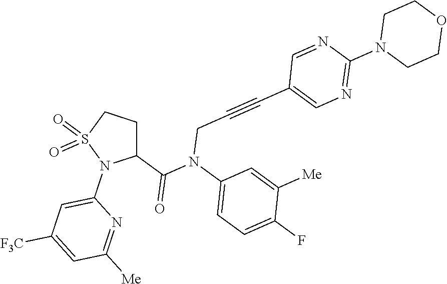 [SMILES: Cc1cc(C(F)(F)F)cc(N2C(C(=O)N(CC#Cc3cnc(N4CCOCC4)nc3)c3ccc(F)c(C)c3)CCS2(=O)=O)n1] |
| 78 | 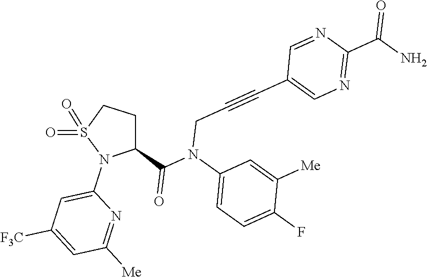 [SMILES: Cc1cc(C(F)(F)F)cc(N2[C@H](C(=O)N(CC#Cc3cnc(C(N)=O)nc3)c3ccc(F)c(C)c3)CCS2(=O)=O)n1] |
| 79 | 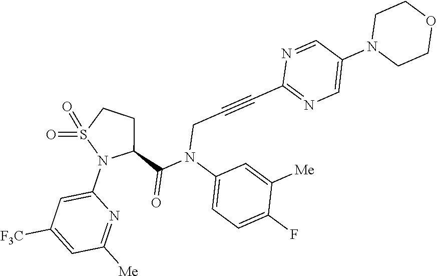 [SMILES: Cc1cc(C(F)(F)F)cc(N2[C@H](C(=O)N(CC#Cc3ncc(N4CCOCC4)cn3)c3ccc(F)c(C)c3)CCS2(=O)=O)n1] |
| 80 | 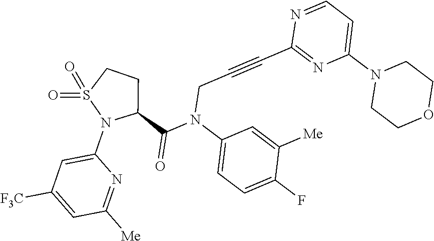 [SMILES: Cc1cc(C(F)(F)F)cc(N2[C@H](C(=O)N(CC#Cc3nccc(N4CCOCC4)n3)c3ccc(F)c(C)c3)CCS2(=O)=O)n1] |
| 81 | 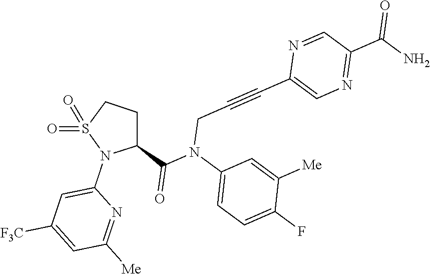 [SMILES: Cc1cc(C(F)(F)F)cc(N2[C@H](C(=O)N(CC#Cc3cnc(C(N)=O)cn3)c3ccc(F)c(C)c3)CCS2(=O)=O)n1] |
| 82 | 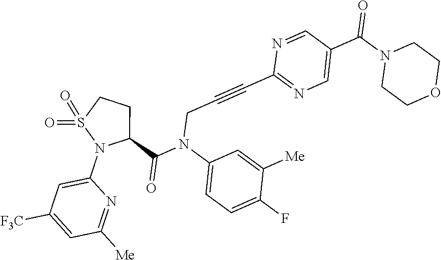 [SMILES: Cc1cc(C(F)(F)F)cc(N2[C@H](C(=O)N(CC#Cc3ncc(C(=O)N4CCOCC4)cn3)c3ccc(F)c(C)c3)CCS2(=O)=O)n1] |
| 83 | 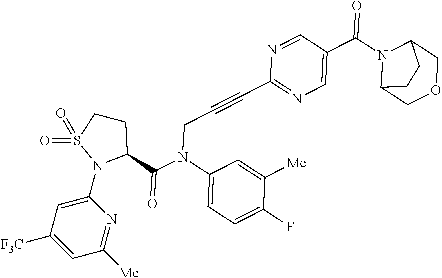 [SMILES: Cc1cc(C(F)(F)F)cc(N2[C@H](C(=O)N(CC#Cc3ncc(C(=O)N4C5CCC4COC5)cn3)c3ccc(F)c(C)c3)CCS2(=O)=O)n1] |
| 84 | 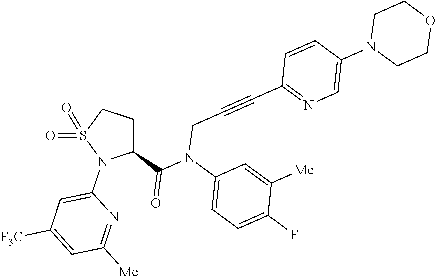 [SMILES: Cc1cc(C(F)(F)F)cc(N2[C@H](C(=O)N(CC#Cc3ccc(N4CCOCC4)cn3)c3ccc(F)c(C)c3)CCS2(=O)=O)n1] |
| 85 | 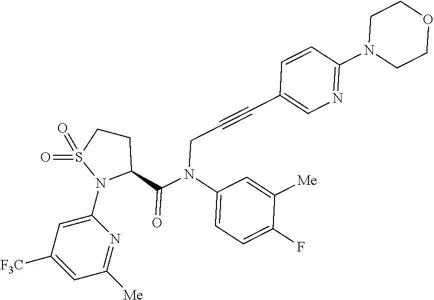 [SMILES: Cc1cc(C(F)(F)F)cc(N2[C@H](C(=O)N(CC#Cc3ccc(N4CCOCC4)nc3)c3ccc(F)c(C)c3)CCS2(=O)=O)n1] |
| 86 | 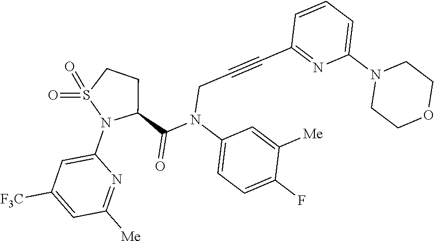 [SMILES: Cc1cc(C(F)(F)F)cc(N2[C@H](C(=O)N(CC#Cc3cccc(N4CCOCC4)n3)c3ccc(F)c(C)c3)CCS2(=O)=O)n1] |
| 87 | 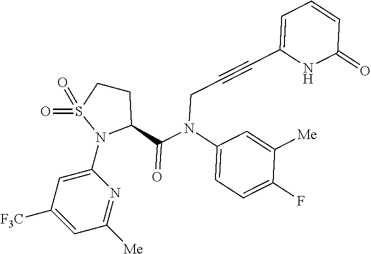 [SMILES: Cc1cc(C(F)(F)F)cc(N2[C@H](C(=O)N(CC#Cc3cccc(=O)[nH]3)c3ccc(F)c(C)c3)CCS2(=O)=O)n1] |
| 88 | 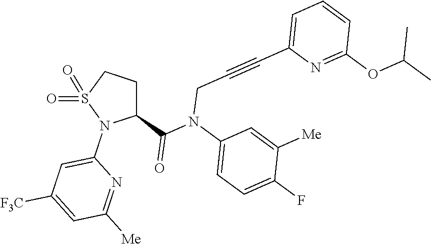 [SMILES: Cc1cc(C(F)(F)F)cc(N2[C@H](C(=O)N(CC#Cc3cccc(OC(C)C)n3)c3ccc(F)c(C)c3)CCS2(=O)=O)n1] |
| 89 | 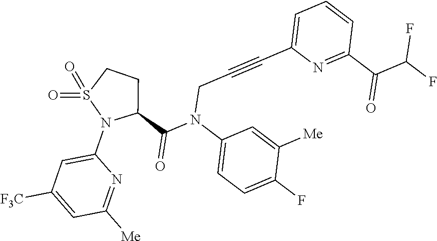 [SMILES: Cc1cc(C(F)(F)F)cc(N2[C@H](C(=O)N(CC#Cc3cccc(C(=O)C(F)F)n3)c3ccc(F)c(C)c3)CCS2(=O)=O)n1] |
| 90 | 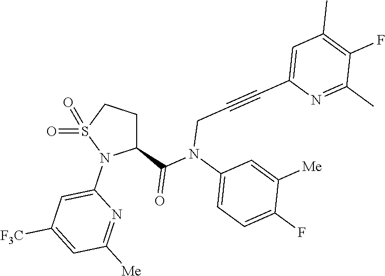 [SMILES: Cc1cc(C(F)(F)F)cc(N2[C@H](C(=O)N(CC#Cc3cc(C)c(F)c(C)n3)c3ccc(F)c(C)c3)CCS2(=O)=O)n1] |
| 91 | 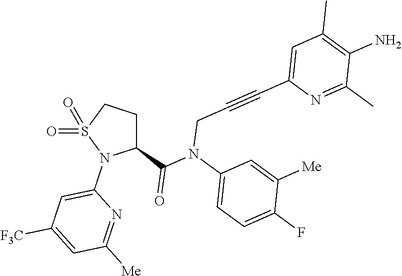 [SMILES: Cc1cc(C(F)(F)F)cc(N2[C@H](C(=O)N(CC#Cc3cc(C)c(N)c(C)n3)c3ccc(F)c(C)c3)CCS2(=O)=O)n1] |
| 92 | 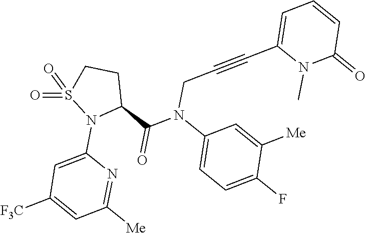 [SMILES: Cc1cc(C(F)(F)F)cc(N2[C@H](C(=O)N(CC#Cc3cccc(=O)n3C)c3ccc(F)c(C)c3)CCS2(=O)=O)n1] |
| 93 | 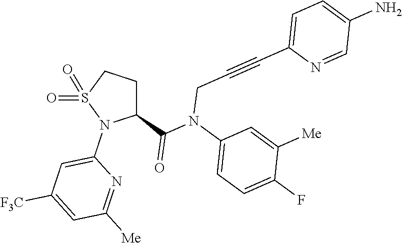 [SMILES: Cc1cc(C(F)(F)F)cc(N2[C@H](C(=O)N(CC#Cc3ccc(N)cn3)c3ccc(F)c(C)c3)CCS2(=O)=O)n1] |
| 94 | 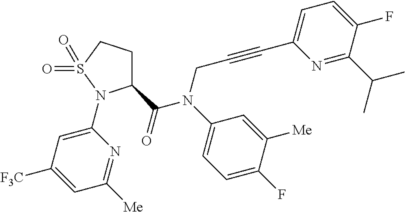 [SMILES: Cc1cc(C(F)(F)F)cc(N2[C@H](C(=O)N(CC#Cc3ccc(F)c(C(C)C)n3)c3ccc(F)c(C)c3)CCS2(=O)=O)n1] |
| 95 | 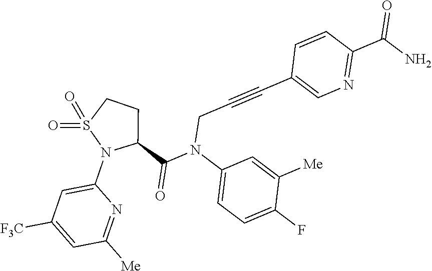 [SMILES: Cc1cc(C(F)(F)F)cc(N2[C@H](C(=O)N(CC#Cc3ccc(C(N)=O)nc3)c3ccc(F)c(C)c3)CCS2(=O)=O)n1] |
| 96 | 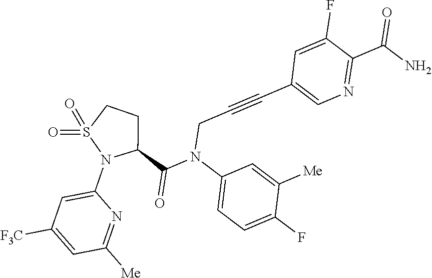 [SMILES: Cc1cc(C(F)(F)F)cc(N2[C@H](C(=O)N(CC#Cc3cnc(C(N)=O)c(F)c3)c3ccc(F)c(C)c3)CCS2(=O)=O)n1] |
| 97 | 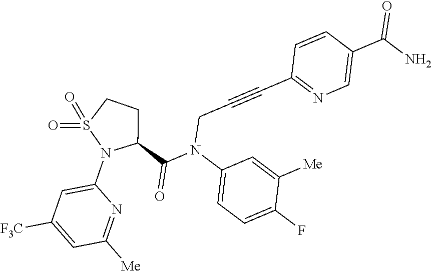 [SMILES: Cc1cc(C(F)(F)F)cc(N2[C@H](C(=O)N(CC#Cc3ccc(C(N)=O)cn3)c3ccc(F)c(C)c3)CCS2(=O)=O)n1] |
| 98 | 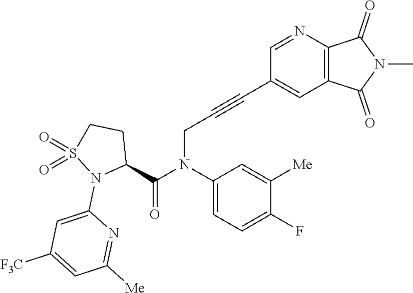 [SMILES: Cc1cc(C(F)(F)F)cc(N2[C@H](C(=O)N(CC#Cc3cnc4c(c3)C(=O)N(C)C4=O)c3ccc(F)c(C)c3)CCS2(=O)=O)n1] |
| 99 | 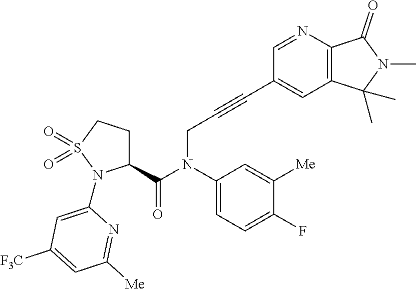 [SMILES: Cc1cc(C(F)(F)F)cc(N2[C@H](C(=O)N(CC#Cc3cnc4c(c3)C(C)(C)N(C)C4=O)c3ccc(F)c(C)c3)CCS2(=O)=O)n1] |
| 100 | 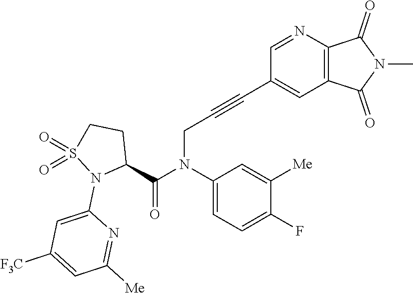 [SMILES: Cc1cc(C(F)(F)F)cc(N2[C@H](C(=O)N(CC#Cc3cnc4c(c3)C(=O)N(C)C4=O)c3ccc(F)c(C)c3)CCS2(=O)=O)n1] |
| 101 | 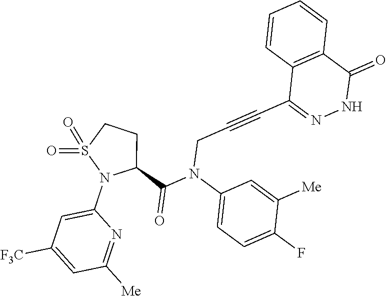 [SMILES: Cc1cc(C(F)(F)F)cc(N2[C@H](C(=O)N(CC#Cc3n[nH]c(=O)c4ccccc34)c3ccc(F)c(C)c3)CCS2(=O)=O)n1] |
| 102 | 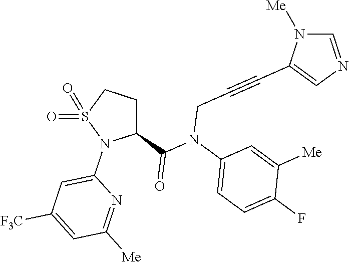 [SMILES: Cc1cc(C(F)(F)F)cc(N2[C@H](C(=O)N(CC#Cc3cncn3C)c3ccc(F)c(C)c3)CCS2(=O)=O)n1] |
| 103 | 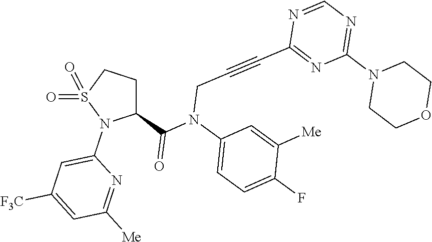 [SMILES: Cc1cc(C(F)(F)F)cc(N2[C@H](C(=O)N(CC#Cc3ncnc(N4CCOCC4)n3)c3ccc(F)c(C)c3)CCS2(=O)=O)n1] |
| 104 | 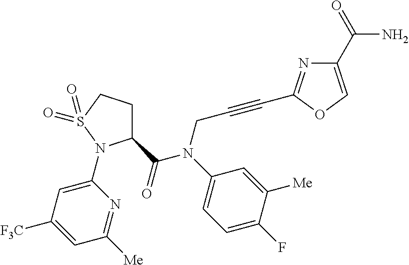 [SMILES: Cc1cc(C(F)(F)F)cc(N2[C@H](C(=O)N(CC#Cc3nc(C(N)=O)co3)c3ccc(F)c(C)c3)CCS2(=O)=O)n1] |
| 105 | 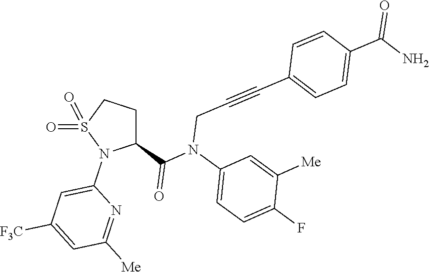 [SMILES: Cc1cc(C(F)(F)F)cc(N2[C@H](C(=O)N(CC#Cc3ccc(C(N)=O)cc3)c3ccc(F)c(C)c3)CCS2(=O)=O)n1] |
| 106 | 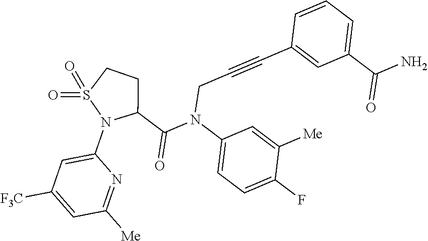 [SMILES: Cc1cc(C(F)(F)F)cc(N2C(C(=O)N(CC#Cc3cccc(C(N)=O)c3)c3ccc(F)c(C)c3)CCS2(=O)=O)n1] |
| 107 | 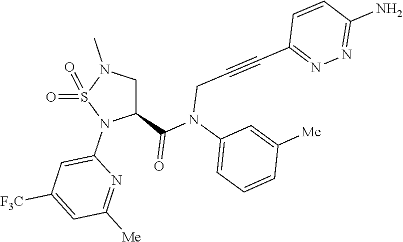 [SMILES: Cc1cccc(N(CC#Cc2ccc(N)nn2)C(=O)[C@@H]2CN(C)S(=O)(=O)N2c2cc(C(F)(F)F)cc(C)n2)c1] |
| 108 | 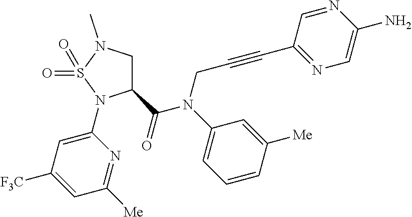 [SMILES: Cc1cccc(N(CC#Cc2cnc(N)cn2)C(=O)[C@@H]2CN(C)S(=O)(=O)N2c2cc(C(F)(F)F)cc(C)n2)c1] |
| 109 | 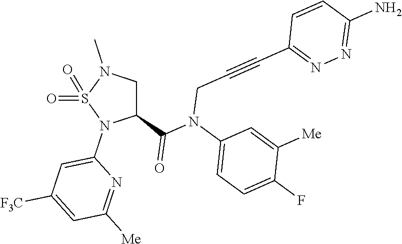 [SMILES: Cc1cc(C(F)(F)F)cc(N2[C@H](C(=O)N(CC#Cc3ccc(N)nn3)c3ccc(F)c(C)c3)CN(C)S2(=O)=O)n1] |
| 110 | 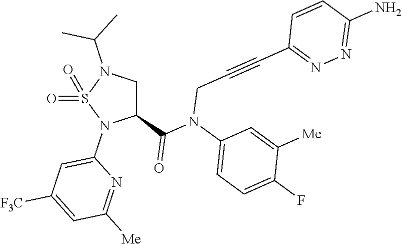 [SMILES: Cc1cc(C(F)(F)F)cc(N2[C@H](C(=O)N(CC#Cc3ccc(N)nn3)c3ccc(F)c(C)c3)CN(C(C)C)S2(=O)=O)n1] |
| 111 | 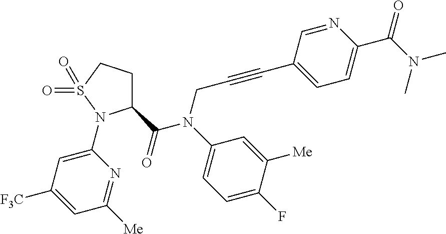 [SMILES: Cc1cc(C(F)(F)F)cc(N2[C@H](C(=O)N(CC#Cc3ccc(C(=O)N(C)C)nc3)c3ccc(F)c(C)c3)CCS2(=O)=O)n1] |
| 112 | 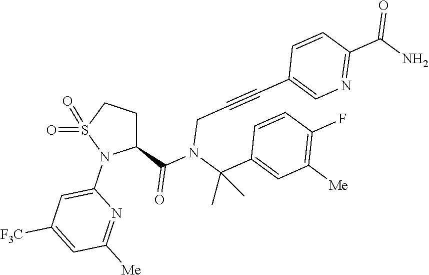 [SMILES: Cc1cc(C(F)(F)F)cc(N2[C@H](C(=O)N(CC#Cc3ccc(C(N)=O)nc3)C(C)(C)c3ccc(F)c(C)c3)CCS2(=O)=O)n1] |
| 113 | 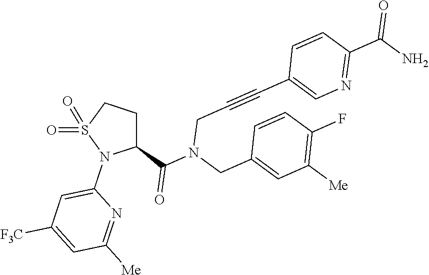 [SMILES: Cc1cc(C(F)(F)F)cc(N2[C@H](C(=O)N(CC#Cc3ccc(C(N)=O)nc3)Cc3ccc(F)c(C)c3)CCS2(=O)=O)n1] |
| 114 | 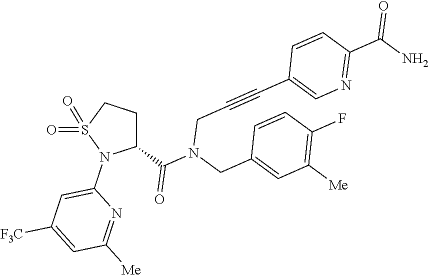 [SMILES: Cc1cc(C(F)(F)F)cc(N2[C@@H](C(=O)N(CC#Cc3ccc(C(N)=O)nc3)Cc3ccc(F)c(C)c3)CCS2(=O)=O)n1] |
| 115 | 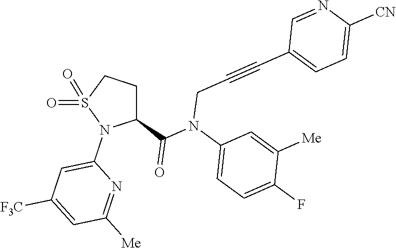 [SMILES: Cc1cc(C(F)(F)F)cc(N2[C@H](C(=O)N(CC#Cc3ccc(C#N)nc3)c3ccc(F)c(C)c3)CCS2(=O)=O)n1] |
| 116 | 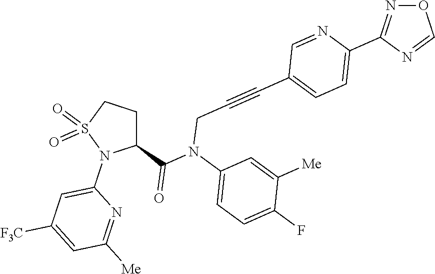 [SMILES: Cc1cc(C(F)(F)F)cc(N2[C@H](C(=O)N(CC#Cc3ccc(-c4ncon4)nc3)c3ccc(F)c(C)c3)CCS2(=O)=O)n1] |
| 117 | 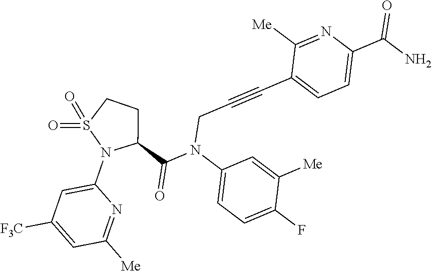 [SMILES: Cc1cc(C(F)(F)F)cc(N2[C@H](C(=O)N(CC#Cc3ccc(C(N)=O)nc3C)c3ccc(F)c(C)c3)CCS2(=O)=O)n1] |
| 118 | 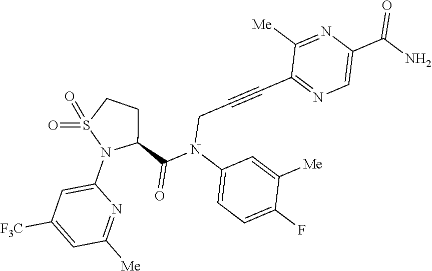 [SMILES: Cc1cc(C(F)(F)F)cc(N2[C@H](C(=O)N(CC#Cc3ncc(C(N)=O)nc3C)c3ccc(F)c(C)c3)CCS2(=O)=O)n1] |
| 119 | 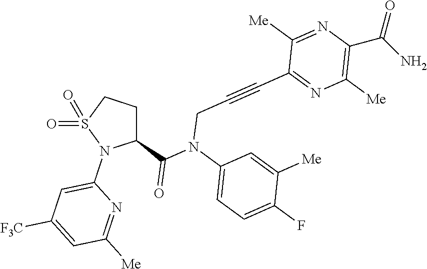 [SMILES: Cc1cc(C(F)(F)F)cc(N2[C@H](C(=O)N(CC#Cc3nc(C)c(C(N)=O)nc3C)c3ccc(F)c(C)c3)CCS2(=O)=O)n1] |
| 120 | 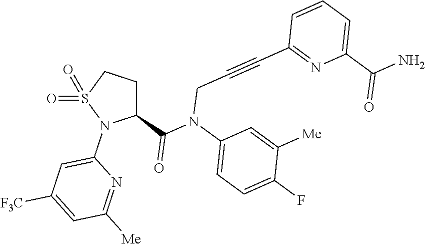 [SMILES: Cc1cc(C(F)(F)F)cc(N2[C@H](C(=O)N(CC#Cc3cccc(C(N)=O)n3)c3ccc(F)c(C)c3)CCS2(=O)=O)n1] |
| 121 | 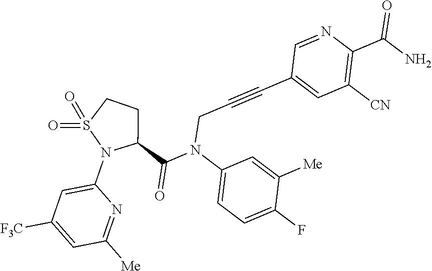 [SMILES: Cc1cc(C(F)(F)F)cc(N2[C@H](C(=O)N(CC#Cc3cnc(C(N)=O)c(C#N)c3)c3ccc(F)c(C)c3)CCS2(=O)=O)n1] |
| 122 | 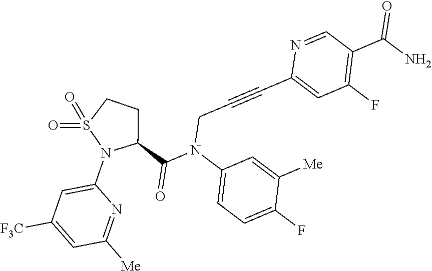 [SMILES: Cc1cc(C(F)(F)F)cc(N2[C@H](C(=O)N(CC#Cc3cc(F)c(C(N)=O)cn3)c3ccc(F)c(C)c3)CCS2(=O)=O)n1] |
| 123 | 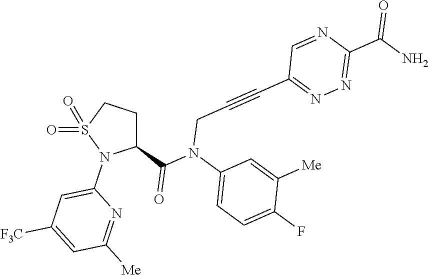 [SMILES: Cc1cc(C(F)(F)F)cc(N2[C@H](C(=O)N(CC#Cc3cnc(C(N)=O)nn3)c3ccc(F)c(C)c3)CCS2(=O)=O)n1] |
| 124 | 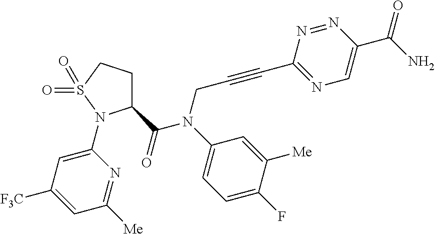 [SMILES: Cc1cc(C(F)(F)F)cc(N2[C@H](C(=O)N(CC#Cc3ncc(C(N)=O)nn3)c3ccc(F)c(C)c3)CCS2(=O)=O)n1] |
| 125 | 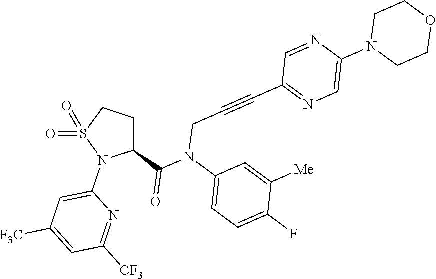 [SMILES: Cc1cc(N(CC#Cc2cnc(N3CCOCC3)cn2)C(=O)[C@@H]2CCS(=O)(=O)N2c2cc(C(F)(F)F)cc(C(F)(F)F)n2)ccc1F] |
| 126 | 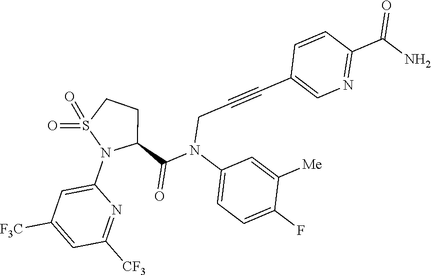 [SMILES: Cc1cc(N(CC#Cc2ccc(C(N)=O)nc2)C(=O)[C@@H]2CCS(=O)(=O)N2c2cc(C(F)(F)F)cc(C(F)(F)F)n2)ccc1F] |
| 127 | 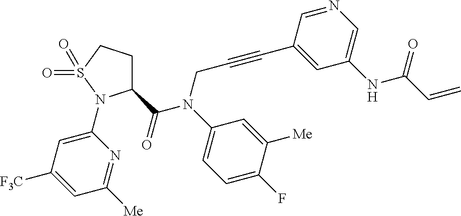 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)c1] |
| 128 | 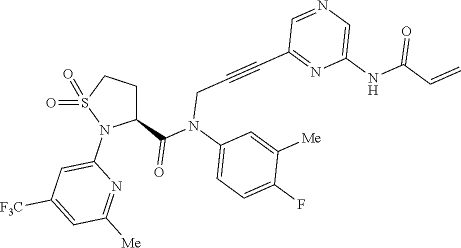 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)n1] |
| 129 | 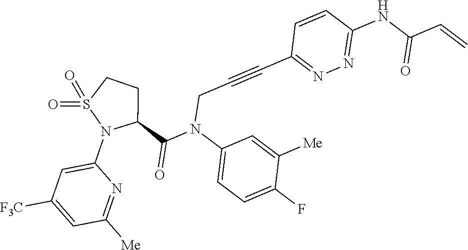 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 130 | 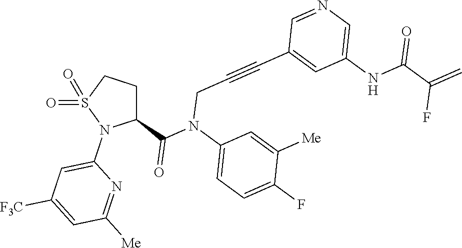 [SMILES: C=C(F)C(=O)Nc1cncc(C#CCN(C(=O)[C@@H]2CCS(=O)(=O)N2c2cc(C(F)(F)F)cc(C)n2)c2ccc(F)c(C)c2)c1] |
| 131 | 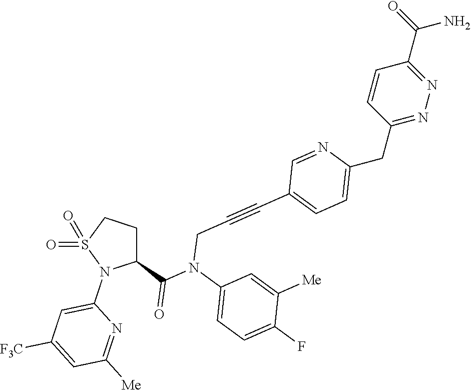 [SMILES: Cc1cc(C(F)(F)F)cc(N2[C@H](C(=O)N(CC#Cc3ccc(Cc4ccc(C(N)=O)nn4)nc3)c3ccc(F)c(C)c3)CCS2(=O)=O)n1] |
| 132 | 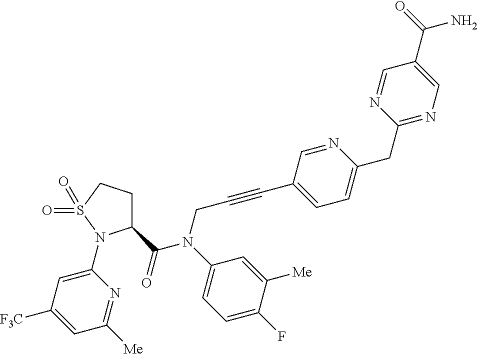 [SMILES: Cc1cc(C(F)(F)F)cc(N2[C@H](C(=O)N(CC#Cc3ccc(Cc4ncc(C(N)=O)cn4)nc3)c3ccc(F)c(C)c3)CCS2(=O)=O)n1] |
| 133 | 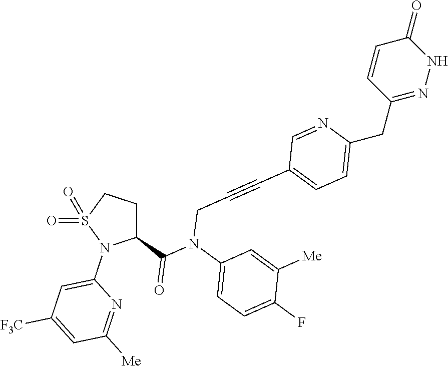 [SMILES: Cc1cc(C(F)(F)F)cc(N2[C@H](C(=O)N(CC#Cc3ccc(Cc4ccc(=O)[nH]n4)nc3)c3ccc(F)c(C)c3)CCS2(=O)=O)n1] |
| 134 | 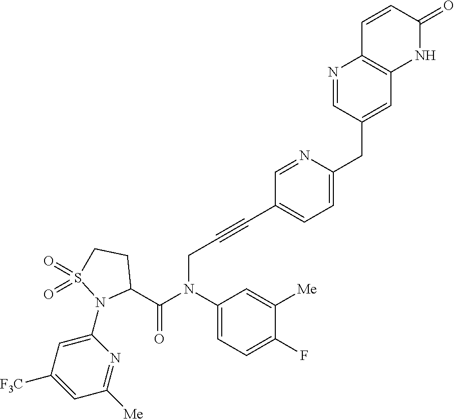 [SMILES: Cc1cc(C(F)(F)F)cc(N2C(C(=O)N(CC#Cc3ccc(Cc4cnc5ccc(=O)[nH]c5c4)nc3)c3ccc(F)c(C)c3)CCS2(=O)=O)n1] |
| 135 | 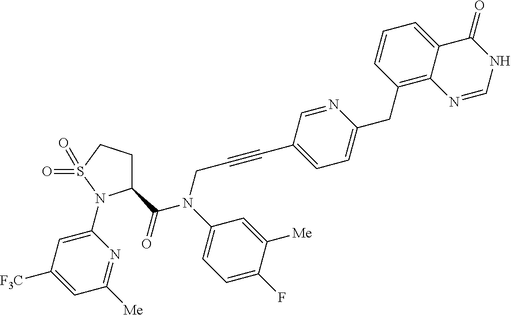 [SMILES: Cc1cc(C(F)(F)F)cc(N2[C@H](C(=O)N(CC#Cc3ccc(Cc4cccc5c(=O)[nH]cnc45)nc3)c3ccc(F)c(C)c3)CCS2(=O)=O)n1] |
| 136 | 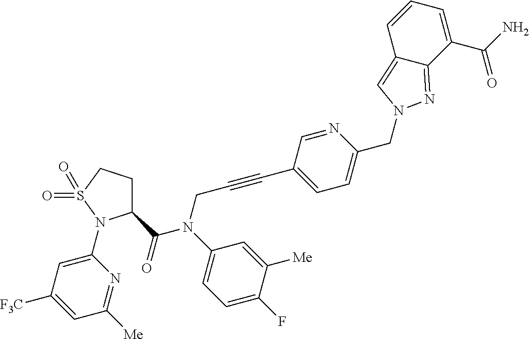 [SMILES: Cc1cc(C(F)(F)F)cc(N2[C@H](C(=O)N(CC#Cc3ccc(Cn4cc5cccc(C(N)=O)c5n4)nc3)c3ccc(F)c(C)c3)CCS2(=O)=O)n1] |
| 137 | 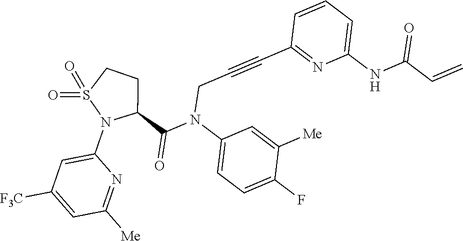 [SMILES: C=CC(=O)Nc1cccc(C#CCN(C(=O)[C@@H]2CCS(=O)(=O)N2c2cc(C(F)(F)F)cc(C)n2)c2ccc(F)c(C)c2)n1] |
| 138 | 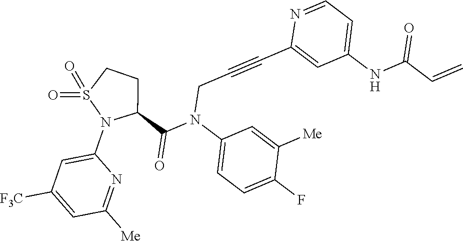 [SMILES: C=CC(=O)Nc1ccnc(C#CCN(C(=O)[C@@H]2CCS(=O)(=O)N2c2cc(C(F)(F)F)cc(C)n2)c2ccc(F)c(C)c2)c1] |
| 139 | 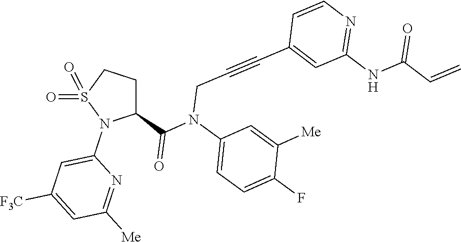 [SMILES: C=CC(=O)Nc1cc(C#CCN(C(=O)[C@@H]2CCS(=O)(=O)N2c2cc(C(F)(F)F)cc(C)n2)c2ccc(F)c(C)c2)ccn1] |
| 140 | 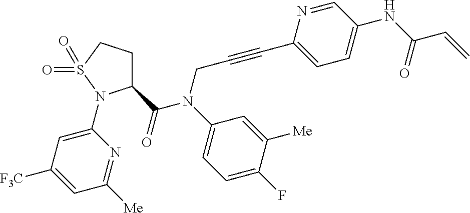 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nc1] |
| 141 | 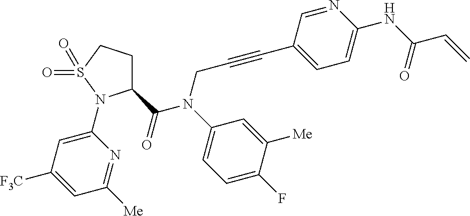 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)cn1] |
| 142 | 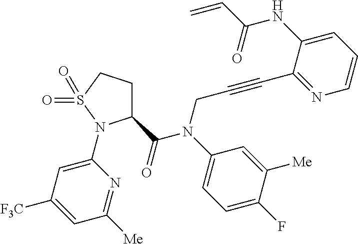 [SMILES: C=CC(=O)Nc1cccnc1C#CCN(C(=O)[C@@H]1CCS(=O)(=O)N1c1cc(C(F)(F)F)cc(C)n1)c1ccc(F)c(C)c1] |
| 143 | 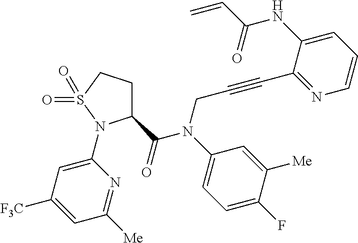 [SMILES: C=CC(=O)Nc1cccnc1C#CCN(C(=O)[C@@H]1CCS(=O)(=O)N1c1cc(C(F)(F)F)cc(C)n1)c1ccc(F)c(C)c1] |
| 144 | 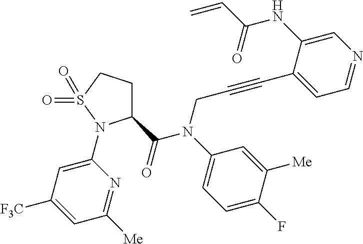 [SMILES: C=CC(=O)Nc1cnccc1C#CCN(C(=O)[C@@H]1CCS(=O)(=O)N1c1cc(C(F)(F)F)cc(C)n1)c1ccc(F)c(C)c1] |
| 145 | 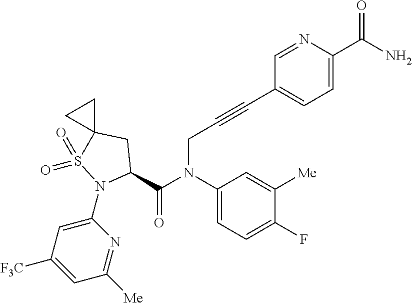 [SMILES: Cc1cc(C(F)(F)F)cc(N2[C@H](C(=O)N(CC#Cc3ccc(C(N)=O)nc3)c3ccc(F)c(C)c3)CC3(CC3)S2(=O)=O)n1] |

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “stereoisomers” refers to compounds that have identical chemical constitution, but differ with regard to the arrangement of the atoms or groups in space. Stereoisomers include diastereomers, enantiomers, conformers and the like.

As used herein, the term “diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may be separated into each stereoisomer under high resolution analytical procedures such as electrophoresis and chromatography such as HPLC.

As used herein, the term “enantiomers” refers to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds may exist in optically active forms, i.e., they have the ability to rotate the plane of polarization of plane-polarized light. For describing an optically active compound, the prefixes D and L, or R and S, are used to denote the absolute configuration of the molecule about its chiral center(s). The prefixes d and 1 or (+) and (−) denote the direction in which plane of the polarized light is rotated. A compound prefixed by (−) or 1 is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

It will be understood by those skilled in the art that the organic compounds can form complexes with solvents in which they are reacted or from which they are precipitated or crystallized. These complexes are known as “solvates.” Where the solvent is water, the complex is known as “hydrate.” The present disclosure encompasses all solvates of the compounds disclosed herein. Conventional solvents include water, methanol, ethanol, acetic acid, DMSO, THF, diethyl ether, etc. The compounds described herein can be prepared, for example, in crystalline form, and can be solvated. Suitable solvates include pharmaceutically acceptable solvates and further include both stoichiometric solvates and non-stoichiometric solvates. In some cases, the solvates will be capable of isolation, for example, when one or more solvent molecules are incorporated into the crystal lattice of a crystalline solid. “Solvate” means both solution-phase and isolatable solvates. Representative solvates include hydrates, ethanolates and methanolates.

The term “hydrate” refers to a compound that is associated with water. Generally, the number of water molecules contained in a hydrate of a compound is in a definite ratio to the number of the compound molecules in the hydrate. Therefore, hydrates of a compound can be represented, for example, by a general formula R·x H₂O, wherein R denotes the compound, and x is a number greater than 0. Given compounds can form more than one type of hydrate, including, for example, monohydrates (x is 1), lower hydrates (x is a number greater than 0 and smaller than 1, for example, hemihydrates (R·0.5 H₂O)) and polyhydrates (x is a number greater than 1, for example, dihydrates (R·2 H₂O) and hexahydrates (R·6 H₂O)).

Compounds disclosed herein may be in an amorphous or crystalline form (crystal form or polymorph). Furthermore, the compounds disclosed herein may exist in one or more crystalline forms. Therefore, the scope of the present disclosure includes all amorphous or crystalline forms of the compounds disclosed herein. The term “polymorph” refers to a crystalline form of a compound (or a salt, hydrate, or solvate thereof) in a particular crystal packing arrangement. All polymorphs have the same elemental composition. Different crystalline forms generally have different X-ray diffraction patterns, infrared spectra, melting points, density, hardness, crystal shapes, optical and electrical properties, stability, and solubility. Recrystallization solvents, rate of crystallization, storage temperatures, and other factors may cause one crystalline form to dominate. Various polymorphs of a compound can be prepared by crystallization under different conditions.

As used herein, the term “isotopically labeled form” of a compound that contains an isotopic form of one or more atoms in the compound that is different from the naturally occurring isotopic distribution of the atom in nature. All isotopic forms are included as options, unless a specific isotopic form is indicated. An “isotopically label form” of a compound can be radiolabeled, that is, contain one or more radioactive isotopes, or can be labeled with non-radioactive isotopes such as for example, deuterium (2H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined by those skilled in the art.

As used herein, the term “prodrug” refers to substances that can be converted, under physiological conditions or through solvolysis, into the compound of the present disclosure having biological activity. The prodrug of the present disclosure is prepared by modifying the functional groups in the compound, and the modification can be removed by conventional operations or removed in vivo, to obtain the compound of the present disclosure. The prodrug includes a compound which is formed by connecting a hydroxyl group or amino group in the compound of the present disclosure to any group. When the prodrug of the compound of the present disclosure is administered to a mammalian individual, the prodrug is dissociated to form a free hydroxyl or amino group.

The term “pharmaceutically acceptable salt” refers to a salt which is, within the scope of sound medical judgment, suitable for use in contact with the tissues of humans and lower animals without undue toxicity, irritation, allergic response and the like, and is commensurate with a reasonable benefit/risk ratio.

Certain compounds disclosed herein can exist in the form of salts, for example acid addition salts, or salts with organic or inorganic bases such as carboxylate, sulfonate and phosphate salts. All such salts are within the scope of this invention, and references to compounds disclosed herein include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base form of the parent compound with the appropriate base or acid in water or in an organic solvent, or in a mixture of the two; generally, non-aqueous media such as ether, ethyl acetate, ethanol, isopropanol, or acetonitrile are used. Acid addition salts (e.g., mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), a-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1, 5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic, valeric acids, and acylated amino acids.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is a hydrochloride salt.

Where the compounds disclosed herein contain an amine function, the compound may form quaternary ammonium salts, for example by reaction with an alkylating agent according to methods well known to those skilled in the art. Such quaternary ammonium compounds are within the scope of the compounds disclosed herein.

The compounds of the invention may exist as mono- or di-salts depending upon the pK_{d} of the acid from which the salt is formed.

It will be appreciated that for use in medicine the salts of the compounds disclosed herein should be pharmaceutically acceptable. Suitable pharmaceutically acceptable salts will be apparent to those skilled in the art. Pharmaceutically acceptable salts include those described by Berge, Bighley and Monkhouse, J. Pharm. Sci. 1977, 66, pp. 1-19. Such pharmaceutically acceptable salts include acid addition salts formed with inorganic acids e.g., hydrochloric, hydrobromic, sulfuric, nitric acid, phosphoric acid sulfuric acid, and perchloric acid and organic acids e.g., succinic, maleic, acetic, oxalic, malonic, fumaric, citric, tartaric, benzoic, p-toluenesulfonic, methanesulfonic or naphthalenesulfonic acid. Other salts e.g., oxalates or formates may be used, for example in the isolation of compounds disclosed herein and are included within the scope of this invention. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Salts formed using conventional methods in the art such as ion exchange are also included. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The compounds disclosed herein may form acid addition salts with one or more equivalents of the acid. The scope of the present invention includes all possible stoichiometric and non-stoichiometric forms.

### Preparation Methods

According to a further aspect of the present disclosure, provided is a process of preparing a compound of Formula (I), or a tautomer, stereoisomer, prodrug, crystal form, isotopically labeled form, pharmaceutically acceptable salt, hydrate, or solvate thereof. The following schemes are examples of synthetic schemes that may be used to synthesize the compound of Formula (I). In the following schemes, reactive groups can be protected with protecting groups and de-protected by well-established techniques in the art. The compound of Formula (I) described in the present disclosure may be prepared by those skilled in the organic synthesis field by using a standard method, which is discussed below in detail.

According to a further aspect of the present disclosure, a process of preparing the compound of Formula (I) as herein defined or a tautomer, stereoisomer, prodrug, crystal form, isotopically labeled form, pharmaceutically acceptable salt, hydrate, or solvate thereof, comprises any one of Processes A and B:
Process A produces the compound of Formula (II) (i.e., the compound of Formula (I), wherein L¹ is methylene and R⁶ is NR⁶¹R⁶²);
Process B produces the compound of Formula (III) (i.e., the compound of Formula (I), wherein L¹ is absent);

### Process A

Process A prepares the compound of Formula (II) as herein defined or a tautomer, stereoisomer, prodrug, crystal form, isotopically labeled form, pharmaceutically acceptable salt, hydrate, or solvate thereof, which comprises:
(i) preparing a compound of Formula (A-1):

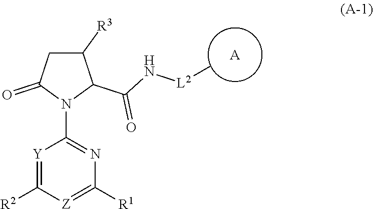

(ii) reacting the compound of Formula (A-1) with a compound of Formula (A-2) to produce a compound of Formula (A-3):

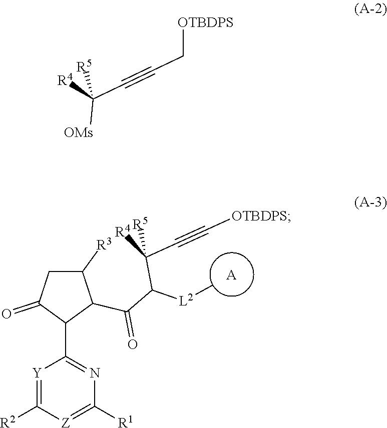

(iii) reacting the compound of Formula (A-3) with tetrabutylammonium fluoride to obtain the compound of Formula (A-4):

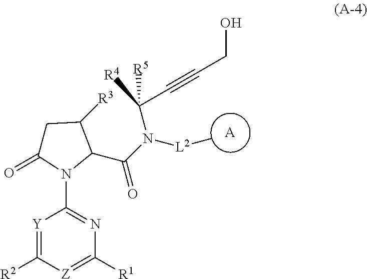

(iv) reacting the compound of Formula (A-4) with Dess-Martin periodinane (DMP) to obtain the compound of Formula (A-5):

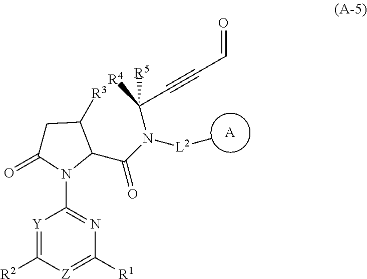

(v) reacting the compound of Formula (A-5) with a compound of Formula (A-6) to obtain the compound of Formula (II):

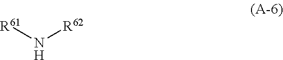

wherein
R¹-R⁵, Y, Z, L², ring A, R⁶¹, and R⁶² are as defined above.

In one embodiment, an exemplary reaction scheme of Process A may be shown as Scheme I:

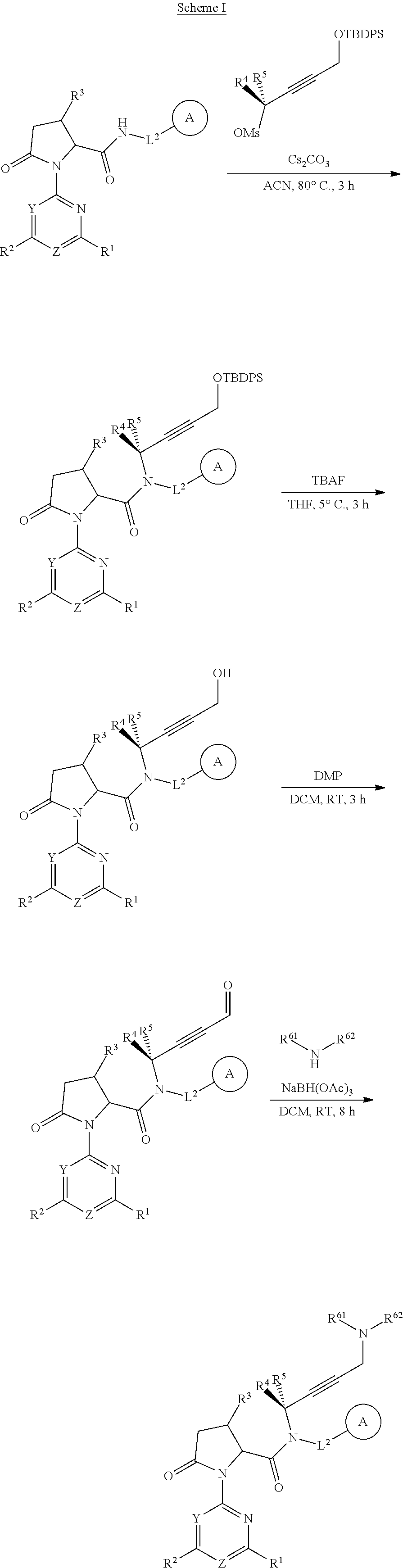

### Process B

Process B prepares the compound of Formula (III) as herein defined or a tautomer, stereoisomer, prodrug, crystal form, isotopically labeled form, pharmaceutically acceptable salt, hydrate, or solvate thereof, which comprises:
(i) preparing a compound of Formula (B-1):

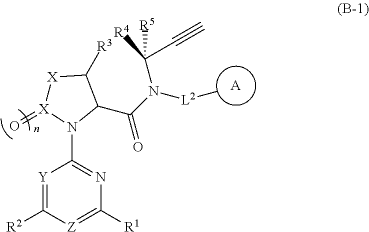

(ii) reacting the compound of Formula (B-1) with a compound of Formula (B-2) to produce the compound of Formula (III):

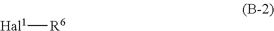

wherein R¹-R⁵, X, Y, Z, L², ring A, R⁶, W, and n are as defined above, and
Hal¹ is a halide, preferably iodide.

In one embodiment, an exemplary reaction scheme of Process B may be shown as Scheme II:

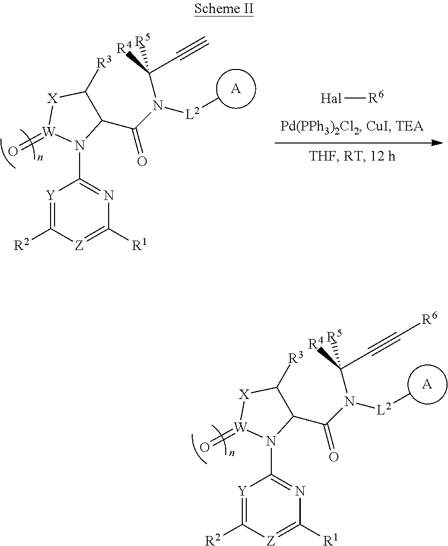

In the embodiment where the compound of Formula (III) is the compound of Formula (IIIa), the step (i) comprises:
(i-1) preparing a compound of Formula (ba-1):

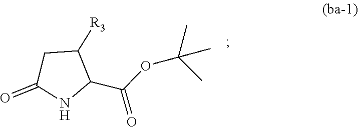

(i-2) reacting the compound of Formula (ba-1) with a compound of Formula (ba-2) to obtain a compound of Formula (ba-3):

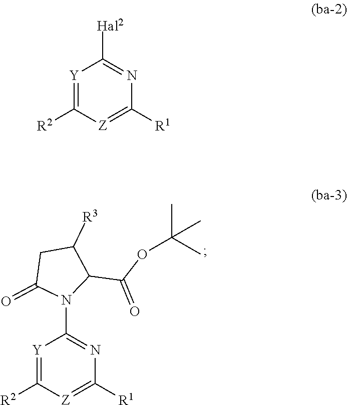

(i-3) reacting the compound of Formula (ba-3) with trifluoroacetic acid (TFA) to obtain a compound of Formula (ba-4):

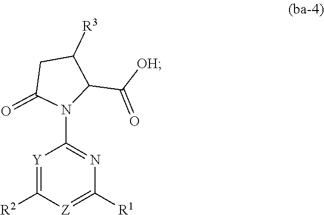

(i-4) reacting the compound of Formula (ba-4) with a compound of Formula (ba-5) to obtain a compound of Formula (ba-6):

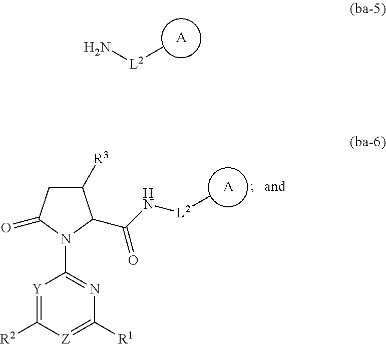

(i-5) reacting the compound of Formula (ba-6) with a compound of Formula (ba-7) to obtain a compound of Formula (IIIa):

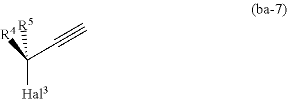

wherein R¹-R⁵, Y, Z, L², and ring A are as defined above, and
each of Hal² and Hal³ is a halide, preferably chloride, or bromide.

In one embodiment, an exemplary reaction scheme of step (i) may be shown as Scheme III:

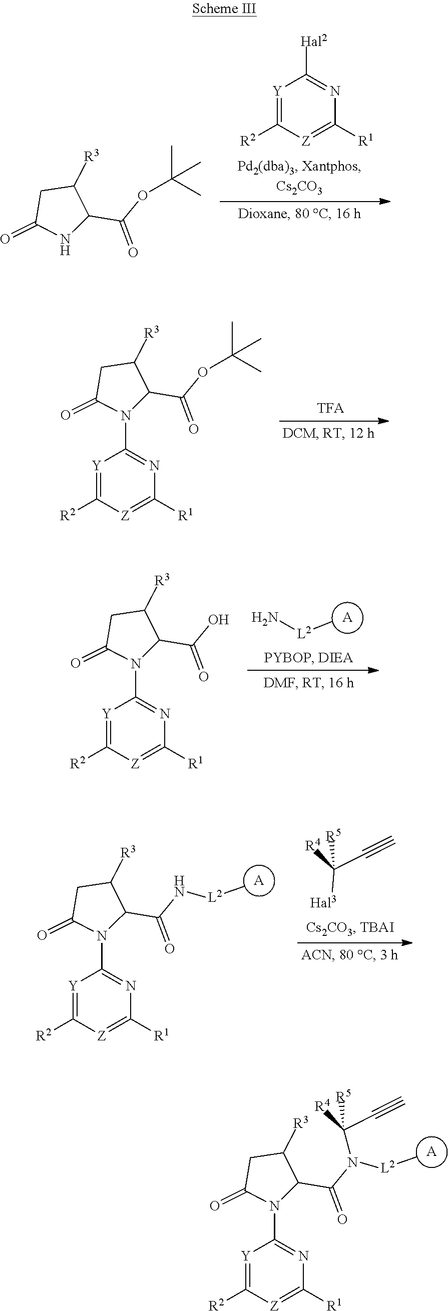

In the embodiment where the compound of Formula (III) is the compound of Formula (IIIb), the step (i) comprises:
(i-1) preparing a compound of Formula (bb-1):

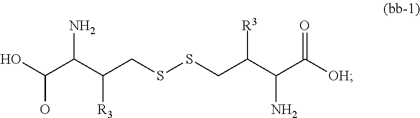

(i-2) reacting the compound of Formula (bb-1) with MeOH and SOCl₂ to obtain a compound of Formula (bb-2):

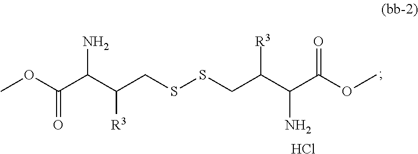

(i-3) introducing Cl₂ gas to the compound of Formula (bb-2) to obtain a compound of Formula (bb-3):

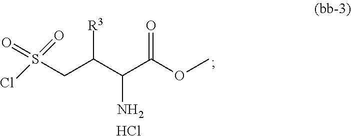

(i-4) reacting the compound of Formula (bb-3) with triethylamine to obtain a compound of Formula (bb-4):

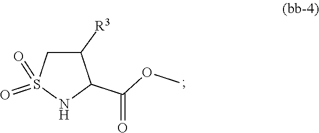

(i-5) reacting the compound of Formula (bb-4) with a compound of Formula (bb-5) to obtain a compound of Formula (bb-6):

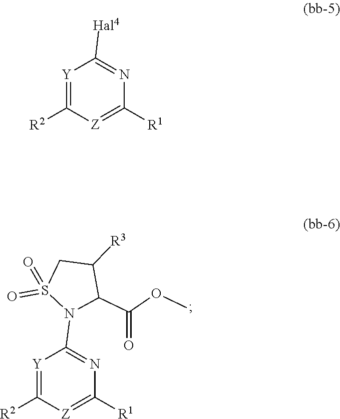

(i-6) reacting the compound of Formula (bb-6) with a compound of Formula (bb-7) to obtain a compound of Formula (bb-8):

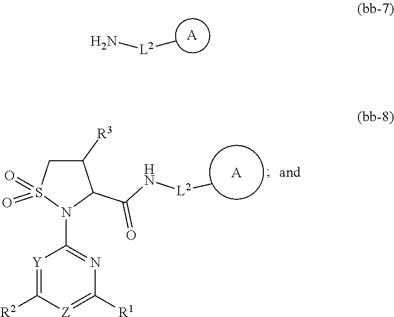

(i-7) reacting the compound of Formula (bb-8) with a compound of Formula (bb-9) to obtain a compound of Formula (IIIb):

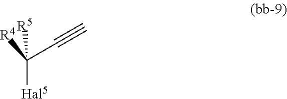

wherein R¹-R⁵, Y, Z, L², and ring A are as defined above, and
each of Hal⁴ and Hal⁵ is a halide, preferably bromide.

In one embodiment, an exemplary reaction scheme of step (i) may be shown as Scheme IV:

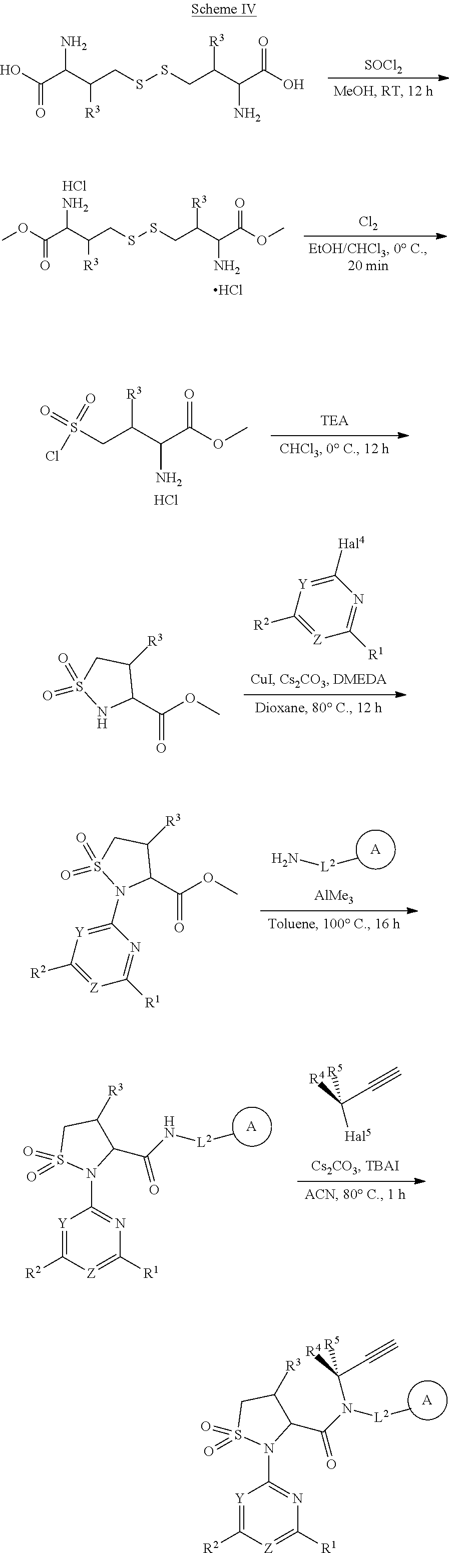

In the embodiment where the compound of Formula (III) is the compound of Formula (IIIc), the step (i) comprises:
(i-1) preparing a compound of Formula (bc-1):

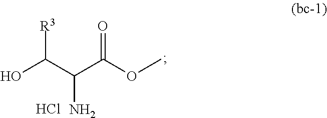

(i-2) reacting the compound of Formula (bc-1) with benzaldehyde to obtain a compound of Formula (bc-2):

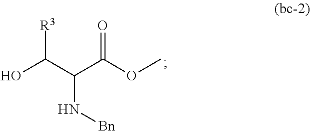

(i-3) reacting the compound of Formula (bc-2) with t-BuOH and N-(oxomethylene)sulfamoyl chloride to obtain a compound of Formula (bc-3):

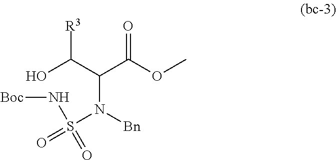

(i-4) reacting the compound of Formula (bc-3) with PPh₃ to obtain a compound of Formula (bc-4):

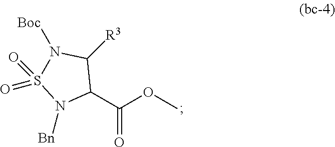

(i-5) reacting the compound of Formula (bc-4) with TFA to obtain a compound of Formula (bc-5):

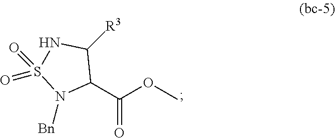

(i-6) reacting the compound of Formula (bc-5) with a compound of Formula (bc-6) to obtain a compound of Formula (bc-7):

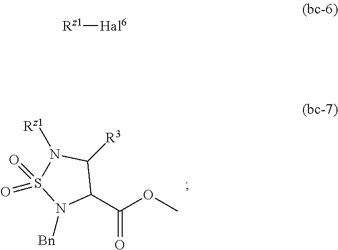

(i-7) reacting the compound of Formula (bc-7) with a compound of Formula (bc-8) to obtain a compound of Formula (bc-9):

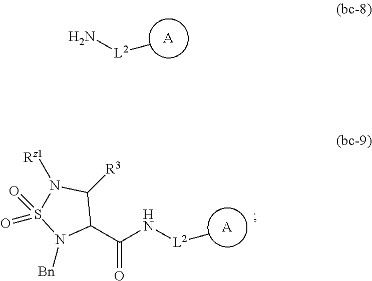

(i-8) reacting the compound of Formula (bc-9) with a compound of Formula (bc-10) to obtain a compound of Formula (bc-11):

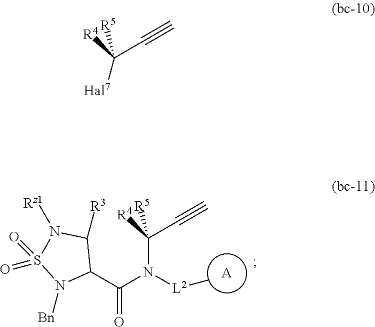

(i-9) reacting the compound of Formula (bc-11) with H₂ under a catalyst of Pd/C to obtain a compound of Formula (bc-12):

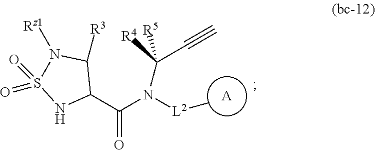

(i-10) reacting the compound of Formula (bc-12) with a compound of Formula (bc-13) to obtain a compound of Formula (IIIc):

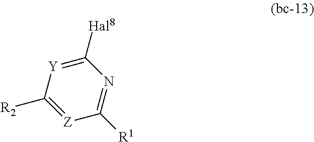

wherein R¹-R⁵, Y, Z, L², R^{z1}, and ring A are as defined above, and
each of Hal⁶, Hal⁷, and Hal⁸ is a halide, preferably iodide, or bromide.

In one embodiment, an exemplary reaction scheme of step (i) may be shown as Scheme V:

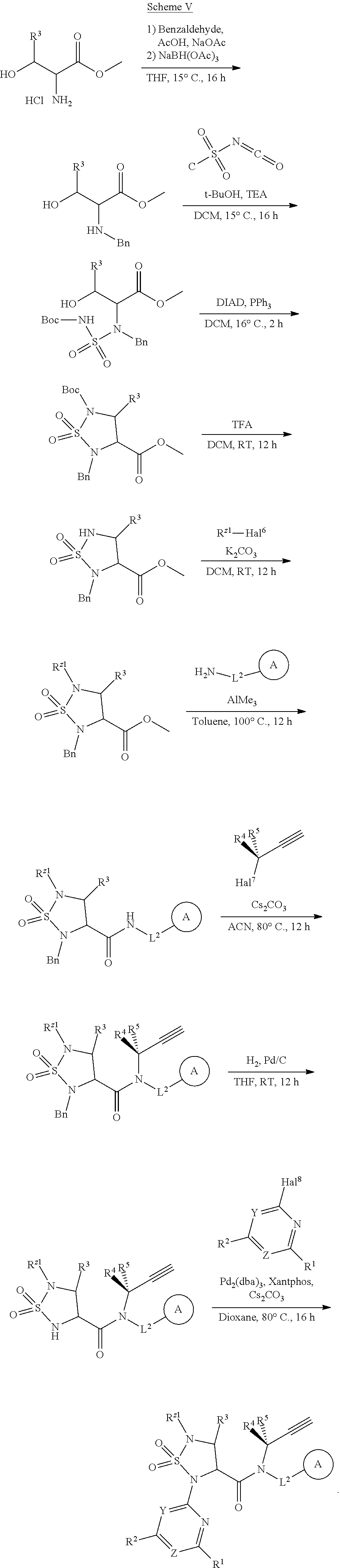

In the embodiment where the compound of Formula (III) is the compound of Formula (IIId), the step (i) comprises:
(i-1) preparing a compound of Formula (bd-1):

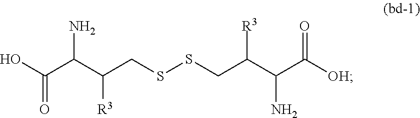

(i-2) reacting the compound of Formula (bd-1) with MeOH and SOCl₂ to obtain a compound of Formula (bd-2):

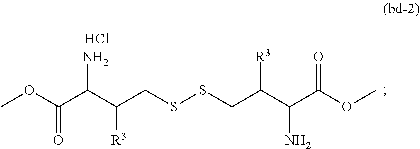

(i-3) introducing Cl₂ gas to the compound of Formula (bd-2) to obtain a compound of Formula (bd-3):

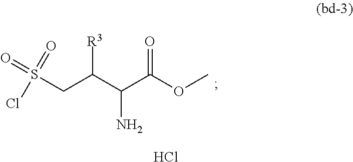

(i-4) reacting the compound of Formula (bd-3) with triethylamine to obtain a compound of Formula (bd-4):

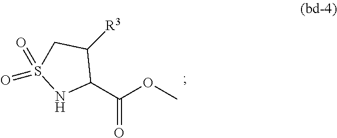

(i-5) reacting the compound of Formula (bd-4) with a compound of Formula (bd-5) to obtain a compound of Formula (bd-6):

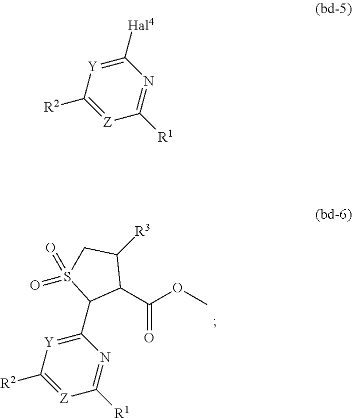

(i-6) reacting the compound of Formula (bd-6) with a compound of Formula (bd-7) to obtain a compound of Formula (bd-8):

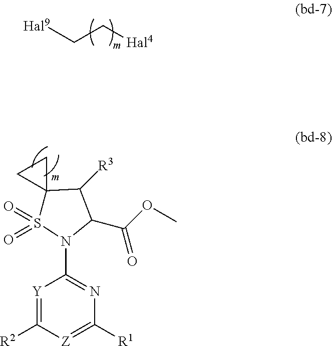

(i-7) reacting the compound of Formula (bd-8) with a compound of Formula (bd-9) to obtain a compound of Formula (bd-10):

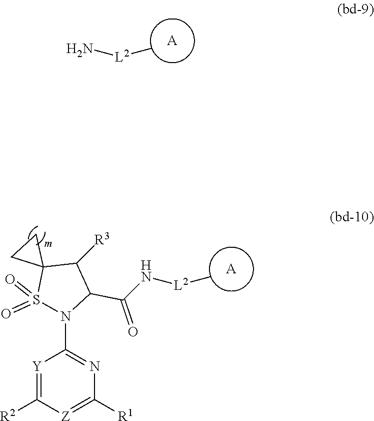

(i-8) reacting the compound of Formula (bd-10) with a compound of Formula (bd-11) to obtain a compound of Formula (IIId):

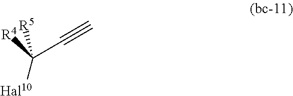

wherein R¹-R⁵, Y, Z, L², m, and ring A are as defined above, and
each of Hal⁴, Hal⁹, and Hal¹⁰ is a halide, preferably bromide.

In one embodiment, an exemplary reaction scheme of step (i) may be shown as Scheme VI:

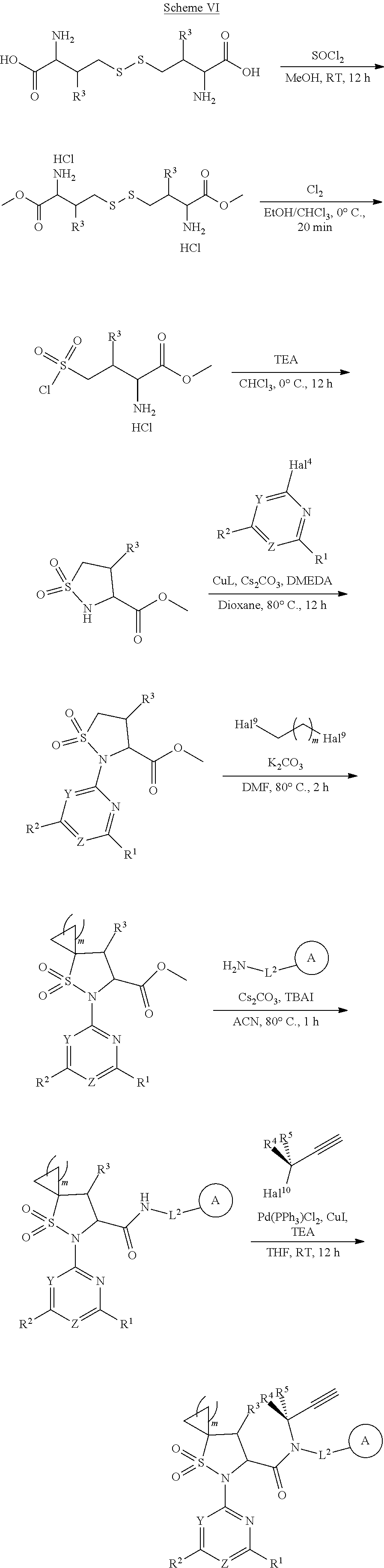

### Therapeutic Utilities

The terms “treat”, “treating”, “treatment” and the like refer to a course of action (such as administering an inhibitor of Polθ or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, “treatment” may also refer to inhibiting (e.g., arresting the development or further development of the disease, disorder or condition or clinical symptoms association therewith) an active disease.

The terms “prevent”, “preventing”, “prevention” and the like refer to a course of action (such as administering a Polθ inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting the progression of the disease, disorder or condition such that it does not reach a harmful or otherwise undesired state.

The terms “inhibiting” and “reducing,” or any variation of these terms in relation of Polθ, may refer to any measurable decrease or complete inhibition to achieve a desired result. For example, there may be a decrease in Polθ activity of about 5%, 10%, 15%, 20%, 25%, 30%, 35%, 40%, 45%, 50%, 55%, 60%, 65%, 70%, 75%, 80%, 85%, 90%, 95%, 99%, or more, compared to a control group. The term “about” as used herein means variations within ±20%, preferably, within ±10%, more preferably within ±5% of a given value.

“Disease”, “disorder” and “condition” are used interchangeably herein.

The present disclosure provides a compound that prevents or treats a disease or disorder mediated by Polθ or a disease or disorder in which Polθ activity is implicated.

For some embodiments, the disease or disorder mediated by Polθ is any of proliferative disorders. The term “proliferative disorders” is used interchangeably herein and pertains to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumors, cancers, leukemias, psoriasis, bone diseases, fibroproliferative disorders (e.g., of connective tissues), and atherosclerosis.

For some embodiments, the disease or disorder mediated by Polθ is any of HR-deficient cancers, including BRCA1- and BRCA2-deficient cancer, such as breast cancer, ovarian cancer (J. Med. Chem. 2022, 65, 19, 13198-13215), prostate cancer (Biochim Biophys Acta Mol Basis Dis. 2020 Dec. 1; 1866(12):165954.), pancreas cancer (Cancers (Basel) 2022 Aug. 23; 14(17):4077), and lung cancer (Cancers 2019, 11(5), 722). The present compound, composition and method may further enhance the efficacy of cancer therapies with at least one of the following modes of action (MOAs): (1) therapeutic mode that induces DNA damage, (2) therapeutic mode that modulates cell cycles, and (3) therapeutic mode that inhibits components involved in DNA damage responses (DDRs).

In a cancer treatment, the therapeutically effective amount of the compound of Formula (I) provided herein is an amount sufficient to provide therapeutic benefits during the course of the treatment, or to delay or minimize one or more symptoms associated with cancer. In a cancer treatment, the therapeutically effective amount of a compound is the amount of the therapeutic agent that, when used alone or in combination with other therapies, provides such therapeutic benefits during the course of the treatment.

Effective amounts of the compound of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. In the context of this disclosure, the patient can be a human or non-human mammal. Typically, dosage regimens are adjusted to provide an optimum therapeutic response, i.e., to optimize safety and efficacy. Accordingly, a therapeutically effective amount is also one of which any undesired collateral effects are outweighed by the beneficial effects of administering the compound as described herein.

### Pharmaceutical Compositions

The terms “combination”, “combined”, and related terms refer to the simultaneous, separate or sequential administration of two or more therapeutic agents or therapies. For example, the compound disclosed herein may be administered with another therapeutic agent or therapy simultaneously or sequentially in separate unit dosage forms, or together in a single unit dosage form. The another therapy may be radiotherapy. The another therapeutic agent may be an anti-cancer agent. The anti-cancer agent may include DNA damage response (DDR) targeting anti-cancer agents. The DNA damage response (DDR) is a collective term for the plethora of different intra- and inter-cellular signaling events and enzyme activities that result from the induction and detection of DNA damage. There are at least three key aspects of DDR that are different in cancers compared with normal cells, which in turn make DDR an attractive source for drug targets that can (and indeed currently are) being exploited to generate new cancer therapies. Loss of one or more DDR pathways, increased replication stress, and higher levels of endogenous DNA damage are all differentiating aspects of cancer DDR that can be targeted therapeutically. A number of anti-cancer agents targeting DDR have been known and developed, e.g., PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (e.g., MK-1775, or AZD1775), PLK1 Inhibitors (e.g., Volasertib (BI 6727), Onvansertib (e.g., PCM-075, NMS-1286937), APE1 inhibitors (e.g., methoxyamine), Topoisomerase inhibitors (e.g., belotecan, CRLX101, irinotecan, LMP 400, LMP 776, NKTR-102, doxorubicin, epirubicin, etoposide, idarubicin, mitoxantrone, teniposide), etc. (see Mark J. O'Connor, Molecular Cell 60, Nov. 19, 2015, p. 547-560, Choi et al., Int J Mol Sci. 2022 February; 23(3): 1701). The compound disclosed herein also belongs to DDR targeting agents.

In some embodiment, provided is a pharmaceutical composition including the compound disclosed herein, or a tautomer, stereoisomer, prodrug, crystal form, isotopically labeled form, pharmaceutically acceptable salt, hydrate, or solvate thereof, and a pharmaceutical acceptable carrier, wherein the composition may be administered simultaneously, separately or sequentially with additional DDR targeting anti-cancer agent described above.

In some embodiment, provided is a method of treating or preventing diseases or disorders mediated by polymerase theta, in a subject, comprising administering to the subject the compound disclosed herein, or a tautomer, stereoisomer, prodrug, crystal form, isotopically labeled form, pharmaceutically acceptable salt, hydrate, or solvate thereof, wherein the method further comprises administering additional DDR targeting anti-cancer agent described above to the subject.

In some embodiment, provided is a kit comprising:
a first pharmaceutical composition or dosage form comprising the compound disclosed herein, or a tautomer, stereoisomer, prodrug, crystal form, isotopically labeled form, pharmaceutically acceptable salt, hydrate, or solvate thereof and, optionally, one or more pharmaceutically acceptable carriers; and
a second pharmaceutical composition or dosage form comprising additional DDR targeting anti-cancer agent described above, or a tautomer, stereoisomer, prodrug, crystal form, isotopically labeled form, pharmaceutically acceptable salt, hydrate, or solvate thereof and, optionally, one or more pharmaceutically acceptable carriers.

In some embodiment, the kit is for use in a method of treating and/or preventing diseases or disorders mediated by polymerase theta. In some embodiment, the first pharmaceutical composition or dosage form may be administered simultaneously, separately or sequentially with second pharmaceutical composition or dosage form. In some embodiment, the kit may further comprise a package insert comprising an instruction for simultaneous, sequential or separate use in the treatment and/or prevention of diseases or disorders mediated by polymerase theta.

The present disclosure further relates to a pharmaceutical composition, comprising a pharmaceutically effective amount of one or more of the compounds disclosed herein, and a pharmaceutically acceptable carrier(s) and/or excipient(s). The composition may further comprise at least one of additional therapeutic agents in amounts effective for achieving the treatment or prevention of diseases or disorders disclosed herein. Pharmaceutically acceptable carriers and excipients are well known in the art, and the choice of carriers and excipients will to a large extent depend on factors such as mode of administration, their effects on solubility and stability, and the nature of dosage form.

### Treatment Method

In another aspect, the present disclosure provides a method of treating or preventing diseases or disorders, such as diseases or disorders mediated by polymerase theta, in a subject in need of treatment or prevention, comprising administering to the subject at least one compound disclosed herein, or a tautomer, stereoisomer, prodrug, crystal form, isotopically labeled form, pharmaceutically acceptable salt, hydrate, or solvate thereof, or a pharmaceutical composition disclosed herein.

The terms “human”, “patient” or “subject” are used interchangeably. A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal.

### Administration

The pharmaceutical composition of the present disclosure can be administered via various routes, including, but not limited to, oral, parenteral (injected), (e.g., intravenous, subcutaneous, intramuscular, intravascular administration, or infusion), sublingual, topical, transdermal, ocular, rectal, nasal, and vaginal administration.

The pharmaceutical composition provided herein is administered in a pharmaceutically effective amount. For example, the pharmaceutically effective amount of the pharmaceutical composition may be in the range of about 0.01 mg to about 500 mg/kg of body weight, or about 10 mg to about 500 mg/kg of body weight. In one embodiment, the amount may be in the range of about 0.1 mg to about 250 mg/kg of body weight, or about 0.1 mg to about 10 mg/kg of body weight, or about 0.1 mg to about 1 mg/kg of body weight. In another embodiment, the amount be in the range of about 1 mg to about 100 mg/kg of body weight, preferably, about 10 mg to about 100 mg/kg of body weight. The amount of the composition to be administered will typically be determined by a physician, in light of the relevant circumstances, including the condition to be treated, the chosen route of administration, the actual compound to be administered, the age, weight, and response of the individual patient, the severity of the patient's symptoms, and the like.

### Formulation

The pharmaceutical composition may, for example, be in a form suitable for oral administration such as a tablet, capsule, pill, powder, sustained release formulations, solution, suspension, for parenteral injection such as a sterile solution, suspension or emulsion, for topical administration such as an ointment or cream, or for rectal administration such as a suppository. The pharmaceutical composition will include a conventional pharmaceutical carrier or excipient, and a compound of the present disclosure as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired. Suitable pharmaceutical carriers include inert diluents or fillers, water and various organic solvents. The pharmaceutical compositions may, if desired, contain additional ingredients such as flavorings, binders, excipients and the like. For oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof. Methods of preparing various pharmaceutical compositions with a specific amount of active compound are known, or will be apparent, to those skilled in the art.

### Dosage

The pharmaceutical composition of the present disclosure may be administered in a single dose or in multiple doses. Dosing may occur one time, two times, three times, four times, five times, six times, or more than six times per day. Dosing may occur once a month, once every two weeks, once a week, or once every other day. In some cases, continuous dosing is achieved and maintained as long as necessary. In some embodiments, the pharmaceutical composition of the present disclosure is administered for more than 1, 2, 3, 4, 5, 6, 7, 14, or 28 days. In some embodiments, the pharmaceutical composition of the present disclosure is administered for less than 28, 14, 7, 6, 5, 4, 3, 2, or 1 day. In some embodiments, the pharmaceutical composition of the present disclosure is administered chronically on an ongoing basis.

It is noted that the compounds, chemical moieties or groups described in conjunction with a particular aspect, embodiment or example of the invention should be understood to applicable to any other aspect, embodiment or example described herein unless incompatible therewith. All of the features disclosed herein (including any accompanying claims, and abstract), and/or all of the steps of any method or process disclosed, may be combined in any combination, except combinations where at least some of such features and/or steps are mutually exclusive. The invention is not restricted to the details of any foregoing embodiments. The invention extends to any novel one, or any novel combination, of the features disclosed herein (including any accompanying claims, and abstract), or to any novel one, or any novel combination, of the steps of any method or process disclosed.

The contents of the articles and documents referred to herein are incorporated herein by reference in its entirety as if all of their contents are described in detail herein.

### EXAMPLE

The present disclosure will be further described below in combination with specific examples. It should be understood that these examples are only used to illustrate the present disclosure and not to limit the scope of the present disclosure. The experimental methods without specific conditions used in the following examples are generally performed under the conventional conditions or the conditions recommended by the manufacturer. Unless otherwise stated, parts and percentages are parts by weight and weight percent.

Generally, in the preparation process, each reaction is carried out in an inert solvent at a temperature from room temperature to reflux temperature (e.g., 0° C. to 100° C., or alternatively 0° C. to 80° C.). The reaction time is usually 0.1-60 hours, or alternatively 0.5-24 hours.

### Abbreviations

The abbreviations as used herein have the following meanings:
Et₃N: Triethylamine
MeOH: Methanol
EtOH: Ethanol
SOCl₂: Thionyl chloride
CHCl₃: Chloroform
Na₂SO₄: Sodium sulfate
LCMS: Liquid chromatography-mass spectrometry
HPLC: High-performance liquid chromatography
Boc: tert-Butyloxycarbonyl
DCM: Dichloromethane
TFA: Trifluoroacetic acid
THF: Tetrahydrofuran
dba: Dibenzylideneacetone
ACN: Acetonitrile
EtOAc: Ethyl acetate
NaOAc: Sodium acetate
NaBH(OAc)₃: Sodium triacetoxyborohydride
AcOH: Acetic acid
TLC: Thin Layer Chromatography
PPh₃: Triphenylphosphine
DIAD: Diisopropyl azodicarboxylate
DIEA: N, N-Diisopropylethylamine
DMF: Dimethylformamide
AlMe₃: Trimethylaluminum
Py: Pyridine
DMSO: Dimethyl sulfoxide
PYBOP: Benzotriazol-1-yloxytripyrrolidinophosphonium hexafluorophosphate
TBAI: Tetra-n-butylammonium iodide

### Materials and Methods

Solvents, reagents and starting materials were purchased from commercial vendors and used as received unless stated otherwise. All reactions were performed at RT unless stated otherwise. Flash column chromatography was carried out using pre-packed columns filled with Merck flash silica gel 60 (40-63 μm) or C18 flash silica on an ISCO Combiflash Nextgen or a Biotage Selekt.

Unless state otherwise, all reagents were used without further purification. ¹H-NMR spectra were obtained in DMSO-d₆ or CDCl₃ at room temperature on a Bruker 400 MHz instrument. When more than one conformer was detected, the chemical shifts for the most abundant one was reported. Chemical shifts of ¹H NMR spectra were recorded in parts per million (ppm) on the δ scale from an internal standard of residual solvent. Splitting patterns are designed as s=singlet, d=doublet, t=triplet, q=quartet, m=multiplet, br=broad, coupling constants (Hz), and integrated and often tabulated; LC-MS conditions are described below:

### LCMS Method A:

LCMS Column: SHIMADZU Xtimate C18 2.1*30 mm, 3 um

Mobile Phase: Solvent A: water (4 L)+TFA (1.5 mL)

Solvent B: acetonitrile (4 L)+TFA (0.75 mL)

Flow Rate: 0.8 mL/min

Run time: gradient 10%-80% (solvent B) over 6 minutes and holding at 80% for 0.5 minutes

Temperature: 50° C.

### LCMS Method B:

LCMS Column: SHIMADZU Nano Chrom 120 C18 3.0*30 mm, 3 um

Mobile Phase: Solvent A: water (4 L)+TFA (1.5 mL)

Solvent B: acetonitrile (4 L)+TFA (0.75 mL)

Flow Rate: 0.8 mL/min

Run time: gradient 10%-80% (solvent B) over 6 minutes and holding at 80% for 0.5 minutes

Temperature: 50° C.

### LCMS Method C:

LCMS Column: SHIMADZU Xtimate C18 2.1*30 mm, 3 um

Mobile Phase: Solvent A: water (4 L)+TFA (1.5 mL)

Solvent B: acetonitrile (4 L)+TFA (0.75 mL)

Flow Rate: 0.8 mL/min

Run time: gradient 30%-90% (solvent B) over 6 minutes and holding at 90% for 0.5 minutes

Temperature: 50° C.

### LCMS Method D:

LCMS Column: SHIMADZU Nano Chrom 120 C18 3.0*30 mm, 3 um

Mobile Phase: Solvent A: water (4 L)+TFA (1.5 mL)

Solvent B: acetonitrile (4 L)+TFA (0.75 mL)

Flow Rate: 0.8 mL/min

Run time: gradient 30%-90% (solvent B) over 6 minutes and holding at 90% for 0.5 minutes

Temperature: 50° C.

### Experimental Procedure

### Intermediate 1: (S)-1-(6-methyl-4-(trifluoromethyl)pyridin-2-yl)-5-oxo-N-(prop-2-yn-1-yl)-N-(m-tolyl)pyrrolidine-2-carboxamide

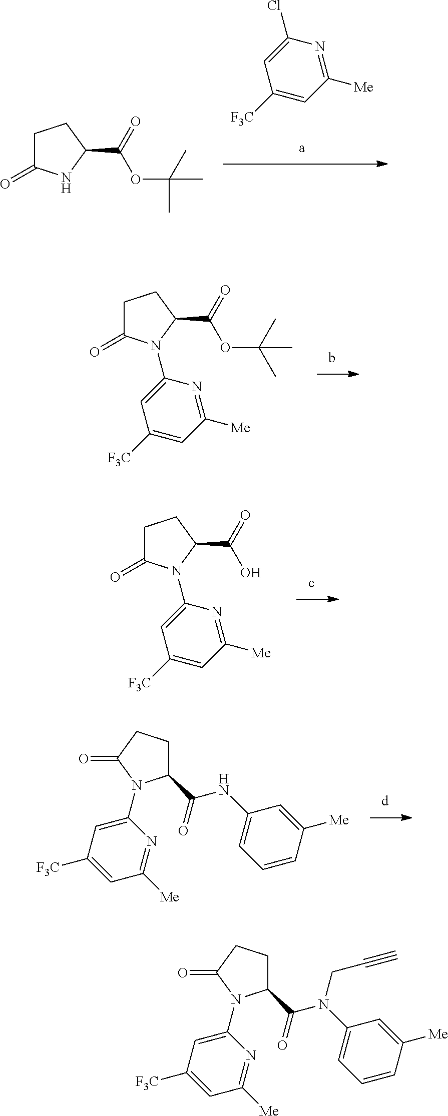 [SMILES: Cc1cc(C(F)(F)F)cc(Cl)n1 ; CC(C)(C)OC(=O)[C@@H]1CCC(=O)N1 ; Cc1cc(C(F)(F)F)cc(N2C(=O)CC[C@H]2C(=O)OC(C)(C)C)n1 ; Cc1cc(C(F)(F)F)cc(N2C(=O)CC[C@H]2C(=O)O)n1 ; Cc1cccc(NC(=O)[C@@H]2CCC(=O)N2c2cc(C(F)(F)F)cc(C)n2)c1 ; C#CCN(C(=O)[C@@H]1CCC(=O)N1c1cc(C(F)(F)F)cc(C)n1)c1cccc(C)c1]

Step a. To a solution of tert-butyl (2S)-5-oxopyrrolidine-2-carboxylate (1.85 g, 9.97 mmol) and 2-chloro-6-methyl-4-(trifluoromethyl)pyridine (1.5 g, 7.67 mmol) in dioxane (250 mL) was added Xantphos (443.80 mg, 766.99 mol), Cs₂CO₃ (6.25 g, 19.17 mmol) and Pd₂(dba)₃ (702.35 mg, 766.99 μmol). The mixture was stirred at 80° C. for 16 hrs under an N₂ atmosphere. LCMS showed the desired mass was observed. The reaction mixture was concentrated under reduced pressure to obtain a residue. The residue was purified by flash silica gel chromatography (ISCO®; 80 g Sepa Flash® Silica Flash Column, Eluent of 0-34% Dichloromethane/Petroleum ether gradient @70 mL/min) to obtain tert-butyl (2S)-1-[6-methyl-4-(trifluoromethyl)-2-pyridyl]-5-oxo-pyrrolidine-2-carboxylate (1.48 g, 4.25 mmol, 27.68% yield, 99% purity) as a yellow oil.

MS (ESI) m/z=345.1 [M+H]⁺; ¹H NMR (DMSO-d₆): δ=8.37 (s, 1H), 7.40 (s, 1H), 4.85 (dd, J=9.6, 3.2 Hz, 1H), 2.72-2.57 (m, 2H), 2.47 (s, 3H), 2.45-2.37 (m, 1H), 2.07-1.92 (m, 1H), 1.39 (s, 9H).

Step b. To a solution of tert-butyl (2S)-1-[6-methyl-4-(trifluoromethyl)-2-pyridyl]-5-oxo-pyrrolidine-2-carboxylate (1.47 g, 4.27 mmol) in DCM (12 mL) was added TFA (9.21 g, 80.78 mmol, 6 mL). The mixture was stirred at 25° C. for 12 hrs. LCMS showed the desired mass was observed. The reaction mixture was concentrated under reduced pressure to obtain a residue. The crude was directly used for the next step without further purification to obtain (2S)-1-[6-methyl-4-(trifluoromethyl)-2-pyridyl]-5-oxo-pyrrolidine-2-carboxylic acid (1.31 g, 3.06 mmol, 71.71% yield, 94% purity, TFA) as a yellow solid.

MS (ESI) m/z=289.0 [M+H]⁺; ¹H NMR (DMSO-d₆): δ=8.40 (s, 1H), 7.41 (s, 1H), 4.96 (dd, J=9.6, 3.2 Hz, 1H), 2.73-2.59 (m, 2H), 2.47 (s, 3H), 2.45-2.36 (m, 1H), 2.07-2.00 (m, 1H).

Step c. To a solution of 3-methylaniline (139.41 mg, 1.30 mmol, 140.96 μL) and (2S)-1-[6-methyl-4-(trifluoromethyl)-2-pyridyl]-5-oxo-pyrrolidine-2-carboxylic acid (250 mg, 867.39 mol) in DMF (6.2 mL) was added PYBOP (541.66 mg, 1.04 mmol) and DIEA (336.30 mg, 2.60 mmol, 453.24 μL). The mixture was stirred at 25° C. for 16 hrs. LCMS showed desired mass was observed. The reaction mixture was diluted with H₂O (20 mL) and extracted with AcOEt (20 mL×3). The combined organic layers were washed with brine (20 mL×3), dried over Na₂SO₄, filtered and concentrated under reduced pressure to obtain a residue. The residue was purified by reversed phase column (column: 80 g C18 reversed phase column; mobile phase: [Water (0.05% TFA)-ACN]; B %: 0%-55%, 30 min) to obtain (2S)-1-[6-methyl-4-(trifluoromethyl)-2-pyridyl]-N-(m-tolyl)-5-oxo-pyrrolidine-2-carboxamide (537.5 mg, 1.42 mmol, 82.11% yield, 100% purity) as a white solid.

MS (ESI) m/z=378.1 [M+H]⁺.

Step d. To a solution of 3-bromoprop-1-yne (378.29 mg, 2.54 mmol, 274.12 μL) and (2S)-1-[6-methyl-4-(trifluoromethyl)-2-pyridyl]-N-(m-tolyl)-5-oxo-pyrrolidine-2-carboxamide (480 mg, 1.27 mmol) in ACN (3.8 mL) was added Cs₂CO₃ (1.24 g, 3.82 mmol) and tetrabutylammonium iodide (28.19 mg, 76.32 μmol). The mixture was stirred at 80° C. for 3 hrs. LCMS showed desired mass was observed. The reaction mixture was diluted with H₂O (5 mL) and extracted with AcOEt (5 mL×3). The combined organic layers were washed with brine (5 mL×3), dried over Na₂SO₄, filtered and concentrated under reduced pressure to obtain a residue. The residue was purified by flash silica gel chromatography (ISCO®; 12 g Sepa Flash® Silica Flash Column, Eluent of 0-10% Ethyl acetate/Petroleum ether gradient @40 mL/min) to obtain (S)-1-(6-methyl-4-(trifluoromethyl)pyridin-2-yl)-5-oxo-N-(prop-2-yn-1-yl)-N-(m-tolyl)pyrrolidine-2-carboxamide (238.3 mg, 546.69 mol, 42.98% yield, 95.3% purity) as a yellow solid.

MS (ESI) m/z=416.1 [M+H]⁺; ¹H NMR (ES23603-144-P1H2, DMSO-d₆): δ=8.40 (s, 1H), 7.52-7.38 (m, 4H), 7.30 (d, J=7.2 Hz, 1H), 4.85 (dd, J=7.8, 4.4 Hz, 1H), 4.62 (dd, J=17.4, 2.4 Hz, 1H), 4.25 (dd, J=17.4, 2.4 Hz, 1H), 3.21 (t, J=2.4 Hz, 1H), 2.70-2.59 (m, 4H), 2.56-2.51 (m, 1H), 2.39 (s, 3H), 2.08-2.00 (m, 2H).

### Intermediate 2: methyl (3S)-2-[6-methyl-4-(trifluoromethyl)-2-pyridyl]-1,1-dioxo-1,2-thiazolidine-3-carboxylate

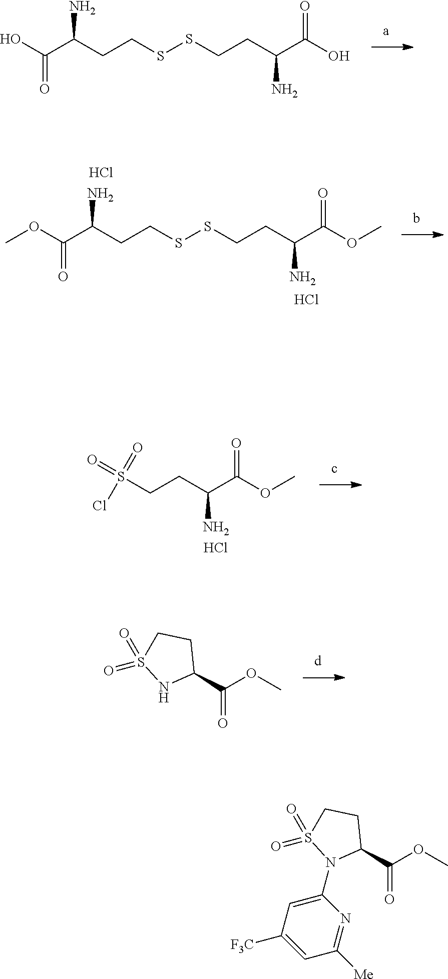

Step a. To a solution of (2S)-2-amino-4-[[(3S)-3-amino-3-carboxy-propyl]disulfanyl]-butanoic acid (20 g, 65.61 mmol, HCl) in MeOH (200 mL) was added SOCl₂ (23.42 g, 196.84 mmol, 14.30 mL) at 0° C. The mixture was stirred at 25° C. for 12 hrs. LCMS showed the desired compound was detected. The reaction mixture was concentrated under reduced pressure to obtain a methyl (2S)-2-amino-4-[[(3S)-3-amino-4-methoxy-4-oxo-butyl]disulfanyl]butanoate (28 g, crude, HCl) as a white solid.

¹H NMR (DMSO-d₆) δ=8.76 (br s, 6H), 4.14-4.10 (m, 2H), 3.75 (s, 6H), 2.99-2.78 (m, 4H), 2.21 (q, J=7.2 Hz, 4H).

Step b. Cl₂ (61 g, 860.25 mmol) gas was introduced into a mixed solution of methyl (2S)-2-amino-4-[[(3S)-3-amino-4-methoxy-4-oxo-butyl]disulfanyl]butanoate (5.3 g, 15.92 mmol, HCl) in EtOH (20 mL) and CHCl₃ (40 mL) for 20 min under 0° C., the reaction mixture was obtained as a white suspension. The white suspension was filtered, and the filter cake was washed with CHCl₃ (100 mL) and collected as a white solid. Methyl (2S)-2-amino-4-chlorosulfonyl-butanoate (4 g, crude, HCl) was obtained as a white solid, which was directly used for the next step without further purification.

¹H NMR (DMSO-d₆) δ=10.05 (br, s, 3H), 4.21 (t, J=7.6 Hz, 1H), 3.67 (s, 3H), 3.21-3.11 (m, 1H), 3.09-2.98 (m, 1H), 2.64-2.55 (m, 1H), 2.40-2.28 (m, 1H).

Step c. To a solution of methyl (2S)-2-amino-4-chlorosulfonyl-butanoate (4 g, 15.87 mmol, HCl) in CHCl₃ (50 mL) was added Et₃N (4.82 g, 47.60 mmol, 6.62 mL) at 0° C. The reaction mixture was stirred at 25° C. for 12 hrs. LCMS showed the desired compound was detected. The white suspension reaction mixture was washed with water (40 mL×3), and dried over anhydrous Na₂SO₄, filtered and concentrated under reduced pressured to obtain methyl (3S)-1,1-dioxo-1,2-thiazolidine-3-carboxylate (2.38 g, 11.95 mmol, 75.34% yield, 90% purity) as a white solid, which was directly used for the next step without further purification.

¹H NMR (DMSO-d₆) δ=7.50 (br d, J=5.6 Hz, 1H), 4.27-4.17 (m, 1H), 3.68 (s, 3H), 3.23-3.11 (m, 1H), 3.09-2.98 (m, 1H), 2.62-2.58 (m, 1H), 2.41-2.25 (m, 1H).

Step d. To a solution of methyl (3S)-1,1-dioxo-1,2-thiazolidine-3-carboxylate (1 g, 5.58 mmol in dioxane (15 mL) was added 2-bromo-6-methyl-4-(trifluoromethyl)pyridine (1.07 g, 4.46 mmol), Cs₂CO₃ (4.55 g, 13.95 mmol), N1,N2-dimethylethane-1,2-diamine (619.83 mg, 7.03 mmol, 756.81 μL) and CuI (1.34 g, 7.03 mmol). The mixture was stirred at 80° C. for 12 hrs. LCMS showed the desired compound was detected. The reaction solution was directly blow-dried, then extracted with water (100 mL) and DCM (150 mL×3). The combined organic phases were dried over Na₂SO₄, filtered and concentrated under reduced pressure to obtain the residue. The residue was purified by reversed phase column (column: 40 g C18 reversed phase column; mobile phase: [Water (0.05% TFA)-ACN]; B %: 0%-58%, 30 min) to obtain methyl (3S)-2-[6-methyl-4-(trifluoromethyl)-2-pyridyl]-1,1-dioxo-1,2-thiazolidine-3-carboxylate (660 mg, 1.95 mmol, 34.96% yield, 100% purity) as a white solid.

MS (ESI) m/z=338.9 [M+H]⁺; ¹H NMR (DMSO-d₆) δ=7.36 (s, 1H), 7.21 (s, 1H), 5.00 (dd, J=7.8, 5.2 Hz, 1H), 3.82-3.72 (m, 1H), 3.69 (s, 3H), 3.67-3.60 (m, 1H), 2.84-2.70 (m, 1H), 2.51-2.46 (m, 1H), 2.44 (s, 3H).

### Intermediate 3: (S)-5-methyl-2-(6-methyl-4-(trifluoromethyl)pyridin-2-yl)-N-(prop-2-yn-1-yl)-N-(m-tolyl)-1,2,5-thiadiazolidine-3-carboxamide 1,1-dioxide

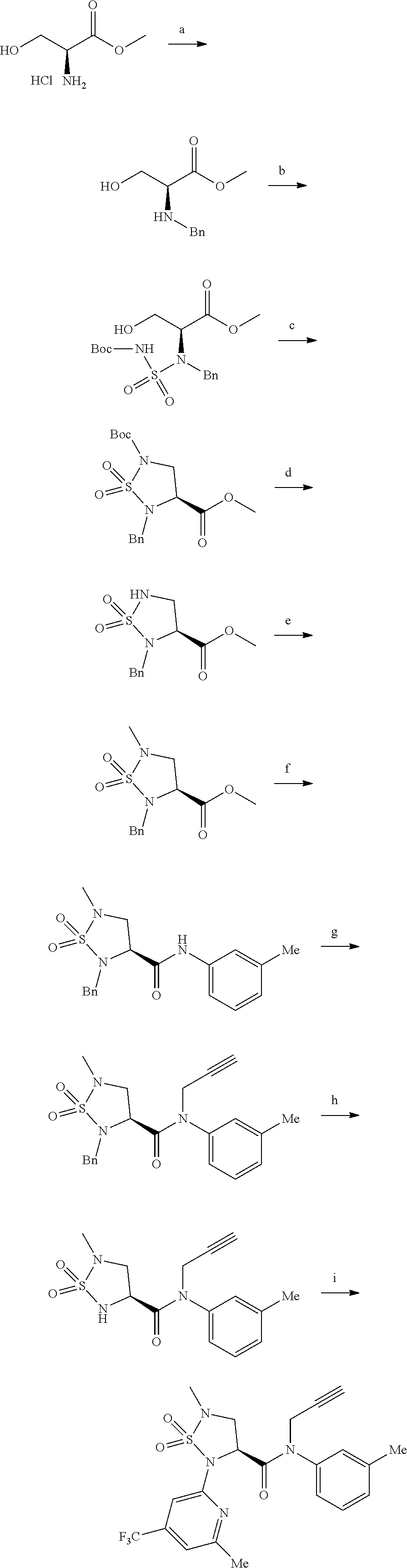

Step a. To a solution of methyl (2S)-2-amino-3-hydroxy-propanoate (5 g, 32.14 mmol, HCl) in THF (50 mL) was added benzaldehyde (2.73 g, 25.71 mmol, 2.60 mL) at 25° C. and the resulting mixture was cooled down to 0° C. AcOH (500.00 mg, 8.33 mmol, 476.64 μL) and NaOAc (2.11 g, 25.71 mmol) was added, and the reaction mixture was stirred for 1 h at 0-5° C. NaBH(OAc)₃ (11.58 g, 54.63 mmol) was added in a portion-wise manner over 30 min and the reaction mixture was stirred for 16 hrs at 15° C. LCMS showed desired mass was observed. To the suspension was added saturated NaHCO₃ solution (175 mL) over 45 min (gas evolution, pH adjusted to 8-9) at 0-5° C., then extracted with EtOAc (100 mL×3), the organic layer was washed with water (100 mL) and dried over Na₂SO₄, filtered, and concentrated under reduced pressure to obtain the residue. The residue was purified by flash silica gel chromatography (ISCO®; 80 g Sepa Flash® Silica Flash Column, Eluent of 0-80% Ethylacetate/Petroleum ether gradient @80 mL/min) to obtain methyl (2S)-2-(benzylamino)-3-hydroxy-propanoate (5.6 g, 25.93 mmol, 80.70% yield, 96.9% purity) as a colorless oil.

MS (ESI) m/z=210.1 [M+H]⁺; ¹H NMR (DMSO-d₆): δ=7.32-7.31 (m, 4H), 7.26-7.21 (m, 1H), 4.84 (t, J=4.8 Hz, 1H), 3.77 (d, J=13.6 Hz, 1H), 3.63 (s, 3H), 3.61 (d, J=6.4 Hz, 1H), 3.59-3.56 (m, 2H), 3.26 (t, J=5.2 Hz, 1H), 2.40 (br s, 1H).

Step b. A solution of t-BuOH (1.66 g, 22.45 mmol, 2.15 mL) in DCM (10 mL) was added dropwise to a solution of N-(oxomethylene)sulfamoyl chloride (2.89 g, 20.41 mmol, 1.78 mL) in DCM (30 mL) at 0° C., then the mixture was stirred at 0° C. for 1 hr under an N₂ atmosphere. Then a solution of methyl (2S)-2-(benzylamino)-3-hydroxy-propanoate (4.27 g, 20.41 mmol) and Et₃N (3.10 g, 30.61 mmol, 4.26 mL) in DCM (30 mL) was added to the above solution at 0° C. The mixture was stirred at 10-19° C. for 16 hrs. LCMS showed desired mass was observed. TLC (Plate 1, SiO₂, Petroleum ether:Ethyl acetate=1:1) showed a new spot (R_{f}=0.8) was observed. The mixture was diluted with water (100 mL) and extracted with dichloromethane (30 mL×3), the combined organic layer was washed with brine (50 mL), dried over Na₂SO₄ and filtered. The filtrate was concentrated under reduced pressure to obtain the residue. The residue was purified by column chromatography on silica gel eluting with 0-41% ethyl acetate in petroleum ether to afford methyl (2S)-2-[benzyl(tert-butoxycarbonyl sulfamoyl)amino]-3-hydroxy-propanoate (1.5 g, 3.63 mmol, 17.79% yield, 94% purity) as a yellow gum.

MS (ESI) m/z=289.0 [M-Boc+H]⁺; ¹H NMR (CDCl₃) δ=7.60-7.39 (m, 3H), 7.38-7.29 (m, 3H), 4.71-4.63 (m, 2H), 4.60-4.53 (m, 1H), 4.00 (d, J=6.8 Hz, 2H), 3.69 (s, 3H), 2.88 (s, 1H), 1.48 (s, 9H).

Step c. To a solution of methyl (2S)-2-[benzyl(tert-butoxycarbonylsulfamoyl)amino]-3-hydroxy-propanoate (3.26 g, 8.39 mmol) and PPh₃ (2.64 g, 10.07 mmol) in DCM (60 mL) was added DIAD (2.04 g, 10.07 mmol, 1.95 mL) at 0° C., then the mixture was stirred at 16° C. for 2 hrs. LCMS showed desired mass was observed. TLC (Plate 1, SiO₂, Petroleum ether:Ethyl acetate=3:1) showed a new spot (R_{f}=0.6) was observed. The mixture was concentrated under reduced pressure to obtain the residue. The residue was purified by column chromatography on silica gel eluting with 0-25% ethyl acetate in petroleum ether to afford 2-(tert-butyl) 4-methyl (S)-5-benzyl-1,2,5-thiadiazolidine-2,4-dicarboxylate 1,1-dioxide (2.56 g, 6.91 mmol, 82.37% yield, 100% purity) as a white solid.

MS (ESI) m/z=393.0 [M+Na]⁺; ¹H NMR (CDCl₃) δ=7.44-7.31 (m, 5H), 4.58-4.41 (m, 2H), 4.07-4.00 (m, 1H), 3.94-3.86 (m, 2H), 3.73 (s, 3H), 1.56 (s, 9H).

Step d. To a solution of 2-(tert-butyl) 4-methyl (S)-5-benzyl-1,2,5-thiadiazolidine-2,4-dicarboxylate 1,1-dioxide (1 g, 2.70 mmol) in DCM (10 mL) was added TFA (7.68 g, 67.31 mmol, 5 mL). The mixture was stirred at 25° C. for 12 hrs. LCMS showed the desired compound was detected. The reaction mixture was concentrated under reduced pressure to obtain methyl (3S)-2-benzyl-1,1-dioxo-1,2,5-thiadiazolidine-3-carboxylate (650 mg, 1.92 mmol, 71.26% yield, 80% purity, crude) as a white solid.

MS (ESI) m/z=292.8 [M+Na]⁺.

Step e. To a solution of methyl (3S)-2-benzyl-1,1-dioxo-1,2,5-thiadiazolidine-3-carboxylate (650 mg, 2.40 mmol) in DMF (7 mL) was added Mel (682.64 mg, 4.81 mmol, 184.98 μL) and K₂CO₃ (997.03 mg, 7.21 mmol). The mixture was stirred at 25° C. for 12 hrs. LCMS showed the desired compound was detected. The residue was purified by reversed phase column (column: 40 g C18 reversed phase column; mobile phase: [Water (0.05% TFA)-ACN]; B %: 0%-55%, 30 min) to obtain methyl (3S)-2-benzyl-5-methyl-1,1-dioxo-1,2,5-thiadiazolidine-3-carboxylate (660 mg, 2.11 mmol, 87.84% yield, 91% purity) as a white solid.

MS (ESI) m/z=325.9 [M+CH₃CN+H]⁺.

Step f. To a solution of 3-methylaniline (422.08 mg, 3.94 mmol, 426.78 μL) in toluene (7 mL) was added AlMe₃ (2 M, 2.95 mL) and methyl (3S)-2-benzyl-5-methyl-1,1-dioxo-1,2,5-thiadiazolidine-3-carboxylate (560 mg, 1.97 mmol). The mixture was stirred at 100° C. for 12 hrs. LCMS showed the desired compound was detected. The residue was purified by reversed phase column (column: 40 g C18 reversed phase column; mobile phase: [Water(0.05% TFA)-ACN]; B %: 0%-55%, 30 min) to obtain (3S)-2-benzyl-5-methyl-N-(m-tolyl)-1,1-dioxo-1,2,5-thiadiazolidine-3-carboxamide (491 mg, 1.27 mmol, 64.50% yield, 93% purity) as a white solid.

MS (ESI) m/z=359.9 [M+H]⁺.

Step g. To a solution of (3S)-2-benzyl-5-methyl-N-(m-tolyl)-1,1-dioxo-1,2,5-thiadiazolidine-3-carboxamide (289 mg, 804.02 mol) in ACN (4 mL) was added Cs₂CO₃ (654.92 mg, 2.01 mmol) and 3-bromoprop-1-yne (191.5 mg, 1.61 mmol). The mixture was stirred at 80° C. for 12 hrs. LCMS showed the desired compound was detected. The residue was purified by flash silica gel chromatography (ISCO®; 20 g SepaFlash® Silica Flash Column, Eluent of 046% Ethyl acetate/Petroleum ether gradient @30 mL/min) to obtain (S)-2-benzyl-5-methyl-N-(prop-2-yn-1-yl)-N-(m-tolyl)-1,2,5-thiadiazolidine-3-carboxamide 1,1-dioxide (250 mg, 0.63 mmol, 78.2% yield, 96% purity) as a colorless oil.

MS (ESI) m/z=398.9 [M+H]⁺.

Step h. To a solution of (S)-2-benzyl-5-methyl-N-(prop-2-yn-1-yl)-N-(m-tolyl)-1,2,5-thiadiazolidine-3-carboxamide 1,1-dioxide (600 mg, 1.61 mmol) in THF (12 mL) was added Pd/C (2 g, 1.88 mmol, 10% purity). The mixture was stirred at 25° C. for 12 hrs under an H₂ atmosphere at 40 Psi. LCMS showed the desired compound was detected. The reaction mixture was filtered, the cake was washed with EtOAc (30 mL×3), then the filtrate was concentrated under reduced pressure to obtain a residue. The residue was purified by flash silica gel chromatography (ISCO®; 12 g SepaFlash® Silica Flash Column, Eluent of 0-9% MeOH/DCM @30 mL/min to obtain (S)-5-methyl-N-(prop-2-yn-1-yl)-N-(m-tolyl)-1,2,5-thiadiazolidine-3-carboxamide 1,1-dioxide (430 mg, 1.3*mmol, 86.8% yield, 91% purity) as a white solid.

MS (ESI) m/z=331.9 [M+Na]⁺.

Step i. The i step was prepared using a method similar to those described in the synthesis of Intermediate 1(a step).

MS (ESI) m/z=467.9 [M+H]⁺.

### Example 1

### (S)—N-(3-(6-aminopyridazin-3-yl)prop-2-yn-1-yl)-1-(6-methyl-4-(trifluoromethyl)pyridin-2-yl)-5-oxo-N-(m-tolyl)pyrrolidine-2-carboxamide

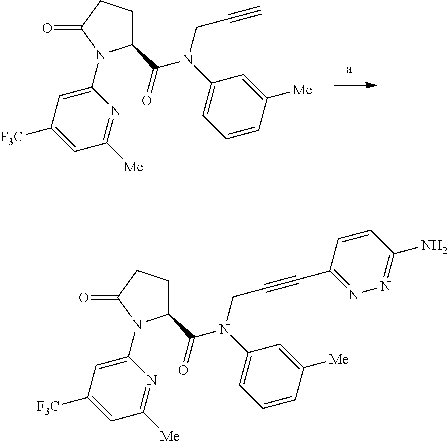 [SMILES: C#CCN(C(=O)[C@@H]1CCC(=O)N1c1cc(C(F)(F)F)cc(C)n1)c1cccc(C)c1 ; Cc1cccc(N(CC#Cc2ccc(N)nn2)C(=O)[C@@H]2CCC(=O)N2c2cc(C(F)(F)F)cc(C)n2)c1]

Step a. To a solution of 6-iodopyridazin-3-amine (75.81 mg, 343.04 μmol) and Intermediate 1 (95 mg, 228.69 μmol) in THF (2.38 mL) was added Pd(PPh₃)₂Cl₂ (32.10 mg, 45.74 μmol), Et₃N (69.42 mg, 686.07 μmol, 95.49 μL) and CuI (4.36 mg, 22.87 μmol). The mixture was stirred at 25° C. for 12 hrs under an N₂ atmosphere. LCMS showed desired mass was observed. The reaction mixture was concentrated under reduced pressure to obtain a residue. The residue was purified by prep-HPLC (column: YMC-Actus Triart C18 150*30 mm*5 um; mobile phase: [water(TFA)-ACN]; gradient:30%-50% B over 10.5 min) to obtain (S)—N-(3-(6-aminopyridazin-3-yl)prop-2-yn-1-yl)-1-(6-methyl-4-(trifluoromethyl)pyridin-2-yl)-5-oxo-N-(m-tolyl)pyrrolidine-2-carboxamide (8.3 mg, 13.33 mol, 2.92% yield, 100% purity, TFA) as a yellow solid.

MS (ESI) m/z=509.2 [M+H]⁺; ¹H NMR (MeOD): δ=8.49 (s, 1H), 7.62 (d, J=9.6 Hz, 1H), 7.57 (br s, 1H), 7.51-7.43 (m, 2H), 7.38-7.33 (m, 2H), 7.24 (s, 1H), 5.06 (dd, J=8.8, 4.0 Hz, 1H), 4.99-4.94 (m, 1H), 4.65-4.58 (m, 1H), 2.85-2.74 (m, 1H), 2.65 (s, 3H), 2.62-2.53 (m, 1H), 2.45 (s, 3H), 2.18-2.10 (m, 2H).

The Examples presented in Table 1 were prepared using methods similar to those described in the synthesis of Example 1.

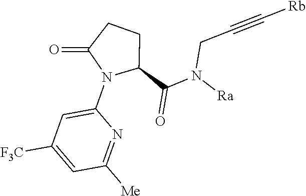

TABLE 1

| | | | | | MS |
|---|---|---|---|---|---|
| Example | | | | | (ESI) |
| Number | Ra | Rb | Name | ¹H NMR (400 MHz) δ PPM | m/z = |
| 2 | 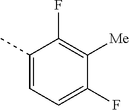 | 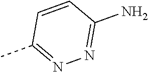 | (S)-N-(3-(6-aminopyridazin-3-yl) prop-2-yn-1-yl)-N-(2,4-difluoro- 3-methylphenyl)-1-(6-methyl- 4-(trifluoromethyl)pyridin-2-yl)- 5-oxopyrrolidine-2-carboxamide | (MeOD-d₄) δ = 8.47 (s, 1H), 7.92-7.79 (m, 1H), 7.71-7.50 (m, 1H), 7.31-7.25 (m, 1H), 7.22 (s, 1H), 7.21-7.08 (m, 1H), 6.89 (d, J = 9.2 Hz, 1H), 5.20 4.97 (m, 2H), 4.53-4.31 (m, 1H), 2.84-2.72 (m, 1H), | 545.0 [M + H]⁺. |
| | | | | 2.63-2.53 (m, 4H), 2.30 (s, | |
| | | | | 3H), 2.27-2.01 (m, 2H). | |
| 3 | 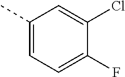 | 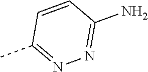 | (S)-N-(3-(6-aminopyridazin-3-yl) prop-2-yn-1-yl)-N-(3-chloro-4- fluorophenyl)-1-(6-methyl-4- (trifluoromethyl)pyridin-2-yl)-5- oxopyrrolidine-2-carboxamide | (MeOD-d₄): δ = 8.51 (s, 1H), 8.05-7.93 (m, 1H), 7.74 (br s, 1H), 7.70-7.55 (m, 1H), 7.51 (t, J = 8.8 Hz, 1H), 7.41-7.29 (m, 1H), 7.25 (s, 1H), | 547.0 [M + H]⁺. |
| | | | | 5.05 (dd, J = 8.0, 4.4 | |
| | | | | Hz, 1H), 5.01-4.96 (m, 1H), | |
| | | | | 4.62-4.59 (m, | |
| | | | | 1H), 2.88-2.71 (m, 1H), 2.68- | |
| | | | | 2.57 (m, 4H), 2.24-2.14 (m, 2H). | |
| 4 | 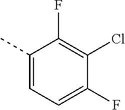 | 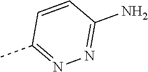 | (S)-N-(3-(6-aminopyridazin-3-yl) prop-2-yn-1-yl)-N-(3-chloro- 2,4-difluorophenyl)-1-(6-methyl- 4-(trifluoromethyl)pyridin-2- yl)-5-oxopyrrolidine-2- carboxamide | (MeOD-d₄): δ = 8.47 (br s, 1H), 7.72-7.65 (m, 1H), 7.45-7.31 (m, 1H), 7.30-7.19 (m, 2H), 6.85 (br d, J = 9.2 Hz, 1H), 5.22-4.97 (m, 2H), 4.60-4.37 (m, 1H), 2.80-2.70 (m, 1H), 2.64-2.53 (m, 4H), 2.40- | 565.0 [M + H]⁺. |
| | | | | 2.11 (m, 2H). | |
| 5 | 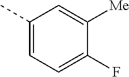 | 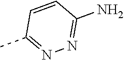 | (2S)-N-(3-(6-aminopyridazin-3- yl)prop-2-ynyl]-N-(4-fluoro-3- methyl-phenyl)-1-[6-methyl-4- (trifluoromethyl)-2-pyridyl]-5- oxo-pyrrolidine-2-carboxamide | (MeOD-d₄): δ = 8.49 (s, 1H), 7.74- 7.47 (m, 2H), 7.33-7.18 (m, 3H), 6.88 (br d, J = 8.8 Hz, 1H), 5.04 (dd, J = 8.8, 3.6 Hz, 1H), 4.95-4.95 (m, 1H), 4.56 (br s, 1H), | 527.1 [M + H]⁺. |
| | | | | 2.90-2.69 (m, 2H), 2.61 (s, 3H), | |
| | | | | 2.36 (s, 3H), 2.16 (br s, 2H). | |
| 6 | 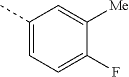 | 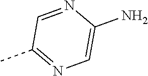 | (2S)-N-[3-(5-aminopyrazin-2-yl) prop-2-ynyl]-N-(4-fluoro-3- methyl-phenyl)-1-[6-methyl-4- (trifluoromethyl)-2-pyridyl]-5- oxo-pyrrolidine-2-carboxamide | (MeOD-d₄): δ = 8.49 (s, 1H), 7.98- 7.82 (m, 2H), 7.71-7.49 (m, 2H), 7.33-7.18 (m, 2H), 5.04 (dd, J = 8.4, 4.0 Hz, 1H), 4.94 (d, J = 17.6 Hz, 1H), 4.53 (d, J = | 527.1 [M + H]⁺. |
| | | | | 17.6 Hz, 1H), 2.85-2.74 (m, 1H), | |
| | | | | 2.61 (s, 3H), 2.60-2.53 (m, 1H), | |
| | | | | 2.36 (d, J = 1.6 Hz, 3H), | |
| | | | | 2.21-2.10 (m, 2H). | |
| 7 | 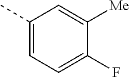 | 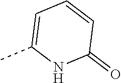 | (2S)-N-(4-fluoro-3-methyl- phenyl)-1-[6-methyl-4- (trifluoromethyl)-2-pyridyl]-5-oxo- N-[3-(6-oxo-1H-pyridin-2-yl)prop- 2-ynyl]pyrrolidine-2-carboxamide | (MeOD-d₄): δ = 8.49 (s, 1H), 7.67- 7.46 (m, 3H), 7.32-7.22 (m, 2H), 6.55 (d, J = 9.2 Hz, 1H), 6.47 (d, J = 6.8 Hz, 1H), 5.04 (dd, J = 8.4, 4.0 Hz, 1H), 4.97 (d, J = | 527.3 [M + H]⁺. |
| | | | | 17.6 Hz, 1H), 4.50 (d, J = 17.6 Hz, | |
| | | | | 1H), 2.85-2.70 (m, 1H), 2.65-2.53 | |
| | | | | (m, 4H), 2.36 (s, 3H), | |
| | | | | 2.20-2.11 (m, 2H). | |
| 8 | 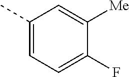 | 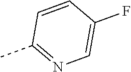 | (S)-N-(4-fluoro-3-methylphenyl)- N-(3-(5-fluoropyridin-2-yl)prop- 2-yn-1-yl)-1-(6-methyl-4- (trifluoromethyl)pyridin-2-yl)-5- oxopyrrolidine-2-carboxamide | (MeOD-d₄) δ = 8.53-8.36 (m, 2H), 7.73-7.53 (m, 3H), 7.50-7.40 (m, 1H), 7.29-7.19 (m, 2H), 5.03 (dd, J = 8.8, 4.0 Hz, 1H), 4.94 (d, J = 17.2 Hz, 1H), 4.56 (d, J = 17.2 Hz, 1H), 2.84-2.72 (m, 1H), 2.63-2.49 (m,4 H), 2.35 (d, J = 1.2 | 529.7 [M + H]⁺. |
| | | | | Hz, 3H), 2.25-2.06 (m, 2H). | |
| 9 | 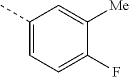 | 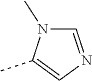 | (S)-N-(4-fluoro-3-methylphenyl)- N-(3-(1-methyl-1H-imidazol- 5-yl)prop-2-yn-1-yl)-1-(6-methyl- 4-(trifluoromethyl)pyridin-2- yl)-5-oxopyrrolidine-2- carboxamide | (MeOD-d₄) δ = 8.81 (br s, 1H), 8.49 (s, 1H), 7.73 (br s, 1H), 7.67-7.51 (m, 2H), 7.31-7.26 (m, 1H), 7.25 (s, 1H), 5.08-5.03 (m, 1H), 4.96 (d, J = 18.0 Hz, 1H), 4.70 (d, J = 17.6 Hz, 1H), 3.73 (s, 3H), 2.84-2.72 | 514.0 [M + H]⁺. |
| | | | | (m, 1H), 2.63-2.53 (m, 4H), 2.36 | |
| | | | | (s, 3H), 2.23-2.09 (m, 2H). | |
| 10 | 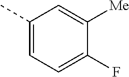 | 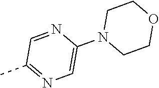 | (S)-N-(4-fluoro-3-methylphenyl)- 1-(6-methyl-4-(trifluoromethyl) pyridin-2-yl)-N-(3-(5- morpholinopyrazin-2-yl)prop-2- yn-1-yl)-5-oxopyrrolidine-2- carboxamide | (MeOD-d4) δ = 8.48 (s, 1H), 8.13 (s, 1H), 8.10 (d, J = 1.2 Hz, 1H), 7.73-7.50 (m, 2H), 7.28-7.24 (m, 1H), 7.22 (s, 1H), 5.04 (dd, J = 8.4, 4.0 Hz, 1H), 4.93 (d, J = 17.6 Hz, 1H), 4.54 (d, J = 17.6 Hz, 1H), | 597.1 [M + H]⁺. |
| | | | | 3.81-3.75 (m, 4H), 3.66-3.60 (m, | |
| | | | | 4H), 2.85-2.74 (m, 1H), 2.61 (s, | |
| | | | | 3H), 2.59 (br d, J = 4.8 | |
| | | | | Hz, 1H), 2.35 (s, 3H), 2.21-2.07 | |
| | | | | (m, 2H). | |
| 11 | 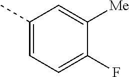 | 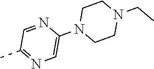 | (S)-N-(3-(5-(4-ethylpiperazin-1- yl)pyrazin-2-yl)prop-2-yn-1-yl)- N-(4-fluoro-3-methylphenyl)-1- (6-methyl-4-(trifluoromethyl) pyridin-2-yl)-5-oxopyrrolidine-2- | (MeOD-d₄) δ = 8.49 (s, 1H), 8.28 (d, J = 1.2 Hz, 1H), 8.17 (d, J = 1.2 Hz, 1H), 7.72-7.49 (m, 2H), 7.29-7.22 (m, 1H), 7.22 (s, 1H), 5.04 (dd, J = 8.0, 4.0 Hz, 1H), | 624.3 [M + H]⁺. |
| | | | carboxamide | 4.93 (d, J = 17.6 Hz, 1H), 4.63 (br s, | |
| | | | | 2H), 4.56 (d, J = 17.6 Hz, 1H), 3.67 | |
| | | | | (br s, 2H), 3.41-3.32 (m, 2H), 3.27 | |
| | | | | (q, J = 7.2 Hz, 2H), 3.14 (br s, | |
| | | | | 2H), 2.85-2.74 (m, 1H), 2.61 (s, | |
| | | | | 3H), 2.60-2.53 (m, 1H), 2.35 (d, | |
| | | | | J = 1.2 Hz, 3H), 2.20-2.12 (m, 2H), | |
| | | | | 1.39 (t, J = 7.2 Hz, 3H). | |
| 12 | 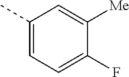 | 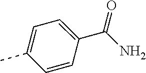 | (S)-5-(3-(N-(4-fluoro-3-methyl- phenyl)-1-(6-methyl-4-(trifluoro- methyl)pyridin-2-yl)-5-oxo- pyrrolidine-2-carboxamido)prop- 1-yn-1-yl)picolinamide | (DMSO-d₆) δ = 8.59 (s, 1H), 8.41 (s, 1H), 8.16 (br s, 1H), 8.05-8.00 (m, 1H), 7.99-7.92 (m, 1H), 7.76 (br s, 1H), 7.69-7.62 (m, 1H), 7.57 (br s, 1H), 7.46-7.35 (m, 2H), 4.96 (d, J = 17.6 Hz, 1H), 4.87 | 554.0 [M + H]⁺. |
| | | | | (dd, J = 8.8, 3.6 Hz, 1H), 4.60 (d, | |
| | | | | J = 18.0 Hz, 1H), 2.75-2.61 | |
| | | | | (m, 1H), 2.59-2.57 (m, 1H), 2.56 | |
| | | | | (s, 3H), 2.33 (s, 3H), 2.16-1.99 (m, | |
| | | | | 2H). | |

### Example 13

### (S)—N-(2,4-difluoro-3-methylphenyl)-N-(4-(4-ethylpiperazin-1-yl)but-2-yn-1-yl)-1-(6-methyl-4-(trifluoromethyl)pyridin-2-yl)-5-oxopyrrolidine-2-carboxamide

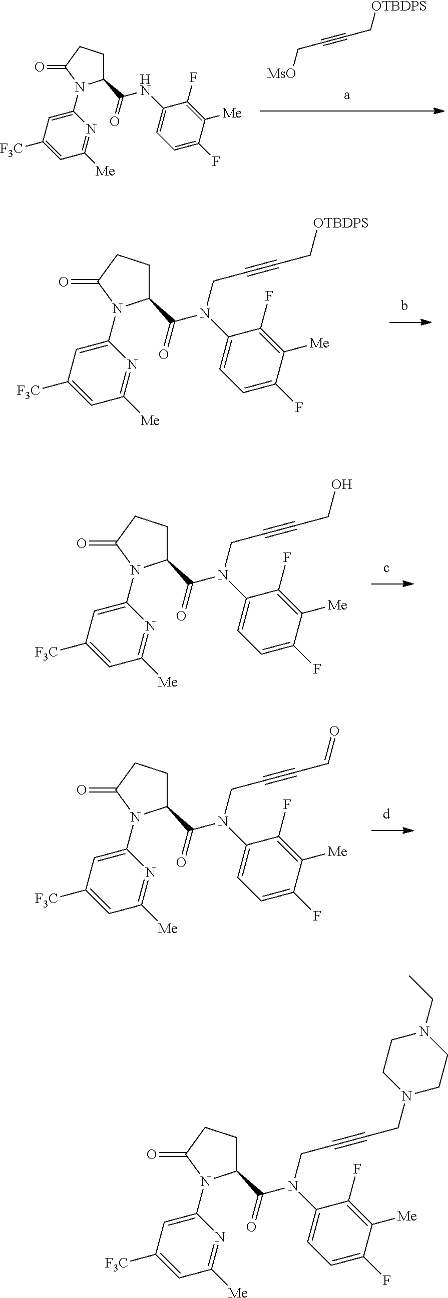

Step a. To a solution of 4-((tert-butyldiphenylsilyl)oxy) but-2-yn-1-yl methane-sulfonate (580 mg, 1.45 mmol) and(S)—N-(2,4-difluoro-3-methylphenyl)-1-(6-methy 1-4-(trifluoromethyl)pyridin-2-yl)-5-oxopyrrolidine-2-carboxamide (500 mg, 1.20 mmol) in ACN (12 mL) was added Cs₂CO₃ (781 mg, 2.4 mmol). The mixture was stirred at 80° C. for 3 hrs. LCMS showed desired mass was observed. The reaction mixture was diluted with H₂O (30 mL) and extracted with AcOEt (30 mL×3). The combined organic layers were washed with brine (10 mL×3), dried over Na₂SO₄, filtered and concentrated under reduced pressure to obtain a residue. The residue was purified by flash silica gel chromatography (ISCO®; 12 g Sepa Flash® Silica Flash Column, Eluent of 0-50% Ethyl acetate/Petroleum ether gradient @40 mL/min) to obtain (S)—N-(4-((tert-butyldiphenylsilyl)oxy)but-2-yn-1-yl)-N-(2,4-difluoro-3-methylphenyl)-1-(6-methyl-4-(trifluoromethyl)pyridin-2-yl)-5-oxopyrrolidine-2-carboxamide (680 mg, 78.1% yield, 95.0% purity) as a yellow oil. MS (ESI) m/z=720.01 [M+H]⁺

Step b. 1,245 mg of intermediate (S)—N-(4-((tert-butyldiphenylsilyl)oxy)but-2-yn-1-yl)-N-(2,4-difluoro-3-methylphenyl)-1-(6-methyl-4-(trifluoromethyl)pyridin-2-yl)-5-oxopyrrolidine-2-carboxamide was dissolved in 10 mL of tetrahydrofuran, the mixture was stirred under nitrogen protection and at a temperature of less than ° C., an appropriate amount of 1M THF in tetrabutylammonium fluoride 2.6 ml was added, and the reaction was carried out for 3 hrs; after the reaction was completed, water was added to the reaction system the reaction system was extracted with ethyl acetate three times, washed with a saturated saline solution, and dried over anhydrous sodium sulfate; filtration and concentration were performed; and then the residue was purified by column chromatography to obtain 680 mg of colorless oily compound with a yield of 81.7%.

MS (ESI) m/z=482.01 [M+H]⁺.

Step c. To a solution of (S)—N-(4-((tert-butyldiphenylsilyl)oxy)but-2-yn-1-yl)-N-(2,4-difluoro-3-methylphenyl)-1-(6-methyl-4-(trifluoromethyl)pyridin-2-yl)-5-oxopyrrolidine-2-carboxamide (570 mg, 1.183 mmol) in DCM (7 mL) was added DMP (753 mg, 1.776 mmol). The mixture was stirred at 25° C. for 3 hrs. LCMS showed the desired compound was detected. The residue was purified by column chromatography to obtain 416 mg of colorless oily compound with a yield of 73.4%. MS (ESI) m/z=480.20 [M+H]⁺.

Step d. To a solution of (S)—N-(2,4-difluoro-3-methylphenyl)-1-(6-methyl-4-(trifluoromethyl)pyridin-2-yl)-5-oxo-N-(4-oxobut-2-yn-1-yl)pyrrolidine-2-carboxamide (50 mg, 0.104 mmol) and 1-ethylpiperazine (14.3 mg, 0.125 mmol) in DCM (1 mL) was added NaBH(OAc)₃ (44 mg, 1.776 mmol). The mixture was stirred at 25° C. for 8 hrs. LCMS showed the desired compound was detected. The residue was purified by column chromatography to obtain 48 mg of white solid compound with a yield of 79.7%.

MS (ESI) m/z=578.09 [M+H]⁺; ¹H NMR (400 MHz, CDCl₃) δ=8.54 (d, J=16.9 Hz, 1H), 7.74-6.83 (m, 3H), 4.93 (m, 5H), 4.23-4.07 (m, 1H), 3.68 (s, 3H), 3.22 (s, 3H), 2.65-2.48 (m, 5H), 2.40-2.45 (m, 4H), 2.30-2.25 (m, 4H), 2.17-1.94 (m, 3H).

### Example 14

### (S)—N-(2,4-difluoro-3-methylphenyl)-1-(6-methyl-4-(trifluoromethyl)pyridin-2-yl)-N-(4-morpholinobut-2-yn-1-yl)-5-oxopyrrolidine-2-carboxamide

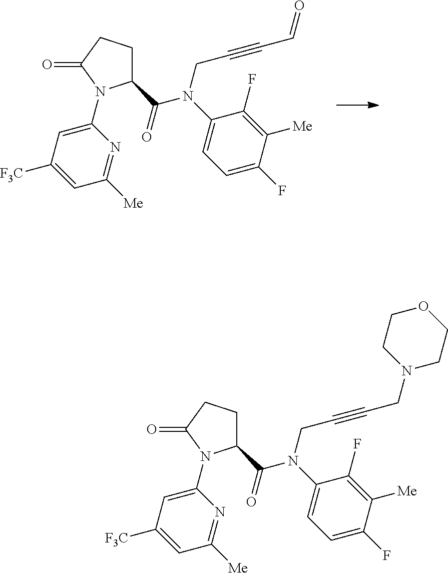 [SMILES: Cc1cc(C(F)(F)F)cc(N2C(=O)CC[C@H]2C(=O)N(CC#CC=O)c2ccc(F)c(C)c2F)n1 ; Cc1cc(C(F)(F)F)cc(N2C(=O)CC[C@H]2C(=O)N(CC#CCN2CCOCC2)c2ccc(F)c(C)c2F)n1]

Example 14 was prepared by a procedure similar to that described for the synthesis of Example 13.

MS (ESI) m/z=551.04 [M+H]⁺;

### Example 15

### (S)—N-(2,4-difluoro-3-methylphenyl)-1-(6-methyl-4-(trifluoromethyl)pyridin-2-yl)-5-oxo-N-(4-(4-(pyrazin-2-yl)piperazin-1-yl)but-2-yn-1-yl)pyrrolidine-2-carboxamide

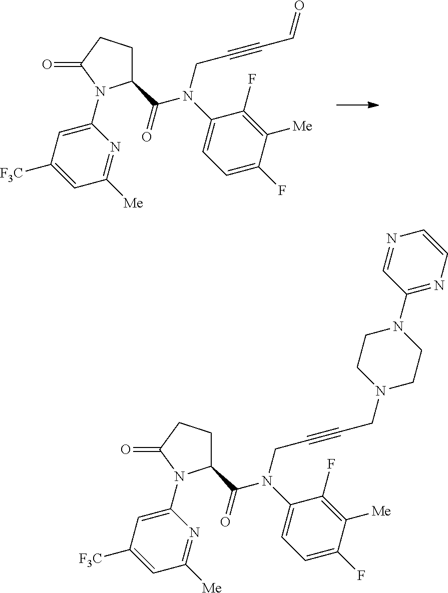 [SMILES: Cc1cc(C(F)(F)F)cc(N2C(=O)CC[C@H]2C(=O)N(CC#CC=O)c2ccc(F)c(C)c2F)n1 ; Cc1cc(C(F)(F)F)cc(N2C(=O)CC[C@H]2C(=O)N(CC#CCN2CCN(c3cnccn3)CC2)c2ccc(F)c(C)c2F)n1]

Example 15 was prepared by a procedure similar to that described for the synthesis of Example 13.

MS (ESI) m/z=628.1 [M+H]⁺;

### Example 16

### (S)—N-(2,4-difluoro-3-methylphenyl)-1-(6-methyl-4-(trifluoromethyl)pyridin-2-yl)-5-oxo-N-(4-(4-(pyrimidin-2-yl)piperazin-1-yl)but-2-yn-1-yl)pyrrolidine-2-carboxamide

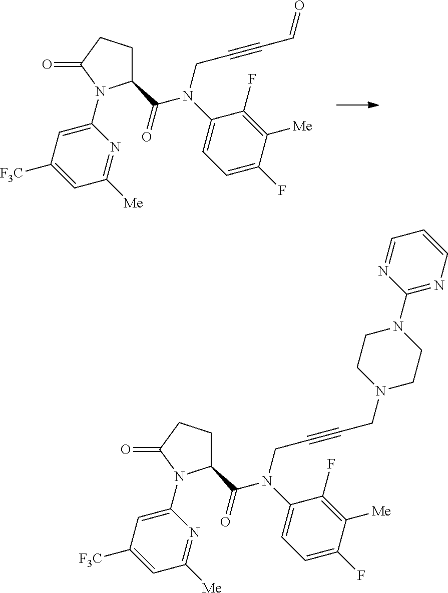 [SMILES: Cc1cc(C(F)(F)F)cc(N2C(=O)CC[C@H]2C(=O)N(CC#CC=O)c2ccc(F)c(C)c2F)n1 ; Cc1cc(C(F)(F)F)cc(N2C(=O)CC[C@H]2C(=O)N(CC#CCN2CCN(c3ncccn3)CC2)c2ccc(F)c(C)c2F)n1]

Example 16 was prepared by a procedure similar to that described for the synthesis of Example 13.

MS (ESI) m/z=628.1 [M+H]⁺.

### Example 17

### (S)—N-(3-(5-aminopyrazin-2-yl)prop-2-yn-1-yl)-N-(2,4-difluoro-3-methylphenyl)-2-(6-methyl-4-(trifluoromethyl)pyridin-2-yl)isothiazolidine-3-carboxamide 1,1-dioxide

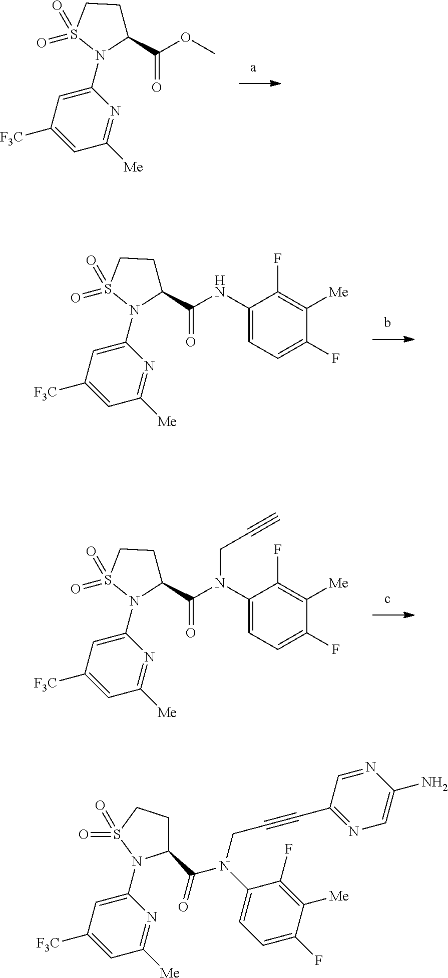 [SMILES: COC(=O)[C@@H]1CCS(=O)(=O)N1c1cc(C(F)(F)F)cc(C)n1 ; Cc1cc(C(F)(F)F)cc(N2[C@H](C(=O)Nc3ccc(F)c(C)c3F)CCS2(=O)=O)n1 ; C#CCN(C(=O)[C@@H]1CCS(=O)(=O)N1c1cc(C(F)(F)F)cc(C)n1)c1ccc(F)c(C)c1F ; Cc1cc(C(F)(F)F)cc(N2[C@H](C(=O)N(CC#Cc3cnc(N)cn3)c3ccc(F)c(C)c3F)CCS2(=O)=O)n1]

Step a. A mixture of Intermediate 2 (1.2 g, 3.55 mmol), 2,4-difluoro-3-methyl-aniline (761.57 mg, 5.32 mmol), AlMe₃ (5 M, 2.13 mL) in toluene (20 mL) was degassed and purged 3 times with N₂, and then the mixture was stirred at 100° C. for 16 hrs under an N₂ atmosphere. LCMS showed desired mass was observed. The reaction mixture was quenched by addition of NH₄Cl (10 mL), then diluted with water (20 mL) and extracted with EtOAc (20 mL×3). The combined organic layers were washed with brine (20 mL), dried over Na₂SO₄, filtered and concentrated under reduced pressure to obtain a residue. The residue was purified by flash silica gel chromatography (ISCO®; 12 g Se pa Flash® Silica Flash Column, Eluent of 0-40% Ethyl acetate:Petroleum ether @40 mL/min) to obtain (3S)—N-(2,4-difluoro-3-methyl-phenyl)-2-[6-methyl-4-(trifluoromethyl)-2-pyridyl]-1,1-dioxo-1,2-thiazolidine-3-carboxamide (1.1 g, 2.20 mmol, 62.11% yield, 90% purity) as a white solid.

MS (ESI) m/z=450.0 [M+H]⁺; ¹H NMR (DMSO-d₆) δ=10.18 (s, 1H), 7.58-7.48 (m, 1H), 7.32 (s, 1H), 7.21 (s, 1H), 7.02 (t, J=9.2 Hz, 1H), 5.16 (dd, J=7.6, 4.8 Hz, 1H), 3.83-3.63 (m, 2H), 2.85-2.73 (m, 1H), 2.46-2.42 (m, 4H), 2.17 (s, 3H).

Step b. To a solution of (3S)—N-(2,4-difluoro-3-methyl-phenyl)-2-[6-methyl-4-(trifluoromethyl)-2-pyridyl]-1,1-dioxo-1,2-thiazolidine-3-carboxamide (300 mg, 667.57 mol) in ACN (3 mL) was added 3-bromoprop-1-yne (248.17 mg, 1.67 mmol, 179.83 μL), TBAI (24.66 mg, 66.76 μmol) and Cs₂CO₃ (652.52 mg, 2.00 mmol). The mixture was stirred at 80° C. for 1 hr. LCMS showed the desired compound was detected. The crude product was further purified by Prep-HPLC (column: Welch Xtimate C18 100*40 mm*3 um; mobile phase: [water (TFA)-ACN]; gradient:45%-75% B over 8 min) to obtain (3S)—N-(2,4-difluoro-3-methyl-phenyl)-2-[6-methyl-4-(trifluoromethyl)-2-pyridyl]-1,1-dioxo-N-prop-2-ynyl-1,2-thiazolidine-3-carboxamide (340 mg, 99.49% purity) as a white solid.

MS (ESI) m/z=488.1 [M+H]⁺.

Step c. To a solution of (3S)—N-(2,4-difluoro-3-methyl-phenyl)-2-[6-methyl-4-(trifluoromethyl)-2-pyridyl]-1,1-dioxo-N-prop-2-ynyl-1,2-thiazolidine-3-carboxamide (80 mg, 164.12 mol) in THF (2 mL) was added 5-iodopyrazin-2-amine (54.41 mg, 246.18 μmol), Pd(PPh₃)₂Cl₂ (23.04 mg, 32.82 μmol), Et₃N (49.82 mg, 492.37 μmol, 68.53 μL) and CuI (3.13 mg, 16.41 μmol). The mixture was stirred at 25° C. for 12 hrs. LCMS showed the desired compound was detected. The crude product was further purified by Prep-HPLC (column: C18 100×40 mm; mobile phase: [water (TFA)-ACN]; gradient: 38%-68% B over 8 min) to obtain (3S)—N-[3-(5-aminopyrazin-2-yl) prop-2-ynyl]-N-(2, 4-difluoro-3-methyl-phenyl)-2-[6-methyl-4-(trifluoromethyl)-2-pyridyl]-1, 1-dioxo-1,2-thiazolidine-3-carboxamide (25.44 mg, 41.19 mol, 25.10% yield, 94% purity) as a white solid.

MS (ESI) m/z=581.1 [M+H]⁺; ¹H NMR (DMSO-d₆) δ=8.00-7.78 (m, 2H), 7.77-7.68 (m, 1H), 7.36-7.27 (m, 2H), 7.24-7.07 (m, 1H), 5.01 (d, J=17.6 Hz, 1H), 4.92-4.79 (m, 1H), 4.70 (dd, J=62.0, 17.6 Hz, 0.5H), 4.27 (d, J=17.6 Hz, 0.5H), 3.65-3.58 (m, 2H), 2.53 (s, 2H), 2.47 (br s, 1H), 2.46-2.37 (m, 1H), 2.26-2.12 (m, 4H).

The Examples presented in Table 2 were prepared using methods similar to those described in the synthesis of Example 17.

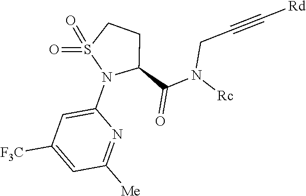

TABLE 2

| | | | | | MS |
|---|---|---|---|---|---|
| Example | | | | | (ESI) |
| Number | Rc | Rd | Name | ¹H NMR (400 MHz) δ PPM | m/z = |
| 18 | 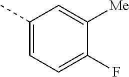 | 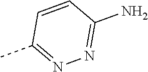 | (S)-N-(3-(6-aminopyridazin-3-yl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 7.89 (br s, 2H), 7.64-7.46 (m, 3H), 7.40-7.29 (m, 2H), 7.20 (s, 1H), 7.11 (d, J = 9.4 Hz, 1H), 4.97 (d, J = 17.6 Hz, 1H), 4.83 (t, J = 6.4 Hz, 1H), 4.49 (d, J = 17.6 Hz, 1H), 3.61-3.55 (m, 2H), 2.56 (s, 3H), 2.45-2.37 (m, 2H), 2.30 (s, 3H). | 563.0 [M + H]⁺. |
| 19 | 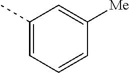 | 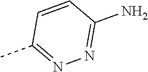 | (S)-N-(3-(6-aminopyridazin-3-yl)prop-2-yn-1- yl)-2-(6-methyl-4-(trifluoromethyl)pyridin-2- yl)-N-(m-tolyl)isothiazolidine-3-carboxamide 1,1-dioxide | (DMSO-d₆) δ = 8.01 (br s, 1H), 7.78-7.58 (m, 1H), 7.57-7.46 (m, 1H), 7.44-7.29 (m, 1H), 7.27-7.06 (m, 2H), 5.03 (br d, J = 17.6 Hz, 0.5H), 4.98-4.84 (m, 1H), 4.80-4.63 (m, 1H), 4.42 (br d, J = 17.6 Hz, 0.5H), 3.67-3.54 (m, 2H), 2.56 (5, 3H), 2.49-2.36 (m, 2H), 2.34-2.13 (m, 3H). | 581.0 [M + H]⁺. |
| 20 | 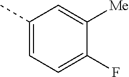 | 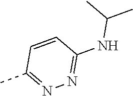 | (S)-N-(4-fluoro-3-methylphenyl)-N-(3-(6- (isopropylaminopyridazin-3-yl)prop-2-yn-1- yl)-2-(6-methyl-4-(trifluoromethyl)pyridin-2- yl)isothiazolidine-3-carboxamide 1,1-dioxide | (MeOD-d₄) δ = 7.64 (br s, 1H), 7.56 (br d, J = 9.2 Hz, 2H), 7.37 (s, 1H), 7.33 (br d, J = 9.8 Hz, 1H), 7.24 (t, J = 8.8 Hz, 1H), 7.14 (s, 1H), 5.02 (d, J = 17.6 Hz, 1H), 4.99-4.95 (m, 1H), 4.54 (d, J = 17.6 Hz, 1H), 3.98 (td, J = 12.8, 6.4 Hz, 1H), 3.60 (td, J = 12.4, 8.4 Hz, 1H), 3.48 (td, J = 12.4, 6.4 Hz, 1H), 2.62 (s, 3H), 2.58-2.45 (m, 2H), 2.34 (d, J = 1.2 Hz, 3H), 1.33 (d, J = 6.4 Hz, 6H). | 605.0 [M + H]⁺. |
| 21 | 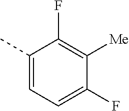 | 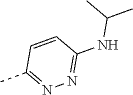 | (S)-N-(2,4-difluoro-3-methylphenyl)-N-(3-(6- (isopropylamino)pyridazin-3-yl)prop-2-yn- 1-yl)-2-(6-methyl-4-(trifluoromethyl)pyridin-2- carboxamide 1,1-dioxideyl)isothiazolidine-3- carboxamide 1,1-dioxide | (MeOD-d₄) δ = 7.91-7.79 (m, 1H), 7.59-7.51 (m, 1H), 7.40-7.28 (m, 1H), 7.21-6.95 (m, 3H), 6.83-6.73 (m, 1H), 5.1 (d, J = 17.6 Hz, 0.5H), 4.99 (d, J = 17.6 Hz, 0.5H), 4.98-4.94 (m, 1H), 4.52 (d, J = 17.6 Hz, 0.3H), 4.34 (d, J = 17.6 Hz, 0.7H), 3.50-3.45 (m, 1H), 2.64-2.49 (m, 4H), 4.18-4.14 (m, 1H), 3.68-3.56 (m, 1H), 3.50-3.45 (m, 1H), 2.64- 2.49 (m, 4H), 2.48-2.38 (m, 1H), 2.34-2.18 (m, 3H), 1.24 (d, J = 6.4 Hz, 6H). | 623.1 [M + H]⁺. |
| 22 | 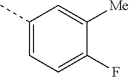 | 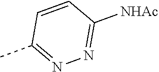 | (S)-N-(3-(6-acetamidopyridazin-3-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (MeOD-d₄) δ = 8.43 (d, J = 9.2 Hz, 1H), 7.71-7.54 (m, 3H), 7.37 (s, 1H), 7.26 (t, J = 9.2 Hz, 1H), 7.18-7.12 (m, 1H), 5.00 (d, J = 17.6 Hz, 1H), 4.97 (br dd, J = 4.8, 2.4 Hz, 1H), 4.62 (d, J = 17.6 Hz, 1H), 3.66-3.58 (m, 1H), 3.52-3.45 (m, 1H), 2.58 (s, 3H), 2.55-2.47 (m, 2H), 2.35 (d, J = 1.2 Hz, 2H), 2.29 (s, 1H), 2.23 (s, 2H), 2.15 (s, 1H). | 605.1 [M + H]⁺. |
| 23 | 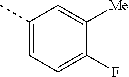 | 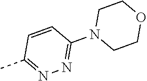 | (S)-N-(4-fluoro-3-methylphenyl)-2-(6-methyl- 4-(trifluoromethyl)pyridin-2-yl)-N-(3-(6- morpholinopyridazin-3-yl)prop-2-yn-1- yl)isothiazolidine-3-carboxamide 1,1-dioxide | (MeOD-d₄) δ = 7.64 (br s, 1H), 7.54 (br d, J = 9.6 Hz, 2H), 7.49-7.44 (m, 1H), 7.36 (s,1H), 7.24 (t, J = 8.8 Hz, 1H), 7.13 (s, 1H), 5.02-4.92 (m, 2H), 4.58 (d, J = 17.6 Hz, 1H), 3.85-3.78 (m, 4H), 3.72-3.66 (m, 4H), 3.61 (td, J = 12.8, 8.4 Hz, 1H), 3.47 (td, J = 12.4, 6.4 Hz, 1H), 2.58 (s, 3H), 2.55-2.47 (m, 2H), 2.34 (s, 3H). | 633.1 [M + H]⁺. |
| 24 | 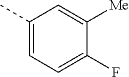 | 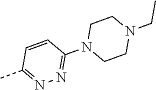 | (S)-N-(3-(6-(4-ethylpiperazin-1-yl)pyridazin-3- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (MeOD-d₄) δ = 7.72-7.54 (m, 2H), 7.48 (br d, J = 9.6 Hz, 1H), 7.39- 7.31 (m, 2H), 7.25 (t, J = 9.2 Hz, 1H), 7.12 (s, 1H), 5.04-4.92 (m, 2H), 4.58 (br d, J = 17.6 Hz, 1H), 3.82-3.56 (m, 3H), 3.50-3.45 (m, 3H), 3.30-3.24 (m, 4H), 3.23-3.20 (m, 2H), 2.58 (s, 3H), 2.56-2.47 (m, 2H), 2.35 (s, 3H), 1.39 (t, J = 7.2 Hz, 3H). | 660.3 [M + H]⁺. |
| 25 | 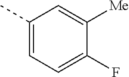 | 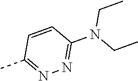 | (S)-N-(3-(6-(diethylamino)pyridazin-3-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (MeOD-d₄) δ = 7.71-7.49 (m, 4H), 7.38 (s, 1H), 7.25 (t, J = 9.2 Hz, 1H), 7.15 (s, 1H), 5.05-4.95 (m, 2H), 4.57 (d, J = 17.6 Hz, 1H), 3.70 (q, J = 7.2 Hz, 4H), 3.64-3.57 (m, 1H), 3.49 (td, J = 12.4, 6.4 Hz, 1H), 2.62 (s, 3H), 2.57-2.46 (m, 2H), 2.35 (s, 3H), 1.28 (t, J = 7.2 Hz, 6H). | 619.1 [M + H]⁺. |
| 26 | 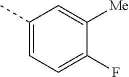 | 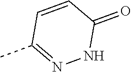 | (S)-N-(4-fluoro-3-methylphenyl)-2-(6-methyl- 4-(trifluoromethyl)pyridin-2-yl)-N-(3-(6-oxo- 1,6-dihydropyridazin-3-yl)prop-2-yn-1- yl)isothiazolidine-3-carboxamide 1,1-dioxide | (DMSO-d₆) δ = 7.67-7.51 (m, 2H), 7.41-7.30 (m, 3H), 7.20 (s, 1H), 6.89 (d, J = 9.6 Hz, 1H), 4.93 (d, J = 17.6 Hz, 1H), 4.83 (t, J = 6.4 Hz, 1H), 4.46 (d, J = 17.6 Hz, 1H), 3.72-3.57 (m, 2H), 2.57 (s, 3H), 2.47- 2.38 (m, 2H), 2.30 (s, 3H). | 564.0 [M + H]⁺. |
| 27 | 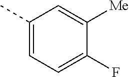 | 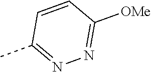 | (S)-N-(4-fluoro-3-methylphenyl)-N-(3-(6- methoxypyridazin-3-yl)prop-2-yn-1-yl)-2-(6- methyl-4-(trifluoromethyl)pyridin-2- yl)isothiazolidine-3-carboxamide 1,1-dioxide | (MeOD-d₄) δ = 7.75-7.58 (m, 2H), 7.56 (d, J = 9.2 Hz, 1H), 7.37 (s, 1H), 7.26 (t, J = 9.2 Hz, 1H), 7.16 (d, J = 9.2 Hz, 1H), 7.14 (s, 1H), 5.00-4.95 (m, 2H), 4.60 (d, J = 17.6 Hz, 1H), 4.11 (s, 3H), 3.66-3.58 (m, 1H), 3.51-3.45 (m, 1H), 2.59 (s, 3H), 2.57-2.48 (m, 2H), 2.35 (s, 3H) | 578.1 [M + H]⁺. |
| 28 | 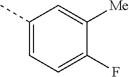 | 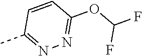 | (S)-N-(3-(6-(difluoromethoxy)pyridazin-3- yl)prop-2-yn-1-yl)-N-(4-fluoro-3- methylphenyl)-2-(6-methyl-4- (trifluoromethyl)pyridin-2-yl)isothiazolidine- 3-carboxamide 1,1-dioxide | (CD₃CN) δ = 7.65 (d, J = 3.6 Hz, 1H), 7.62 (t, J = 84.4 Hz, 1H), 7.62- 7.49(m, 2H), 7.30-7.19 (m, 3H), 7.14 (s, 1H), 4.97 (d, J = 18.0 Hz, 1H), 4.94 (t, J = 5.6 Hz, 1H), 4.55 (d, J = 18.0 Hz, 1H), 3.59-3.49 (m, 1H), 3.42-3.32 (m, 1H), 2.57 (s, 3H), 2.47-2.42 (m, 2H), 2.32 (s, 3H). | 614.0 [M + H]⁺. |
| 29 | 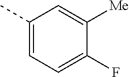 | 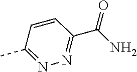 | (S)-N-(3-(6-carbamoylpyridazin-3-yl)prop- 2-yn-1-yl)-N-(4-fluoro-3-methylphenyl)-2-(6- methyl-4-yl)isothiazolidine-3- (trifluoromethyl)pyridin-2-yl)isothiazolidine- 3-carboxamide 1,1-dioxide | (DMSO-d6) δ = 8.65 (s, 1H), 8.20 (d, J = 8.8 Hz, 1H), 8.02 (s, 1H), 7.90 (d, J = 8.8 Hz, 1H), 7.76-7.53 (m, 2H), 7.39 (t, J = 9.2 Hz, 1H), 7.32 (s, 1H), 7.21 (s, 1H), 5.08 (d, 1H), 4.60 (d, J = 18.0 Hz, 1H), 3.72-3.63 (m, 2H), 2.55 (s, 3H), 2.48-2.39 (m, 2H), 2.31 (s, 3H). | 591.0 [M + H]⁺. |
| 30 | 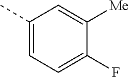 | 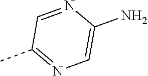 | (S)-N-(3-(5-aminopyrazin-2-yl)prop-2-yn-1-yl)- N-(4-fluoro-3-methylphenyl)-2-(6-methyl-4- (trifluoromethyl)pyridin-2-yl)isothiazolidine- 3-carboxamide 1,1-dioxide | (DMSO-d₆) δ = 7.96-7.77 (m, 2H), 7.66-7.50 (m, 2H), 7.41-7.30 (m, 2H), 7.19 (s, 1H), 6.89 (br s, 2H), 4.93 (d, J = 17.6 Hz, 1H), 4.81 (t, J = 6.4 Hz, 1H), 4.39 (d, J = 17.6 Hz, 1H), 3.57-3.49 (m, 2H), 2.55 (s, 3H), 2.45-2.38 (m, 2H), 2.29 (s, 3H). | 563.0 [M + H]⁺. |
| 32 | 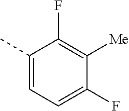 | 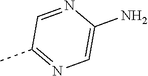 | (S)-N-(3-(5-aminopyrazin-2-yl)prop-2-yn-1-yl)- N-(2,4-difluoro-3-methylphenyl)-2-(6-methyl- 4-(trifluoromethyl)pyridin-2-yl)isothiazolidine- 3-carboxamide 1,1-dioxide | (MeOD-d₄) δ = 7.93-7.81 (m, 2H), 7.77-7.45 (m, 1H), 7.42-7.31 (m, 1H), 7.23-7.01 (m, 2H), 5.34 (t, J = 4.8 Hz, 0.5H), 5.15 (d, J = 17.6 Hz, 0.5H), 5.00-4.95 (m, 1H), 4.51 (d, J = 17.6 Hz, 0.5H), 4.31 (d, J = 17.5 Hz, 1H), 3.67-3.57 (m, 1H), 3.53-3.43 (m, 1H), 2.58-2.53 (m, 3H), 2.34-2.25 (m, 3H), 2.23-2.12 (m, 1H), 2.07-2.01 (m, 1H). | 581.0 [M + H]⁺. |
| 33 | 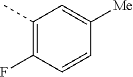 | 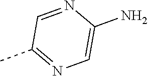 | (S)-N-(3-(5-aminopyrazin-2-yl)prop-2-yn-1-yl)- N-(2-fluoro-5-methylphenyl)-2-(6-methyl-4- (trifluoromethyl)pyridin-2-yl)isothiazolidine-3- carboxamide 1,1-dioxide | (MeOD-d₄) δ = 7.88 (s, 1H), 7.84-7.79 (m, 1.7H), 7.46 (br d, J = 8.0 Hz, 0.3H), 7.41-7.32 (m, 2H), 7.31-7.23 (m, 1H), 7.18-7.12 (m, 1H), 5.10 (d, J = 17.6 Hz, 0.7H), 5.02-4.90 (m, 1.3H), 4.53 (d, J = 17.6 Hz, 0.3H), 4.32 (d, J = 17.6 Hz, 0.7H), 3.68-3.57 (m, 1H), 3.52-3.43 (m, 1H), 2.62-2.50 (m, 4H), 2.50-2.38 (m, 4H). | 562.9 [M + H]⁺. |
| 34 | 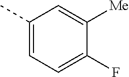 | 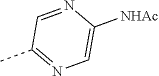 | (S)-N-(3-(5-acetamidopyrazin-2-yl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 10.97 (s, 1H), 9.28 (s, 1H), 8.40 (d, J = 1.2 Hz, 1H), 7.72- 7.51 (m, 2H), 7.39 (t, J = 9.2 Hz, 1H), 7.33 (s, 1H), 7.20 (s, 1H), 4.98 (d, J = 17.6 Hz, 1H), 4.84 (t, J = 6.4 Hz, 1H), 4.49 (d, J = 17.6 Hz, 1H), 3.71- 3.64 (m, 2H), 2.56 (s, 3H), 2.48-2.39 (m, 2H), 2.31 (s, 3H), 2.15 (s, 3H). | 605.0 [M + H]⁺. |
| 35 | 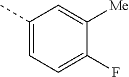 | 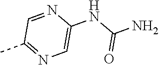 | (S)-N-(4-fluoro-3-methylphenyl)-2-(6-methyl-4- (trifluoromethyl)pyridin-2-yl)-N-(3-(5- ureidopyrazin-2-yl)prop-2-yn-1- yl)isothiazolidine-3-carboxamide 1,1-dioxide | (DMSO-d₆) δ = 9.59 (s, 1H), 8.91 (d, J = 1.2 Hz, 1H), 8.26 (d, J = 1.2 Hz, 1H), 7.72-7.52 (m, 2H), 7.39 (t, J = 9.2 Hz, 1H), 7.33 (s, 1H), 7.20 (s, 1H), 6.81 (br s, 2H), 4.98 (br d, J = 17.6 Hz, 1H), 4.83 (t, J = 6.4 Hz, 1H), 4.46 (br d, J = 17.6 Hz, 1H), 3.72-3.56 (m, 2H), 2.56 (s, 3H), 2.48-2.38 (m, 2H), 2.31 (s, 3H). | 606.2 [M + H]⁺. |
| 36 | 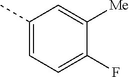 | 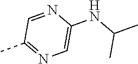 | (S)-N-(4-fluoro-3-methylphenyl)-N-(3-(5- (isopropylamino)pyrazin-2-yl)prop-2-yn-1-yl)- 2-(6-methyl-4-(trifluoromethyl)pyridin-2- yl)isothiazolidine-3-carboxamide 1,1-dioxide | (DMSO-d₆) δ = 7.96 (d, J = 1.6 Hz, 1H), 7.82 (d, J = 1.6 Hz, 1H), 7.67- 7.58 (br s, 1H), 7.54 (br s, 1H), 7.44-7.34 (m, 2H), 7.32 (s, 1H), 7.19 (s, 1H), 4.94 (d, J = 17.6 Hz, 1H), 4.81 (t, J = 6.4 Hz, 1H), 4.39 (d, J = 17.6 Hz, 1H), 4.05-3.92 (m, 1H), 3.69-3.63 (m, 2H), 2.55 (s, 3H), 2.47-2.36 (m, 2H), 2.29 (s, 3H), 1.15 (d,J = 6.8 Hz, 6H). | 605.2 [M + H]⁺. |
| 37 | 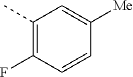 | 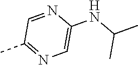 | (S)-N-(2-fluoro-5-methylphenyl)-N-(3-(5- (isopropylamino)pyrazin-2-yl)prop-2-yn-1-yl)- 2-(6-methyl-4-(trifluoromethyl)pyridin-2- yl)isothiazolidine-3-carboxamide 1,1-dioxide | (MeOD-d₄) δ = 7.90 (s, 1H), 7.81 (br d, J = 6.8 Hz, 1H), 7.74 (br s, 1H), 7.41-7.32 (m, 2H), 7.27 (br t, J = 8.8 Hz, 1H), 7.19-7.10 (m, 1H), 5.10 (d, J = 17.6 Hz, 0.7H), 5.02-4.92 (m, 1.3H), 4.58 (d, J = 17.6 Hz, 0.3H), 4.32 (d, J = 17.6 Hz, 0.7H), 4.13-3.99 (m, 1H), 3.62-3.58 (m, 1H), 3.52-3.42 (m, 1H), 2.67-2.49 (m, 4H),2.46-2.38 (m, 4H), 1.21 (d, J = 6.4 Hz, 6H). | 605.0 [M + H]⁺. |
| 38 | 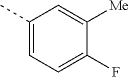 | 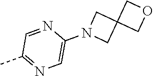 | (S)-N-(3-(5-(2-oxa-6-azaspiro[3.3]heptan-6- yl)pyrazin-2-yl)prop-2-yn-1-yl)-N-(4-fluoro-3- methylphenyl)-2-(6-methyl-4- (trifluoromethyl)pyridin-2-yl)isothiazolidine-3- carboxamide 1,1-dioxide | (DMSO-d₆) δ = 8.05 (d, J = 1.2 Hz, 1H), 7.82 (d, J = 1.2 Hz, 1H), 7.65- 7.51 (m, 2H), 7.36 (t, J = 9.2 Hz, 1H), 7.31 (s, 1H), 7.19 (s, 1H), 4.96 (d, J = 17.6 Hz, 1H), 4.81 (t, J = 6.4 Hz, 1H), 4.72 (s, 4H), 4.39 (d, J = 17.6 Hz, 1H), 4.25 (s, 4H), 3.69-3.57 (m, 2H), 2.55 (s, 3H), 2.46-2.37 (m, 2H), 2.29 (s, 3H). | 645.1 [M + H]⁺. |
| 39 | 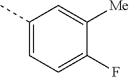 | 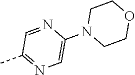 | (S)-N-(4-fluoro-3-methylphenyl)-2-(6-methyl-4- (trifluoromethyl)pyridin-2-yl)-N-(3-(5- orpholinopyrazin-2-yl)prop-2-yn-1- yl)isothiazolidine-3-carboxamide 1,1-dioxide | (DMSO-d₆) δ = 8.29 (d, J = 1.2 Hz, 1H), 8.11 (d, J = 1.2 Hz, 1H), 7.67- 7.51 (m, 2H), 7.38 (t, J = 9.2 Hz, 1H), 7.32 (s, 1H), 7.19 (s, 1H), 4.97 (d, J = 17.6 Hz, 1H), 4.81 (t, J = 6.4 Hz, 1H), 4.41 (d, J =17.6 Hz, 1H), 3.71-3.68 (m, 4H), 3.67-3.61 (m, 2H), 3.59-3.57 (m, 4H), 2.55 (s, 3H), 2.45-2.40 (m, 2H), 2.29 (s, 3H). | 633.1 [M + H]⁺. |
| 40 | 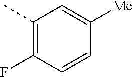 | 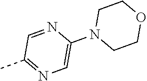 | (S)-N-(2-fluoro-5-methylphenyl)-2-(6-methyl- 4-(trifluoromethyl)pyridin-2-yl)-N-(3-(5- morpholinopyrazin-2-yl)prop-2-yn-1- yl)isothiazolidine-3-carboxamide 1,1-dioxide | (MeOD-d₄) δ = 8.15-8.09 (m, 1H), 8.05 (s, 1H), 7.84-7.79 (m, 1H), 7.41- 7.32 (m, 2H),7.31-7.23 (m, 1H), 7.18-7.10 (m, 1H), 5.13 (d, J = 17.6 Hz, 1H), 5.03-4.97 (m, 1H), 4.35 (d, J = 17.6 Hz, 1H), 3.84-3.73 (m, 4H), 3.68-3.57 (m, 5H), 3.53-3.43 (m, 1H), 2.64 (br s, 3H), 2.54-2.44 (m, 2H), 2.43 (br s, 3H). | 633.1 [M + H]⁺. |
| 42 | 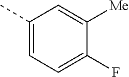 | 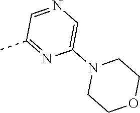 | (S)-N-(4-fluoro-3-methylphenyl)-2-(6-methyl- 4-(trifluoromethyl)pyridin-2-yl)-N-(3-(6- morpholinopyrazin-2-yl)prop-2-yn-1- yl)isothiazolidine-3-carboxamide 1,1-dioxide | (DMSO-d₆) δ = 8.31 (s, 1H), 7.85 (s, 1H), 7.67-7.52 (m, 2H), 7.38 (t, J = 9.2 Hz, 1H), 7.33 (s, 1H), 7.20 (s, 1H), 5.01 (d, J = 17.6 Hz, 1H), 4.82 (t, J = 6.4 Hz, 1H), 4.42 (d, J = 17.6 Hz, 1H), 3.73-3.68 (m, 4H), 3.67-3.56 (m, 2H), 3.53-3.47 (m, 4H), 2.57 (s, 3H), 2.47-2.38 (m, 2H), 2.30 (s, 3H). | 633.1 [M + H]⁺. |
| 43 | 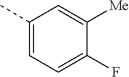 | 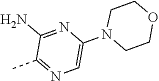 | (S)-N-(3-(3-amino-5-morpholinopyrazin-2- yl)prop-2-yn-1-yl)-N-(4-fluoro-3- methylphenyl)-2-(6-methyl-4- (trifluoromethyl)pyridin-2-ylisothiazolidine-3- carboxamide 1,1-dioxide | (DMSO-d₆) δ = 7.67-7.58 (m, 1H), 7.57-7.49 (m, 1H), 7.44 (s, 1H), 7.35 (t, J = 9.2 Hz, 1H), 7.30 (s, 1H), 7.19 (s, 1H), 5.94 (br s, 2H), 4.93 (d, J = 17.6 Hz, 1H), 4.81 (t, J = 7.2 Hz, 1H), 4.47 (d, J = 17.6 Hz, 1H), 3.69-3.56 (m, 6H), 3.50-3.44 (m, 4H), 2.54 (s, 3H), 2.46-2.37 (m, 2H), 2.29 (s, 3H). | 648.3 [M + H]⁺. |
| 44 | 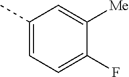 | 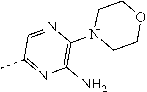 | (S)-N-(3-(6-amino-5-morpholinopyrazin-2- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (DMSO-d₆) δ = 7.61 (br s, 1H), 7.57-7.50 (m, 2H), 7.38 (t, J = 9.2 Hz, 1H), 7.32 (s, 1H), 7.20 (s, 1H), 6.50-5.90 (m, 2H), 4.92 (br d, J = 18.0 Hz, 1H), 4.82 (t, J = 6.0 Hz, 1H), 4.42 (br d, J = 17.6 Hz, 1H), 3.79-3.71 (m, 6H), 3.10-3.03 (m, 4H), 2.57 (s, 3H), 2.45-2.37 (m, 2H), 2.30 (s, 3H). | 648.3 [M + H]⁺. |
| 45 | 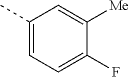 | 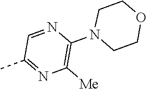 | (S)-N-(4-fluoro-3-methylphenyl)-2-(6-methyl- 4-(trifluoromethyl)pyridin-2-yl)-N-(3-(6- methyl-5-morpholinopyrazin-2-yl)prop-2-yn-1- yl)isothiazolidine-3-carboxamide 1,1-dioxide | (MeOD-d₄) δ = 8.08 (s, 1H), 7.73-7.49 (m, 2H), 7.38 (s, 1H), 7.25 (t, J = 9.2 Hz, 1H), 7.14 (s, 1H), 5.00-4.94 (m, 2H), 4.54 (d, J = 17.6 Hz, 1H), 3.82 (t, J = 4.8 Hz, 4H), 3.66-3.57 (m, 1H), 3.51-3.44 (m, 1H), 3.30-3.25 (m, 4H), 2.60 (s, 3H), 2.57-2.50 (m, 2H), 2.49 (s, 3H), 2.35 (s, 3H). | 647.2 [M + H]⁺. |
| 46 | 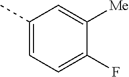 | 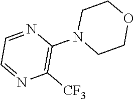 [SMILES: FC(F)(F)c1nccnc1N1CCOCC1] | (S)-N-(4-fluoro-3-methylphenyl)-2-(6-methyl- 4-(trifluoromethyl)pyridin-2-yl)-N-(3-(5- morpholino-6-(trifluoromethyl)pyrazin-2- yl)prop-2-yn-1-yl)isothiazolidine-3-carboxamide 1,1-dioxide | (DMSO-d₆) δ = 8.46 (s, 1H), 7.65 (br d, J = 3.2 Hz, 1H), 7.56 (br s, 1H), 7.37 (t, J = 9.2 Hz, 1H), 7.31 (s, 1H), 7.20 (s, 1H), 5.02 (d, J = 17.6 Hz, 1H), 4.83 (t, J = 6.8 Hz, 1H), 4.45 (d, J = 17.6 Hz, 1H), 3.72-3.68 (m, 4H), 3.68-3.56 (m, 2H), 3.47-3.41 (m, 4H), 2.56 (s, 3H), 2.47-2.38 (m, 2H), 2.30 (s, 3H). | 701.2 [M + H]⁺. |
| 47 | 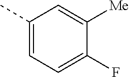 | 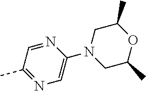 | (S)-N-(3-(5-((2S,6R)-2,6- dimethylmorpholino)pyrazin-2-yl)prop-2-yn-1- yl)-N-(4-fluoro-3-methylphenyl)-2-(6-methyl- 4-(trifluoromethyl)pyridin-2-yl)isothiazolidine- 3-carboxamide 1,1-dioxide | (DMSO-d₆) δ = 8.30 (d, J = 1.2 Hz, 1H), 8.09 (d, J = 1.2 Hz, 1H), 7.67- 7.51 (m, 2H), 7.37 (t, J = 9.2 Hz, 1H), 7.32 (s, 1H), 7.20 (s, 1H), 4.97 (d, J = 17.6 Hz, 1H), 4.82 (t, J = 6.4 Hz, 1H), 4.41 (d, J = 17.6 Hz, 1H), 4.30-4.21 (m, 2H), 3.72-3.56 (m, 4H), 2.59-2.52 (m, 5H), 2.47-2.36 (m, 2H), 2.30 (s, 3H), 1.15 (d, J = 6.4 Hz, 6H). | 661.2 [M + H]⁺. |
| 48 | 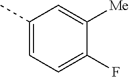 | 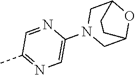 | (3S)-N-(3-(5-(8-oxa-3-azabicyclo[3.2.1]octan-3- yl)pyrazin-2-yl)prop-2-yn-1-yl)-N-(4-fluoro-3- methylphenyl)-2-(6-methyl-4- (trifluoromethyl)pyridin-2-yl)isothiazolidine-3- carboxamide 1,1-dioxide | (MeOD-d₄) δ = 8.14-7.99 (m, 2H), 7.70-7.50 (m, 2H), 7.38 (s, 1H), 7.25 (t, J = 8.0 Hz, 1H), 7.11 (s, 1H), 4.99 4.93 (m, 2H), 4.56-4.47 (m, 3H), 4.01-3.90 (m, 2H), 3.66-3.58 (m, 1H), 3.54-3.50 (s, 1H), 3.19-3.13 (m, 2H), 2.56 (s, 3H), 2.55-2.48 (m, 2H), 2.35 (s. 3H), 2.01-1.95 (m, 2H), 1.86-1.80 (m, 2H). | 659.2 [M + H]⁺. |
| 49 | 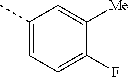 | 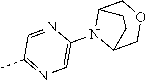 | (3S)-N-(3-(5-(3-oxa-8-azabicyclo[3.2.1]octan-8- yl)pyrazin-2-yl)prop-2-yn-1-yl)-N-(4-fluoro-3- methylphenyl)-2-(6-methyl-4- (trifluoromethyl)pyridin-2-yl)isothiazolidine-3- carboxamide 1,1-dioxide | (MeOD-d₄) δ = 8.07 (s, 2H), 7.66 (br s, 1H), 7.58 (br s, 1H), 7.37 (s, 1H), 7.25 (t, J = 9.2 Hz, 1H), 7.14 (s, 1H), 5.00-4.89 (m, 2H), 4.61-4.56 (m, 2H), 4.55-4.49 (d, J = 17.6 Hz, 1H), 3.77-3.71 (m, 2H), 3.66-3.57 (m, 3H), 3.47 (td, J = 12.8, 6.4 Hz, 1H), 2.60 (s, 3H), 2.57-2.46 (m, 2H), 2.37-2.33 (m, 3H), 2.16-2.07 (m, 2H), 2.07-1.99 (m, 2H). | 659.2 [M + H]⁺. |
| 50 | 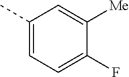 | 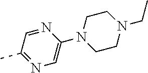 | (S)-N-(3-(5-(4-ethylpiperazin-1-yl)pyrazin-2- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (DMSO-d₆) δ = 9.68 (s, 1H), 8.42 (s, 1H), 8.16 (d, J = 1.2 Hz, 1H), 7.63 (d, J = 5.6 Hz, 1H), 7.55 (br s, 1H), 7.38 (t, J = 8.8 Hz, 1H), 7.31 (s, 1H), 7.20 (s, 1H), 4.98 (d, J = 17.6 Hz, 1H), 4.81 (t, J = 6.4 Hz, 1H), 4.56-4.47 (m, 2H), 4.42 (d, J = 17.6 Hz, 1H), 3.64 (q, J = 7.2 Hz, 2H), 3.60-3.55 (m, 2H), 3.30-3.22 (m, 2H), 3.21-3.14 (m, 2H), 3.11-2.99 (m, 2H), 2.56 (s, 3H), 2.46-2.37 (m, 2H), 2.29 (s, 3H), 1.24 (t, J = 7.2 Hz, 3H). | 660.3 [M + H]⁺. |
| 51 | 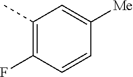 | 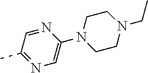 | (S)-N-(3-(5-(4-ethylpiperazin-1-yl)pyrazin-2- yl)prop-2-yn-1-yl)-N-(2-fluoro-5-methylphenyl)- 2-(6-methyl-4-(trifluoromethyl)pyridin-2- yl)isothiazolidine-3-carboxamide 1,1-dioxide | (MeOD-d₄) δ = 8.29-8.25 (m, 1H), 8.13 (s, 1H), 7.81 (d, J = 6.8 Hz, 1H), 7.41-7.24 (m, 3H), 7.13 (s, 1H), 5.11 (d, J = 17.6 Hz, 1H), 5.00 (dd, J = 7.6, 4.4 Hz, 1H), 4.70-4.54 (m, 2H), 4.38 (d, J = 17.6 Hz, 1H), 3.76-3.55 (m, 3H), 3.54-3.43 (m, 1H), 3.30-3.23 (m, 4H), 3.13 (br s, 2H), 2.61 (s, 3H), 2.53-2.43 (m, 2H), 2.40 (s, 3H), 1.39 (t, J = 7.2 Hz, 3H). | 660.2 [M + H]⁺. |
| 52 | 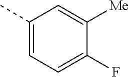 | 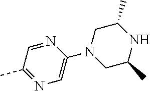 | (S)-N-(3-(5-((3S,5S)-3,5-dimethylpiperazin-1- yl)pyrazin-2-yl)prop-2-yn-1-yl)-N-(4-fluoro-3- methylphenyl)-2-(6-methyl-4- (trifluoromethyl)pyridin-2-yl)isothiazolidine-3- carboxamide 1,1-dioxide | (DMSO-d₆) δ = 8.26 (s, 1H), 8.03 (s, 1H), 7.68-7.51 (m, 2H), 7.37 (t, J = 8.8 Hz, 1H), 7.32 (s, 1H), 7.19 (s, 1H), 4.96 (br d, J = 17.6 Hz, 1H), 4.81 (t, J = 6.4 Hz, 1H), 4.41 (br d, J = 17.6 Hz, 1H), 3.76-3.51 (m, 4H), 3.28-3.22 (m, 2H), 2.55 (s, 3H), 2.45-2.38 (m, 2H), 2.29 (s, 3H), 1.00 (d, J = 6.0 Hz, 6H). | 660.2 [M + H]⁺. |
| 53 | 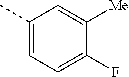 | 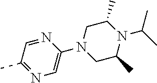 | (S)-N-(4-fluoro-3-methylphenyl)-N-(3-(5- ((3S,5S)-4-isopropyl-3,5-dimethylpiperazin-1- yl)pyrazin-2-yl)prop-2-yn-1-yl)-2-(6-methyl-4- (trifluoromethyl)pyridin-2-yl)isothiazolidine-3- carboxamide 1,1-dioxide | (DMSO-d₆) δ = 8.31 (br s, 1H), 8.05 (br s, 1H), 7.68-7.50 (m, 2H), 7.37 (t, J = 9.2 Hz, 1H), 7.31 (s, 1H), 7.19 (s, 1H), 4.96 (d, J = 17.6 Hz, 1H), 4.81 (t, J = 6.4 Hz, 1H), 4.40 (d, J = 17.6 Hz, 1H), 3.70-3.56 (m, 4H), 3.48-3.39 (m, 3H), 3.29-3.18 (m, 2H), 2.55 (s, 3H), 2.46-2.37 (m, 2H), 2.29 (s, 3H), 1.25-0.89 (m, 12H). | 702.2 [M + H]⁺. |
| 55 | 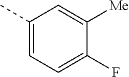 | 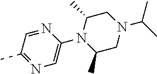 | (S)-N-(4-fluoro-3-methylphenyl)-N-(3-(5- ((2R,6R)-4-isopropyl-2,6-dimethylpiperazin-1- yl)pyrazin-2-yl)prop-2-yn-1-yl)-2-(6-methyl-4- (trifluoromethyl)pyridin-2-yl)isothiazolidine-3- carboxamide 1,1-dioxide | (DMSO-d₆) δ = 9.48 (br s, 1H), 8.34 (d, J = 1.2 Hz, 1H), 8.24 (d, J = 1.2 Hz, 1H), 7.66-7.50 (m, 2H), 7.37 (t, J = 9.2 Hz, 1H), 7.31 (s, 1H), 7.19 (s, 1H), 4.96 (d, J = 17.6 Hz, 1H), 4.83 (t, J = 6.4 Hz, 1H), 4.45 (d, J = 17.6 Hz, 2H), 4.14 (br s. 1H), 3.70-3.53 (m, 6H), 3.23 (br s, 1H), 2.55 (s, 3H), 2.47-2.38 (m, 2H), 2.29 (s, 3H), 1.38-1.18 (m, 12H). | 702.2 [M + H]⁺. |
| 56 | 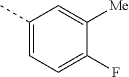 | 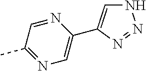 | (S)-N-(3-(5-(1H-1,2,3-triazol-4-yl)pyrazin-2- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (MeOD-d₄) δ = 9.18 (s, 1H), 8.61 (s, 1H), 8.42 (s, 1H), 7.75-7.55 (m, 2H), 7.38 (s, 1H), 7.27 (t, J = 8.8 Hz, 1H), 7.14 (s, 1H), 5.04 (d, J = 17.6 Hz, 1H), 5.00-4.96 (m, 1H), 4.60 (d, J = 17.6 Hz, 1H), 3.67-3.58 (m, 1H), 3.52-3.45 (m, 1H), 2.62 (s, 3H), 2.58-2.48 (m, 2H), 2.36 (s. 3H). | 615.2 [M + H]⁺. |
| 57 | 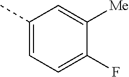 | 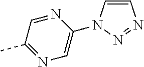 | (S)-N-(3-(5-(1H-1,2,3-triazol-1-yl)pyrazin-2- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (MeOD-d₄) δ = 9.40 (s, 1H), 8.79 (s, 1H), 8.58 (s, 1H), 7.96 (s, 1H), 7.72- 7.57 (m, 2H), 7.38 (s, 1H), 7.27 (t, J = 8.8 Hz, 1H), 7.15 (s, 1H), 5.06 (d, J = 17.6 Hz, 1H), 4.99 (dd, J = 7.2, 5.2 Hz, 1H), 4.60 (d, J = 17.6 Hz., 1H), 3.68-3.57 (m, 1H), 3.53-3.44 (m, 1H), 2.63 (s, 3H), 2.60-2.48 (m, 2H), 2.36 (s, 3H). | 615.2 [M + H]⁺. |
| 58 | 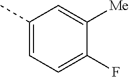 | 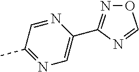 | (S)-N-(3-(5-(1,2,4-oxadiazol-3-yl)pyrazin-2- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (CD₃CN) δ = 9.25 (s, 1H), 9.17 (s, 1H), 8.72 (s, 1H), 7.61 (br s, 1H), 7.54 (br s, 1H), 7.29 (s, 1H), 7.24 (t, J = 9.2 Hz, 1H), 7.14 (s, 1H), 5.00 (d, J = 17.6 Hz, 1H), 4.95 (t, J = 6.0 Hz, 1H), 4.57 (J = 17.6 Hz, 1H), 3.59-3.51 (m, 1H), 3.43-3.34 (m, 1H), 2.59 (s, 3H), 2.49-2.42 (m, 2H), 2.33 (s, 3H), | 616.3 [M + H]⁺. |
| 59 | 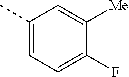 | 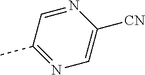 | (S)-N-(3-(5-cyanopyrazin-2-yl)prop-2-yn-1-yl)- N-(4-fluoro-3-methylphenyl)-2-(6-methyl-4- (trifluoromethyl)pyridin-2-yl)isothiazolidine-3- carboxamide 1,1-dioxide | (CD₃CN) δ = 8.91 (d, J = 1.2 Hz, 1H), 8.66 (d, J = 1.2 Hz, 1H), 7.58 (br s, 1H), 7.52 (br s, 1H), 7.29 (s, 1H), 7.23 (t, J = 9.2 Hz, 1H), 7.14 (s, 1H), 4.98 (d, J = 18.0 Hz, 1H), 4.94 (t, J = 6.0 Hz, 1H), 4.57 (d, J = 18.0 Hz, 1H), 3.59-3.48 (m, 1H), 3.42-3.33 (m, 1H), 2.57 (s, 3H), 2.47-2.41 (m, 2H), 2.32 (s, 3H). | 573.0 [M + H]⁺. |
| 60 | 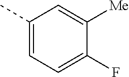 | 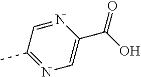 | (S)-5-(3-(N-(4-fluoro-3-methylphenyl)-2-(6- methyl-4-(trifluoromethyl)pyridin-2-yl)- 1,1-dioxidoisothiazolidine-3- carboxamido)prop-1-yn-1- yl)pyrazine-2-carboxylic acid | (DMSO-d₆) δ = 9.09-8.45 (m, 1H), 7.69 7.53 (m, 2H), 7.39 (br t, J = 9.2 Hz, 1H), 7.32 (s, 1H), 7.28-7.09 (m, 3H), 5.01 (d, J = 17.6 Hz, 1H), 4.84 (t, J = 6.4 Hz, 1H), 4.56 (d, J = 17.6 Hz, 1H), 3.72-3.58 (m, 2H), 2.56 (s, 3H), 2.46-2.38 (m, 2H), 2.31 (m, 3H). | 592.1 [M + H]⁺. |
| 61 | 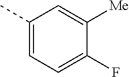 | 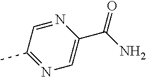 | (S)-N-(3-(5-carbamoylpyrazin-2-yl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 9.13 (d, J = 1.2 Hz, 1H), 8.69 (d, J = 1.2 Hz, 1H), 8.4 (s, 1H), 7.92 (br s, 1H), 7.69-7.52 (m, 2H), 7.39 (t, J = 8.8 Hz, 1H), 7.32 (s, 1H), 7.20 (s, 1H), 5.03 (d, J = 17.6 Hz, 1H), 4.85 (t, J = 6.4 Hz, 1H), 4.58 (d, J = 17.6 Hz, 1H), 3.71-3.59 (m, 2H), 2.56 (s, 3H), 2.47-2.40 (m, 2H), 2.31 (m, 3H). | 591.0 [M + H]⁺. |
| 62 | 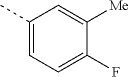 | 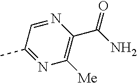 | (S)-N-(3-(5-carbamoyl-6-methylpyrazin-2- yl)prop-2-yn-1-yl)-N-(4-fluoro-3- methylphenyl)-2-(6-methyl-4- (trifluoromethyl)pyridin-2- yl)isothiazolidine-3- carboxamide 1,1-dioxide | (DMSO-d₆) δ = 8.50 (s, 1H), 8.12 (s, 1H), 7.77 (s, 1H), 7.66 (br d, J = 4.8 Hz, 1H), 7.58 (br d, J = 2.0 Hz, 1H), 7.38 (t, J = 9.2 Hz, 1H), 7.33 (s, 1H), 7.20 (s, 1H), 5.04 (d, J = 17.6 Hz, 1H), 4.85 (dd, J = 7.2, 5.6 Hz, 1H), 4.51 (d, J = 17.6 Hz, 1H), 3.73-3.56 (m, 2H), 2.70 (s, 3H), 2.58 (s, 3H), 2.48-2.39 (m, 2H), 2.30 (s, 3H). | 605.0 [M + H]⁺. |
| 63 | 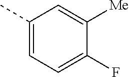 | 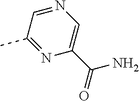 | (S)-N-(3-(6-carbamoylpyrazin-2-yl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 9.12 (s, 1H), 8.85 (s, 1H), 8.17 (br s, 1H), 7.92 (br s, 1H), 7.67-7.56 (m, 2H), 7.39 (t, J = 9.2 Hz, 1H), 7.31 (s, 1H), 7.20 (s, 1H), 4.99 (d, J = 18.0 Hz, 1H), 4.84 (t, J = 6.8 Hz, 1H), 4.60 (d, J = 18.0 Hz, 1H), 3.71-3.61 (m, 2H), 2.54 (s, 3H), 2.46-2.40 (m, 2H), 2.31 (s, 3H). | 613.1 [M + H]⁺. |
| 64 | 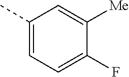 | 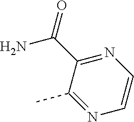 | (S)-N-(3-(3-carbamoylpyrazin-2-yl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 8.76 (d, J = 2.4 Hz, 1H), 8.64 (d, J = 2.4 Hz, 1H), 8.14 (br s, 1H), 7.78 (br s, 1H), 7.74 (br d, J = 6.4 Hz, 1H), 7.66-7.58 (m, 1H), 7.36 (t, J = 9.2 Hz, 1H), 7.32 (s, 1H), 7.20 (s, 1H), 4.85 (dd, J = 7.6, 5.2 Hz, 1H), 4.80-4.66 (m, 2H), 3.71-3.68 (m, 2H), 2.58 (s, 3H), 2.48-2.40 (m, 2H), 2.32 (s, 3H). | 591.1 [M + H]⁺. |
| 65 | 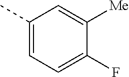 | 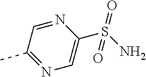 | (S)-N-(4-fluoro-3-methylphenyl)-2-(6-methyl-4- (trifluoromethyl)pyridin-2-yl)-N-(3-(5- sulfamoylpyrazin-2-yl)prop-2-yn-1- yl)isothiazolidine-3-carboxamide 1,1-dioxide | (CD₃CN) δ = 9.05 (s, 1H), 8.65 (s, 1H), 7.63-7.51 (m, 2H), 7.29 (s, 1H), 7.24 (t, J = 9.2 Hz, 1H), 7.15 (s, 1H), 5.99 (s, 2H), 4.98 (d, J = 18.0 Hz, 1H), 4.95 (t, J = 6.0 Hz, 1H), 4.57 (d, J = 18.0 Hz, 1H), 3.59-3.48 (m, 1H), 3.43-3.34 (m, 1H), 2.59 (s, 3H), 2.48-2.40 (m, 2H), 2.32 (s, 3H). | 627.0 [M + H]⁺. |
| 66 | 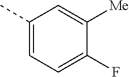 | 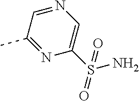 | (S)-N-(4-fluoro-3-methylphenyl)-2-(6-methyl-4- (trifluoromethyl)pyridin-2-yl)-N-(3-(6- sulfamoylpyrazin-2-yl)prop-2-yn-1- yl)isothiazolidine-3-carboxamide 1,1-dioxide | (DMSO-d₆) δ = 9.07 (s, 1H), 8.92 (s, 1H), 7.84 (s, 2H), 7.65 (br d, J = 6.0 Hz, 1H), 7.62-7.55 (m, 1H), 7.39 (t, J = 9.2 Hz, 1H), 7.30 (s, 1H), 7.20 (s, 1H), 5.00 (d, J = 18.0 Hz, 1H), 4.85 (t, J = 6.4 Hz, 1H), 4.61 (d, J = 18.0 Hz, 1H), 3.72-3.69 (m, 2H), 2.54 (s, 3H), 2.47-2.42 (m, 2H), 2.30 (s, 3H). | 627.1 [M + H]⁺. |
| 67 | 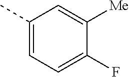 | 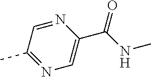 | (S)-N-(4-fluoro-3-methylphenyl)-2-(6-methyl-4- (trifluoromethyl)pyridin-2-yl)-N-(3-(5- (methylcarbamoyl)pyrazin-2-yl)prop-2-yn-1- yl)isothiazolidine-3-carboxamide 1,1-dioxide | (MeOD-d4) δ = 8.84 (br s, 1H), 8.79-8.36 (m, 1H), 7.78-7.53 (m, 1H), 7.49-7.33 (m, 1H), 7.30-7.15 (m, 2H), 7.15-6.92 (m, 2H), 5.36-4.95 (m, 1H), 4.87-4.72 (m, 2H), 3.86-3.44 (m, 2H), 2.94 (s, 4H), 2.83-2.56 (m, 4H), 2.43-2.10 (m, 3H). | 605.1 [M + H]⁺. |
| 68 | 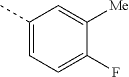 | 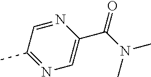 | (S)-N-(3-(5-(dimethylcarbamoyl)pyrazin-2-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (DMSO-d₆) δ = 8.80 (d, J = 1.2 Hz, 1H), 8.66 (d, J = 1.2 Hz, 1H), 7.65 (br d, J = 5.2 Hz, 1H), 7.58 (br s, 1H), 7.39 (t, J = 9.2 Hz, 1H), 7.32 (s, 1H), 7.20 (s, 1H), 5.01 (d, J = 18.0 Hz, 1H), 4.85 (t, J = 6.4 Hz, 1H), 4.58 (d, J = 18.0 Hz, 1H), 3.71-3.57 (m, 2H), 3.03 (s, 3H), 2.97 (s, 3H), 2.57 (s, 3H), 2.48-2.38 (m, 2H), 2.30 (s, 3H). | 619.2 [M + H]⁺. |
| 69 | 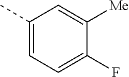 | 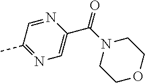 | (S)-N-(4-fluoro-3-methylphenyl)-2-(6-methyl-4- (trifluoromethyl)pyridin-2-yl)-N-(3-(5- (morpholine-4-carbonyl)pyrazin-2-yl)prop-2-yn-1- yl)isothiazolidine-3-carboxamide 1,1-dioxide | (MeOD-d₄) δ = 8.85 (d, J = 1.2 Hz, 1H), 8.61 (d, J = 1.2 Hz, 1H), 7.78- 7.55 (m, 2H), 7.40 (s, 1H), 7.28 (t, J = 8.8 Hz, 1H), 7.17 (s, 1H), 5.05 (d, J = 17.6 Hz, 1H), 5.00 (dd, J = 7.2, 4.8 Hz, 1H), 4.63 (d, J = 17.6 Hz, 1H), 3.80 (s, 4H), 3.73-3.67 (m, 2H), 3.66-3.64 (m, 2H), 3.63-3.60 (m, 1H), 3.50 (td, J = 12.4, 6.4 Hz, 1H), 2.63 (s, 3H), 2.59-2.50 (m, 2H), 2.38 (s, 3H). | 661.2 [M + H]⁺. |
| 70 | 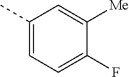 | 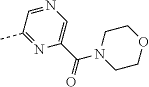 | (S)-N-(4-fluoro-3-methylphenyl)-2-(6-methyl-4- (trifluoromethyl)pyridin-2-yl)-N-(3-(6- (morpholine-4-carbonyl)pyrazin-2-yl)prop-2- yn-1-yl)isothiazolidine-3-carboxamide 1,1-dioxide | (DMSO-d₆) δ = 8.81 (s, 1H), 8.74 (s, 1H), 7.65 (br d, J = 5.6 Hz, 1H), 7.59 (br d, J = 3.2 Hz, 1H), 7.38 (t, J = 9.2 Hz, 1H), 7.32 (s, 1H), 7.20 (s, 1H), 5.01 (d, J = 17.6 Hz, 1H), 4.84 (t, J = 6.4 Hz, 1H), 4.56 (d, J = 17.6 Hz, 1H), 3.72-3.60 (m, 6H), 3.58-3.53 (m, 2H), 3.41 3.38 (m, 2H), 2.56 (s, 3H), 2.48-2.39 (m, 2H), 2.30 (s, 3H). | 661.0 [M + H]⁺. |
| 71 | 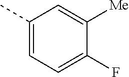 | 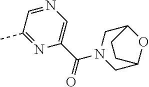 | (3S)-N-(3-(6-(8-oxa-3-azabicyclo[3.2.1]octane-3- carbonyl)pyrazin-2-yl)prop-2-yn-1-yl)-N-(4- fluoro-3-methylphenyl)-2-(6-methyl-4- (trifluoromethyl)pyridin-2-yl)isothiazolidine-3- carboxamide 1,1-dioxide | (DMSO-d₆) δ = 8.80 (s, 1H), 8.75 (s, 1H), 7.70-7.55 (m, 2H), 7.38 (t. J = 9.2 Hz, 1H), 7.32 (d, J = 4.0 Hz, 1H), 7.21 (s, 1H), 5.03 (d, J = 18.0 Hz, 1H), 4.85 (t, J = 6.4 Hz, 1H), 4.57 (br dd, J = 18.0, 2.0 Hz, 1H), 4.43 (br d, J = 6.0 Hz, 1H), 4.24 (br s, 1H), 4.15 (br d, J = 12.8 Hz, 1H), 3.72- 3.57 (m, 2H), 3.32 (br s, 2H), 3.06 (br d, J = 13.2 Hz, 1H), 2.56 (s, 3H), 2.47-2.41 (m, 2H), 2.31 (s, 3H), 1.90-1.65 (m, 4H). | 687.2 [M + H]⁺. |
| 72 | 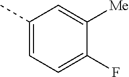 | 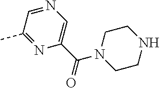 | (S)-N-(4-fluoro-3-methylphenyl)-2-(6-methyl-4- (trifluoromethyl)pyridin-2-yl)-N-(3-(6-(piperazine- 1-carbonyl)pyrazin-2-yl)prop-2-yn-1- yl)isothiazolidine-3-carboxamide 1,1-dioxide | (DMSO-d₆) δ = 8.75 (br d, J = 18.4 Hz, 2H), 7.74-7.52 (m, 2H), 7.38 (br t, J = 8.4 Hz, 1H), 7.32 (s, 1H), 7.21 (s, 1H), 5.02 (br d, J = 17.6 Hz, 1H), 4.85 (br t, J = 6.4 Hz, 1H), 4.56 (br d, J = 17.6 Hz, 1H), 3.70-3.59 (m, 2H), 3.47 (br d, J = 2.8 Hz, 8H), 2.56 (s, 3H), 2.47-2.40 (m, 2H), 2.31 (s, 3H). | 660.1 [M + H]⁺. |
| 73 | 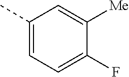 | 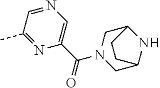 | (3S)-N-(3-(6-(3,8-diazabicyclo[3.2.1]octane-3- carbonyl)pyrazin-2-yl)prop-2-yn-1-yl)-N-(4- fluoro-3-methylphenyl)-2-(6-methyl-4- (trifluoromethyl)pyridin-2-yl)isothiazolidine-3- carboxamide 1,1-dioxide | (MeOD-d₄) δ = 8.78 (s, 1H), 8.68 (s, 1H), 7.71-7.53 (m, 2H), 7.37 (s, 1H), 7.27 (t, J = 8.8 Hz, 1H), 7.15 (d, J = 2.0 Hz, 1H), 5.04 (d, J = 17.6 Hz, 1H), 5.00-4.97 (m, 1H), 4.59 (d, J = 17.6 Hz, 1H), 4.53-4.30 (m, 1H), 4.07-3.71 (m, 2H), 3.67-3.36 (m, 4H), 3.29-3.10 (m, 1H), 2.59 (s, 3H), 2.56-2.49 (m, 2H), 2.36 (s, 3H), 2.13-1.74 (m, 4H). | 686.3 [M + H]⁺. |
| 74 | 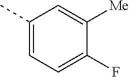 | 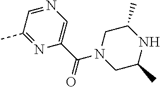 | (S)-N-(3-(6-((3S,5S)-3,5-dimethylpiperazine-1- carbonyl)pyrazin-2-yl)prop-2-yn-1-yl)-N-(4-fluoro- 3-methylphenyl)-2-(6-methyl-4- (trifluoromethyl)pyridin-2-yl)isothiazolidine-3- carboxamide 1,1-dioxide | (DMSO-d₆) δ = 9.00-8.88 (m, 2H), 8.85 (s, 1H), 8.79 (s, 1H), 7.69-7.54 (m, 2H), 7.38 (t. J = 9.2 Hz, 1H), 7.32 (s, 1H), 7.20 (s, 1H), 5.00 (d, J = 18.0 Hz, 1H), 4.85 (t, J = 6.4 Hz, 1H), 4.58 (d, J = 18.0 Hz, 1H), 3.88 (br d, J = 11.2 Hz, 1H), 3.78-3.49 (m, 7H), 2.55 (s, 3H), 2.47-2.40 (m, 2H), 2.30 (s, 3H), 1.29 (br d, J = 6.4 Hz, 3H), 1.13 (br d, J = 6.4 Hz, 3H). | 688.2 [M + H]⁺. |
| 75 | 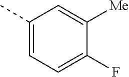 | 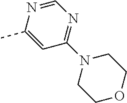 | (S)-N-(4-fluoro-3-methylphenyl)-2-(6-methyl-4- (trifluoromethyl)pyridin-2-yl)-N-(3-(6- morpholinopyrimidin-4-yl)prop-2-yn-1- yl)isothiazolidine-3-carboxamide 1,1-dioxide | (DMSO-d₆) δ = 8.45 (s, 1H), 7.66-7.52 (m, 2H), 7.38 (t, J = 9.2 Hz, 1H), 7.33 (s, 1H), 7.20 (s, 1H), 6.83 (s, 1H), 4.94 (d, J = 17.6 Hz, 1H), 4.83 (t, J = 6.4 Hz, 1H), 4.47 (d, J = 17.6 Hz, 1H), 3.70-3.55 (m, 10H), 2.58 (s, 3H), 2.46-2.39 (m, 2H), 2.30 (s, 3H). | 633.1 [M + H]⁺. |
| 76 | 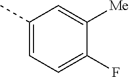 | 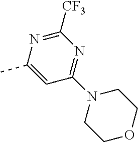 | (S)-N-(4-fluoro-3-methylphenyl)-2-(6-methyl-4- (trifluoromethyl)pyridin-2-yl)-N-(3-(6-morpholino- 2-(trifluoromethyl)pyrimidin-4-yl)prop-2-yn-1- yl)isothiazolidine-3-carboxamide 1,1-dioxide | (DMSO-d₆) δ = 7.66 (br s, 1H), 7.55 (br s, 1H), 7.37 (t, J = 9.2 Hz, 1H), 7.31 (s, 1H), 7.20 (s, 1H), 7.09 (s, 1H), 5.00 (d, J = 17.6 H7, 1H), 4.84 (t, J = 6.4 Hz, 1H), 4.45 (d, J = 17.6 Hz, 1H), 3.64-3.55 (m, 10H), 2.29 (s, 3H), 2.43 (q, J = 7.2 Hz, 2H), 2.29 (s, 3H). | 701.2 [M + H]⁺. |
| 77 | 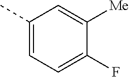 | 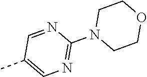 | (S)-N-(4-fluoro-3-methylphenyl)-2-(6-methyl-4- (trifluoromethyl)pyridin-2-yl)-N-(3-(2- morpholinopyrimidin-5-yl)prop-2-yn-1- yl)isothiazolidine-3-carboxamide 1,1-dioxide | (DMSO-d₆) δ = 8.38 (s, 2H), 7.64-7.51 (m, 2H), 7.37 (t, J = 9.2 Hz, 1H), 7.33 (s, 1H), 7.19 (s, 1H), 4.94 (d, J = 17.6 Hz, 1H), 4.80 (t, J = 6.4 Hz, 1H), 4.41 (d, J = 17.6 Hz, 1H), 3.75-3.70 (m, 4H), 3.69-3.64 (m, 4H), 3.64-3.56 (m, 2H), 2.56 (s, 3H), 2.46-2.36 (m, 2H), 2.30 (s, 3H). | 633.1 [M + H]⁺. |
| 78 | 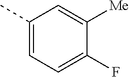 | 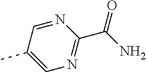 | (S)-N-(3-(2-carbamoylpyrimidin-5-yl)prop-2-yn-1-yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 8.94 (s, 2H), 8.23 (s. 1H), 7.89 (s, 1H), 7.64 (d, J = 6.4 Hz, 1H), 7.61-7.55 (m, 1H), 7.39 (t, J = 9.2 Hz, 1H), 7.33 (s, 1H), 7.20 (s, 1H), 4.98 (d, J = 18.0 Hz, 1H), 4.84 (dd, J = 7.6, 6.0 Hz, 1H), 4.61 (d, J = 18.0 Hz, 1H), 3.72-3.57 (m, 2H), 2.56 (s, 3H), 2.48-2.39 (m, 2H), 2.31 (s, 3H). | 591.2 [M + H]⁺. |
| 79 | 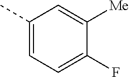 | 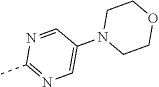 | (S)-N-(4-fluoro-3-methylphenyl)-2-(6-methyl-4- (trifluoromethyl)pyridin-2-yl)-N-(3-(5- morpholinopyrimidin-2-yl)prop-2-yn-1- yl)isothiazolidine-3-carboxamide 1,1-dioxide | (MeOD-d₄) δ = 8.38 (s, 2H), 7.81-7.54 (m, 2H), 7.37 (s, 1H), 7.23 (t, J = 8.8 Hz, 1H), 7.13 (s, 1H), 4.96 (t, J = 6.4 Hz, 1H), 4.92 (d, J = 17.6 Hz, 1H), 4.56 (d, J = 17.6 Hz, 1H), 3.84 (t, J = 4.8 Hz, 4H), 3.66-3.58 (m, 1H), 3.50-3.44 (m, 1H), 3.35-3.31 (m, 4H), 2.62 (s, 3H), 2.58-2.45 (m, 2H), 2.34 (s, 3H). | 633.2 [M + H]⁺. |
| 80 | 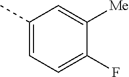 | 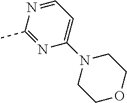 | (S)-N-(4-fluoro-3-methylphenyl)-2-(6-methyl-4- (trifluoromethyl)pyridin-2-yl)-N-(3-(4- morpholinopyrimidin-2-yl)prop-2-yn-1- yl)isothiazolidine-3-carboxamide 1,1-dioxide | (DMSO-d₆) δ = 8.16 (d, J = 6.4 Hz, 1H), 7.72-7.50 (m, 2H), 7.43-7.28 (m, 2H), 7.20 (s, 1H), 6.81 (d, J = 6.0 Hz, 1H), 4.99 (d, J = 17.6 Hz, 1H), 4.82 (t, J = 6.8 Hz, 1H), 4.34 (d, J = 17.6 Hz, 1H), 3.71-3.61 (m, 6H), 3.59-3.54 (m, 4H), 2.59 (s, 3H), 2.46-2.39 (m, 2H), 2.29 (s, 3H). | 633.2 [M + H]⁺. |
| 81 | 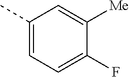 | 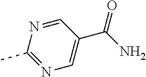 | (S)-N-(3-(5-carbamoylpyrimidin-2-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (DMSO-d₆) δ = 9.15 (s, 2H), 8.33 (s, 1H), 7.88 (s, 1H), 7.74-7.53 (m, 2H), 7.39 (t, J = 9.2 Hz, 1H), 7.33 (s, 1H), 7.21 (s, 1H), 5.05 (d, J = 17.6 Hz, 1H), 4.86 (t, J = 6.4 Hz, 1H), 4.50 (d, J = 17.6 Hz, 1H), 3.71-3.59 (m, 2H), 2.59 (s, 3H), 2.45-2.41 (m, 2H), 2.31 (s, 3H). | 591.0 [M + H]⁺. |
| 82 | 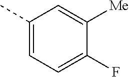 | 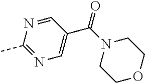 | (S)-N-(4-fluoro-3-methylphenyl)-2-(6-methyl-4- (trifluoromethyl)pyridin-2-yl)-N-(3-(5-(morpholine- 4-carbonyl)pyrimidin-2-yl)prop-2-yn-1- yl)isothiazolidine-3-carboxamide 1,1-dioxide | (MeOD-d₄) δ = 8.84 (s, 2H), 7.80-7.55 (m, 2H), 7.38 (s, 1H), 7.24 (t, J = 8.8 Hz, 1H), 7.15 (s, 1H), 5.04-4.99 (m, 1H), 4.99-4.95 (m, 1H), 4.62- 4.54 (m, 1H), 3.77-3.58 (m, 7H), 3.53-3.43 (m, 3H), 2.66 (s, 3H), 2.59- 2.49 (m, 2H), 2.36 (s, 3H). | 661.1 [M + H]⁺. |
| 83 | 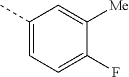 | 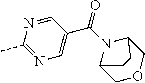 | (3S)-N-(3-(5-(3-oxa-8-azabicyclo[3.2.1]octane-8- carbonyl)pyrimidin-2-yl)prop-2-yn-1-yl)-N-(4- fluoro-3-methylphenyl)-2-(6-methyl-4- (trifluoromethyl)pyridin-2-yl)isothiazolidine-3- carboxamide 1,1-dioxide | (MeOD-d₄) δ = 8.90 (s, 2H), 7.77-7.53 (m, 2H), 7.37 (s, 1H), 7.25 (t, J = 8.8 Hz, 1H), 7.15 (s, 1H), 5.03 (d, J = 17.6 Hz, 1H), 4.99-4.96 (m, 1H), 4.66 (s, 1H), 4.59 (d, J = 17.6 Hz, 1H), 4.04 (s, 1H), 3.85-3.64 (m, 4H), 3.63-3.58 (m, 1H), 3.52-3.44 (m, 1H), 2.66 (s, 3H), 2.59-2.48 (m, 2H), 2.36 (s, 3H), 2.10-2.00 (m, 4H). | 687.2 [M + H]⁺. |
| 84 | 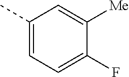 | 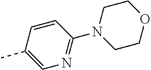 | (S)-N-(4-fluoro-3-methylphenyl)-2-(6-methyl-4- (trifluoromethyl)pyridin-2-yl)-N-(3-(5- morpholinopyridin-2-yl)prop-2-yn-1- yl)isothiazolidine-3-carboxamide 1,1-dioxide | (DMSO-d₆) δ = 8.26 (d, J = 2.4 Hz, 1H), 7.67-7.52 (m, 2H), 7.37 (t, J = 9.2 Hz, 1H), 7.33-7.24 (m, 3H), 7.20 (s, 1H), 4.94 (d, J = 17.6 Hz, 1H), 4.82 (t, J = 6.4 Hz, 1H), 4.41 (br d, J = 17.6 Hz, 1H), 3.76-3.71 (m, 4H), 3.69-3.58 (m, 2H), 3.26-3.20 (m, 4H), 2.55 (s, 3H), 2.46-2.39 (m, 2H), 2.30 (s, 3H). | 632.2 [M + H]⁺. |
| 85 | 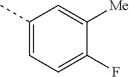 | 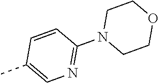 | (S)-N-(4-fluoro-3-methylphenyl)-2-(6-methyl-4- (trifluoromethyl)pyridin-2-yl)-N-(3-(6- morpholinopyridin-3-yl)prop-2-yn-1- yl)isothiazolidine-3-carboxamide 1,1-dioxide | (MeOD-d₄) δ = 8.03 (d, J = 2.0 Hz, 1H), 7.71-7.66 (m, 1H), 7.65-7.48 (m, 2H), 7.36 (s, 1H), 7.24 (t, J = 9.2 Hz, 1H), 7.13 (s, 1H), 7.10-7.00 (m, 1H), 4.99-4.92 (m, 2H), 4.45 (d, J = 17.6 Hz, 1H), 3.83-3.78 (m, 4H), 3.65-3.55 (m, SH), 3.51-3.42 (m, 1H), 2.57 (s, 3H), 2.53-2.45 (m, 2H), 2.34 (d, J = 1.2 Hz, 3H). | 632.2 [M + H]⁺. |
| 86 | 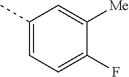 | 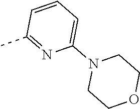 | (S)-N-(4-fluoro-3-methylphenyl)-2-(6-methyl-4- (trifluoromethyl)pyridin-2-yl)-N-(3-(6- morpholinopyridin-2-yl)prop-2-yn-1- yl)isothiazolidine-3-carboxamide 1,1-dioxide | (DMSO-d₆) δ = 7.65 (br d, J = 3.6 Hz, 1H), 7.53 (br dd, J = 8.4, 7.6 Hz, 2H), 7.37 (t, J = 9.2 Hz, 1H), 7.33 (s, 1H), 7.20 (s, 1H), 6.85 (d, J = 8.8 Hz, 1H), 6.70 (d, J = 7.6 Hz, 1H), 4.98 (br d, J = 17.6 Hz, 1H), 4.85-4.79 (m, 1H), 4.39 (d, 1H), 3.70-3.66 (m, 4H), 3.66-3.55 (m, 2H), 3.44-3.39 (m, 4H), 2.56 (s, 3H), 2.46-2.38 (m, 2H), 2.29 (s, 3H). | 632.1 [M + H]⁺. |
| 87 | 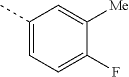 | 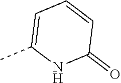 | (S)-N-(4-fluoro-3-methylphenyl)-2-(6-methyl-4- (trifluoromethyl)pyridin-2-yl)-N-(3-(6-oxo-1,6- dihydropyridin-2-yl)prop-2-yn-1-yl)isothiazolidine- 3-carboxamide 1,1-dioxide | (DMSO-d₆) δ = 7.65-7.53 (m, 2H), 7.45-7.31 (m, 3H), 7.20 (s, 1H), 6.47- 6.32 (m, 2H), 4.89-4.76 (m, 2H), 4.55 (br d, J = 17.6 Hz, 1H), 3.64-3.56 (m, 2H), 2.56 (s, 3H), 2.47-2.38 (m, 2H), 2.31 (s, 3H). | 563.0 [M + H]⁺. |
| 88 | 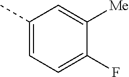 | 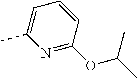 | (S)-N-(4-fluoro-3-methylphenyl)-N-(3-(6- isopropoxypyridin-2-yl)prop-2-yn-1-yl)-2-(6- methyl-4-(trifluoromethyl)pyridin-2- yl)isothiazolidine-3-carboxamide 1,1-dioxide | (MeOD-d₄) δ = 7.74-7.62 (m, 1H), 7.56 (t, J = 7.6 Hz, 2H), 7.37 (s, 1H), 7.24 (1, J = 8.8 Hz, 1H), 7.12 (s, 111), 6.92 (d, J = 7.2 Hz, 1H), 6.68 (d, J = 8.4 Hz, 1H), 5.29-5.15 (m, 1H), 5.05 (d, J = 17.6 Hz, 1H), 5.00-4.93 (m, 1H), 4.45 (d, J = 17.6 Hz, 1H), 3.66-3.57 (m, 1H), 3.50-3.44 (m, 1H), 2.58 (s, 3H), 2.56-2.48 (m, 2H), 2.35 (s, 3H), 1.30 (d, J = 6.0 Hz, 6H). | 605.1 [M + H]⁺. |
| 89 | 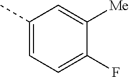 | 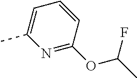 | (S)-N-(3-(6-(difluoromethoxy)pyridin-2- yl)prop-2-yn-1-yl)-N-(4-fluoro-3-methylphenyl)-2- (6-methyl-4-(trifluoromethyl)pyridin-2- yl)isothiazolidine-3-carboxamide 1,1-dioxide | (DMSO-d₆) δ = 7.93 (t, J = 8.0 Hz, 1H), 7.67-7.54 (m, 2H), 7.65 (t, J = 57.2 Hz, 1H), 7.38 (t, J = 9.2 Hz, 1H), 7.33-7.28 (m, 2H), 7.20 (s, 1H), 7.12 (d, J = 8.4 Hz, 1H), 5.00 (d, J = 17.6 Hz, 1H), 4.83 (t, J = 6.4 Hz, 1H), 4.46 (d, J = 17.6 Hz, 1H), 3.72-3.54 (m, 2H), 2.55 (s, 3H), 2.47- 2.38 (m, 2H), 2.30 (s, 3H). | 613.0 [M + H]⁺. |
| 90 | 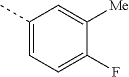 | 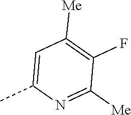 | (S)-N-(4-fluoro-3-methylphenyl)-N-(3-(5-fluoro- 4,6-dimethylpyridin-2-yl)prop-2-yn-1-yl)-2-(6-methyl-4-(trifluoromethyl)pyridin-2- yl)isothiazolidine-3-carboxamide 1,1-dioxide | (MeOD-d₄) δ = 7.78-7.50 (m, 2H), 7.38 (s, 1H), 7.33-7.20 (m, 2H), 7.14 (s, 1H), 5.71-5.65 (m, 2H), 4.55 (br d, J = 17.2 Hz, 1H), 3.68-3.57 (m, 1H), 3.52-3.46 (m, 1H), 2.58 (s 3H), 2.55-2.50 (m, 2H), 2.45 (br d, J = 3.2 Hz, 3H), 2.35 (br s, 3H), 2.29 (s, 3H) | 615.1 [M + H]⁺. |
| 91 | 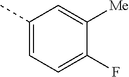 | 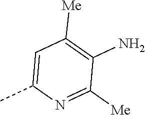 | (S)-N-(3-(5-amino-4,6-dimethylpyridin-2-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (MeOD-d₄) δ = 7.71 7.51 (m, 2H), 7.40 (s, 1H), 7.35 (t, J = 8.0 1Iz, 1H), 7.22 (s, 1H), 6.95 (s, 1H), 4.95 (dd, J = 7.6, 4.8 Hz, 1H), 4.90 (d, J = 17.2 Hz, 1H), 4.52 (d, J = 17.2 Hz, 1H), 3.67-3.56 (m, 1H), 3.52-3.43 (m, 1H), 2.56 (s, 3H), 2.54-2.45 (m, 2H), 2.35 (d, J = 1.2 Hz, 3H), 2.32 (s, 3H), 2.13 (s, 3H). | 590.2 [M + H]⁺. |
| 92 | 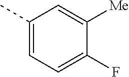 | 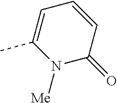 | (S)-N-(4-fluoro-3-methylphenyl)-2-(6-methyl-4- (trifluoromethyl)pyridin-2-yl)-N-(3-(1-methyl-6-oxo-1,6-dihydropyridin-2-yl)prop-2-yn- 1-yl)isothiazolidine-3-carboxamide 1,1-dioxide | (MeOD-d₄) δ = 7.62 (br s, 1H), 7.59-7.52 (m, 1H), 7.47-7.41 (m, 1H), 7.41-7.36 (m, 1H), 7.27 (t, J = 8.8 Hz, 1H), 7.15 (s, H), 6.61-6.55 (m, 2H), 5.06 4.95 (m, 2H), 4.61 (d, J = 17.6 Hz, 1H), 3.67-3.55 (m, 1H), 3.52-3.45 (m, 4H), 2.56 (s, 3H), 2.55-2.47 (m, 2H), 2.36 (d, J = 1.6 Hz, 3H). | 577.0 [M + H]⁺. |
| 93 | 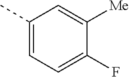 | 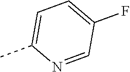 | (S)-N-(4-fluoro-3-methylphenyl)-N-(3-(5- fluoropyridin-2-yl)prop-2-yn-1-yl)-2-(6-methyl-4- (trifluoromethyl)pyridin-2-yl)isothiazolidine-3- carboxamide 1,1-dioxide | (D)MSO-d₆) δ = 8.56 (d, J = 3.2 Hz, 1H), 7.79-7.71 (m, 1H), 7.64 (br d, J = 5.2 Hz, 1H), 7.60-7.48 (m, 2H), 7.37 (t, J = 9.2 Hz, 1H), 7.31 (s, 1H), 7.20 (s, 1H), 4.96 (br d, J = 17.6 1Iz, 1H), 4.83 (t, J = 6.4 Hz, 1H), 4.46 (br d, J = 17.6 Hz, 1H), 3.69-3.57 (m, 2H), 2.55 (s, 3H), 2.47-2.36 (m, 2H), 2.30 (s, 3H) | 564.9 [M + H]⁺. |
| 94 | 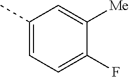 | 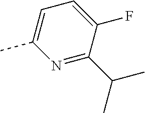 | (S)-N-(4-fluoro-3-methylphenyl)-N-(3-(5-fluoro- 6-isopropylpyridin-2-yl)prop-2-yn-1-yl)-2-(6- methyl-4-(trifluoromethyl)pyridin-2- yl)isothiazolidine-3-carboxamide 1,1-dioxide | (MeOD-d₄) δ = 7.81-7.55 (m, 2H), 7.48 (t, J = 8.4 Hz, 1H), 7.37 (s, 1H), 7.34-7.23 (m, 2H), 7.15 (s, 1H), 5.00 (d, J = 17.6 Hz, 1H), 4.94 (t, J = 6.4 Hz, 1H), 4.50 (d, J = 17.6 Hz, 1H), 3.68-3.59 (m, 1H), 3.49-3.45 (m, 1H), 3.42-3.35 (m, 1H), 2.60 (s, 3H), 2.58-2.50 (m, 2H), 2.37 (d. J = 1.6 Hz, 3H), 1.30 (d, J = 6.8 Hz, 6H). | 607.2 [M + H]⁺. |
| 95 | 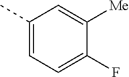 | 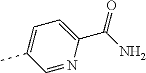 | (S)-N-(3-(6-carbamoylpyridin-3-yl)prop-2-yn-1- yl)-N-(4-fluoro-3-methylphenyl)-2-(6- methyl-4-(trifluoromethyl)pyridin-2- yl)isothiazolidine-3-carboxamide 1,1-dioxide | (DMSO-d₆) δ = 8.57 (d, J = 1.2 Hz, 1H), 8.15 (br s, 1H), 8.05-8.00 (m, 1H), 7.97-7.92 (m, 1H), 7.75 (br s, 1H), 7.67-7.55 (m, 2H), 7.39 (t, J = 9.2 Hz, 1H), 7.32 (s, 1H), 7.20 (s, 1H), 4.98 (d, J = 17.6 Hz, 1H), 4.83 (dd, J = 7.2, 5.6 Hz, 1H), 4.54 (d, J = 17.6 Hz, 1H), 3.63-3.56 (m, 2H), 2.54 (s, 3H), 2.48-2.40 (m, 2H), 2.31 (s, 3H). | 590.2 [M + H]⁺. |
| 96 | 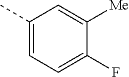 | 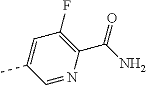 | (S)-N-(3-(6-carbamoyl-5-fluoropyridin-3-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (DMSO-d₆) δ = 8.40 (s, 1H), 8.06 (br s, 1H), 7.91 (dd, J = 11.2, 1.6 Hz, 1H), 7.75 (s, 1H), 7.67-7.60 (m, 1H), 7.59-7.52 (m, 1H), 7.39 (t, J = 9.2 Hz, 1H), 7.33 (s, 1H), 7.20 (s, 1H), 4.95 (d, J = 18.0 Hz, 1H), 4.88-4.78 (m, 2H), 4.58 (d, J = 18.0 Hz, 1H), 3.71-3.57 (m, 2H), 2.55 (s, 3H), 2.47- 2.39 (m, 2H), 2.31 (s, 3H). | 608.2 [M + H]⁺. |
| 97 | 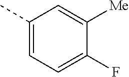 | 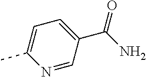 | (S)-N-(3-(5-carbamoylpyridin-2-yl)prop-2-yn-1-yl)-N-(4-fluoro-3-methylphenyl)-2-(6- methyl-4-(trifluoromethyl)pyridin-2-yl)isothiazolidine-3-carboxamide 1,1-dioxide | (MeOD-d₄) δ = 8.98 (br s, 1H), 8.25 (dd, J = 8.4, 2.4 Hz, 1H), 7.77-7.47 (m, 3H), 7.37 (s, 1H), 7.25 (br t, J = 8.8 Hz, 1H), 7.13 (s, 1H), 5.00-4.94 (m, 2H), 4.60 (br d, J = 17.6 Hz, 1H), 3.67-3.56 (m, 1H), 3.53-3.43 (m, 1H), 2.58 (s, 3H), 2.56 2.47 (m, 2H), 2.38-2.32 (m, 3H). | 590.1 [M + H]⁺. |
| 98 | 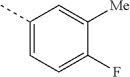 | 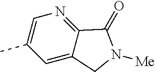 | (S)-N-(4-fluoro-3-methylphenyl)-2-(6-methyl-4- (trifluoromethyl)pyridin-2-yl)-N-(3-(6-methyl-7- oxo-6,7-dihydro-5H-pyrrolo[3,4-b]pyridin-3- yl)prop-2-yn-1-yl)isothiazolidine-3-carboxamide 1,1-dioxide | (DMSO-d₆) δ = 8.63 (d, J = 2.0 Hz, 1H), 8.04 (d, J = 1.6 Hz, 1H), 7.64 (br d, J = 6.0 Hz, 1H), 7.61-7.55 (m, 1H), 7.39 (1, J = 8.8 Hz, 1H), 7.31 (s, 1H), 7.20 (s, 1H), 4.96 (d, J = 17.6 Hz, 1H), 4.84 (dd, J = 7.2, 5.6 Hz, 1H), 4.56 (d, J = 17.6 Hz, 1H), 4.47 (s, 2H), 3.73 3.55 (m, 2H), 3.11 (s, 3H), 2.54 (s, 3H), 2.48-2.39 (m, 2H), 2.31 (s, 3H). | 616.0 [M + H]⁺. |
| 99 | 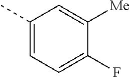 | 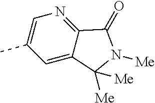 | (S)-N-(4-fluoro-3-methylphenyl)-2-(6-methyl-4- (trifluoromethyl)pyridin-2-yl)-N-(3-(5,5,6- trimethyl-7-oxo-6.7-dihydro-5H-pyrrolo[3,4- b]pyridin-3-yl)prop-2-yn-1-yl)isothiazolidine-3- carboxamide 1,1-dioxide | (DMSO-d₆) δ = 8.63 (d, J = 2.0 Hz, 1H), 8.23 (d, J = 2.0 Hz, 1H), 7.68- 7.62 (m, 1H), 7.62-7.55 (m, 1H), 7.40 (t, J = 8.8 Hz, 1H), 7.31 (s, 1H), 7.21 (s. 1H), 4.96 (d, J = 17.6 Hz, 1H), 4.78-4.88 (m, 1H), 4.55 (d, J = 17.6 Hz, 1H), 3.73-3.56 (m, 2H), 2.96 (s, 3H), 2.53 (s, 3H), 2.47-2.39 (m, 2H), 2.31 (s, 3H), 1.45 (s, 6H). | 644.2 [M + H]⁺. |
| 100 | 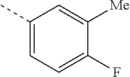 | 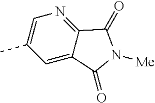 | (S)-N-(4-fluoro-3-methylphenyl)-2-(6-methyl-4- (trifluoromethyl)pyridin-2-yl)-N-(3-(6-methyl-5,7- dioxo-6,7-dihydro-5H-pyrrolo[3,4-b]pyridin-3- yl)prop-2-yn-1-yl)isothiazolidine-3-carboxamide 1,1-dioxide | (DMSO-d₆) δ = 8.88 (d, J = 1.8 Hz, 1H), 8.24 (d, J = 1.8 Hz, 1H), 7.69- 7.56 (m, 2H), 7.39 (t, J = 9.2 Hz, 1H), 7.32 (s, 1H), 7.21 (s, 1H), 4.92 (d, J = 18.0 Hz, 1H), 4.85 (dd, J = 7.2, 5.2 Hz, 1H), 4.66 (d, J = 18.0 Hz, 1H), 3.73-3.68 (m, 2H), 3.07 (s, 3H), 2.56 (s, 3H), 2.48-2.41 (m. 2H), 2.31 (s, 3H). | 630.2 [M + H]⁺. |
| 101 | 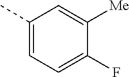 | 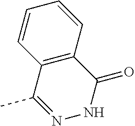 | (S)-N-(4-fluoro-3-methylphenyl)-2-(6-methyl-4- (trifluoromethyl)pyridin-2-yl)-N-(3-(4-oxo-3,4- dihydrophthalazin-1-yl)prop-2-yn-1- yl)isothiazolidine-3-carboxamide 1,1-dioxide | (DMSO-d₆) δ = 12.93 (br s, 1H), 8.22 (d, J = 7.6 Hz, 1H), 7.85 (br t, J = 7.6 Hz, 1H), 7.76 (br t, J = 7.6 Hz, 1H), 7.67 (br d, J = 7.6 Hz, 2H), 7.59 (br s, 1H), 7.39 (br t, J = 8.8 Hz, 1H), 7.30 7.12 (m, 2H), 5.07 (d, J = 17.6 Hz, 1H), 4.88 (br t, J = 6.4 Hz, 1H), 4.57 (br d, J = 17.6 Hz, 1H), 3.79-3.54 (m, 2H), 2.41 (s, 3H), 2.29 (s, 3H). | 614.0 [M + H]⁺. |
| 102 | 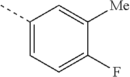 | 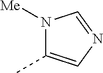 | (S)-N-(4-fluoro-3-methylphenyl)-N-(3-(1-methyl- 1H-imidazol-5-yl)prop-2-yn-1-yl)-2-(6-methyl-4- (trifluoromethyl)pyridin-2-yl)isothiazolidine-3- carboxamide 1,1-dioxide | (DMSO-d₆) δ = 8.04-7.89 (m, 1H), 7.64-7.49 (m, 2H), 7.41-7.30 (m, 3H), 7.20 (s, 1H), 4.96 (d, J = 17.6 Hz, 1H), 4.83 (t, J = 6.4 Hz, 1H), 4.54 (d, J = 17.6 Hz, 1H), 3.68-3.59 (m, 2H), 3.49 (s, 3H), 2.52 (br s, 3.H), 2.45- 2.40 (m, 2H), 2.29 (s, 3H). | 550.0 [M + H]⁺. |
| 103 | 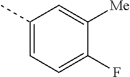 | 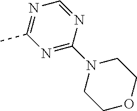 | (S)-N-(4-fluoro-3-methylphenyl)-2-(6-methyl-4- (trifluoromethyl)pyridin-2-yl)-N-(3-(4-morpholino- 1,3,5-triazin-2-yl)prop-2-yn-1-yl)isothiazolidine-3- carboxamide 1,1-dioxide | (MeOD-d₄) δ = 8.45 (s, 1H), 7.74-7.52 (m, 2H), 7.37 (s, 1H), 7.25 (t, J = 8.8 Hz, 1H), 7.16 (s, 1H), 5.00-4.94 (m, 2H), 4.53 (d, J = 18.0 Hz, 1H), 3.85 (d, J = 15.2 Hz, 4H), 3.75-3.70 (m, 4H), 3.65-3.57 (m, 1H), 3.51-3.4 (m, 1H), 2.65 (s, 3H), 2.59-2.45 (m, 2H), 2.35 (d, J = 1.2 Hz, 3H). | 634.2 [M + H]⁺. |
| 104 | 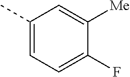 | 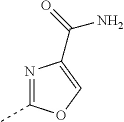 | (S)-2-(3-(N-(4-fluoro-3-methylphenyl)-2-(6- methyl-4-(trifluoromethyl)pyridin-2-yl)- 1,1-dioxidoisothiazolidine-3-carboxamido)prop- 1-yn-1-yl)oxazole-4-carboxamide | (DMSO-d₆) δ = 8.65 (s, 1H), 7.74 (br s, 1H), 7.64-7.57 (m, 2H), 7.56-7.49 (m, 1H), 7.39 (t, J = 9.2 Hz, 1H), 7.33 (s, 1H), 7.20 (s, 1H), 5.01 (d, J = 18.0 Hz, 1H), 4.84 (t, J = 6.8 Hz, 1H), 4.54 (d, J = 18.0 Hz, 1H), 3.66-3.57 (m, 2H), 2.55 (s, 3H), 2.46-2.37 (m, 2H), 2.30 (s, 3H). | 580.1 [M + H]⁺. |
| 105 | 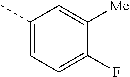 | 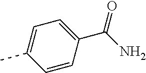 | (S)-N-(3-(4-carbamoylphenyl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (DMSO-d₆) δ = 8.03 (br s, 1H), 7.85 (d, J = 8.4 Hz, 2H), 7.63 (br d, J = 6.0 Hz, 1H), 7.57 (br s, 1H), 7.46 (br s, 1H), 7.43-7.36 (m, 3H), 7.32 (s, 1H), 7.20 (s, 1H), 4.96 (d, J = 17.6 Hz, 1H), 4.83 (t, J = 6.4 Hz, 1H), 4.46 (d, J = 17.6 Hz, 1H), 3.58 (br d, J = 7.6 Hz, 2H), 2.54 (s, 3H), 2.47- 2.39 (m, 2H), 2.31 (s, 3H). | 589.1 [M + H]⁺. |
| 106 | 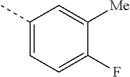 | 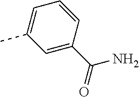 | (S)-N-(3-(3-carbamoylphenyl)prop-2-yn-1- yl)-N-(4-fluoro-3-methylphenyl)-2-(6-methyl-4- (trifluoromethyl)pyridin-2-yl)isothiazolidine-3- carboxamide 1,1-dioxide | (MeOD-d₄) δ = 8.08 (s, 1H), 7.73-7.49 (m, 2H), 7.38 (s, 1H), 7.25 (t, J = 9.2 Hz, 1H), 7.14 (s, 1H), 5.00-4.94 (m, 2H), 4.54 (d, J = 17.6 Hz, 1H), 3.82 (t, J = 4.8 Hz, 4H), 3.66-3.57 (m, 1H), 3.51-3.44 (m, 1H), 3.30-3.25 (m, 4H), 2.60 (s, 3H), 2.57-2.50 (m, 2H), 2.49 (s, 3H), 2.35 (s, 3H). | 647.2 [M + H]⁺. |
| 111 | 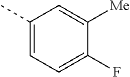 | 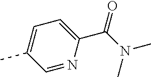 | (S)-N-(3-(6-(dimethylcarbamoyl)pyridin-3- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (DMSO-d₆) δ = 8.55 (d, J = 1.2 Hz, 1H), 7.88 (br d, J = 2.0 Hz, 1H), 7.64 (br d, J = 6.4 Hz, 1H), 7.57 (d, J = 8.0 Hz, 2H), 7.40 (t, J = 9.2 Hz, 1H), 7.33 (s, 1H), 7.20 (s, 1H), 4.97 (d, J = 17.6 Hz, 1H), 4.84 (t, J = 6.4 Hz, 1H), 4.54 (d, J = 17.6 Hz, 1H), 3.69-3.60 (m, 2H), 3.01 (s, 3H), 2.93 (s, 3H), 2.55 (s, 3H), 2.48-2.40 (m, 2H), 2.32 (s, 3H). | 618.1 [M + H]⁺. |
| 112 | 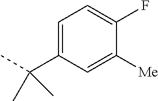 | 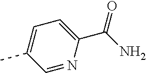 | (S)-N-(3-(6-carbamoylpyridin-3-yl)prop-2-yn-1- yl)-N-(2-(4-fluoro-3-methylphenyl)propan- 2-yl)-2-(6-methyl-4-(trifluoromethyl)pyridin-2- yl)isothiazolidine-3-carboxamide 1,1-dioxide | (MeOD-d₄) δ = 8.75 (s, 1H), 8.16-8.11 (m, 1H), 8.11-8.05 (m, 1H), 7.36 (s, 1H), 7.22 (br d, J = 7.2 Hz, 2H), 7.15 (s, 1H), 6.90 (t, J = 8.8 Hz, 1H), 5.56 (dd, J = 6.8, 3.2 Hz, 1H), 5.32 (d, J =19.6 Hz, 1H), 4.75 (d, J = 19.6 Hz, 1H), 3.51 (dd, J = 8.0, 6.4 Hz, 2H), 2.96-2.81 (m, 1H), 2.52 (s, 3H), 2.50-2.40 (m, 1H), 2.17 (s, 3H), 1.82-1.79 (m, 6H). | 632.2 [M + H]⁺. |
| 113 | 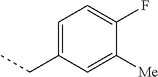 | 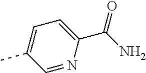 | (S)-N-(3-(6-carbamoylpyridin-3-yl)prop-2-yn-1- yl)-N-(4-fluoro-3-methylbenzyl)-2-(6-methyl-4- (trifluoromethyl)pyridin-2-yl)isothiazolidine-3- carboxamide 1,1-dioxide | (MeOD-d₄) δ = 8.83-8.72 (m, 1H), 8.35-8.22 (m, 2H), 7.47-7.33 (m, 2H), 7.21-6.92 (m, 3H), 5.67-5.51 (m, 1H), 5.16-4.94 (m, 2H), 4.67-4.31 (m, 2H), 3.86-3.46 (m, 2H), 3.00-2.86 (m, 0.5H), 2.69-2.57 (m, 1H), 2.47 (s, 1H), 2.45-2.34 (m, 0.5H), 2.29 (d, J = 1.6 Hz, 1H), 2.21 (s, 2H), 2.16 (d, J = 1.6 Hz, 2H). | 604.1 [M + H]⁺. |
| 115 | 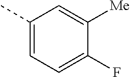 | 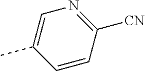 | (S)-N-(3-(6-cyanopyridin-3-yl)prop-2-yn-1-yl)- N-(4-fluoro-3-methylphenyl)-2-(6-methyl-4- (trifluoromethyl)pyridin-2-yl)isothiazolidine-3- carboxamide 1,1-dioxide | (MeOD-d₄) δ = 8.81-8.62 (m, 1H), 8.20-7.97 (m, 2H), 7.63 (d, J = 6.8 Hz, 1H), 7.61-7.54 (m, 1H). 7.39 (t. J = 9.2 Hz, 1H), 7.32 (s, 1H), 7.20 (s, 1H), 4.97 (d. J = 17.6 Hz, 1H), 4.84 (dd, J = 7.2, 5.6 Hz, 1H), 4.60 (d, J = 17.6 Hz, 1H), 3.73-3.55 (m, 2H), 2.55 (s, 3H), 2.49-2.40 (m, 2H), 2.31 (s, 3H). | 572.0 [M + H]⁺. |
| 116 | 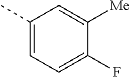 | 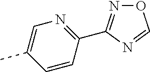 | (S)-N-(3-(6-(1,2,4-oxadiazol-3-yl)pyridin-3- vl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (DMSO-d₆) δ = 9.80 (s, 1H), 8.73 (d, J = 1.6 Hz, 1H), 8.12 (d, J = 8.0 Hz, 1H), 8.01 (dd, J = 8.0, 2.0 Hz, 1H), 7.65 (br d, J = 6.4 Hz, 1H), 7.62-7.55 (m, 1H), 7.40 (t, J = 9.2 Hz, 1H), 7.32 (s, 1H), 7.20 (s, 1H), 4.99 (d, J = 17.6 Hz, 1H), 4.84 (dd, J = 7.2, 5.6 Hz, 1H), 4.55 (d, J = 17.6 Hz, 1H), 3.73-3.56 (m, 2H), 2.55 (s, 3H), 2.47-2.38 (m, 2H), 2.32 (s, 3H). | 615.1 [M + H]⁺. |
| 117 | 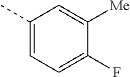 | 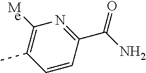 | (S)-N-(3-(6-carbamoyl-2-methylpyridin-3- yl)prop-2-yn-1-yl)-N-(4-fluoro-3-methylphenyl)- 2-(6-methyl-4-(trifluoromethyl)pyridin-2- yl)isothiazolidine-3-carboxamide 1,1-dioxide | (MeOD-d₄) δ = 7.90 (d, J = 7.6 Hz, 1H), 7.83 (d, J = 7.6 Hz, 1H), 7.69- 7.60 (m, 1H), 7.60-7.52 (m, 1H), 7.38 (s, 1H), 7.27 (t, J = 9.2 Hz, 1H), 7.13 (s, 1H), 5.05 (d, J = 17.6 Hz, 1H), 5.00 (t, J = 6.0 Hz, 1H), 4.60 (d, J = 17.6 Hz, 1H), 3.67-3.56 (m, 1H), 3.53-3.43 (m, 1H), 2.56-2.48 (m, 8H), 2.37 (s, 3H). | 604.1 [M + H]⁺. |
| 118 | 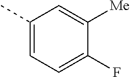 | 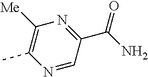 | (S)-N-(3-(5-carbamoyl-3-methylpyrazin-2-yl)prop- 2-yn-1-yl)-N-(4-fluoro-3-methylphenyl)-2-(6- methyl-4-(trifluoromethyl)pyridin-2- yl)isothiazolidine-3-carboxamide 1,1-dioxide | (MeOD-d₄) δ = 9.00 (s, 1H), 7.68 (br s, 1H), 7.59 (br s, 1H), 7.38 (s, 1H), 7.26 (t, J = 8.8 Hz, 1H), 7.13 (s, 1H), 5.06 (d, J = 17.6 Hz, 1H), 5.01 (dd, J = 7.2, 5.2 Hz, 1H), 4.66 (d, J = 17.6 Hz, 1H), 3.67-3.58 (m, 1H), 3.53- 3.44 (m, 1H), 2.57 (s, 3H), 2.56 (s, 3H), 2.55 2.46 (m, 2H), 2.36 (d, J = 1.2 Hz, 3H). | 605.2 [M + H]⁺. |
| 119 | 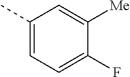 | 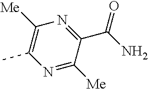 | (S)-N-(3-(5-carbamoyl-3,6- dimethylpyrazin-2-yl)prop-2- yn-1-yl)-N-(4-fluoro-3- methylphenyl)-2-(6-methyl-4- (trifluoromethyl)pyridin-2- yl)isothiazolidine-3- carboxamide 1,1-dioxide | — | 619.1 [M + H]⁺. |
| 120 | 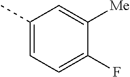 | 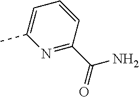 | (S)-N-(3-(6-carbamoylpyridin- 2-yl)prop-2-yn-1-yl)-N-(4- fluoro-3-methylphenyl)-2-(6- methyl-4- (trifluoromethyl)pyridin-2- yl)isothiazolidine-3- carboxamide 1,1-dioxide | — | 590.2 [M + H]⁺. |
| 121 | 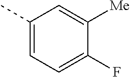 | 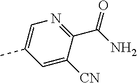 | (S)-N-(3-(6-carbamoyl-5- cyanopyridin-3-yl)prop-2-yn- 1-yl)-N-(4-fluoro-3- methylphenyl)-2-(6-methyl-4- (trifluoromethyl)pyridin-2- yl)isothiazolidine-3- carboxamide 1,1-dioxide | — | 615.2 [M + H]⁺. |
| 122 | 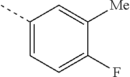 | 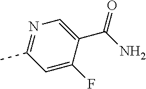 | (S)-N-(3-(5-carbamoyl-4- fluoropyridin-2-yl)prop-2-yn- 1-yl)-N-(4-fluoro-3- methylphenyl)-2-(6-methyl-4 (trifluoromethyl)pyridin-2- yl)isothiazolidine-3- carboxamide 1,1-dioxide | — | 608.1 [M + H]⁺. |
| 123 | 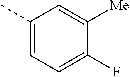 | 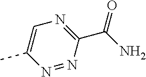 | (S)-N-(3-(3-carbamoyl-1,2,4- triazin-6-yl)prop-2-yn-1-yl)-N- (4-fluoro-3-methylphenyl)-2- (6-methyl-4- (trifluoromethyl)pyridin-2- yl)isothiazolidine-3- carboxamide 1,1-dioxide | — | 592.1 [M + H]⁺. |
| 124 | 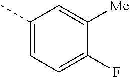 | 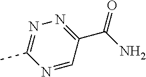 | (S)-N-(3-(6-carbamoyl-1,2,4- triazin-3-yl)prop-2-yn-1-yl)-N- (4-fluoro-3-methylpheny])-2- (6-methyl-4- (trifluoromethyl)pyridin-2- yl)isothiazolidine-3- carboxamide 1,1-dioxide | — | 592.1 [M + H]⁺. |
| 127 | 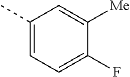 | 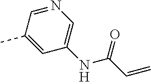 | (S)-N-(3-(5-acrylamidopyridin- 3-yl)prop-2-yn-1-yl)-N-(4- fluoro-3-methylphenyl)-2-(6- methyl-4- (trifluoromethyl)pyridin-2- yl)isothiazolidine-3- carboxamide 1,1-dioxide | | 616.2 |
| 128 | 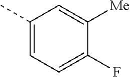 | 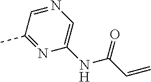 | (S)-N-(3-(6- acrylamidopyrazin-2-yl)prop- 2-yn-1-yl)-N-(4-fluoro-3- methylphenyl)-2-(6-methyl-4- (trifluoromethyl)pyridin-2- yl)isothiazolidine-3- carboxamide 1,1-dioxide | — | 617.1 |
| 129 | 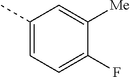 | 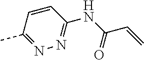 | (S)-N-(3-(6- acrylamidopyridazin-3- yl)prop-2-yn-1-yl)-N-(4- fluoro-3-methylphenyl)-2-(6- methyl-4- (trifluoromethyl)pyridin-2- yl)isothiazolidine-3- carboxamide 1,1-dioxide | — | 617.1 |
| 130 | 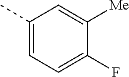 | 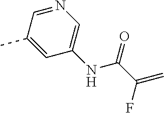 | (S)-N-(4-fluoro-3- methylphenyl)-N-(3-(5-(2- fluoroacry lamido)pyridin-3- yl)prop-2-yn-1-yl)-2-(6- methyl-4- (trifluoromethyl)pyridin-2- yl)isothiazolidine-3- carboxamide 1,1-dioxide | — | 634.2 |
| 131 | 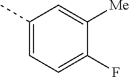 | 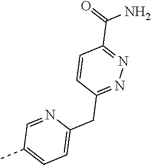 | (S)-N-(3-(6-((6- carbamoylpyridazin-3- yl)methyl)pyridin-3-yl)prop-2- yn-1-yl)-N-(4-fluoro-3- methylphenyl)-2-(6-methyl-4- (trifluoromethyl)pyridin-2- yl)isothiazolidine-3- carboxamide 1,1-dioxide | | 682.1 |
| 132 | 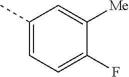 | 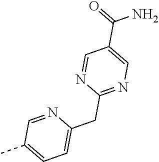 | (S)-N-(3-(6-((5- carbamoylpyrimidin-2- yl)methyl)pyridin-3-yl)prop-2- yn-1-yl)-N-(4-fluoro-3- methylphenyl)-2-(6-methyl-4- (trifluoromethyl)pyridin-2- yl)isothiazolidine-3- carboxamide 1,1-dioxide | — | 682.2 |
| 133 | 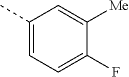 | 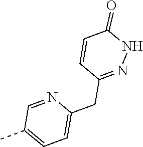 | (S)-N-(4-fluoro-3- methylphenyl)-2-(6-methyl-4- (trifluoromethyl)pyridin-2-yl)- N-(3-(6-((6-oxo-1,6- dihydropyridazin-3- yl)methyl)pyridin-3-yl)prop-2- yn-1-yl)isothiazolidine-3- carboxamide 1,1-dioxide | | 655.1 |
| 134 | 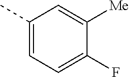 | 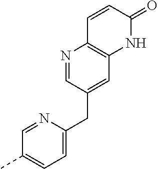 | (S)-N-(4-fluoro-3- methylphenyl)-2-(6-methyl-4- (trifluoromethyl)pyridin-2-yl)- N-(3-(6-((6-oxo-5,6-dihydro- 1,5-naphthyridin-3- yl)methyl)pyridin-3-yl)prop-2- yn-1-yl)isothiazolidine-3- carboxamide 1,1-dioxide | | 705.2 |
| 135 | 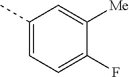 | 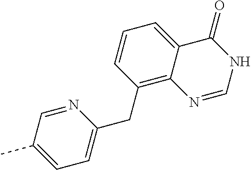 | (S)-N-(4-fluoro-3- methylphenyl)-2-(6-methyl-4- (trifluoromethyl)pyridin-2-yl)- N-(3-(6-((4-oxo-3,4- dihydroquinazolin-8- yl)methyl)pyridin-3-yl)prop-2- yn-1-yl)isothiazolidine-3- carboxamide 1,1-dioxide | | 705.2 |
| 136 | 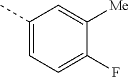 | 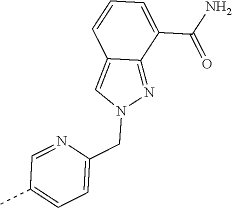 | (S)-N-(3-(6-((7-carbamoyl-2H- indazol-2-yl)methyl)pyridin-3- yl)prop-2-yn-1-yl)-N-(4- fluoro-3-methylphenyl)-2-(6- methyl-4- (trifluoromethyl)pyridin-2- yl)isothiazolidine-3- carboxamide 1,1-dioxide | | 720.2 |

The Examples presented in Table 3 were prepared using methods similar to those described in the synthesis of Example 17.

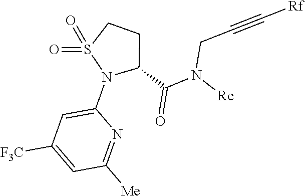

| Exa- | | | | | |
|---|---|---|---|---|---|
| mple | | | | | MS |
| Num- | | | | | (ESI) |
| ber | Re | Rf | Name | ¹H NMR (400 MHz) δ PPM | m/z = |
| 31 | 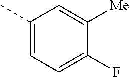 | 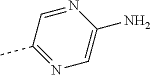 | (R)-N-(3-(5-aminopyrazin- 2-yl)prop-2-yn-1-yl)-N- (2,4-diffuoro-3- methylphenyl)- 2-(6-methyl-4- | (MeOD-d₄) δ = 7.94-7.79 (m, 3H), 7.42- 7.24 (m, 1H), 7.21-7.00 (m. 2H), 5.15 (d, J = 17.5 Hz, 1H), 4.99-4.91 (m, H), 4.51 (d, J = 17.5 Hz, 0.5H), 4.30 (d, J = 17.5 Hz, 0.5H), 3.69-3.56 (m, 1H), 3.52-3.42 | 581.0 [M + H]⁺. |
| | | | (trifluoromethyl)pyridin- | (m, 1H), 2.63-2.54 (m, 3H), 2.53-2.38 (m, | |
| | | | 2-yl)isothiazolidine-3- | 2H), 2.33-2.24 (m, 3H). | |
| | | | carboxamide 1,1-dioxide | | |
| 41 | 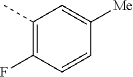 | 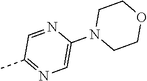 | (R)-N-(2-fluoro-5- methylphenyl)- 2-(6-methyl-4- (trifluoromethyl)pyridin- 2-yl)-N-(3-(5- morpholinopyrazin-2- | (MeOD-d₄) δ = 8.15-8.09 (m, 1H), 8.05 (s, 1H), 7.84-7.79 (m, 1H), 7.41-7.32 (m, 2H), 7.31-7.23 (m, 1H), 7.18-7.10 (m, 1H), 5.13 (d, J = 17.6 Hz, 1H), 5.03- 4.97 (m, 1H), 4.35 (d, J = 17.6 Hz, 1H), 3.84-3.73 (m, 4H), 3.68-3.57 (m, 5H), | 633.1 [M + H]⁺. |
| | | | yl)prop-2-yn-1-yl) | 3.53-3.43 (m, 1H), 2.64 (br s, 3H), 2.54- | |
| | | | isothiazolidine-3- | 2.44 (m, 2H), 2.43 (br s, 3H). | |
| | | | carboxamide 1,1-dioxide | | |
| 54 | 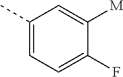 | 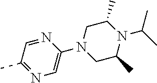 | (R)-N-(4-fluoro-3- methylphenyl)- N-(3-(5-((3S,5S)-4- isopropyl-3,5- dimethylpiperazin-1- yl)pyrazin-2- | (DMSO-d₆) δ = 8.29 (br s, 1H), 8.03 (s, 1H), 7.69-7.50 (m, 2H), 7.36 (t, J = 9.2 Hz, 1H), 7.31 (s, 1H), 7.19 (s, 1H), 4.96 (d, J = 17.6 Hz, 1H), 4.81 (t, J = 6.4 Hz, 1H), 4.40 (d, J = 17.6 Hz, 1H), 3.73-3.55 (m, 9H), 2.55 (s. 3H), 2.45-2.37 (m, 2H), | 702.2 [M + H]⁺. |
| | | | yl)prop-2-yn-1-yl)-2-(6- | 2.29 (s, 3H), 1.27-1.08 (m, 5H), 1.04-0.92 | |
| | | | methyl-4- | (m, 7H). | |
| | | | (trifluoromethyl)pyridin- | | |
| | | | 2-yl)isothiazolidine-3- | | |
| | | | carboxamide 1,1-dioxide | | |
| 114 | 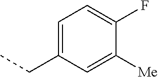 | 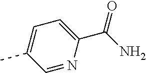 | (R)-N-(3- (6-carbamoylpyridin- 3-yl)prop-2-yn-1-yl)- N-(4-fluoro-3- methylbenzyl)-2-(6- methyl-4-(trifluoromethyl) | (MeOD-d₄) δ = 8.84-8.70 (m, 1H), 8.35- 8.20 (m, 2H), 7.46-7.35 (m, 2H), 7.21-6.91 (m, 3H), 5.65-5.57 (m, 1H), 5.15-4.95 (m, 2H), 4.66-4.32 (m, 2H), 3.85-3.47 (m, 2H), 3.00-2.88 (m, 0.5H), 2.70-2.57 (m, 1H), 2.47 (s, 1H), 2.45-2.35 (m, 0.5H), 2.29 (d, J = 1.2 | 604.1 [M + H]⁺. |
| | | | pyridin-2-yl) | Hz, 1H), 2.21 (s, 2H), 2.16 (d, J = 1.6 Hz, 2H). | |
| | | | isothiazolidine-3- | | |
| | | | carboxamide 1,1-dioxide | | |

The Examples presented in Table 4 were prepared using methods similar to those described in the synthesis of Example 17.

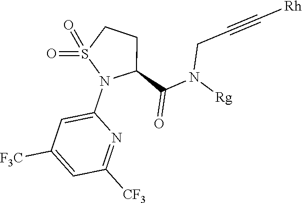

| Example | | | | | MS (ESI) |
|---|---|---|---|---|---|
| Number | Rg | Rh | Name | ¹H NMR (400 MHz) δ PPM | m/z = |
| 125 | 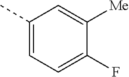 | 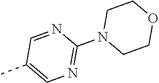 | (S)-2-(4,6-bis(trifluoromethyl) pyridin-2-yl)-N-(4-fluoro-3- methylphenyl)-N-(3-(5- morpholinopyrazin-2-yl)prop- 2-yn-1-yl)isothiazolidine-3- | (DMSO-d₆) δ = 8.14 (br s, 1H), 8.11 (s, 1H), 7.85 (s, 1H), 7.74 (s, 1H), 7.62 (br d, J = 5.2 Hz, 1H), 7.52 (br d, J = 1.2 Hz, 1H), 7.24 (t, J = 9.2 Hz, 1H), 5.04 (t, J = 7.2 Hz, 1H), 4.80 (d, J = 17.6 Hz, 1H), 4.70 (d, J = | 687.0 [M + H]⁺. |
| | | | carboxamide 1,1-dioxide | 17.6 Hz, 1H), 3.85-3.76 (m, 4H), 3.71-3.61 | |
| | | | | (m, 5H), 3.61-3.52 (m, 1H), 2.65-2.50 (m, | |
| | | | | 2H), 2.35 (s, 3H). | |
| 126 | 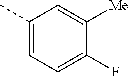 | 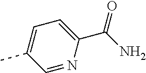 | (S)-2-(4,6-bis(trifluoromethyl) pyridin-2-yl)-N-(3-(6- carbamoylpyridin-3-yl)prop- 2-yn-1-yl)-N-(4-fluoro-3- methylphenyl)isothiazolidine- 3-carboxamide 1,1-dioxide | — | 644.1 [M + H]⁺. |

The Examples presented in Table 5 were prepared using methods similar to those described in the synthesis of Intermediate 3 & Example 17.

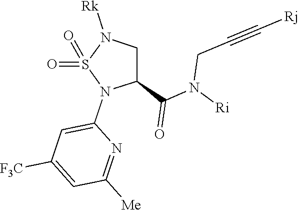

| Exa- | | | | | | |
|---|---|---|---|---|---|---|
| mple | | | | | | MS |
| Num- | | | | | | (ESI) |
| ber | Ri | Rj | Rk | Name | ¹H NMR (400 MHz) δ PPM | m/z = |
| 107 | 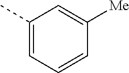 | 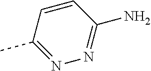 | Me | (3S)-N-[3-(6-aminopyridazin- 3-yl)prop-2-ynyl]-5-methyl-2- [6-methyl-4-(trifluoromethyl)- 2-pyridyl]-N-(m-tolyl)-1,1- | (MeOD-d₄) δ = 7.61-7.43 (m, 3H), 7.41-7.3 2 (m, 2H), 7.27 (d, J = 9.2 Hz, 1H), 7.18 (s, 1H), 6.85 (d, J = 9.2 Hz, 1H), 4.98 (dd, J = 7.6, 5.2 Hz, 2H), 4.59 (d, J = 17.6 Hz, | 560.0 [M + H]⁺. |
| | | | | dioxo-1,2,5-thiadiazolidine-3- | 1H), 3.61-3.55 (m, 1H), 3.53-3.46 (m, 1H), | |
| | | | | carboxamide | 2.82 (s, 3H), 2.59 (s, 3H), 2.45 (s, 3H). | |
| 108 | 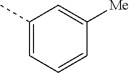 | 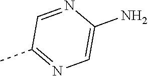 | Me | (S)-N-(3-(5-aminopyrazin-2- yl)prop-2-yn-1-yl)-5-methyl- 2-(6-methyl-4-(trifluoromethyl) pyridin-2-yl)-N-(m-tolyl)-1,2,5- thiadiazolidine-3-carboxamide | (MeOD-d₄) δ = 7.90 (dd, J = 6.8, 1.6 Hz, 2H), 7.54 (br s, 1H), 7.47 (br s, 2H), 7.38- 7.33 (m, 2H), 7.18 (s, 1H), 4.98 (dd, J = 8.0, 5.6 Hz, 1H), 4.92 (d, J = 17.6 Hz, 1H), 4.54 (d, J = 17.6 Hz, 1H), 3.60-3.53 (m, 1H), | 560.0 [M + H]⁺. |
| | | | | 1,1-dioxide | 3.51-3.45 (m, 1H), 2.82 (s, 3H), 2.59 (s, | |
| | | | | | 3H), 2.45 (s, 3H). | |
| 109 | 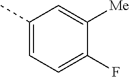 | 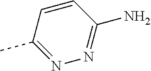 | Me | (S)-N-(3-(6-aminopyridazin-3- yl)prop-2-yn-1-yl)-N-(4-fluoro- 3-methylphenyl)-5-methyl-2- (6-methyl-4-(trifluoromethyl) pyridin-2-yl)-1,2,5- | (MeOD-d₄) δ = 7.70-7.48 (m, 3H), 7.44-7.32 (m, 2H), 7.25 (t, J = 8.8 Hz, 1H), 7.22-7.16 (m, 1H), 5.16-5.02 (m, 1H), 5.02-4.95 (m, 1H), 4.68-4.52 (m, 1H), 3.60-3.48 (m, 2H), 2.82 (s, 3H), 2.61 (s, 3H), 2.36 (s, 3H). | 578.0 [M + H]⁺. |
| | | | | thiadiazolidine-3- | | |
| | | | | carboxamide 1,1-dioxide | | |
| 110 | 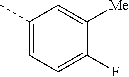 | 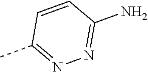 | ⁱPr | (S)-N-(3-(6-aminopyridazin- 3-yl)prop-2-yn-1-yl)-N-(4- fluoro-3-methylphenyl)-5- isopropyl-2-(6-methyl-4- (trifluoromethyl)pyridin-2- | (MeOD-d₄) δ = 7.74 7.51 (m, 2H), 7.33 (s, H), 7.31-7.22 (m, 2H), 7.17 (s, 1H), 6.93 (br s, 1H), 4.99-4.96 (m, 1H), 4.95-4.92 (m, 1H), 4.58 (br d, J = 17.2 Hz, 1H), 3.78 (t d, J = 13.2, 6.8 Hz, 1H), 3.57-3.46 (m, | 606.2 [M + H]⁺. |
| | | | | yl)-1,2,5-thiadiazolidine- | 2H), 2.58 (s, 3H), 2.36 (s, 3H), 1.30 (d, J = | |
| | | | | 3-carboxamide 1,1-dioxide | 6.4 Hz, 3H), 1.22 (d, J = 6.8 Hz, 3H). | |

The Examples presented in Table 2 were prepared using methods similar to those described in the synthesis of Example 17.

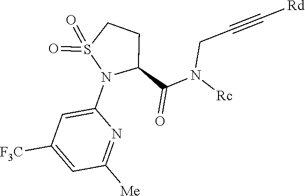

| Example | | | | H NMR (400 | MS (ESI) |
|---|---|---|---|---|---|
| Number | Rc | Rd | Name | MHz) δ PPM | m/z = |
| 137 | 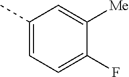 | 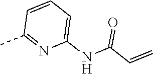 | (S)-N-(3-(6-acrylamidopyridin-2-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |
| 138 | 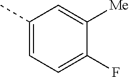 | 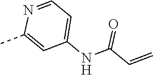 | (S)-N-(3-(4-acrylamidopyridin-2-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |
| 139 | 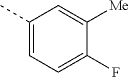 | 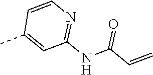 | (S)-N-(3-(2-acrylamidopyridin-4-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |
| 140 | 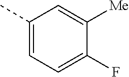 | 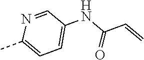 | (S)-N-(3-(5-acrylamidopyridin-2-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.2 [M + H]⁺. |
| 141 | 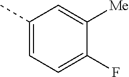 | 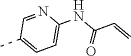 | (S)-N-(3-(6-acrylamidopyridin-3-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |
| 142 | 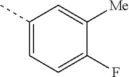 | 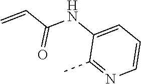 | (S)-N-(3-(3-acrylamidopyridin-2-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.2 [M + H]⁺. |
| 143 | 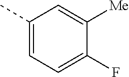 | 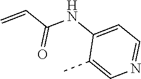 | (S)-N-(3-(4-acrylamidopyridin-3-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |
| 144 | 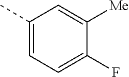 | 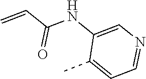 | (S)-N-(3-(3-acrylamidopyridin-4-yl)prop-2-yn-1-yl)-N-(4- fluoro- 3-methylphenyl)-2-(6-methyl-4-(trifluoromethyl)pyridin-2- yl)isothiazolidine-3-carboxamide 1,1-dioxide | — | 616.1 [M + H]⁺. |

### Example 145

### (S)—N-(3-(6-carbamoylpyridin-3-yl)prop-2-yn-1-yl)-N-(4-fluoro-3-methylphenyl)-5-(6-methyl-4-(trifluoromethyl)pyridin-2-yl)-4-thia-5-azaspiro [2,4]heptane-6-carboxamide 4,4-dioxide

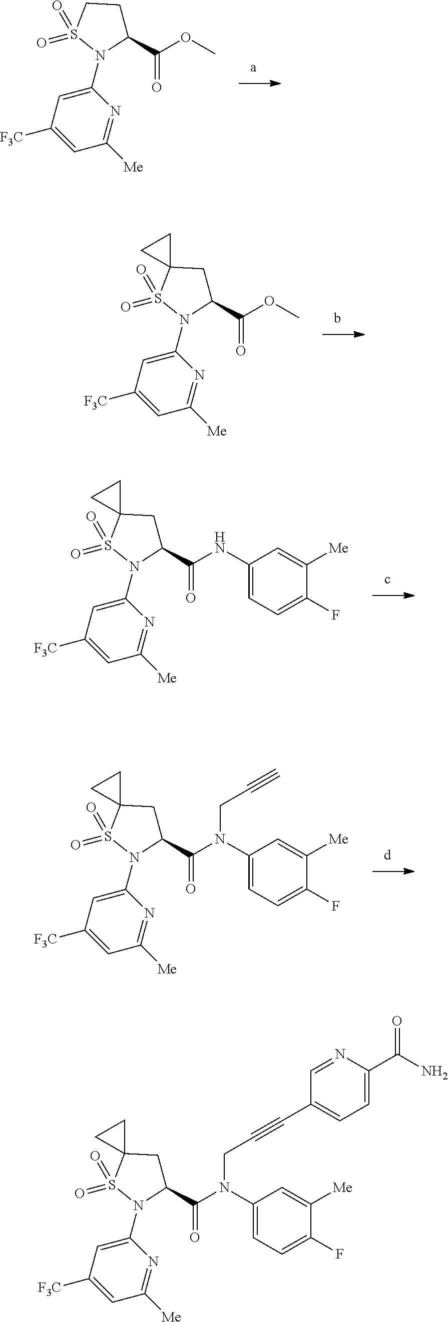 [SMILES: COC(=O)[C@@H]1CCS(=O)(=O)N1c1cc(C(F)(F)F)cc(C)n1 ; COC(=O)[C@@H]1CC2(CC2)S(=O)(=O)N1c1cc(C(F)(F)F)cc(C)n1 ; Cc1cc(C(F)(F)F)cc(N2[C@H](C(=O)Nc3ccc(F)c(C)c3)CC3(CC3)S2(=O)=O)n1 ; C#CCN(C(=O)[C@@H]1CC2(CC2)S(=O)(=O)N1c1cc(C(F)(F)F)cc(C)n1)c1ccc(F)c(C)c1 ; Cc1cc(C(F)(F)F)cc(N2[C@H](C(=O)N(CC#Cc3ccc(C(N)=O)nc3)c3ccc(F)c(C)c3)CC3(CC3)S2(=O)=O)n1]

Step a. To a solution of Intermediate 2 (500 mg, 1.47 mmol) in DMF (7 mL) was added K₂CO₃ (2.03 g, 14.7 mmol) and 1,2-dibromoethane (828 mg, 4.41 mmol). Then the mixture was stirred at 80° C. for 2 hrs. LCMS showed the desired mass was detected. The reaction mixture was dissolved in EtOAc (5 mL), washed with water (5 mL) and brine (5 mL), dried over anhydrous Na₂SO₄ and concentrated under reduced pressure to obtain the residue. The residue was purified by flash silica gel chromatography (ISCO®; 12 g Sepa Flash® Silica Flash Column, Eluent of 0-50% Ethyl acetate/Petroleum ether gradient @40 mL/min) to obtain methyl (S)-5-(6-methyl-4-(trifluoromethyl)pyridin-2-yl)-4-thia-5-azaspiro[2.4]heptane-6-carboxylate 4,4-dioxide (210 mg, 39.1% yield, 95.0% purity) as a yellow oil. MS (ESI) m/z=365.01 [M+H]⁺.

Step b. A mixture of methyl (S)-5-(6-methyl-4-(trifluoromethyl)pyridin-2-yl)-4-thia-5-azaspiro[2.4]heptane-6-carboxylate 4,4-dioxide (200 mg, 0.547 mmol), 4-fluoro-3-methyl-aniline (102.6 mg, 0.82 mmol), AlMe₃ (5 M, 0.65 mL) in toluene (5.4 mL) was degassed and purged 3 times with N₂, and then the mixture was stirred at 100° C. for 16 hrs under an N₂ atmosphere. LCMS showed desired mass was observed. The reaction mixture was quenched by addition of NH₄Cl (10 mL), then diluted with water (20 mL) and extracted with EtOAc (20 mL×3). The combined organic layers were washed with brine (20 mL), dried over Na₂SO₄, filtered and concentrated under reduced pressure to obtain a residue. The residue was purified by flash silica gel chromatography (ISCO®; 12 g Se pa Flash® Silica Flash Column, Eluent of 0-40% Ethyl acetate:Petroleum ether @40 mL/min) to obtain (S)—N-(4-fluoro-3-methylphenyl)-5-(6-methyl-4-(trifluoromethyl)pyridin-2-yl)-4-thia-5-azaspiro[2.4]heptane-6-carboxamide 4,4-dioxide (120 mg, 0.26 mmol, 47.9% yield, 90% purity) as a white solid. MS (ESI) m/z=458.01 [M+H]⁺.

Step c. To a solution of (S)—N-(4-fluoro-3-methylphenyl)-5-(6-methyl-4-(trifluoromethyl)pyridin-2-yl)-4-thia-5-azaspiro[2.4]heptane-6-carboxamide 4,4-dioxide (100 mg, 0.218 mmol) in ACN (2 mL) was added 3-bromoprop-1-yne (64.8 mg, 0.545 mmol), TBAI (8.1 mg, 21.8 μmol) and Cs₂CO₃ (213 mg, 0.65 mmol). The mixture was stirred at 80° C. for 1 h. LCMS showed the desired compound was detected. The crude product was further purified by Prep-HPLC (column: Welch Xtimate C18 100*40 mm*3 um; mobile phase: [water (TFA)-ACN]; gradient:45%-75% B over 8 min) to obtain (S)—N-(4-fluoro-3-methylphenyl)-5-(6-methyl-4-(trifluoromethyl)pyridin-2-yl)-N-(prop-2-yn-1-yl)-4-thia-5-azaspiro[2.4]heptane-6-carboxamide 4,4-dioxide (85 mg, 78.6% yield, 95.0% purity) was obtained as a white solid. MS (ESI) m/z=496.01 [M+H]⁺.

Step d. To a solution of (S)—N-(4-fluoro-3-methylphenyl)-5-(6-methyl-4-(trifluoromethyl)pyridin-2-yl)-N-(prop-2-yn-1-yl)-4-thia-5-azaspiro[2.4]hepta ne-6-carboxamide 4,4-dioxide (50 mg, 0.10 mmol) in THF (2 mL) was added 5-iodopicolinamide (37.2 mg, 0.15 mmol), Pd(PPh₃)₂Cl₂ (7 mg, 0.01 mmol), Et₃N (30.3 mg, 0.3 mmol, 41.8 μL) and CuI (3.8 mg, 0.02 mmol). The mixture was stirred at 25° C. for 12 hrs. LCMS showed the desired compound was detected. The crude product was further purified by Prep-HPLC (column: C18 100×40 mm; mobile phase: [water (TFA)-ACN]; gradient: 38%-68% B over 8 min) to obtain (S)—N-(3-(6-carbamoylpyridin-3-yl)prop-2-yn-1-yl)-N-(4-fluoro-3-methylphenyl)-5-(6-methyl-4-(trifluoromethyl)pyridin-2-yl)-4-thia-5-azaspiro[2.4]heptane-6-carboxamide 4,4-dioxide (15 mg, 0.024 mmol, 24.3% yield, 95% purity) as a white solid.

MS (ESI) m/z=616.01 [M+H]¹.

### Biological Assays

### Expression and Purification of Pol^{θ} Polymerase Domain

The DNA sequence of the polQ gene (Genebank ID #NM 199420.4) was chemically synthesized. The DNA sequence encoding the polymerase domain (amino acids 1792-2590) was subcloned into an E. coli expression plasmid, resulting in the fusion of 10×Histidine-SUMO tag at the N terminus of the polymerase domain.

The protein was expressed in E. coli. and purified using a 2-step IMAC (immobilized metal chelate affinity chromatography) procedure. Briefly stated, the overexpressed E. coli lysate was subjected to Ni-NTA purification, and an N-terminal tag was digested by a SUMO protease. Then, the excised fragment of 10×Histidine-SUMO tag was removed using Ni-NTA beads. The resulting protein was used for the Picogreen assay, as described below.

### Polθ Polymerase Activity Assay

### 1) DNA Complex Preparation

The methods of DNA complex preparation were slightly modified from the protocol previously reported (Zatreanu et al., 2021, Nature Communications, 12:3636). Briefly stated, polθ substrate was prepared by mixing Oligo1 (5′-GCG GCT GTC ATA AG-3′) and Oligo2 (5′-GCT ACA TTG ACA ATG GCA TCA AAT CTC AGA TTG CGT CTT ATG ACA GCC GCG-3′) in an annealing buffer (20 mM Tris-Cl, pH 7.5, 50 mM NaCl) to the final concentration of 22 uM and 20 uM, respectively. The Polθ product was prepared by mixing Oligo3 (5′-CGC GGC TGT CAT AAG ACG CAA TCT GAG ATT TGA TGC CAT TGT CAA TGT AGC-3′) and Oligo2 (5′-GCT ACA TTG ACA ATG GCA TCA AAT CTC AGA TTG CGT CTT ATG ACA GCC GCG-3′) to the final concentration of 20 uM. The polθ product was used for the top signal control in the Picogreen assay. Both substrate and product were heated to 95° C. minutes and cooled slowly to room temperature.

### 2) Picogreen Assay

To evaluate the ability of test compounds to inhibit the activity of polθ in vitro, a Picogreen assay was performed to measure polθ IC₅₀ (nM).

The reactions were performed at room temperature in an assay buffer (25 mM Tris-HCl, pH 7.5, 12.5 mM NaCl, 0.5 mM MgCl₂, 5% (v/v) glycerol, 0.01% (v/v) Triton X-100, 0.01% (w/v) Bovine serum albumin, 1 mM DTT). Thirty microliters of 2×polymerase mixture (8 nM Polθ enzyme in an assay buffer) were distributed to 96 well plates, test compounds were prepared by dilution in 100% DMSO to obtain the correct dose range for a 12-point concentration response and 1 μl was dispensed into each well, and then 30 microliters of 2×substrate mix (100 nM DNA and 40 uM dNTPs in an assay buffer) was added. After 90 minutes of incubation, the reaction was stopped and DNA was stained by adding 40 microliters of Picogreen solution (1:80 dilution of Picogreen, Invitrogen, Cat #P7581, 25 mM Tris-HCl, pH 7.5, 10 mM EDTA, pH 7.5).

The fluorescence intensity was measured at excitation/emission=485 nm/520 nm using an Ensight plate reader (Perkin Elmer). The data was analyzed by GraphPad Prism software (Version 8.4.3) to calculate IC₅₀.

The compounds of Examples 1 to 145 were tested in the above-mentioned assay, and the results are shown in the following table:

| Example Number | PolQ IC₅₀ (nM) |
|---|---|
| 1 | *** |
| 2 | *** |
| 3 | *** |
| 4 | *** |
| 5 | *** |
| 6 | *** |
| 7 | *** |
| 8 | *** |
| 9 | *** |
| 10 | *** |
| 11 | *** |
| 12 | * |
| 13 | *** |
| 14 | *** |
| 15 | *** |
| 16 | *** |
| 17 | *** |
| 18 | *** |
| 19 | *** |
| 20 | *** |
| 21 | *** |
| 22 | *** |
| 23 | *** |
| 24 | *** |
| 25 | *** |
| 26 | *** |
| 27 | *** |
| 28 | *** |
| 29 | *** |
| 30 | *** |
| 31 | * |
| 32 | *** |
| 33 | *** |
| 34 | *** |
| 35 | *** |
| 36 | *** |
| 37 | *** |
| 38 | *** |
| 39 | *** |
| 40 | *** |
| 41 | * |
| 42 | *** |
| 43 | *** |
| 44 | *** |
| 45 | *** |
| 46 | *** |
| 47 | *** |
| 48 | *** |
| 49 | *** |
| 50 | *** |
| 51 | *** |
| 52 | *** |
| 53 | *** |
| 54 | * |
| 55 | *** |
| 56 | *** |
| 57 | *** |
| 58 | * |
| 59 | * |
| 60 | *** |
| 61 | * |
| 62 | *** |
| 63 | *** |
| 64 | *** |
| 65 | * |
| 66 | ** |
| 67 | *** |
| 68 | *** |
| 69 | *** |
| 70 | *** |
| 71 | *** |
| 72 | *** |
| 73 | *** |
| 74 | * |
| 75 | *** |
| 76 | ** |
| 77 | *** |
| 78 | *** |
| 79 | *** |
| 80 | *** |
| 81 | * |
| 82 | ** |
| 83 | *** |
| 84 | *** |
| 85 | *** |
| 86 | *** |
| 87 | ** |
| 88 | * |
| 89 | ** |
| 90 | ** |
| 91 | *** |
| 92 | *** |
| 93 | *** |
| 94 | ** |
| 95 | *** |
| 96 | *** |
| 97 | *** |
| 98 | *** |
| 99 | *** |
| 100 | *** |
| 101 | ** |
| 102 | *** |
| 103 | * |
| 104 | *** |
| 105 | *** |
| 106 | *** |
| 107 | *** |
| 108 | ** |
| 109 | *** |
| 110 | *** |
| 111 | *** |
| 112 | * |
| 113 | * |
| 114 | * |
| 115 | *** |
| 116 | *** |
| 117 | *** |
| 118 | *** |
| 119 | *** |
| 120 | *** |
| 121 | *** |
| 122 | *** |
| 123 | *** |
| 124 | *** |
| 125 | *** |
| 126 | *** |
| 127 | *** |
| 128 | *** |
| 129 | *** |
| 130 | *** |
| 131 | *** |
| 132 | *** |
| 133 | *** |
| 134 | *** |
| 135 | *** |
| 136 | *** |
| 137 | *** |
| 138 | *** |
| 139 | *** |
| 140 | *** |
| 141 | *** |
| 142 | *** |
| 143 | ** |
| 144 | ** |
| 145 | *** |

Note:
1 nM <***<50 nM; 51 nM <**<100 nM; and *>101 nM

As shown in the above Table, it is demonstrated that the tested compounds exhibit good activity in inhibiting the Polθ polymerase.

### 3) Cell Viability Assay

DLD1-BRCA2 KG (Horizon Discovery, Cat #HD 105-007) cells and DLD1-parental cells were maintained in culture media (RPMI-1640 including 2 mM L-glutamin and 25 mM sodium bicarbonate, supplemented with 10% FBS), at 37° C. in a humidified atmosphere with 5% CO₂. Cells were seeded at the final concentration of 1,500 cells/well (DLD1-BRCA2 KG) and 600 cells/well (DLD1-parental) in a 96-well plate. Four hours later, test compounds were added (the final concentration of DMSO was 1%) to the cells and media were replaced every 3-4 days for 14 days. To measure viability, cells were incubated in culture media supplemented with 0.1 mg/ml of Resazurin (Merck, Cat #R7017) for 4 hours. The fluorescence was measured at Ex/Em=560 nm/590 nm using EnSight multimode plate reader (Perkin Elmer). Data analysis and IC₅₀ calculation was done using GraphPad Prism (Version 10.0.2).

Results: IC₅₀ values determined for selected compounds of the invention are presented in Table below.

| | IC₅₀ in DLD-1 |
|---|---|
| Example | BRCA2(−/−) |
| Number | Cell (uM) |
| 1 | *** |
| 2 | *** |
| 3 | *** |
| 4 | *** |
| 5 | *** |
| 6 | *** |
| 7 | *** |
| 8 | *** |
| 9 | *** |
| 10 | *** |
| 11 | *** |
| 12 | *** |
| 13 | ** |
| 14 | ** |
| 15 | ** |
| 16 | ** |
| 17 | *** |
| 18 | *** |
| 19 | ** |
| 20 | *** |
| 21 | *** |
| 22 | ** |
| 23 | ** |
| 24 | *** |
| 25 | ** |
| 26 | *** |
| 27 | *** |
| 28 | *** |
| 29 | *** |
| 30 | ** |
| 31 | * |
| 32 | *** |
| 33 | *** |
| 34 | ** |
| 35 | ** |
| 36 | *** |
| 37 | ** |
| 38 | ** |
| 39 | *** |
| 40 | ** |
| 41 | * |
| 42 | *** |
| 43 | ** |
| 44 | ** |
| 45 | *** |
| 46 | ** |
| 47 | ** |
| 48 | *** |
| 49 | *** |
| 50 | *** |
| 51 | *** |
| 52 | *** |
| 53 | *** |
| 54 | * |
| 55 | *** |
| 56 | *** |
| 57 | *** |
| 58 | *** |
| 59 | *** |
| 60 | * |
| 61 | * |
| 62 | *** |
| 63 | *** |
| 64 | *** |
| 65 | *** |
| 66 | *** |
| 67 | * |
| 68 | *** |
| 69 | *** |
| 70 | ** |
| 71 | *** |
| 72 | *** |
| 73 | *** |
| 74 | *** |
| 75 | *** |
| 76 | *** |
| 77 | *** |
| 78 | *** |
| 79 | *** |
| 80 | *** |
| 81 | *** |
| 82 | *** |
| 83 | *** |
| 84 | *** |
| 85 | *** |
| 86 | *** |
| 87 | *** |
| 88 | ** |
| 89 | ** |
| 90 | ** |
| 91 | ** |
| 92 | *** |
| 93 | *** |
| 94 | ** |
| 95 | *** |
| 96 | *** |
| 97 | *** |
| 98 | *** |
| 99 | ** |
| 100 | * |
| 101 | ** |
| 102 | ** |
| 103 | *** |
| 104 | *** |
| 105 | ** |
| 106 | ** |
| 107 | ** |
| 108 | ** |
| 109 | ** |
| 110 | ** |
| 111 | *** |
| 112 | — |
| 113 | — |
| 114 | — |
| 115 | *** |
| 116 | *** |
| 117 | *** |
| 118 | — |
| 119 | — |
| 120 | |
| 121 | — |
| 122 | — |
| 123 | — |
| 124 | — |
| 125 | — |
| 126 | — |
| 127 | — |
| 128 | — |
| 129 | — |
| 130 | — |
| 131 | — |
| 132 | — |
| 133 | — |
| 134 | — |
| 135 | — |
| 136 | — |
| 137 | — |
| 138 | — |
| 139 | — |
| 140 | — |
| 141 | — |
| 142 | — |
| 143 | — |
| 144 | — |
| 145 | — |

Note:
***<1 uM; 1.1 uM <**<5 uM; and *>5.1 uM

While the compounds, compositions, formulations, and methods of this disclosure have been described in terms of specific embodiments, it will be apparent to those skilled in the art that variations or modifications may be applied to the compounds, compositions, formulations, and methods described herein without departing from the spirit and scope of this disclosure. All such variations or modifications apparent to those skilled in the art are deemed to be within the spirit and scope of this disclosure as defined by the appended claims.

The present disclosure provides the following embodiments:

Embodiment 1. A compound of the following Formula (I):

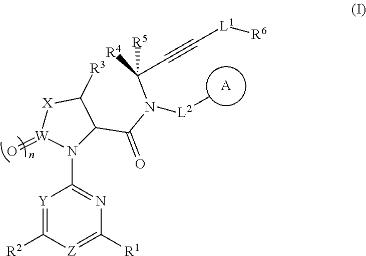

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L¹ is selected from the group consisting of absent, —(C₁-C₆)alkylene-, and —(C₁-C₆)alkylene-NH—;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

Embodiment 2. The compound according to embodiment 1, wherein the compound is a compound represented by Formula (II):

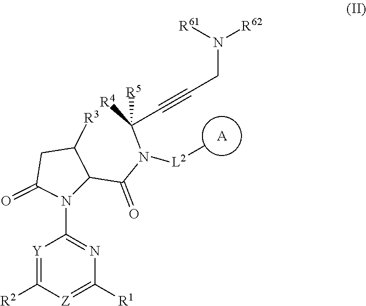

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl.

Embodiment 3. The compound according to any one of embodiments 1 to 2, wherein the compound is a compound represented by Formula (IIa-1):

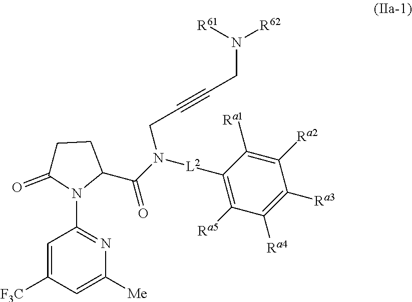

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

Embodiment 4. The compound according to any one of embodiments 2 to 3, wherein each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen.

Embodiment 5. The compound according to embodiment 1, wherein the compound is a compound represented by Formula (III):

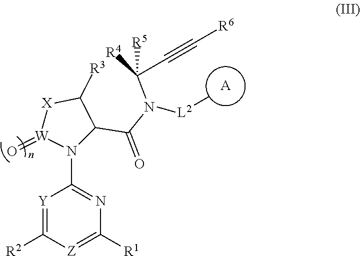

wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z1}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl.

Embodiment 6. The compound according to any one of embodiments 1 and 5, wherein the compound is a compound represented by Formula (IIIa-1):

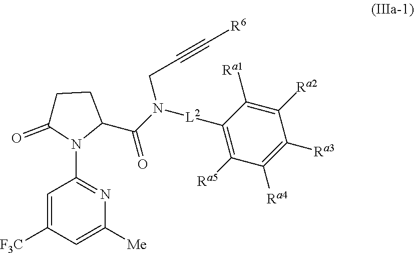

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

Embodiment 7. The compound according to any one of embodiments 1 and 5, wherein the compound is a compound represented by Formula (IIIb-1):

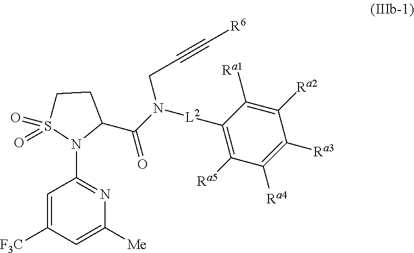

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

Embodiment 8. The compound according to any one of embodiments 1 and 5, wherein the compound is a compound represented by Formula (IIIc-1):

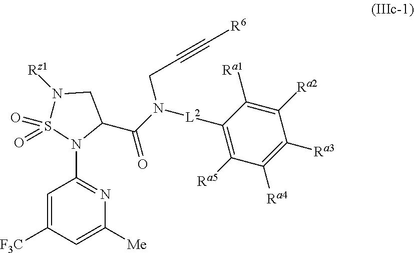

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

Embodiment 9. The compound according to any one of embodiments 1 and 5, wherein the compound is a compound represented by Formula (IIId-1):

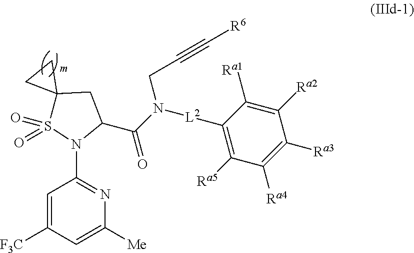

wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
m is an integer from 1 to 4;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

Embodiment 10. The compound according to any one of embodiments 3 and 6 to 9, wherein each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo.

Embodiment 11. The compound according to any one of embodiments 1 and 5 to 10, wherein R⁶ is selected from the group consisting of:

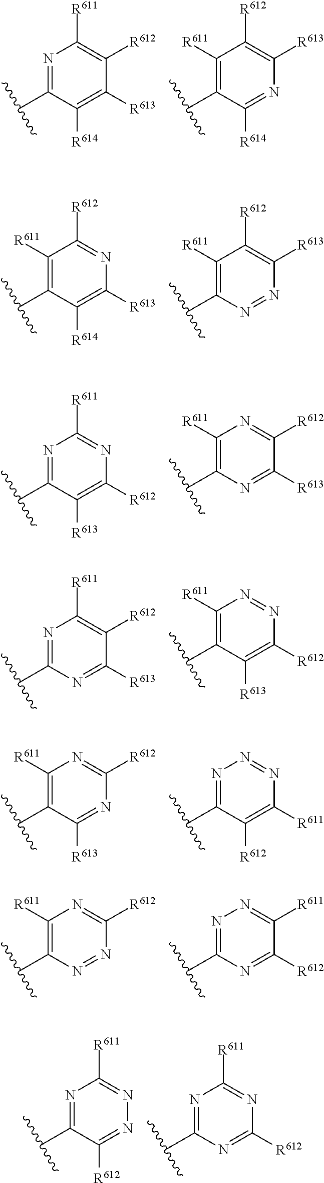

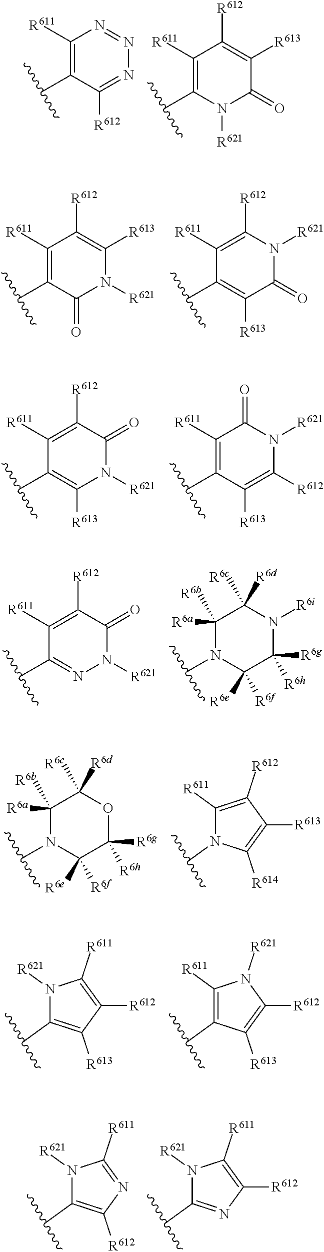

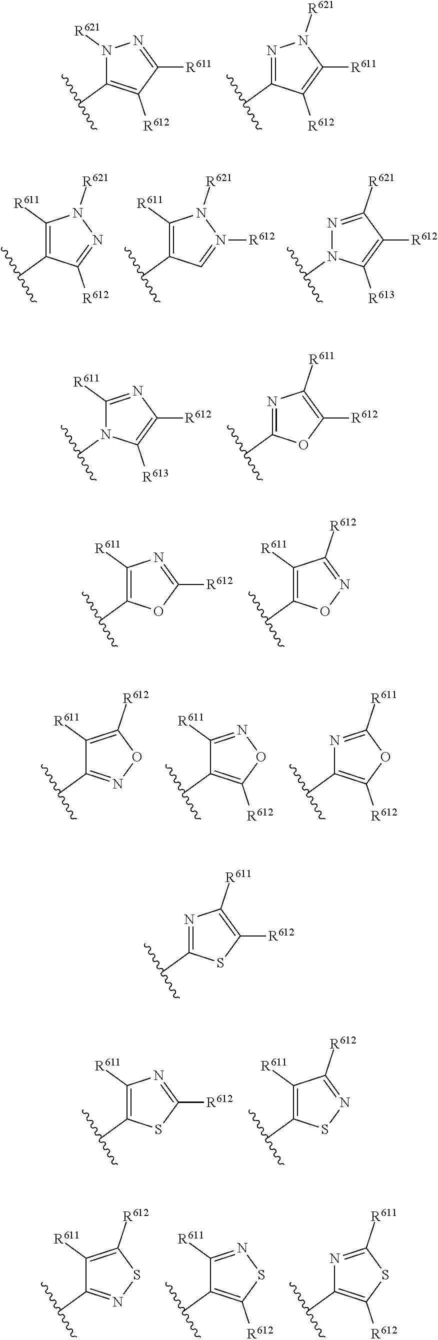

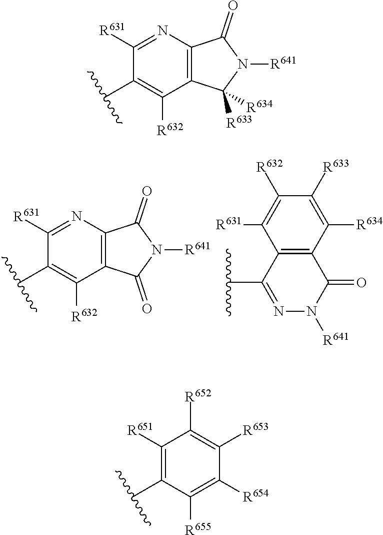

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

Embodiment 12. The compound according to any one of embodiments 1 and 5 to 11, wherein R⁶ is selected from the group consisting of:

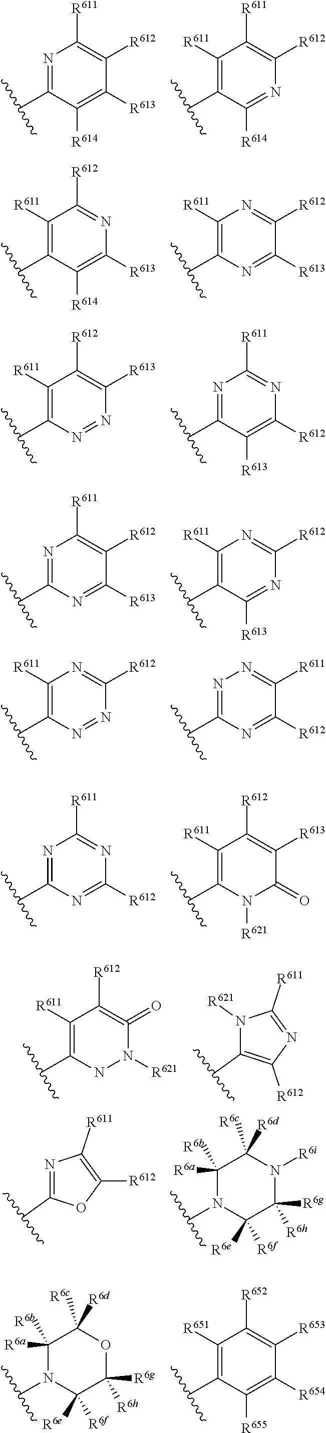

each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, —C(O)NR⁶⁴R⁶⁵, —CH₂—R⁶⁶,

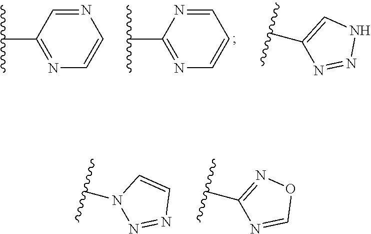

each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is:

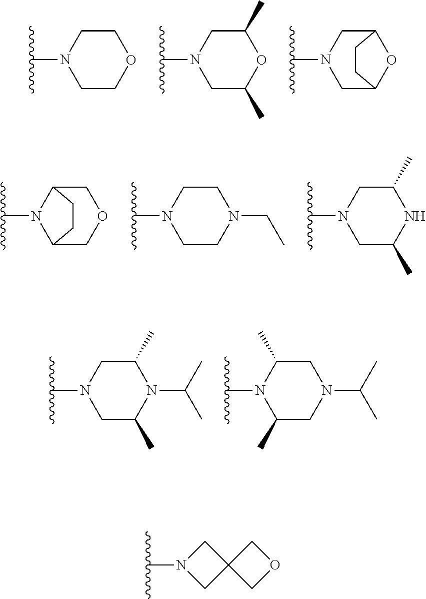

R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is:

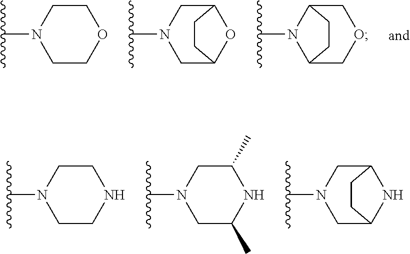

R⁶⁶ is a 6-10 membered heterocyclyl group, which is:

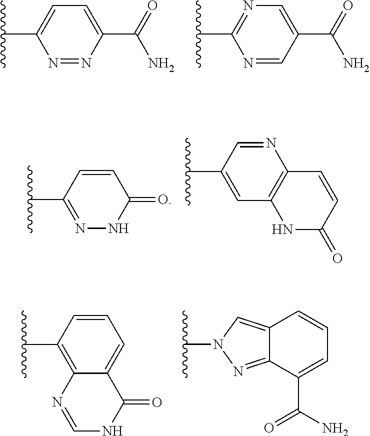

Embodiment 13. The compound according to any one of preceding embodiments, wherein the compound is selected from the group consisting of:

| NO. | Structure |
|---|---|
| 1 | 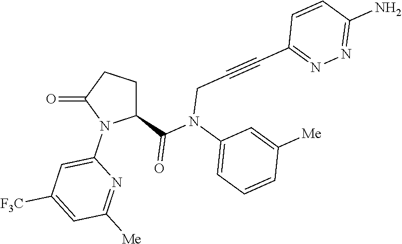 [SMILES: Cc1cccc(N(CC#Cc2ccc(N)nn2)C(=O)[C@@H]2CCC(=O)N2c2cc(C(F)(F)F)cc(C)n2)c1] |
| 2 | 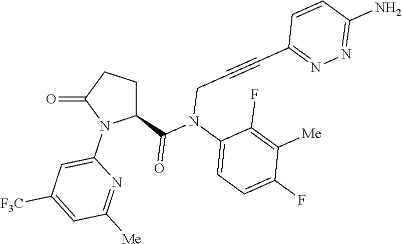 [SMILES: Cc1cc(C(F)(F)F)cc(N2C(=O)CC[C@H]2C(=O)N(CC#Cc2ccc(N)nn2)c2ccc(F)c(C)c2F)n1] |
| 3 | 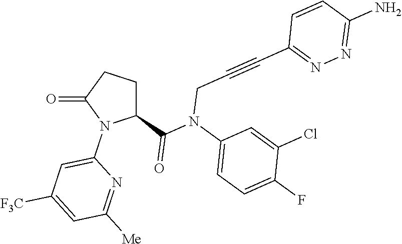 [SMILES: Cc1cc(C(F)(F)F)cc(N2C(=O)CC[C@H]2C(=O)N(CC#Cc2ccc(N)nn2)c2ccc(F)c(Cl)c2)n1] |
| 4 | 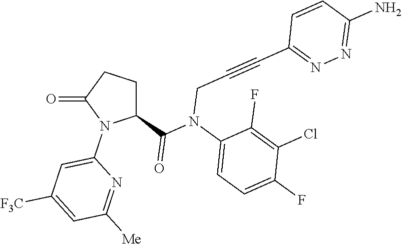 [SMILES: Cc1cc(C(F)(F)F)cc(N2C(=O)CC[C@H]2C(=O)N(CC#Cc2ccc(N)nn2)c2ccc(F)c(Cl)c2F)n1] |
| 5 | 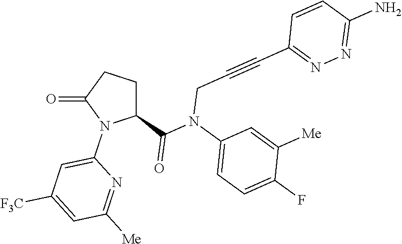 [SMILES: Cc1cc(C(F)(F)F)cc(N2C(=O)CC[C@H]2C(=O)N(CC#Cc2ccc(N)nn2)c2ccc(F)c(C)c2)n1] |
| 6 | 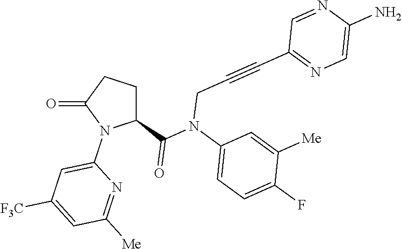 [SMILES: Cc1cc(C(F)(F)F)cc(N2C(=O)CC[C@H]2C(=O)N(CC#Cc2cnc(N)cn2)c2ccc(F)c(C)c2)n1] |
| 7 | 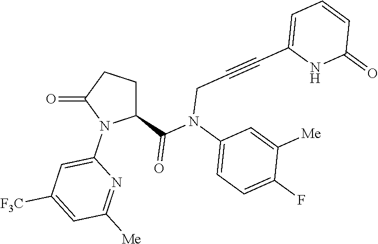 [SMILES: Cc1cc(C(F)(F)F)cc(N2C(=O)CC[C@H]2C(=O)N(CC#Cc2cccc(=O)[nH]2)c2ccc(F)c(C)c2)n1] |
| 8 | 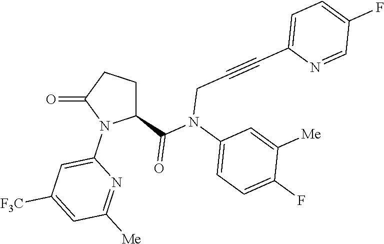 [SMILES: Cc1cc(C(F)(F)F)cc(N2C(=O)CC[C@H]2C(=O)N(CC#Cc2ccc(F)cn2)c2ccc(F)c(C)c2)n1] |
| 9 | 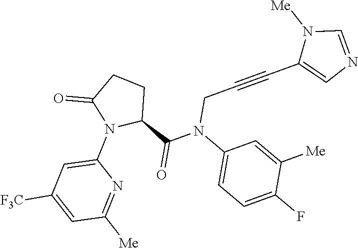 [SMILES: Cc1cc(C(F)(F)F)cc(N2C(=O)CC[C@H]2C(=O)N(CC#Cc2cncn2C)c2ccc(F)c(C)c2)n1] |
| 10 | 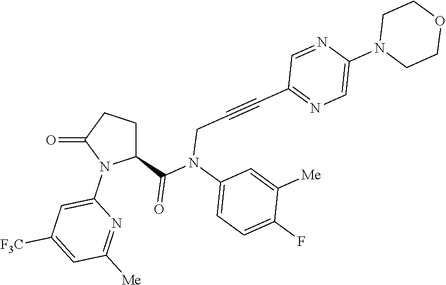 [SMILES: Cc1cc(C(F)(F)F)cc(N2C(=O)CC[C@H]2C(=O)N(CC#Cc2cnc(N3CCOCC3)cn2)c2ccc(F)c(C)c2)n1] |
| 11 | 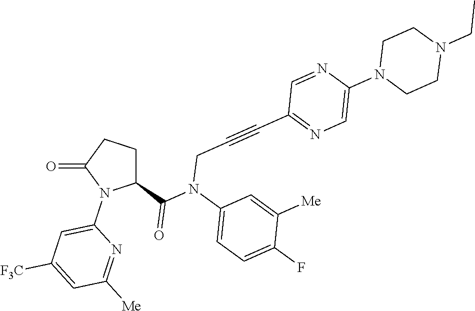 [SMILES: CCN1CCN(c2cnc(C#CCN(C(=O)[C@@H]3CCC(=O)N3c3cc(C(F)(F)F)cc(C)n3)c3ccc(F)c(C)c3)cn2)CC1] |
| 12 | 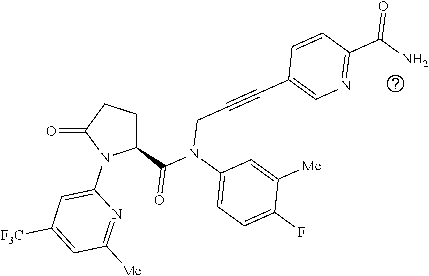 [SMILES: Cc1cc(C(F)(F)F)cc(N2C(=O)CC[C@H]2C(=O)N(CC#Cc2ccc(C(N)=O)nc2)c2ccc(F)c(C)c2)n1] |
| 13 | 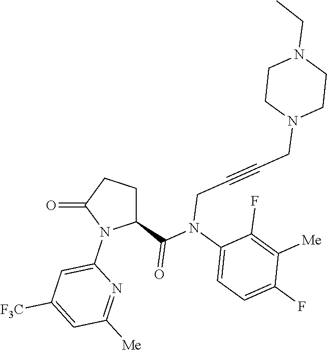 [SMILES: CCN1CCN(CC#CCN(C(=O)[C@@H]2CCC(=O)N2c2cc(C(F)(F)F)cc(C)n2)c2ccc(F)c(C)c2F)CC1] |
| 14 | 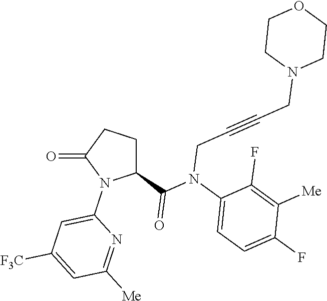 [SMILES: Cc1cc(C(F)(F)F)cc(N2C(=O)CC[C@H]2C(=O)N(CC#CCN2CCOCC2)c2ccc(F)c(C)c2F)n1] |
| 15 | 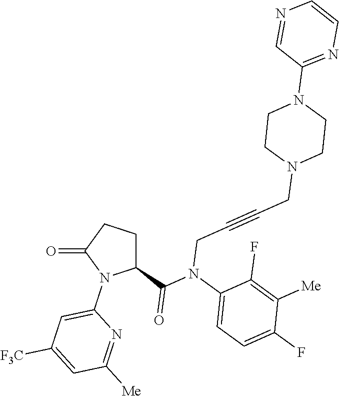 [SMILES: Cc1cc(C(F)(F)F)cc(N2C(=O)CC[C@H]2C(=O)N(CC#CCN2CCN(c3cnccn3)CC2)c2ccc(F)c(C)c2F)n1] |
| 16 | 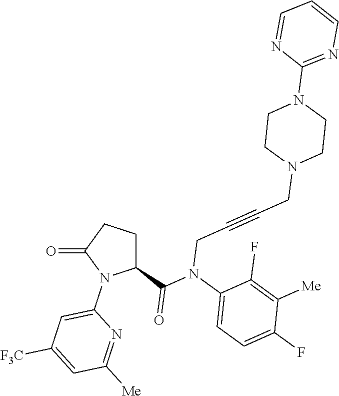 [SMILES: Cc1cc(C(F)(F)F)cc(N2C(=O)CC[C@H]2C(=O)N(CC#CCN2CCN(c3ncccn3)CC2)c2ccc(F)c(C)c2F)n1] |
| 17 | 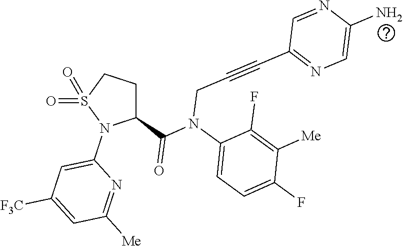 [SMILES: Cc1cc(C(F)(F)F)cc(N2[C@H](C(=O)N(CC#Cc3cnc(N)cn3)c3ccc(F)c(C)c3F)CCS2(=O)=O)n1] |
| 18 | 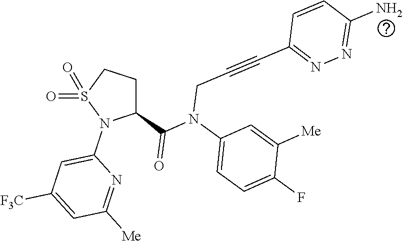 [SMILES: Cc1cc(C(F)(F)F)cc(N2[C@H](C(=O)N(CC#Cc3ccc(N)nn3)c3ccc(F)c(C)c3)CCS2(=O)=O)n1] |
| 19 | 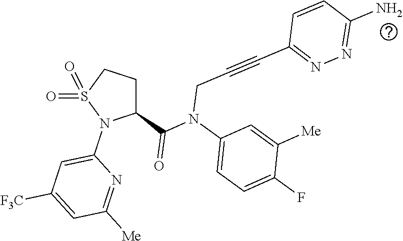 [SMILES: Cc1cc(C(F)(F)F)cc(N2[C@H](C(=O)N(CC#Cc3ccc(N)nn3)c3ccc(F)c(C)c3)CCS2(=O)=O)n1] |
| 20 | 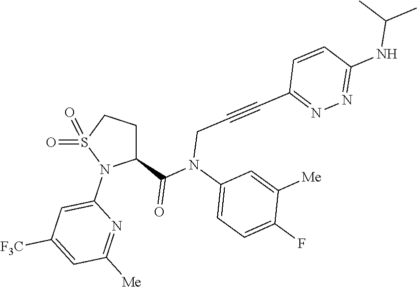 [SMILES: Cc1cc(C(F)(F)F)cc(N2[C@H](C(=O)N(CC#Cc3ccc(NC(C)C)nn3)c3ccc(F)c(C)c3)CCS2(=O)=O)n1] |
| 21 | 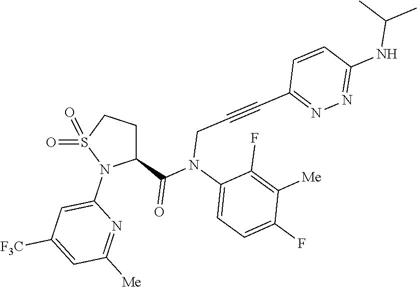 [SMILES: Cc1cc(C(F)(F)F)cc(N2[C@H](C(=O)N(CC#Cc3ccc(NC(C)C)nn3)c3ccc(F)c(C)c3F)CCS2(=O)=O)n1] |
| 22 | 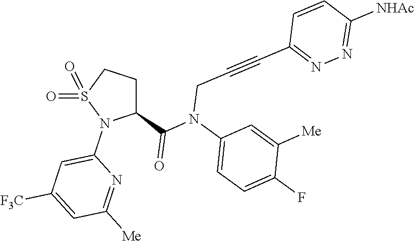 [SMILES: CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 23 | 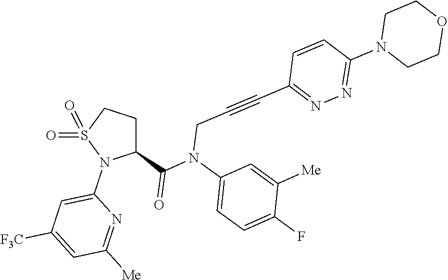 [SMILES: Cc1cc(C(F)(F)F)cc(N2[C@H](C(=O)N(CC#Cc3ccc(N4CCOCC4)nn3)c3ccc(F)c(C)c3)CCS2(=O)=O)n1] |
| 24 | 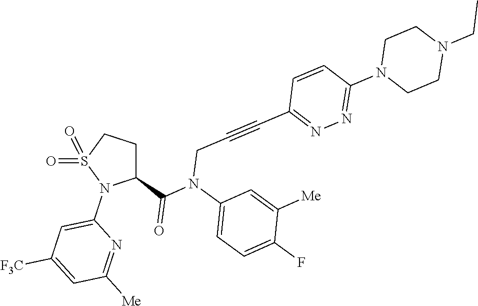 [SMILES: CCN1CCN(c2ccc(C#CCN(C(=O)[C@@H]3CCS(=O)(=O)N3c3cc(C(F)(F)F)cc(C)n3)c3ccc(F)c(C)c3)nn2)CC1] |
| 25 | 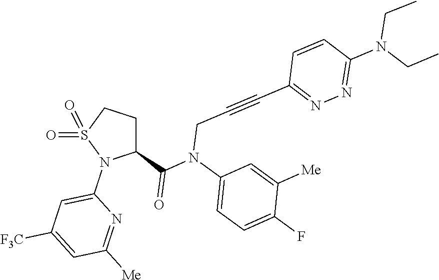 [SMILES: CCN(CC)c1ccc(C#CCN(C(=O)[C@@H]2CCS(=O)(=O)N2c2cc(C(F)(F)F)cc(C)n2)c2ccc(F)c(C)c2)nn1] |
| 26 | 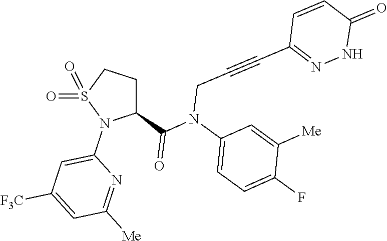 [SMILES: Cc1cc(C(F)(F)F)cc(N2[C@H](C(=O)N(CC#Cc3ccc(=O)[nH]n3)c3ccc(F)c(C)c3)CCS2(=O)=O)n1] |
| 27 | 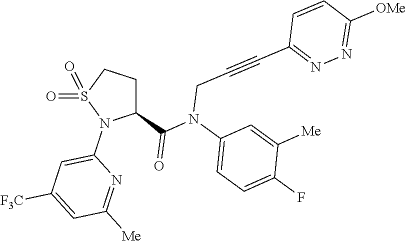 [SMILES: COc1ccc(C#CCN(C(=O)[C@@H]2CCS(=O)(=O)N2c2cc(C(F)(F)F)cc(C)n2)c2ccc(F)c(C)c2)nn1] |
| 28 | 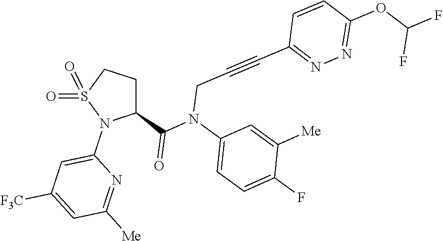 [SMILES: Cc1cc(C(F)(F)F)cc(N2[C@H](C(=O)N(CC#Cc3ccc(OC(F)F)nn3)c3ccc(F)c(C)c3)CCS2(=O)=O)n1] |
| 29 | 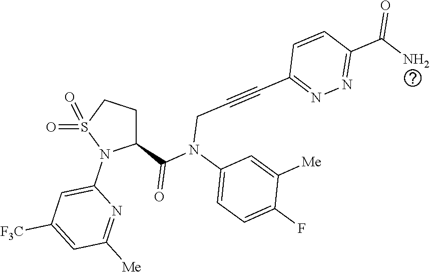 [SMILES: Cc1cc(C(F)(F)F)cc(N2[C@H](C(=O)N(CC#Cc3ccc(C(N)=O)nn3)c3ccc(F)c(C)c3)CCS2(=O)=O)n1] |
| 30 | 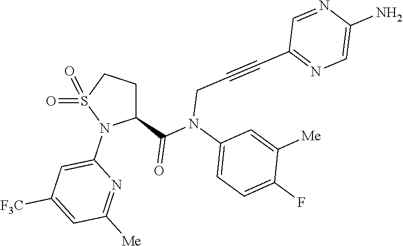 [SMILES: Cc1cc(C(F)(F)F)cc(N2[C@H](C(=O)N(CC#Cc3cnc(N)cn3)c3ccc(F)c(C)c3)CCS2(=O)=O)n1] |
| 31 | 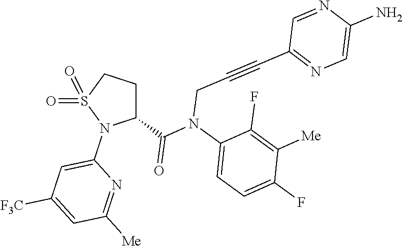 [SMILES: Cc1cc(C(F)(F)F)cc(N2[C@@H](C(=O)N(CC#Cc3cnc(N)cn3)c3ccc(F)c(C)c3F)CCS2(=O)=O)n1] |
| 32 | 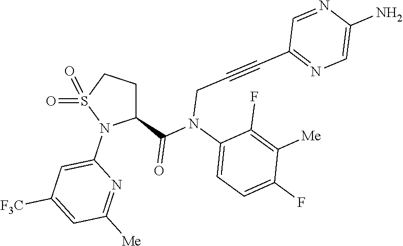 [SMILES: Cc1cc(C(F)(F)F)cc(N2[C@H](C(=O)N(CC#Cc3cnc(N)cn3)c3ccc(F)c(C)c3F)CCS2(=O)=O)n1] |
| 33 | 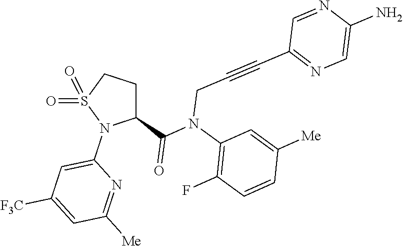 [SMILES: Cc1ccc(F)c(N(CC#Cc2cnc(N)cn2)C(=O)[C@@H]2CCS(=O)(=O)N2c2cc(C(F)(F)F)cc(C)n2)c1] |
| 34 | 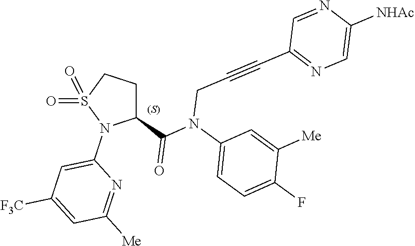 |
| 35 | 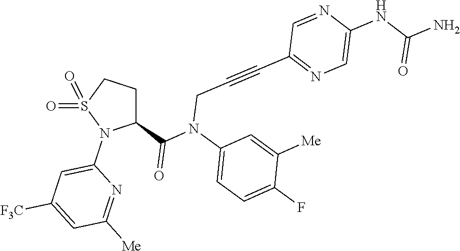 [SMILES: Cc1cc(C(F)(F)F)cc(N2[C@H](C(=O)N(CC#Cc3cnc(NC(N)=O)cn3)c3ccc(F)c(C)c3)CCS2(=O)=O)n1] |
| 36 | 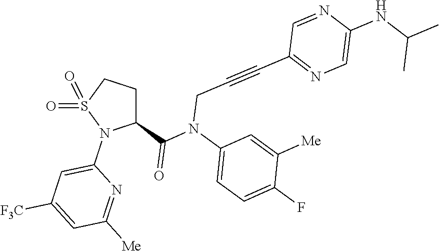 [SMILES: Cc1cc(C(F)(F)F)cc(N2[C@H](C(=O)N(CC#Cc3cnc(NC(C)C)cn3)c3ccc(F)c(C)c3)CCS2(=O)=O)n1] |
| 37 | 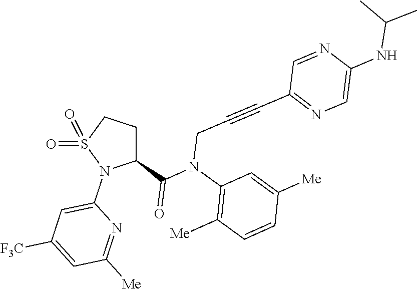 [SMILES: Cc1ccc(C)c(N(CC#Cc2cnc(NC(C)C)cn2)C(=O)[C@@H]2CCS(=O)(=O)N2c2cc(C(F)(F)F)cc(C)n2)c1] |
| 38 | 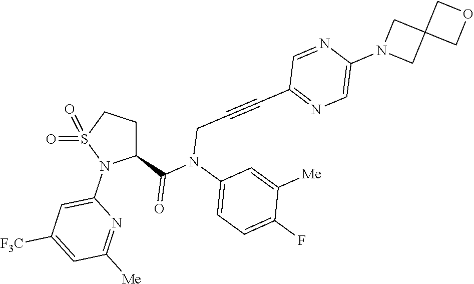 [SMILES: Cc1cc(C(F)(F)F)cc(N2[C@H](C(=O)N(CC#Cc3cnc(N4CC5(COC5)C4)cn3)c3ccc(F)c(C)c3)CCS2(=O)=O)n1] |
| 39 | 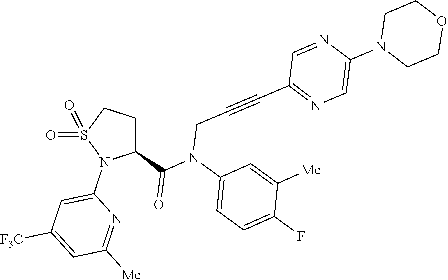 [SMILES: Cc1cc(C(F)(F)F)cc(N2[C@H](C(=O)N(CC#Cc3cnc(N4CCOCC4)cn3)c3ccc(F)c(C)c3)CCS2(=O)=O)n1] |
| 40 | 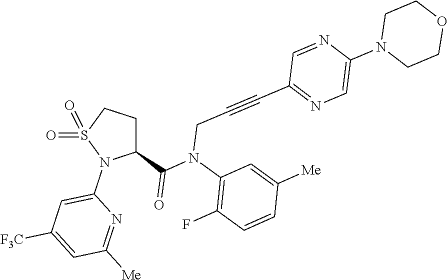 [SMILES: Cc1ccc(F)c(N(CC#Cc2cnc(N3CCOCC3)cn2)C(=O)[C@@H]2CCS(=O)(=O)N2c2cc(C(F)(F)F)cc(C)n2)c1] |
| 41 | 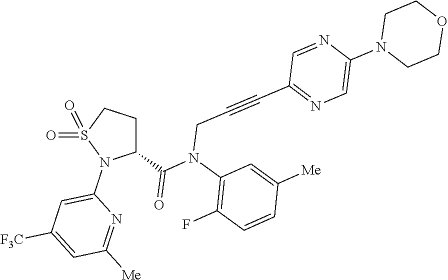 [SMILES: Cc1ccc(F)c(N(CC#Cc2cnc(N3CCOCC3)cn2)C(=O)[C@H]2CCS(=O)(=O)N2c2cc(C(F)(F)F)cc(C)n2)c1] |
| 42 | 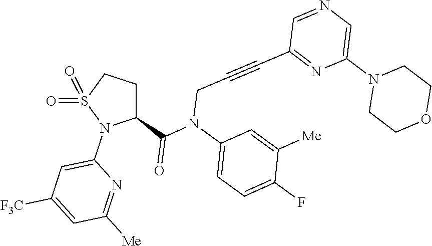 [SMILES: Cc1cc(C(F)(F)F)cc(N2[C@H](C(=O)N(CC#Cc3cncc(N4CCOCC4)n3)c3ccc(F)c(C)c3)CCS2(=O)=O)n1] |
| 43 | 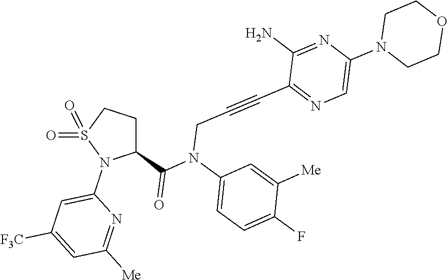 [SMILES: Cc1cc(C(F)(F)F)cc(N2[C@H](C(=O)N(CC#Cc3ncc(N4CCOCC4)nc3N)c3ccc(F)c(C)c3)CCS2(=O)=O)n1] |
| 44 | 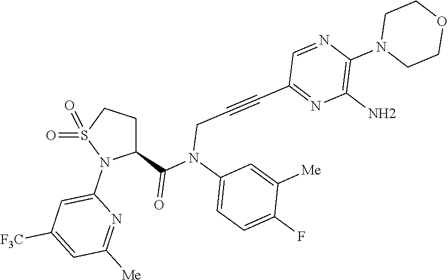 [SMILES: Cc1cc(C(F)(F)F)cc(N2[C@H](C(=O)N(CC#Cc3cnc(N4CCOCC4)c(N)n3)c3ccc(F)c(C)c3)CCS2(=O)=O)n1] |
| 45 | 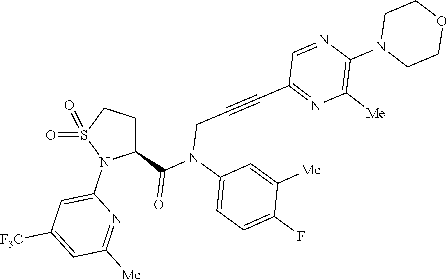 [SMILES: Cc1cc(C(F)(F)F)cc(N2[C@H](C(=O)N(CC#Cc3cnc(N4CCOCC4)c(C)n3)c3ccc(F)c(C)c3)CCS2(=O)=O)n1] |
| 46 | 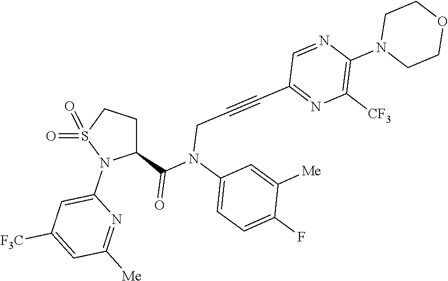 [SMILES: Cc1cc(C(F)(F)F)cc(N2[C@H](C(=O)N(CC#Cc3cnc(N4CCOCC4)c(C(F)(F)F)n3)c3ccc(F)c(C)c3)CCS2(=O)=O)n1] |
| 47 | 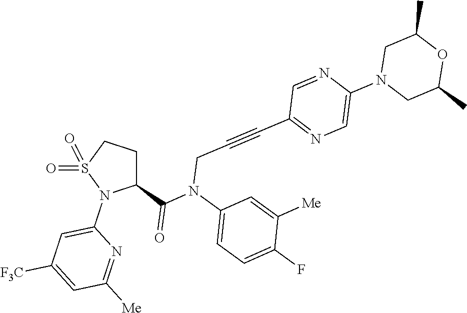 [SMILES: Cc1cc(C(F)(F)F)cc(N2[C@H](C(=O)N(CC#Cc3cnc(N4C[C@@H](C)O[C@@H](C)C4)cn3)c3ccc(F)c(C)c3)CCS2(=O)=O)n1] |
| 48 | 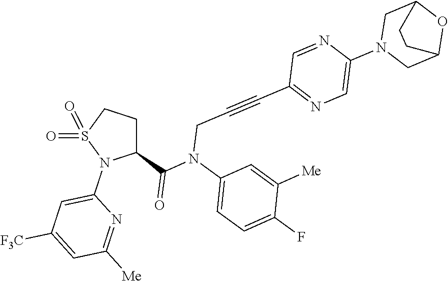 [SMILES: Cc1cc(C(F)(F)F)cc(N2[C@H](C(=O)N(CC#Cc3cnc(N4CC5CCC(C4)O5)cn3)c3ccc(F)c(C)c3)CCS2(=O)=O)n1] |
| 49 | 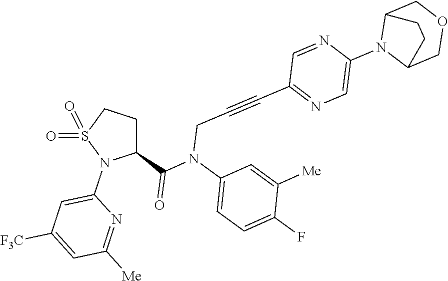 [SMILES: Cc1cc(C(F)(F)F)cc(N2[C@H](C(=O)N(CC#Cc3cnc(N4C5CCC4COC5)cn3)c3ccc(F)c(C)c3)CCS2(=O)=O)n1] |
| 50 | 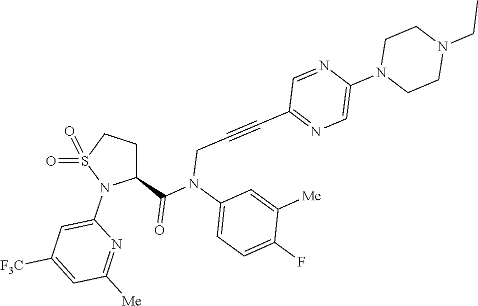 [SMILES: CCN1CCN(c2cnc(C#CCN(C(=O)[C@@H]3CCS(=O)(=O)N3c3cc(C(F)(F)F)cc(C)n3)c3ccc(F)c(C)c3)cn2)CC1] |
| 51 | 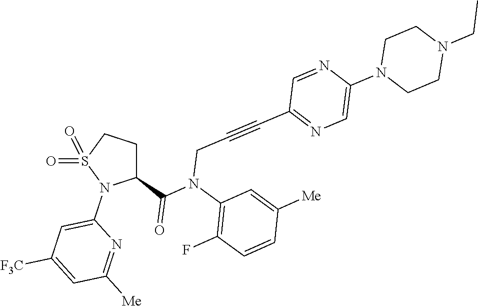 [SMILES: CCN1CCN(c2cnc(C#CCN(C(=O)[C@@H]3CCS(=O)(=O)N3c3cc(C(F)(F)F)cc(C)n3)c3cc(C)ccc3F)cn2)CC1] |
| 52 | 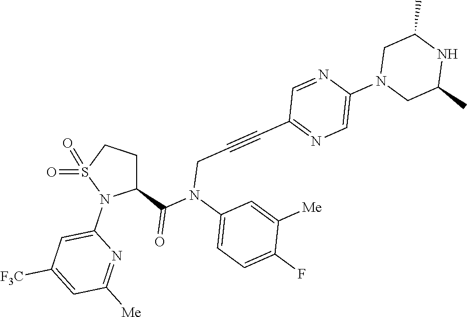 [SMILES: Cc1cc(C(F)(F)F)cc(N2[C@H](C(=O)N(CC#Cc3cnc(N4C[C@H](C)N[C@@H](C)C4)cn3)c3ccc(F)c(C)c3)CCS2(=O)=O)n1] |
| 53 | 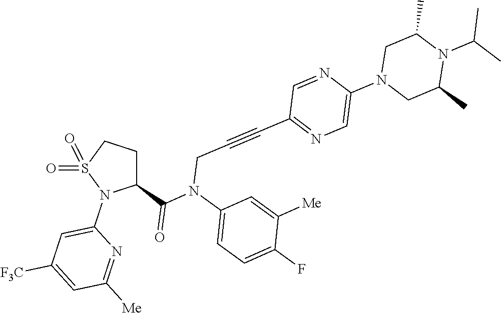 [SMILES: Cc1cc(C(F)(F)F)cc(N2[C@H](C(=O)N(CC#Cc3cnc(N4C[C@H](C)N(C(C)C)[C@@H](C)C4)cn3)c3ccc(F)c(C)c3)CCS2(=O)=O)n1] |
| 54 | 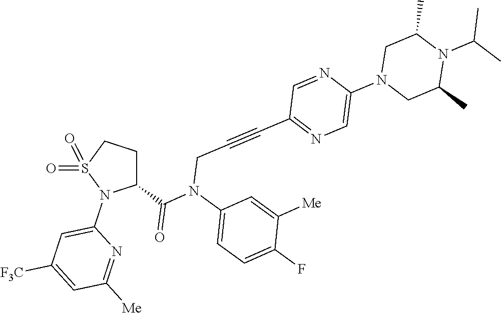 [SMILES: Cc1cc(C(F)(F)F)cc(N2[C@@H](C(=O)N(CC#Cc3cnc(N4C[C@H](C)N(C(C)C)[C@@H](C)C4)cn3)c3ccc(F)c(C)c3)CCS2(=O)=O)n1] |
| 55 | 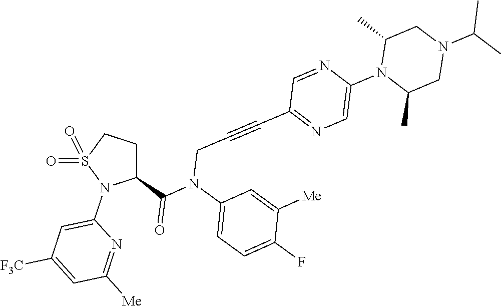 [SMILES: Cc1cc(C(F)(F)F)cc(N2[C@H](C(=O)N(CC#Cc3cnc(N4[C@H](C)CN(C(C)C)C[C@H]4C)cn3)c3ccc(F)c(C)c3)CCS2(=O)=O)n1] |
| 56 | 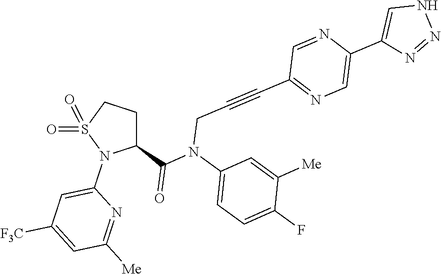 [SMILES: Cc1cc(C(F)(F)F)cc(N2[C@H](C(=O)N(CC#Cc3cnc(-c4c[nH]nn4)cn3)c3ccc(F)c(C)c3)CCS2(=O)=O)n1] |
| 57 | 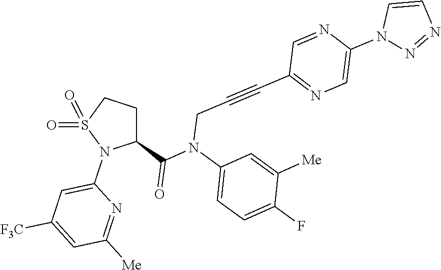 [SMILES: Cc1cc(C(F)(F)F)cc(N2[C@H](C(=O)N(CC#Cc3cnc(-n4ccnn4)cn3)c3ccc(F)c(C)c3)CCS2(=O)=O)n1] |
| 58 | 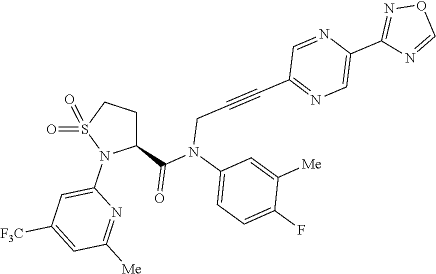 [SMILES: Cc1cc(C(F)(F)F)cc(N2[C@H](C(=O)N(CC#Cc3cnc(-c4ncon4)cn3)c3ccc(F)c(C)c3)CCS2(=O)=O)n1] |
| 59 | 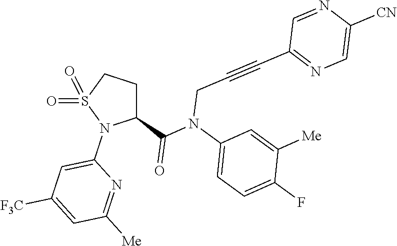 [SMILES: Cc1cc(C(F)(F)F)cc(N2[C@H](C(=O)N(CC#Cc3cnc(C#N)cn3)c3ccc(F)c(C)c3)CCS2(=O)=O)n1] |
| 60 | 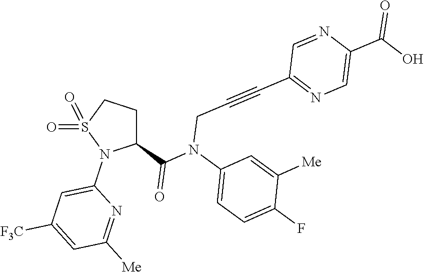 [SMILES: Cc1cc(C(F)(F)F)cc(N2[C@H](C(=O)N(CC#Cc3cnc(C(=O)O)cn3)c3ccc(F)c(C)c3)CCS2(=O)=O)n1] |
| 61 | 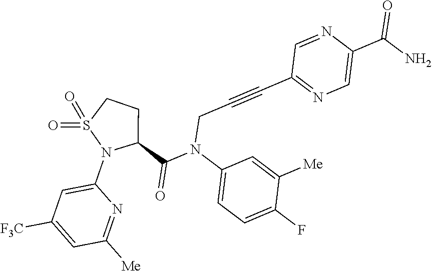 [SMILES: Cc1cc(C(F)(F)F)cc(N2[C@H](C(=O)N(CC#Cc3cnc(C(N)=O)cn3)c3ccc(F)c(C)c3)CCS2(=O)=O)n1] |
| 62 | 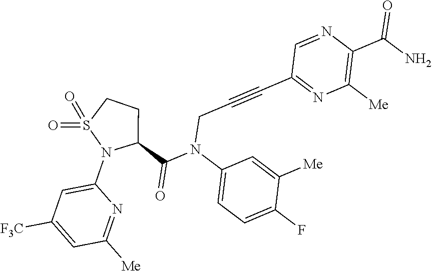 [SMILES: Cc1cc(C(F)(F)F)cc(N2[C@H](C(=O)N(CC#Cc3cnc(C(N)=O)c(C)n3)c3ccc(F)c(C)c3)CCS2(=O)=O)n1] |
| 63 | 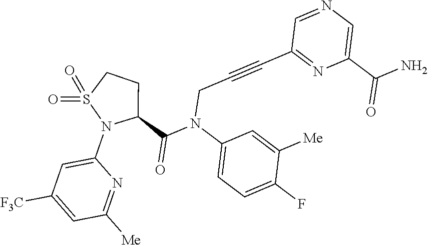 [SMILES: Cc1cc(C(F)(F)F)cc(N2[C@H](C(=O)N(CC#Cc3cncc(C(N)=O)n3)c3ccc(F)c(C)c3)CCS2(=O)=O)n1] |
| 64 | 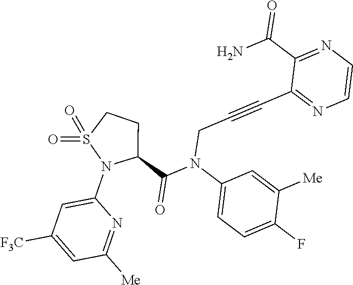 [SMILES: Cc1cc(C(F)(F)F)cc(N2[C@H](C(=O)N(CC#Cc3nccnc3C(N)=O)c3ccc(F)c(C)c3)CCS2(=O)=O)n1] |
| 65 | 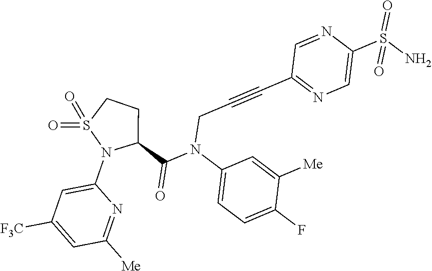 [SMILES: Cc1cc(C(F)(F)F)cc(N2[C@H](C(=O)N(CC#Cc3cnc(S(N)(=O)=O)cn3)c3ccc(F)c(C)c3)CCS2(=O)=O)n1] |
| 66 | 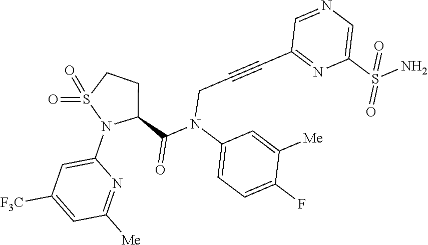 [SMILES: Cc1cc(C(F)(F)F)cc(N2[C@H](C(=O)N(CC#Cc3cncc(S(N)(=O)=O)n3)c3ccc(F)c(C)c3)CCS2(=O)=O)n1] |
| 67 | 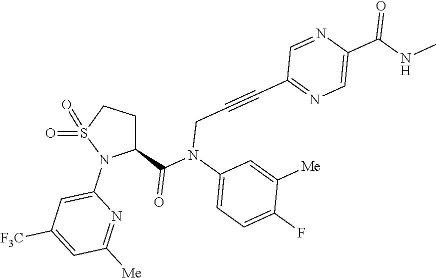 [SMILES: CNC(=O)c1cnc(C#CCN(C(=O)[C@@H]2CCS(=O)(=O)N2c2cc(C(F)(F)F)cc(C)n2)c2ccc(F)c(C)c2)cn1] |
| 68 | 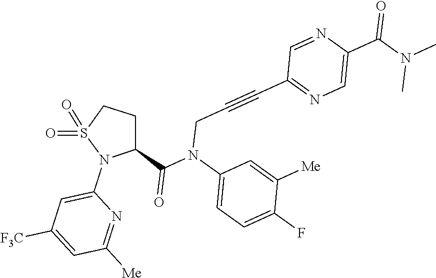 [SMILES: Cc1cc(C(F)(F)F)cc(N2[C@H](C(=O)N(CC#Cc3cnc(C(=O)N(C)C)cn3)c3ccc(F)c(C)c3)CCS2(=O)=O)n1] |
| 69 | 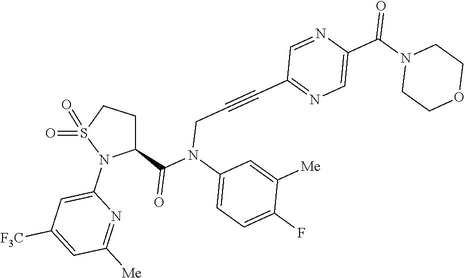 [SMILES: Cc1cc(C(F)(F)F)cc(N2[C@H](C(=O)N(CC#Cc3cnc(C(=O)N4CCOCC4)cn3)c3ccc(F)c(C)c3)CCS2(=O)=O)n1] |
| 70 | 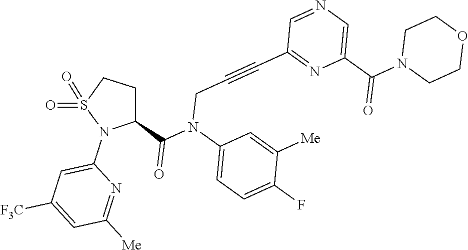 [SMILES: Cc1cc(C(F)(F)F)cc(N2[C@H](C(=O)N(CC#Cc3cncc(C(=O)N4CCOCC4)n3)c3ccc(F)c(C)c3)CCS2(=O)=O)n1] |
| 71 | 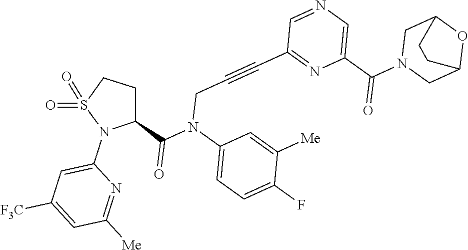 [SMILES: Cc1cc(C(F)(F)F)cc(N2[C@H](C(=O)N(CC#Cc3cncc(C(=O)N4CC5CCC(C4)O5)n3)c3ccc(F)c(C)c3)CCS2(=O)=O)n1] |
| 72 | 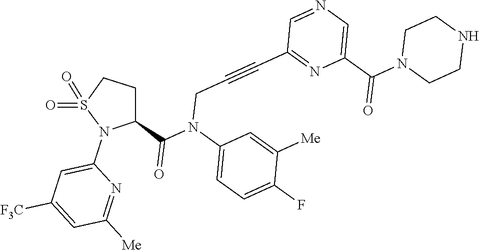 [SMILES: Cc1cc(C(F)(F)F)cc(N2[C@H](C(=O)N(CC#Cc3cncc(C(=O)N4CCNCC4)n3)c3ccc(F)c(C)c3)CCS2(=O)=O)n1] |
| 73 | 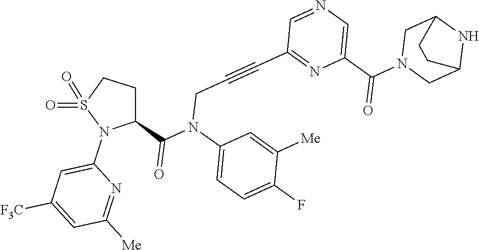 [SMILES: Cc1cc(C(F)(F)F)cc(N2[C@H](C(=O)N(CC#Cc3cncc(C(=O)N4CC5CCC(C4)N5)n3)c3ccc(F)c(C)c3)CCS2(=O)=O)n1] |
| 74 | 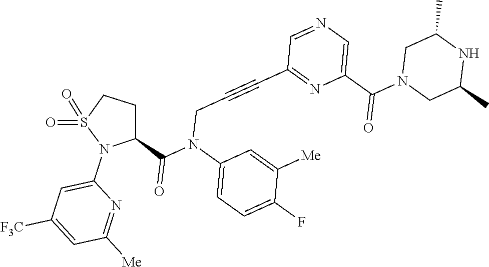 [SMILES: Cc1cc(C(F)(F)F)cc(N2[C@H](C(=O)N(CC#Cc3cncc(C(=O)N4C[C@H](C)N[C@@H](C)C4)n3)c3ccc(F)c(C)c3)CCS2(=O)=O)n1] |
| 75 | 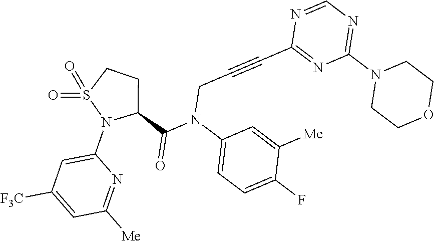 [SMILES: Cc1cc(C(F)(F)F)cc(N2[C@H](C(=O)N(CC#Cc3ncnc(N4CCOCC4)n3)c3ccc(F)c(C)c3)CCS2(=O)=O)n1] |
| 76 | 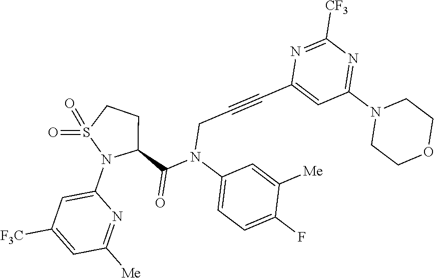 [SMILES: Cc1cc(C(F)(F)F)cc(N2[C@H](C(=O)N(CC#Cc3cc(N4CCOCC4)nc(C(F)(F)F)n3)c3ccc(F)c(C)c3)CCS2(=O)=O)n1] |
| 77 | 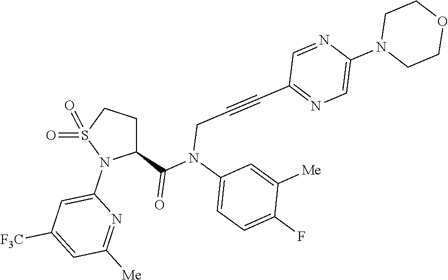 [SMILES: Cc1cc(C(F)(F)F)cc(N2[C@H](C(=O)N(CC#Cc3cnc(N4CCOCC4)cn3)c3ccc(F)c(C)c3)CCS2(=O)=O)n1] |
| 78 | 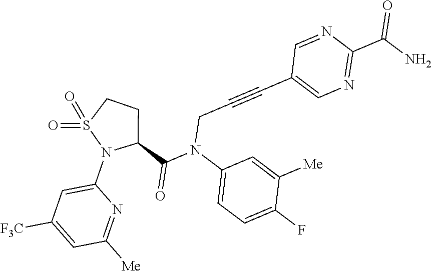 [SMILES: Cc1cc(C(F)(F)F)cc(N2[C@H](C(=O)N(CC#Cc3cnc(C(N)=O)nc3)c3ccc(F)c(C)c3)CCS2(=O)=O)n1] |
| 79 | 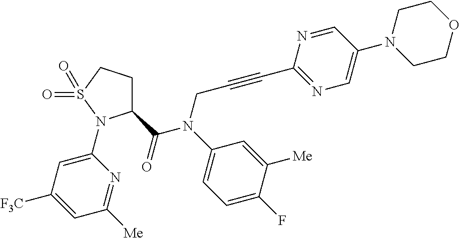 [SMILES: Cc1cc(C(F)(F)F)cc(N2[C@H](C(=O)N(CC#Cc3ncc(N4CCOCC4)cn3)c3ccc(F)c(C)c3)CCS2(=O)=O)n1] |
| 80 | 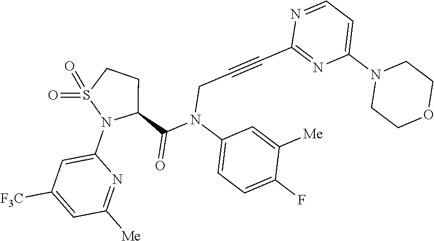 [SMILES: Cc1cc(C(F)(F)F)cc(N2[C@H](C(=O)N(CC#Cc3nccc(N4CCOCC4)n3)c3ccc(F)c(C)c3)CCS2(=O)=O)n1] |
| 81 | 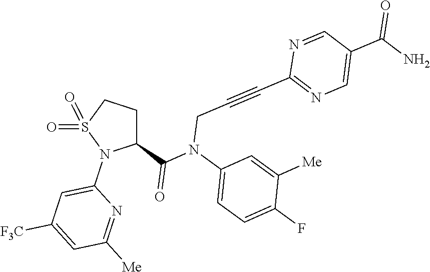 [SMILES: Cc1cc(C(F)(F)F)cc(N2[C@H](C(=O)N(CC#Cc3ncc(C(N)=O)cn3)c3ccc(F)c(C)c3)CCS2(=O)=O)n1] |
| 82 | 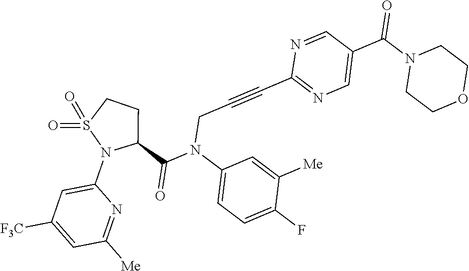 [SMILES: Cc1cc(C(F)(F)F)cc(N2[C@H](C(=O)N(CC#Cc3ncc(C(=O)N4CCOCC4)cn3)c3ccc(F)c(C)c3)CCS2(=O)=O)n1] |
| 83 | 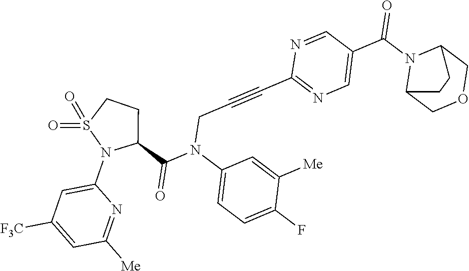 [SMILES: Cc1cc(C(F)(F)F)cc(N2[C@H](C(=O)N(CC#Cc3ncc(C(=O)N4C5CCC4COC5)cn3)c3ccc(F)c(C)c3)CCS2(=O)=O)n1] |
| 84 | 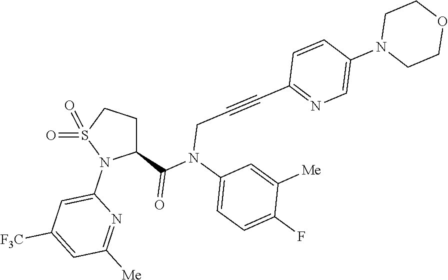 [SMILES: Cc1cc(C(F)(F)F)cc(N2[C@H](C(=O)N(CC#Cc3ccc(N4CCOCC4)cn3)c3ccc(F)c(C)c3)CCS2(=O)=O)n1] |
| 85 | 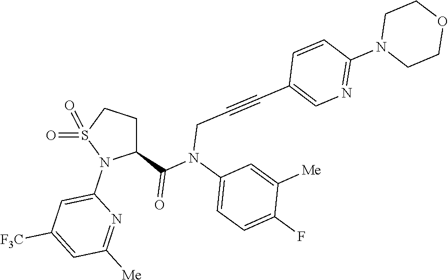 [SMILES: Cc1cc(C(F)(F)F)cc(N2[C@H](C(=O)N(CC#Cc3ccc(N4CCOCC4)nc3)c3ccc(F)c(C)c3)CCS2(=O)=O)n1] |
| 86 | 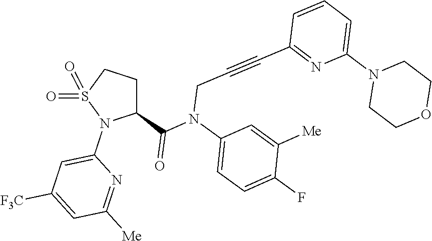 [SMILES: Cc1cc(C(F)(F)F)cc(N2[C@H](C(=O)N(CC#Cc3cccc(N4CCOCC4)n3)c3ccc(F)c(C)c3)CCS2(=O)=O)n1] |
| 87 | 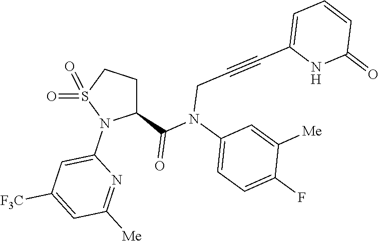 [SMILES: Cc1cc(C(F)(F)F)cc(N2[C@H](C(=O)N(CC#Cc3cccc(=O)[nH]3)c3ccc(F)c(C)c3)CCS2(=O)=O)n1] |
| 88 | 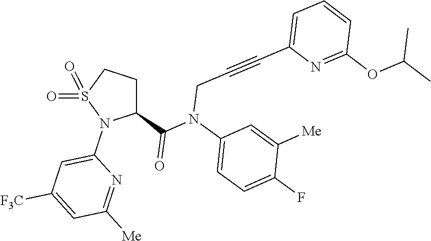 [SMILES: Cc1cc(C(F)(F)F)cc(N2[C@H](C(=O)N(CC#Cc3cccc(OC(C)C)n3)c3ccc(F)c(C)c3)CCS2(=O)=O)n1] |
| 89 | 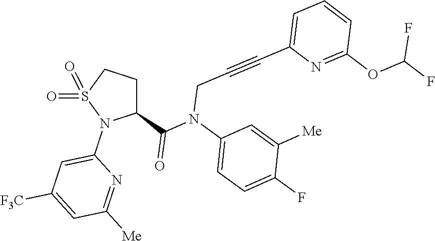 [SMILES: Cc1cc(C(F)(F)F)cc(N2[C@H](C(=O)N(CC#Cc3cccc(OC(F)F)n3)c3ccc(F)c(C)c3)CCS2(=O)=O)n1] |
| 90 | 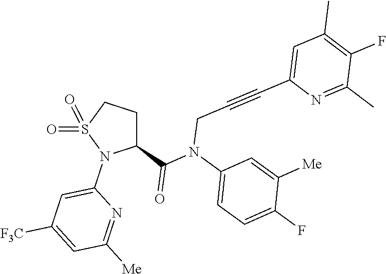 [SMILES: Cc1cc(C(F)(F)F)cc(N2[C@H](C(=O)N(CC#Cc3cc(C)c(F)c(C)n3)c3ccc(F)c(C)c3)CCS2(=O)=O)n1] |
| 91 | 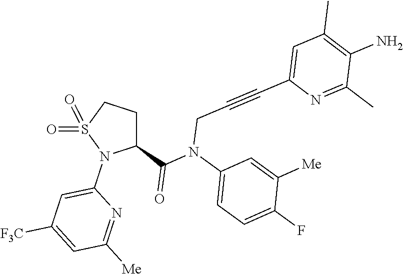 [SMILES: Cc1cc(C(F)(F)F)cc(N2[C@H](C(=O)N(CC#Cc3cc(C)c(N)c(C)n3)c3ccc(F)c(C)c3)CCS2(=O)=O)n1] |
| 92 | 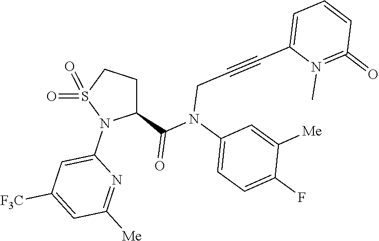 |
| 93 | 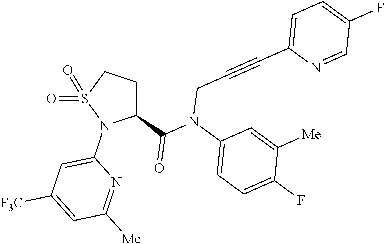 [SMILES: Cc1cc(C(F)(F)F)cc(N2[C@H](C(=O)N(CC#Cc3ccc(F)cn3)c3ccc(F)c(C)c3)CCS2(=O)=O)n1] |
| 94 | 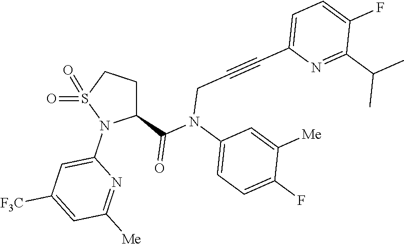 [SMILES: Cc1cc(C(F)(F)F)cc(N2[C@H](C(=O)N(CC#Cc3ccc(F)c(C(C)C)n3)c3ccc(F)c(C)c3)CCS2(=O)=O)n1] |
| 95 | 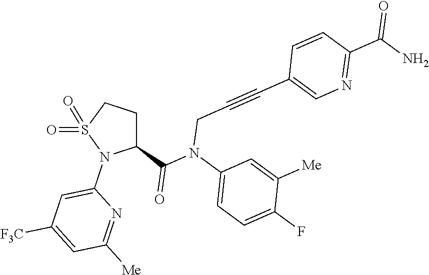 [SMILES: Cc1cc(C(F)(F)F)cc(N2[C@H](C(=O)N(CC#Cc3ccc(C(N)=O)nc3)c3ccc(F)c(C)c3)CCS2(=O)=O)n1] |
| 96 | 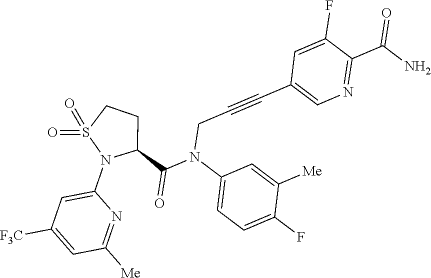 [SMILES: Cc1cc(C(F)(F)F)cc(N2[C@H](C(=O)N(CC#Cc3cnc(C(N)=O)c(F)c3)c3ccc(F)c(C)c3)CCS2(=O)=O)n1] |
| 97 | 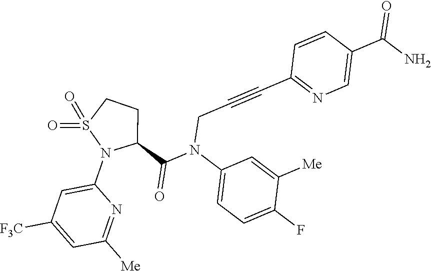 [SMILES: Cc1cc(C(F)(F)F)cc(N2[C@H](C(=O)N(CC#Cc3ccc(C(N)=O)cn3)c3ccc(F)c(C)c3)CCS2(=O)=O)n1] |
| 98 | 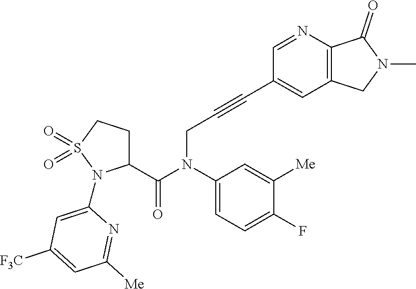 [SMILES: Cc1cc(C(F)(F)F)cc(N2C(C(=O)N(CC#Cc3cnc4c(c3)CN(C)C4=O)c3ccc(F)c(C)c3)CCS2(=O)=O)n1] |
| 99 | 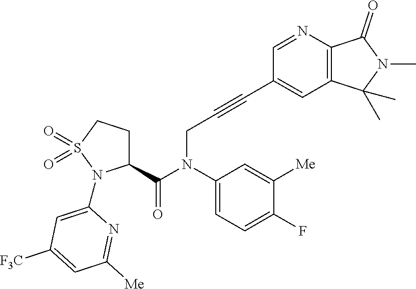 [SMILES: Cc1cc(C(F)(F)F)cc(N2[C@H](C(=O)N(CC#Cc3cnc4c(c3)C(C)(C)N(C)C4=O)c3ccc(F)c(C)c3)CCS2(=O)=O)n1] |
| 100 | 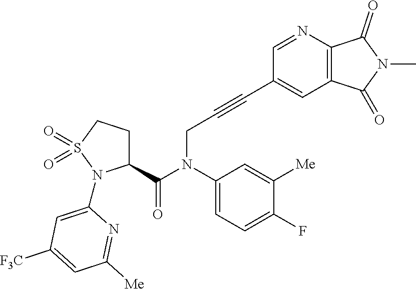 [SMILES: Cc1cc(C(F)(F)F)cc(N2[C@H](C(=O)N(CC#Cc3cnc4c(c3)C(=O)N(C)C4=O)c3ccc(F)c(C)c3)CCS2(=O)=O)n1] |
| 101 | 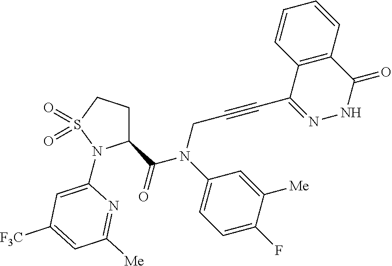 [SMILES: Cc1cc(C(F)(F)F)cc(N2[C@H](C(=O)N(CC#Cc3n[nH]c(=O)c4ccccc34)c3ccc(F)c(C)c3)CCS2(=O)=O)n1] |
| 102 | 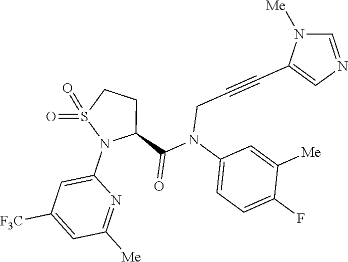 [SMILES: Cc1cc(C(F)(F)F)cc(N2[C@H](C(=O)N(CC#Cc3cncn3C)c3ccc(F)c(C)c3)CCS2(=O)=O)n1] |
| 103 | 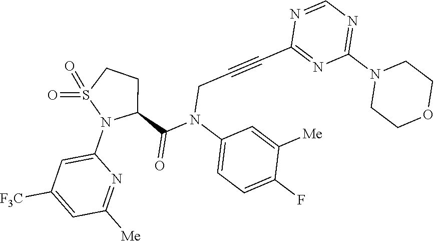 [SMILES: Cc1cc(C(F)(F)F)cc(N2[C@H](C(=O)N(CC#Cc3ncnc(N4CCOCC4)n3)c3ccc(F)c(C)c3)CCS2(=O)=O)n1] |
| 104 | 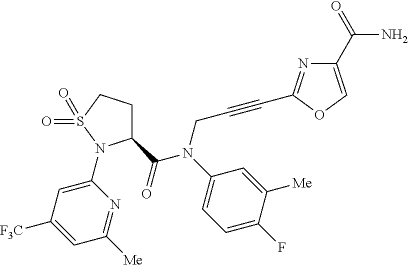 [SMILES: Cc1cc(C(F)(F)F)cc(N2[C@H](C(=O)N(CC#Cc3nc(C(N)=O)co3)c3ccc(F)c(C)c3)CCS2(=O)=O)n1] |
| 105 | 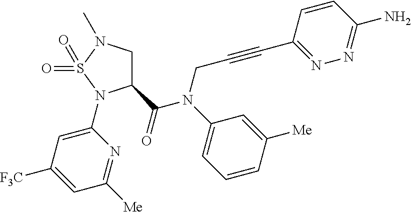 [SMILES: Cc1cccc(N(CC#Cc2ccc(N)nn2)C(=O)[C@@H]2CN(C)S(=O)(=O)N2c2cc(C(F)(F)F)cc(C)n2)c1] |
| 106 | 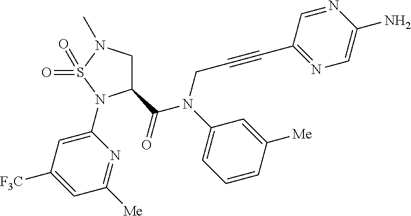 [SMILES: Cc1cccc(N(CC#Cc2cnc(N)cn2)C(=O)[C@@H]2CN(C)S(=O)(=O)N2c2cc(C(F)(F)F)cc(C)n2)c1] |
| 107 | 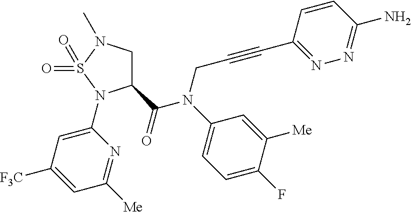 [SMILES: Cc1cc(C(F)(F)F)cc(N2[C@H](C(=O)N(CC#Cc3ccc(N)nn3)c3ccc(F)c(C)c3)CN(C)S2(=O)=O)n1] |
| 108 | 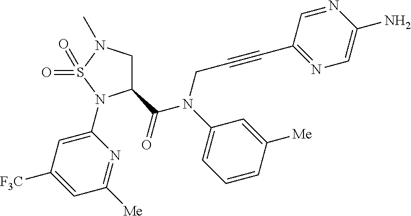 [SMILES: Cc1cccc(N(CC#Cc2cnc(N)cn2)C(=O)[C@@H]2CN(C)S(=O)(=O)N2c2cc(C(F)(F)F)cc(C)n2)c1] |
| 109 | 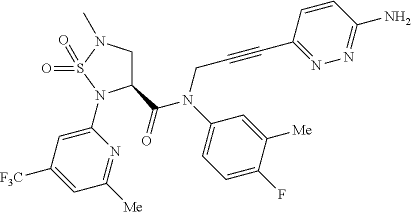 [SMILES: Cc1cc(C(F)(F)F)cc(N2[C@H](C(=O)N(CC#Cc3ccc(N)nn3)c3ccc(F)c(C)c3)CN(C)S2(=O)=O)n1] |
| 110 | 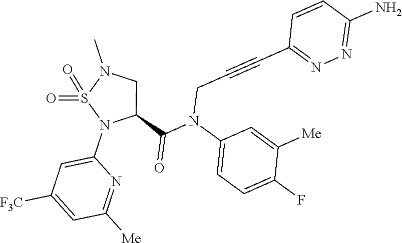 [SMILES: Cc1cc(C(F)(F)F)cc(N2[C@H](C(=O)N(CC#Cc3ccc(N)nn3)c3ccc(F)c(C)c3)CN(C)S2(=O)=O)n1] |
| 111 | 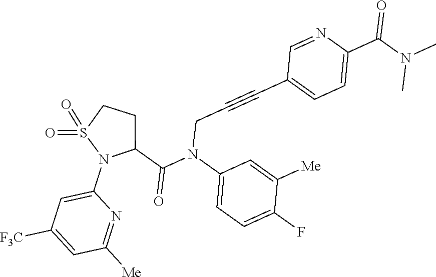 [SMILES: Cc1cc(C(F)(F)F)cc(N2C(C(=O)N(CC#Cc3ccc(C(=O)N(C)C)nc3)c3ccc(F)c(C)c3)CCS2(=O)=O)n1] |
| 112 | 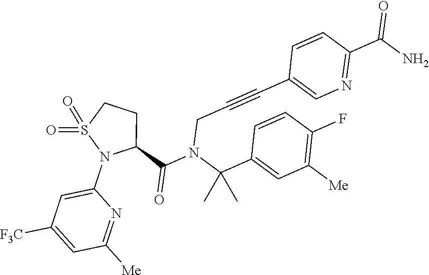 [SMILES: Cc1cc(C(F)(F)F)cc(N2[C@H](C(=O)N(CC#Cc3ccc(C(N)=O)nc3)C(C)(C)c3ccc(F)c(C)c3)CCS2(=O)=O)n1] |
| 113 | 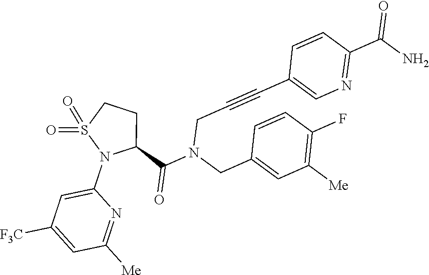 [SMILES: Cc1cc(C(F)(F)F)cc(N2[C@H](C(=O)N(CC#Cc3ccc(C(N)=O)nc3)Cc3ccc(F)c(C)c3)CCS2(=O)=O)n1] |
| 114 | 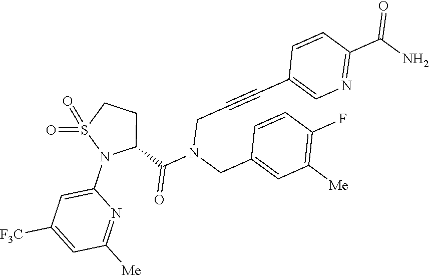 [SMILES: Cc1cc(C(F)(F)F)cc(N2[C@@H](C(=O)N(CC#Cc3ccc(C(N)=O)nc3)Cc3ccc(F)c(C)c3)CCS2(=O)=O)n1] |
| 115 | 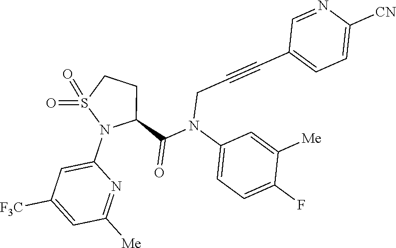 [SMILES: Cc1cc(C(F)(F)F)cc(N2[C@H](C(=O)N(CC#Cc3ccc(C#N)nc3)c3ccc(F)c(C)c3)CCS2(=O)=O)n1] |
| 116 | 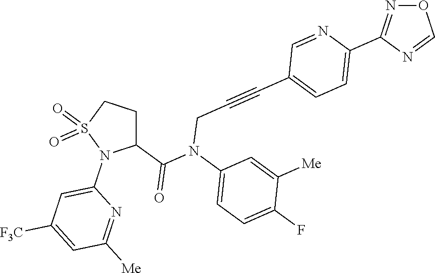 [SMILES: Cc1cc(C(F)(F)F)cc(N2C(C(=O)N(CC#Cc3ccc(-c4ncon4)nc3)c3ccc(F)c(C)c3)CCS2(=O)=O)n1] |
| 117 | 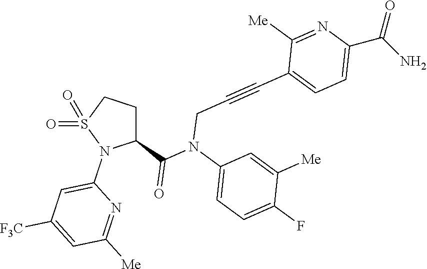 [SMILES: Cc1cc(C(F)(F)F)cc(N2[C@H](C(=O)N(CC#Cc3ccc(C(N)=O)nc3C)c3ccc(F)c(C)c3)CCS2(=O)=O)n1] |
| 118 | 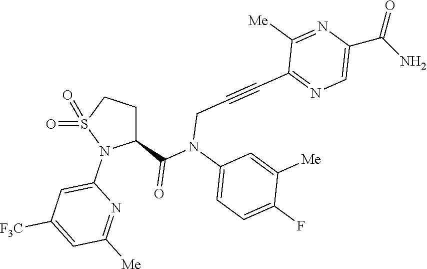 [SMILES: Cc1cc(C(F)(F)F)cc(N2[C@H](C(=O)N(CC#Cc3ncc(C(N)=O)nc3C)c3ccc(F)c(C)c3)CCS2(=O)=O)n1] |
| 119 | 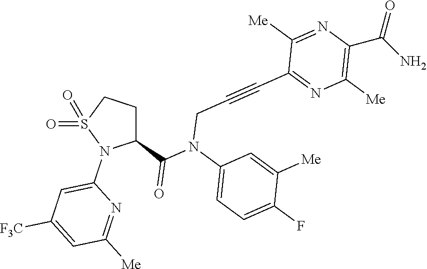 [SMILES: Cc1cc(C(F)(F)F)cc(N2[C@H](C(=O)N(CC#Cc3nc(C)c(C(N)=O)nc3C)c3ccc(F)c(C)c3)CCS2(=O)=O)n1] |
| 120 | 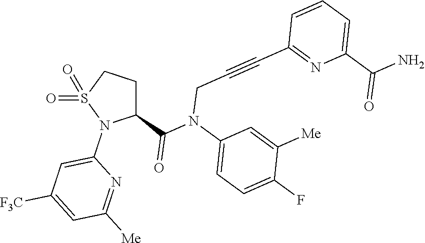 [SMILES: Cc1cc(C(F)(F)F)cc(N2[C@H](C(=O)N(CC#Cc3cccc(C(N)=O)n3)c3ccc(F)c(C)c3)CCS2(=O)=O)n1] |
| 121 | 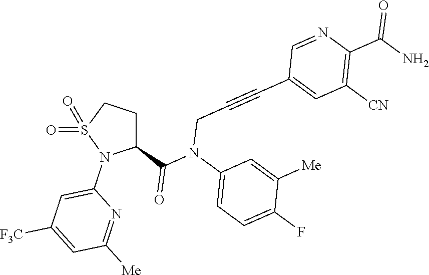 [SMILES: Cc1cc(C(F)(F)F)cc(N2[C@H](C(=O)N(CC#Cc3cnc(C(N)=O)c(C#N)c3)c3ccc(F)c(C)c3)CCS2(=O)=O)n1] |
| 122 | 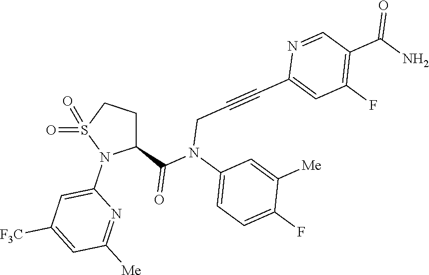 [SMILES: Cc1cc(C(F)(F)F)cc(N2[C@H](C(=O)N(CC#Cc3cc(F)c(C(N)=O)cn3)c3ccc(F)c(C)c3)CCS2(=O)=O)n1] |
| 123 | 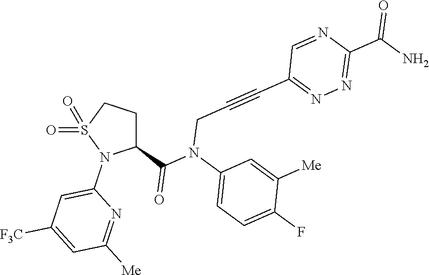 [SMILES: Cc1cc(C(F)(F)F)cc(N2[C@H](C(=O)N(CC#Cc3cnc(C(N)=O)nn3)c3ccc(F)c(C)c3)CCS2(=O)=O)n1] |
| 124 | 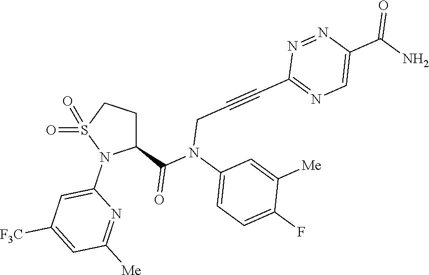 [SMILES: Cc1cc(C(F)(F)F)cc(N2[C@H](C(=O)N(CC#Cc3ncc(C(N)=O)nn3)c3ccc(F)c(C)c3)CCS2(=O)=O)n1] |
| 125 | 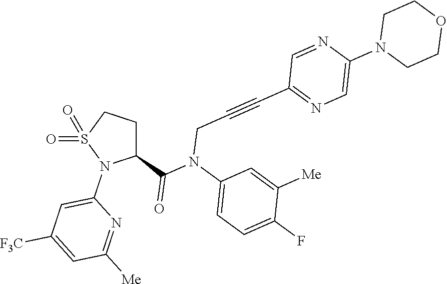 [SMILES: Cc1cc(C(F)(F)F)cc(N2[C@H](C(=O)N(CC#Cc3cnc(N4CCOCC4)cn3)c3ccc(F)c(C)c3)CCS2(=O)=O)n1] |
| 126 | 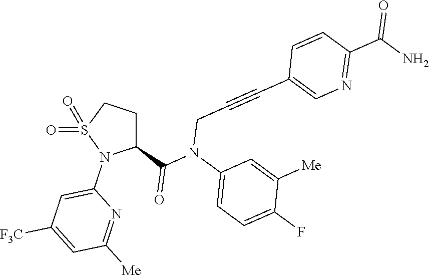 [SMILES: Cc1cc(C(F)(F)F)cc(N2[C@H](C(=O)N(CC#Cc3ccc(C(N)=O)nc3)c3ccc(F)c(C)c3)CCS2(=O)=O)n1] |
| 127 | 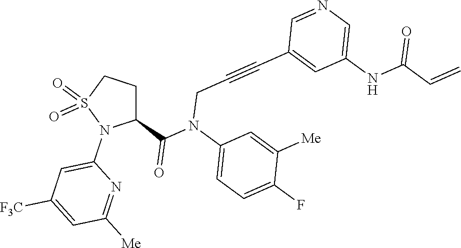 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)c1] |
| 128 | 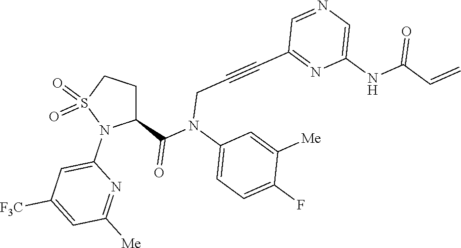 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)n1] |
| 129 | 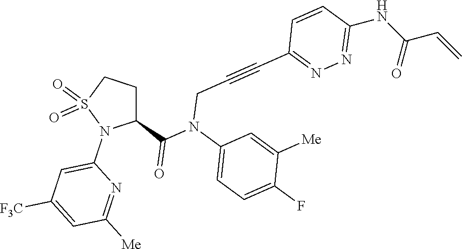 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 130 | 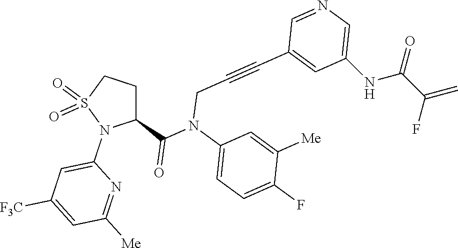 [SMILES: C=C(F)C(=O)Nc1cncc(C#CCN(C(=O)[C@@H]2CCS(=O)(=O)N2c2cc(C(F)(F)F)cc(C)n2)c2ccc(F)c(C)c2)c1] |
| 131 | 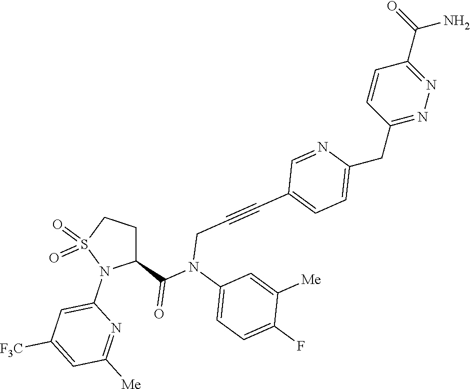 [SMILES: Cc1cc(C(F)(F)F)cc(N2[C@H](C(=O)N(CC#Cc3ccc(Cc4ccc(C(N)=O)nn4)nc3)c3ccc(F)c(C)c3)CCS2(=O)=O)n1] |
| 132 | 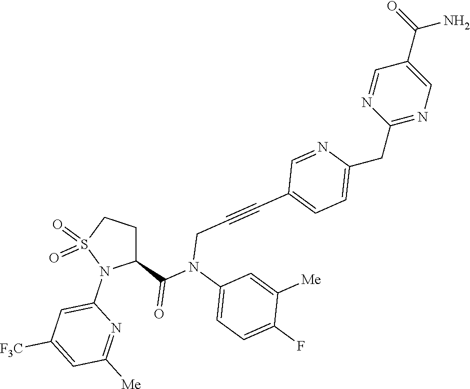 [SMILES: Cc1cc(C(F)(F)F)cc(N2[C@H](C(=O)N(CC#Cc3ccc(Cc4ncc(C(N)=O)cn4)nc3)c3ccc(F)c(C)c3)CCS2(=O)=O)n1] |
| 133 | 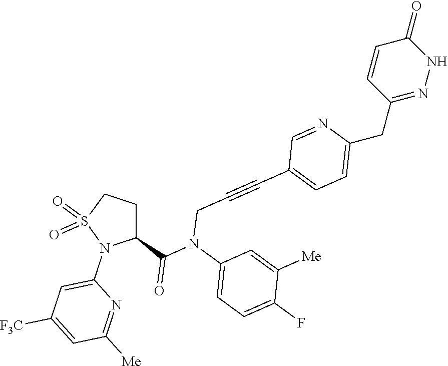 [SMILES: Cc1cc(C(F)(F)F)cc(N2[C@H](C(=O)N(CC#Cc3ccc(Cc4ccc(=O)[nH]n4)nc3)c3ccc(F)c(C)c3)CCS2(=O)=O)n1] |
| 134 | 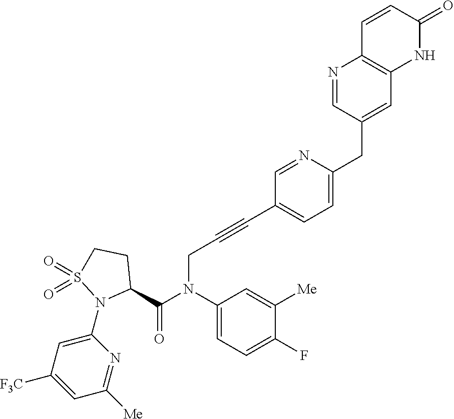 [SMILES: Cc1cc(C(F)(F)F)cc(N2[C@H](C(=O)N(CC#Cc3ccc(Cc4cnc5ccc(=O)[nH]c5c4)nc3)c3ccc(F)c(C)c3)CCS2(=O)=O)n1] |
| 135 | 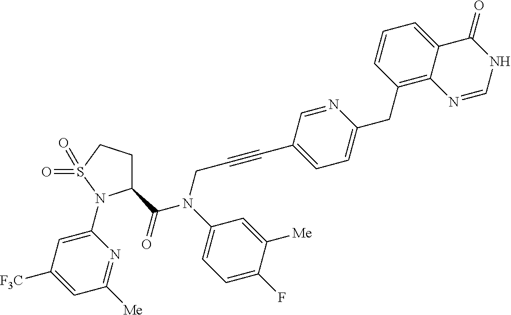 [SMILES: Cc1cc(C(F)(F)F)cc(N2[C@H](C(=O)N(CC#Cc3ccc(Cc4cccc5c(=O)[nH]cnc45)nc3)c3ccc(F)c(C)c3)CCS2(=O)=O)n1] |
| 136 | 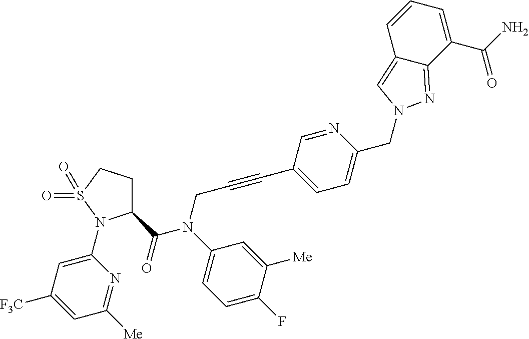 [SMILES: Cc1cc(C(F)(F)F)cc(N2[C@H](C(=O)N(CC#Cc3ccc(Cn4cc5cccc(C(N)=O)c5n4)nc3)c3ccc(F)c(C)c3)CCS2(=O)=O)n1] |
| 137 | 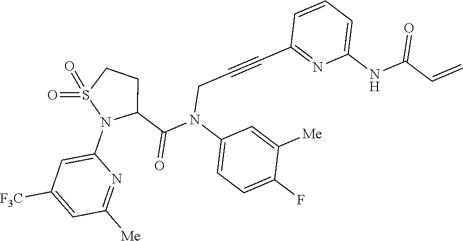 [SMILES: C=CC(=O)Nc1cccc(C#CCN(C(=O)C2CCS(=O)(=O)N2c2cc(C(F)(F)F)cc(C)n2)c2ccc(F)c(C)c2)n1] |
| 138 | 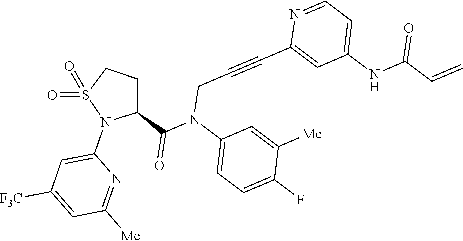 [SMILES: C=CC(=O)Nc1ccnc(C#CCN(C(=O)[C@@H]2CCS(=O)(=O)N2c2cc(C(F)(F)F)cc(C)n2)c2ccc(F)c(C)c2)c1] |
| 139 | 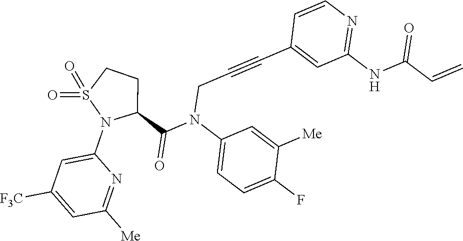 [SMILES: C=CC(=O)Nc1cc(C#CCN(C(=O)[C@@H]2CCS(=O)(=O)N2c2cc(C(F)(F)F)cc(C)n2)c2ccc(F)c(C)c2)ccn1] |
| 140 | 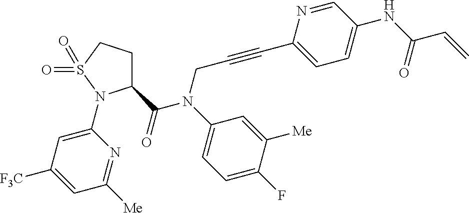 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nc1] |
| 141 | 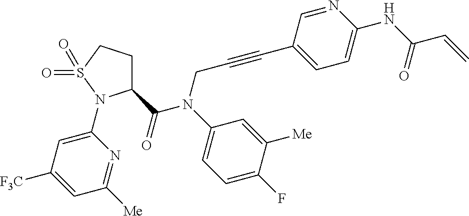 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)cn1] |
| 142 | 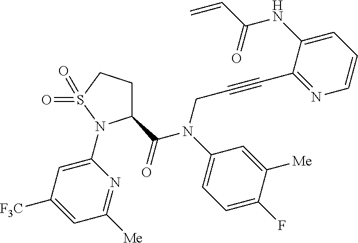 [SMILES: C=CC(=O)Nc1cccnc1C#CCN(C(=O)[C@@H]1CCS(=O)(=O)N1c1cc(C(F)(F)F)cc(C)n1)c1ccc(F)c(C)c1] |
| 143 | 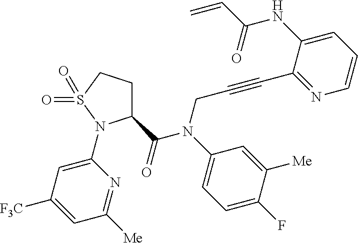 [SMILES: C=CC(=O)Nc1cccnc1C#CCN(C(=O)[C@@H]1CCS(=O)(=O)N1c1cc(C(F)(F)F)cc(C)n1)c1ccc(F)c(C)c1] |
| 144 | 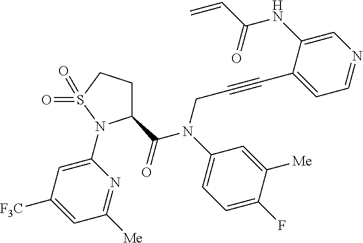 [SMILES: C=CC(=O)Nc1cnccc1C#CCN(C(=O)[C@@H]1CCS(=O)(=O)N1c1cc(C(F)(F)F)cc(C)n1)c1ccc(F)c(C)c1] |
| 145 | 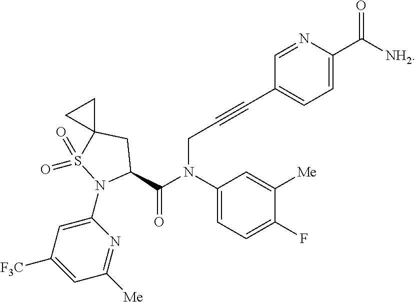 [SMILES: Cc1cc(C(F)(F)F)cc(N2[C@H](C(=O)N(CC#Cc3ccc(C(N)=O)nc3)c3ccc(F)c(C)c3)CC3(CC3)S2(=O)=O)n1] |

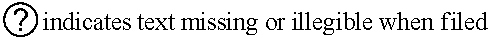

Embodiment 14. A pharmaceutical composition for treating or preventing diseases or disorders mediated by polymerase theta, preferably cancer, more preferably breast cancer, ovarian cancer, prostate cancer, pancreas cancer, or lung cancer, comprising the compound according to any one of preceding embodiments, or a tautomer, stereoisomer, prodrug, crystal form, isotopically labeled form, pharmaceutically acceptable salt, hydrate, or solvate thereof; and a pharmaceutically acceptable carrier(s) or excipient(s).

Embodiment 15. The pharmaceutical composition according to claim 14, wherein the composition is administered separately, sequentially or simultaneously with additional DNA damage response (DDR) targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (e.g., nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (e.g., MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).

Embodiment 16. A compound according to any one of embodiments 1 to 13, or a tautomer, stereoisomer, prodrug, crystal form, isotopically labeled form, pharmaceutically acceptable salt, hydrate, or solvate thereof for use in the prevention or treatment of diseases or disorders mediated by polymerase theta, preferably cancer, more preferably breast cancer, ovarian cancer, prostate cancer, pancreas cancer, or lung cancer.

Embodiment 17. The compound according to embodiment 16, wherein the compound is administered separately, sequentially or simultaneously with additional DNA damage response (DDR) targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).

Embodiment 18. A method of treating or preventing diseases or disorders mediated by polymerase theta in a subject, comprising administering to the subject at least one compound according to any one of embodiments 1 to 13, or a tautomer, stereoisomer, prodrug, crystal form, isotopically labeled form, pharmaceutically acceptable salt, hydrate, or solvate thereof.

Embodiment 19. The method of embodiment 18, wherein the diseases or disorders mediated by polymerase theta are cancer, preferably breast cancer, ovarian cancer, prostate cancer, pancreas cancer, or lung cancer.

Embodiment 20. The method of any one of embodiments 18 to 19, further comprising administering to the subject additional DDR targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).

1. A compound of the following Formula (I): 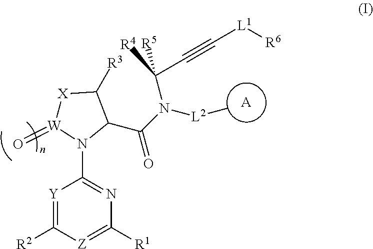
wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L¹ is selected from the group consisting of absent, —(C₁-C₆)alkylene-, and —(C₁-C₆)alkylene-NH—;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl,
or a tautomer, stereoisomer, prodrug, crystal form, isotopically labeled form, pharmaceutically acceptable salt, hydrate, or solvate thereof.

2. The compound according to claim 1, wherein the compound is a compound represented by Formula (II): 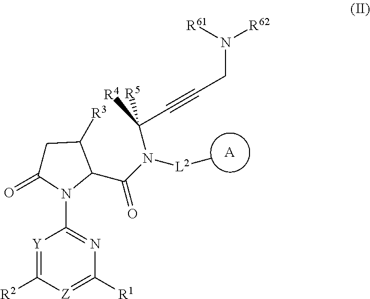
wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl.

3. The compound according to claim 2, wherein the compound is a compound represented by Formula (IIa-1): 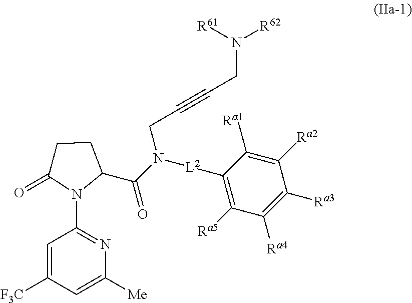
wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

4. The compound according to claim 2, wherein each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen.

5. The compound according to claim 1, wherein the compound is a compound represented by Formula (III): 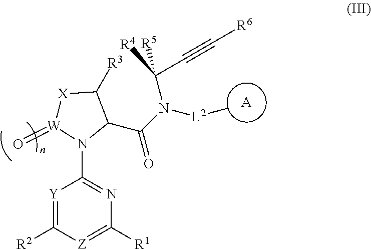
wherein:
each of R¹, R², R³, R⁴ and R⁵ is independently selected from the group consisting of hydrogen, C₁-C₆ alkyl, C₂-C₆ alkenyl, C₁-C₆ alkoxy, halogen, 3-10 membered cycloalkyl, cyano, hydroxy, and —NR^{x}R^{y}, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
X is selected from the group consisting of methylene, ethylene, n-propylene, —NH—, —NR^{z}—, and —CR^{z2}R^{z3}—, wherein each of said methylene, ethylene, and n-propylene is optionally substituted with one or more selected from C₁-C₆ alkyl, halogen, cyano, deuterium, hydroxy, amino, mercapto, and carbamoyl;
each of Y and Z is independently selected from the group consisting of —N— and —CR^{w}—;
W is selected from the group consisting of carbon and sulfur;
n is 1 or 2, provided that when W is carbon, n is 1; and when W is sulfur, n is 2;
A is selected from the group consisting of 5-12 membered monocyclic or bicyclic aryl or heteroaryl, wherein each of said aryl and heteroaryl is independently optionally substituted with one or more selected from C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
R^{z2} and R^{z3}, taken together with the carbon atom to which they are attached, form a 3-6 membered cycloalkane ring, wherein said cycloalkane ring is optionally substituted with one or more selected from C₁-C₆ alkyl and halogen; and
R^{w} is selected from the group consisting of hydrogen, C₁-C₆ alkyl, halogen, cyano, deuterium, C₂-C₆ alkenyl, C₁-C₆ alkoxy, 3-10 membered cycloalkyl, —NR^{x}R^{y}, hydroxy, amino, mercapto, and carbamoyl.

6. The compound according to claim 5, wherein the compound is a compound represented by Formula (IIIa-1): 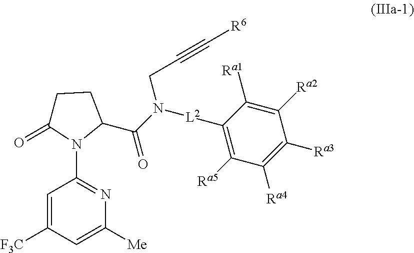
wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

7. The compound according to claim 5, wherein the compound is a compound represented by Formula (IIIb-1): 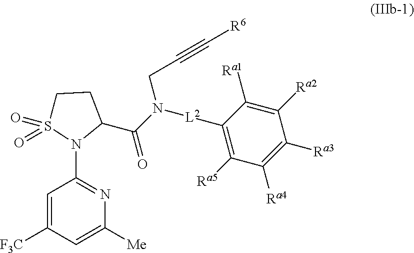
wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

8. The compound according to claim 5, wherein the compound is a compound represented by Formula (IIIc-1): 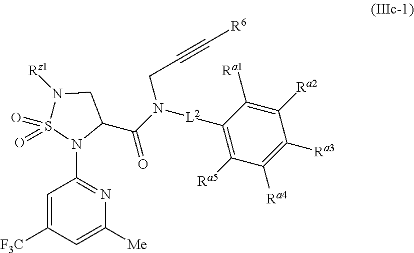
wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
R^{z1} is C₁-C₆ alkyl, wherein said alkyl is optionally substituted with one or more selected from C₁-C₆ alkyl, halogen, cyano, deuterium, hydroxy, amino, mercapto, and carbamoyl;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino;
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

9. The compound according to claim 5, wherein the compound is a compound represented by Formula (IIId-1): 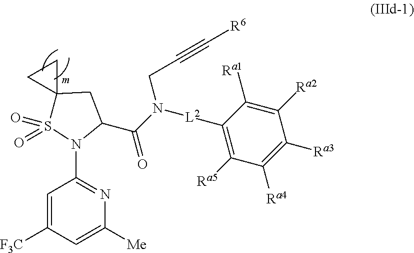
wherein:
each of R^{a1}, R^{a2}, R^{a3}, R^{a4}, and R^{a5} is selected from the group consisting of hydrogen, C₁-C₆ alkyl, halogen, cyano, deuterium, C₂-C₆ alkenyl, hydroxy, C₁-C₆ alkoxy, halogen, 3-10 membered cycloalkyl, —NR^{x}R^{y}, amino, mercapto, and carbamoyl, wherein each of said alkyl, alkenyl, alkoxy, and cycloalkyl is independently optionally substituted with one or more selected from C₁-C₆ alkyl, halogen, cyano, deuterium, hydroxy, amino, mercapto, and carbamoyl;
m is an integer from 1 to 4;
L² is selected from the group consisting of absent, and —(C₁-C₆)alkylene-, wherein said alkylene is optionally substituted with one or more selected from C₁-C₆ alkyl, and halogen;
R⁶ is selected from the group consisting of phenyl and a 3-10 membered heterocyclyl group, wherein each of said phenyl and heterocyclyl group is optionally substituted with one or more selected from C₁-C₆ alkyl, halogen, cyano, deuterium, oxo, carbamoyl, hydroxy, C₁-C₆ alkoxy, mercapto, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R⁶⁶, wherein each of said alkyl, alkoxy, and heterocyclyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶³ is selected from the group consisting of hydrogen, C₁-C₆ alkyl, NH₂, and C₂-C₆ alkenyl, wherein each of said alkyl and alkenyl is optionally substituted with one or more selected from C₁-C₆ alkyl and halogen;
each of R⁶⁴ and R⁶⁵ is selected from the group consisting of hydrogen and C₁-C₆ alkyl, wherein said alkyl is optionally substituted with C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with one or more selected from C₁-C₆ alkyl and halogen, wherein said alkyl is optionally substituted with one or more selected from C₁-C₆ alkyl and halogen;
R⁶⁶ is a 5-10 membered heterocyclyl group, wherein said heterocyclyl group is optionally substituted with one or more selected from C₁-C₆ alkyl, halogen, oxo, carbamoyl, and amino; and
each of R^{x} and R^{y} is independently selected from the group consisting of hydrogen and C₁-C₆ alkyl, wherein said alkyl is optionally substituted with one or more selected from C₁-C₆ alkyl, halogen, cyano, deuterium, hydroxy, amino, mercapto, and carbamoyl.

10. The compound according to any one of claims 3 and 6 to 9, wherein each of R^{a1}, R^{a2}, R^{a3}, R^{a4}, and R^{a5} is selected from the group consisting of hydrogen, methyl, ethyl, n-propyl, isopropyl, fluoromethyl, difluoromethyl, trifluoromethyl, fluoro, chloro, bromo, and iodo.

11. The compound according to claim 1, wherein R⁶ is selected from the group consisting of: 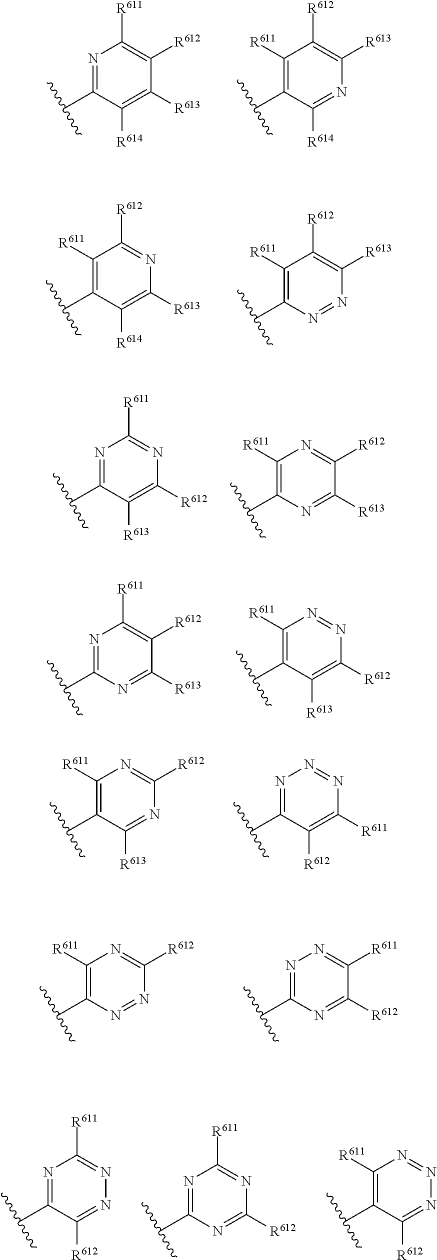 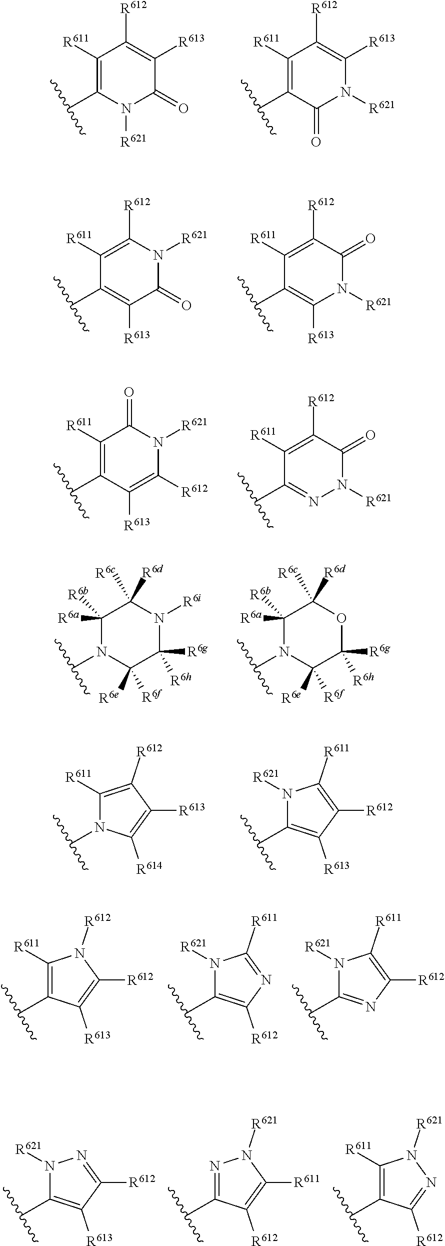 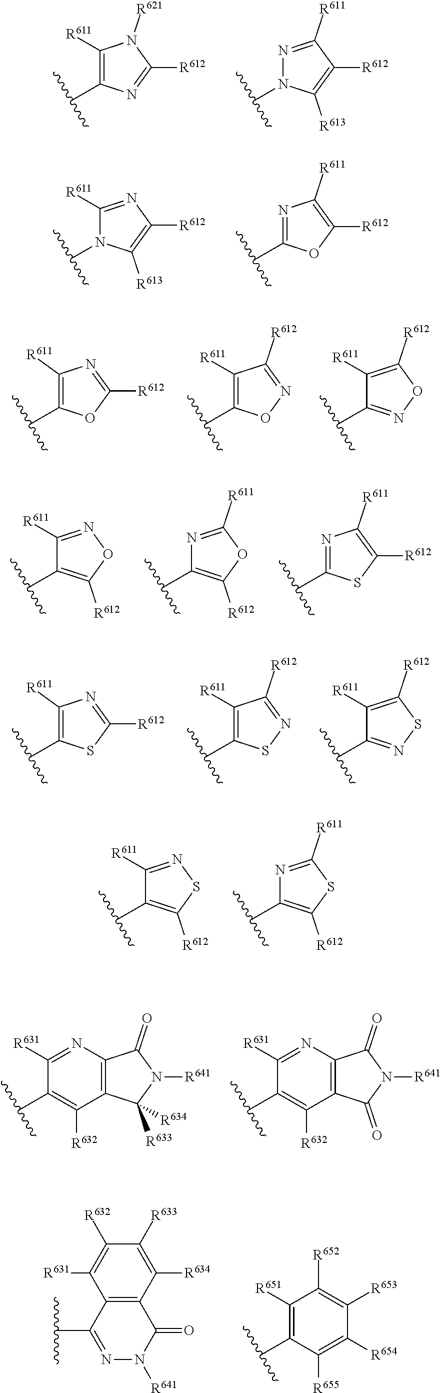
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, C₁-C₆ alkyl, halogen, cyano, oxo, carbamoyl, C₁-C₆ alkoxy, amino, —COOH, —S(O)₂NH₂, —NR⁶¹R⁶², —NHC(O)R⁶³, —C(O)NR⁶⁴R⁶⁵, 5-6 membered heterocyclyl, and —CH₂—R, wherein each of said alkyl, alkoxy, and heterocyclyl is optionally substituted with one or more selected from C₁-C₆ alkyl, halogen, and deuterium;
each of R⁶⁵¹, R⁶⁵², R⁶⁵³, R⁶⁵⁴, and R⁶⁵⁵ is independently selected from the group consisting of hydrogen, C₁-C₆ alkyl, halogen, cyano, deuterium, oxo, carbamoyl, hydroxy, and —C(O)NR⁶⁴R⁶⁵, wherein said alkyl is optionally substituted with one or more selected from C₁-C₆ alkyl, halogen, and deuterium;
each of R⁶¹ and R⁶² is selected from the group consisting of hydrogen, and C₁-C₆ alkyl, wherein said alkyl is optionally substituted with C₁-C₆ alkyl, or
R⁶¹ and R⁶², taken together with the nitrogen atom to which they are attached, form a nitrogen-containing 6-8 membered heterocyclic group, wherein said heterocyclic group is optionally substituted with C₁-C₆ alkyl, wherein said alkyl is optionally substituted with one or more selected from C₁-C₆ alkyl and halogen;
R⁶³ is selected from the group consisting of C₁-C₆ alkyl, NH₂, and C₂-C₆ alkenyl, wherein each of said alkyl and alkenyl is optionally substituted with halogen;
each of R⁶⁴ and R⁶⁵ is selected from the group consisting of hydrogen and C₁-C₆ alkyl, or
R⁶⁴ and R⁶⁵, taken together with the nitrogen atom to which they are attached, form a nitrogen-containing 5-8 membered heterocyclic group, wherein said heterocyclic group is optionally substituted with C₁-C₆ alkyl; and
R⁶⁶ is a 6-10 membered heterocyclyl group, wherein said heterocyclyl group is optionally substituted with one or more selected from oxo, and carbamoyl.

12. The compound according to claim 1, wherein R⁶ is selected from the group consisting of: 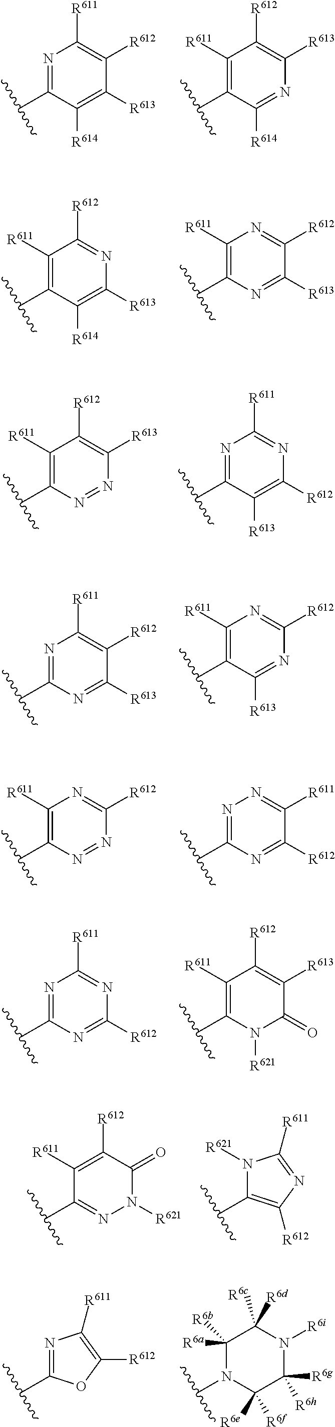 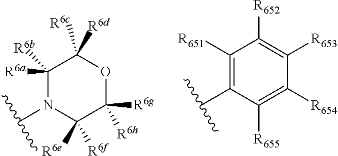
each of R⁶¹¹, R⁶¹², R⁶¹³, R⁶¹⁴, R⁶²¹, R⁶³¹, R⁶³², R⁶³³, R⁶³⁴, R⁶⁴¹, R^{6a}, R^{6b}, R^{6c}, R^{6d}, R^{6e}, R^{6f}, R^{6g}, R^{6h}, and R⁶ⁱ is independently selected from the group consisting of hydrogen, methyl, ethyl, isopropyl, trifluoromethyl, fluoro, cyano, oxo, carbamoyl, methoxy, difluoromethoxy, isopropoxy, amino, —COOH, —S(O)₂NH₂, —NR⁶¹R⁶², —NHC(O)R⁶³, —C(O)NR⁶⁴R⁶⁵, —CH₂—R⁶⁶, 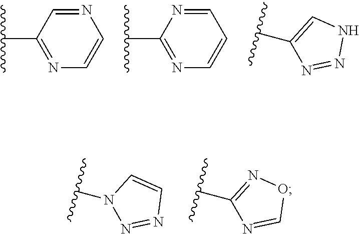
each of R⁶⁵¹, R⁶⁵², R⁶⁵³, R⁶⁵⁴, and R⁶⁵⁵ is independently selected from the group consisting of hydrogen, and carbamoyl;
each of R⁶¹ and R⁶² is selected from the group consisting of hydrogen, ethyl, and isopropyl, or
R⁶¹ and R⁶², taken together with the nitrogen atom to which they are attached, form a nitrogen-containing 6-8 membered heterocyclic group, which is: 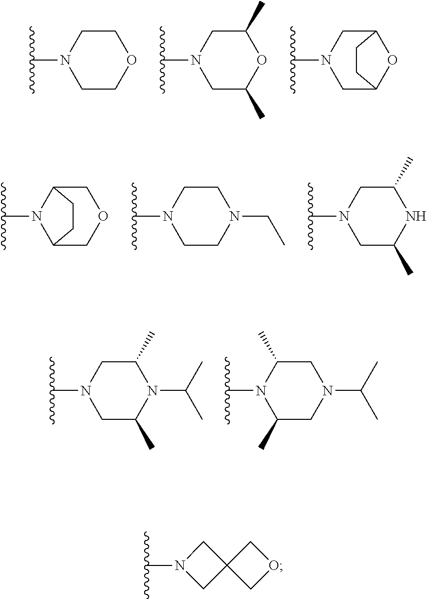
R⁶³ is selected from the group consisting of methyl, NH₂, ethenyl, and 1-fluoroethenyl;
each of R⁶⁴ and R⁶⁵ is selected from the group consisting of hydrogen, and methyl, or
R⁶⁴ and R⁶⁵, taken together with the nitrogen atom to which they are attached, form a nitrogen-containing 6-8 membered heterocyclic group, which is: 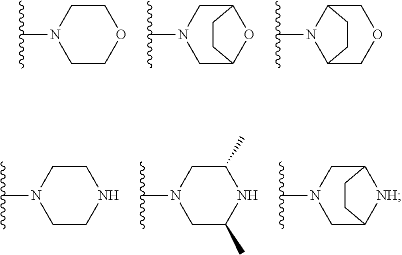
and
R⁶⁶ is a 6-10 membered heterocyclyl group, which is:

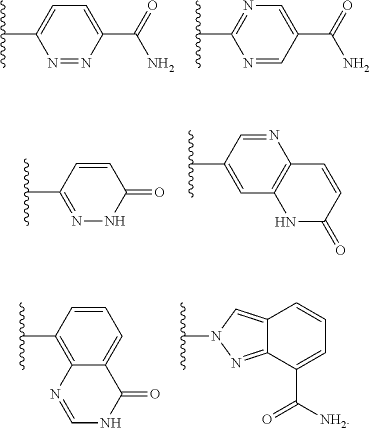

13. The compound according to claim 1, wherein the compound is selected from the group consisting of:

| NO. | Structure |
|---|---|
| 1 | 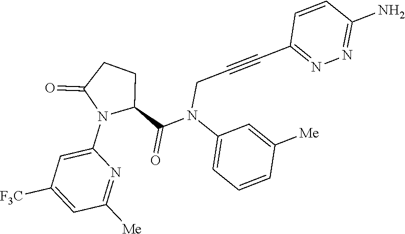 [SMILES: Cc1cccc(N(CC#Cc2ccc(N)nn2)C(=O)[C@@H]2CCC(=O)N2c2cc(C(F)(F)F)cc(C)n2)c1] |
| 2 | 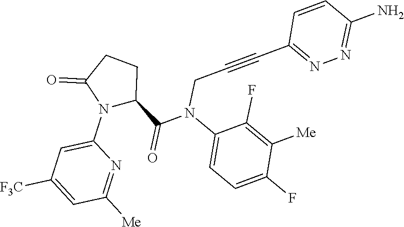 [SMILES: Cc1cc(C(F)(F)F)cc(N2C(=O)CC[C@H]2C(=O)N(CC#Cc2ccc(N)nn2)c2ccc(F)c(C)c2F)n1] |
| 3 | 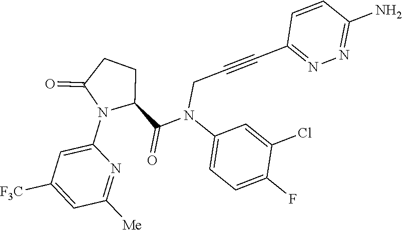 [SMILES: Cc1cc(C(F)(F)F)cc(N2C(=O)CC[C@H]2C(=O)N(CC#Cc2ccc(N)nn2)c2ccc(F)c(Cl)c2)n1] |
| 4 | 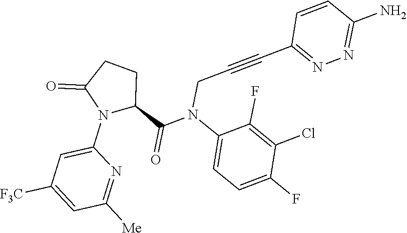 [SMILES: Cc1cc(C(F)(F)F)cc(N2C(=O)CC[C@H]2C(=O)N(CC#Cc2ccc(N)nn2)c2ccc(F)c(Cl)c2F)n1] |
| 5 | 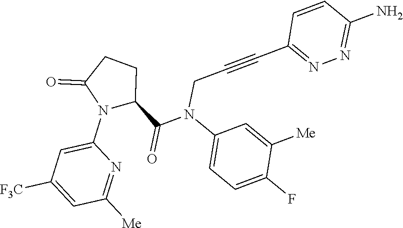 [SMILES: Cc1cc(C(F)(F)F)cc(N2C(=O)CC[C@H]2C(=O)N(CC#Cc2ccc(N)nn2)c2ccc(F)c(C)c2)n1] |
| 6 | 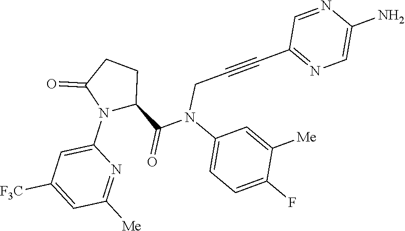 [SMILES: Cc1cc(C(F)(F)F)cc(N2C(=O)CC[C@H]2C(=O)N(CC#Cc2cnc(N)cn2)c2ccc(F)c(C)c2)n1] |
| 7 | 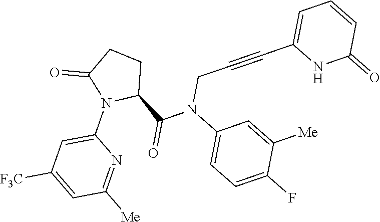 [SMILES: Cc1cc(C(F)(F)F)cc(N2C(=O)CC[C@H]2C(=O)N(CC#Cc2cccc(=O)[nH]2)c2ccc(F)c(C)c2)n1] |
| 8 | 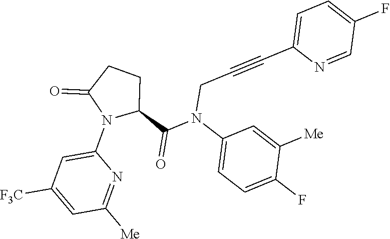 [SMILES: Cc1cc(C(F)(F)F)cc(N2C(=O)CC[C@H]2C(=O)N(CC#Cc2ccc(F)cn2)c2ccc(F)c(C)c2)n1] |
| 9 | 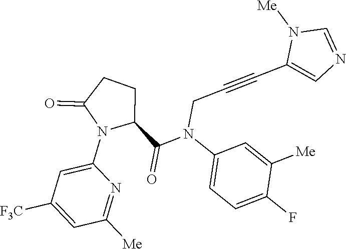 [SMILES: Cc1cc(C(F)(F)F)cc(N2C(=O)CC[C@H]2C(=O)N(CC#Cc2cncn2C)c2ccc(F)c(C)c2)n1] |
| 10 | 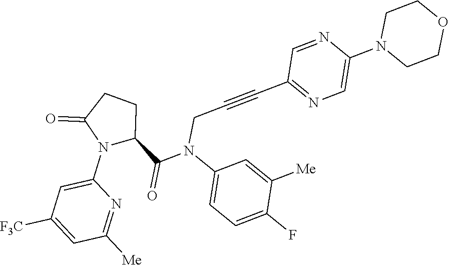 [SMILES: Cc1cc(C(F)(F)F)cc(N2C(=O)CC[C@H]2C(=O)N(CC#Cc2cnc(N3CCOCC3)cn2)c2ccc(F)c(C)c2)n1] |
| 11 | 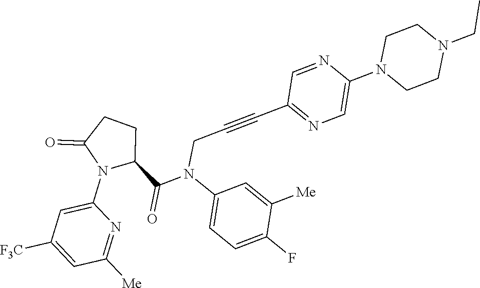 [SMILES: CCN1CCN(c2cnc(C#CCN(C(=O)[C@@H]3CCC(=O)N3c3cc(C(F)(F)F)cc(C)n3)c3ccc(F)c(C)c3)cn2)CC1] |
| 12 | 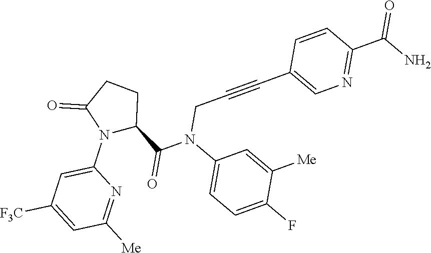 [SMILES: Cc1cc(C(F)(F)F)cc(N2C(=O)CC[C@H]2C(=O)N(CC#Cc2ccc(C(N)=O)nc2)c2ccc(F)c(C)c2)n1] |
| 13 | 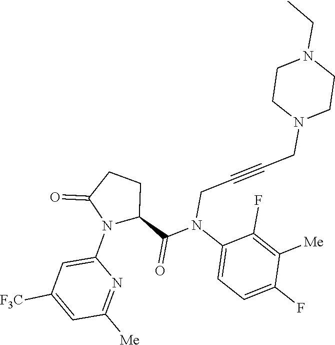 [SMILES: CCN1CCN(CC#CCN(C(=O)[C@@H]2CCC(=O)N2c2cc(C(F)(F)F)cc(C)n2)c2ccc(F)c(C)c2F)CC1] |
| 14 | 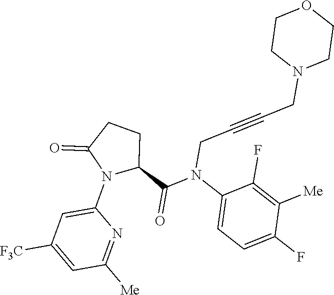 [SMILES: Cc1cc(C(F)(F)F)cc(N2C(=O)CC[C@H]2C(=O)N(CC#CCN2CCOCC2)c2ccc(F)c(C)c2F)n1] |
| 15 | 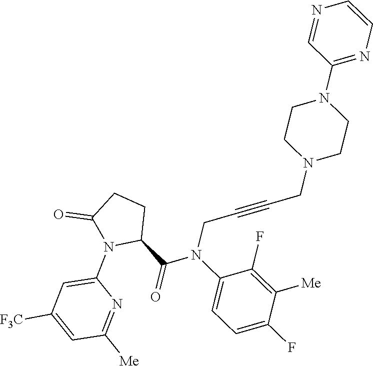 [SMILES: Cc1cc(C(F)(F)F)cc(N2C(=O)CC[C@H]2C(=O)N(CC#CCN2CCN(c3cnccn3)CC2)c2ccc(F)c(C)c2F)n1] |
| 16 | 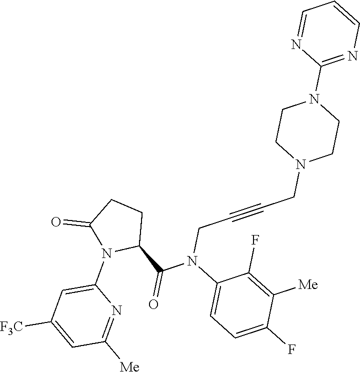 [SMILES: Cc1cc(C(F)(F)F)cc(N2C(=O)CC[C@H]2C(=O)N(CC#CCN2CCN(c3ncccn3)CC2)c2ccc(F)c(C)c2F)n1] |
| 17 | 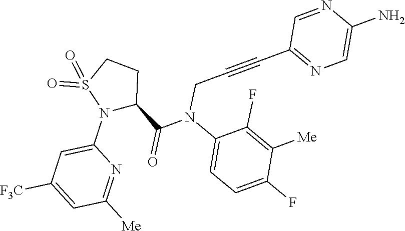 [SMILES: Cc1cc(C(F)(F)F)cc(N2[C@H](C(=O)N(CC#Cc3cnc(N)cn3)c3ccc(F)c(C)c3F)CCS2(=O)=O)n1] |
| 18 | 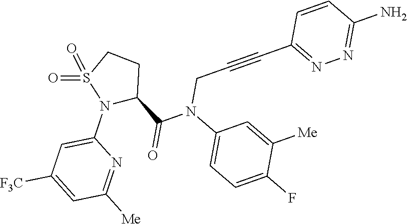 [SMILES: Cc1cc(C(F)(F)F)cc(N2[C@H](C(=O)N(CC#Cc3ccc(N)nn3)c3ccc(F)c(C)c3)CCS2(=O)=O)n1] |
| 19 | 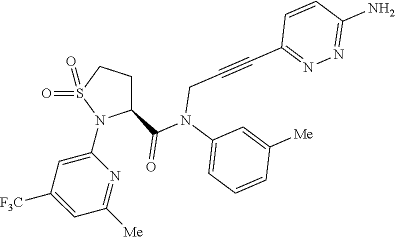 [SMILES: Cc1cccc(N(CC#Cc2ccc(N)nn2)C(=O)[C@@H]2CCS(=O)(=O)N2c2cc(C(F)(F)F)cc(C)n2)c1] |
| 20 | 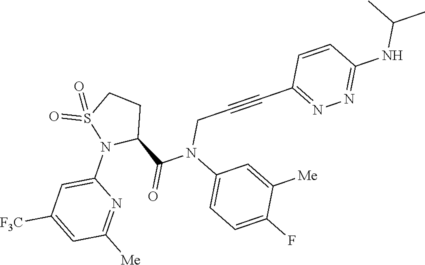 [SMILES: Cc1cc(C(F)(F)F)cc(N2[C@H](C(=O)N(CC#Cc3ccc(NC(C)C)nn3)c3ccc(F)c(C)c3)CCS2(=O)=O)n1] |
| 21 | 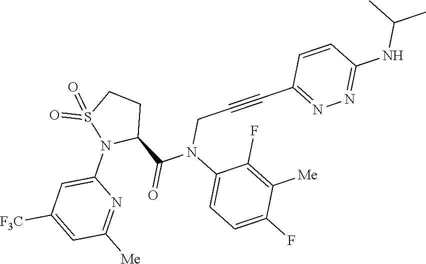 [SMILES: Cc1cc(C(F)(F)F)cc(N2[C@H](C(=O)N(CC#Cc3ccc(NC(C)C)nn3)c3ccc(F)c(C)c3F)CCS2(=O)=O)n1] |
| 22 | 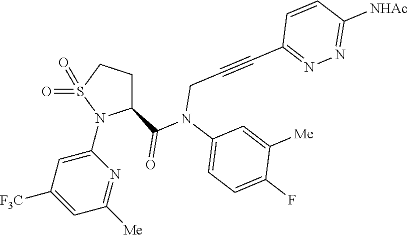 [SMILES: CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 23 | 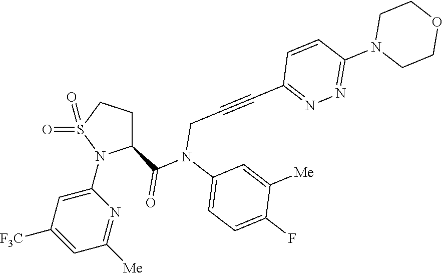 [SMILES: Cc1cc(C(F)(F)F)cc(N2[C@H](C(=O)N(CC#Cc3ccc(N4CCOCC4)nn3)c3ccc(F)c(C)c3)CCS2(=O)=O)n1] |
| 24 | 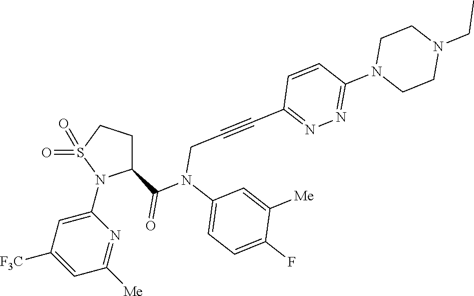 [SMILES: CCN1CCN(c2ccc(C#CCN(C(=O)[C@@H]3CCS(=O)(=O)N3c3cc(C(F)(F)F)cc(C)n3)c3ccc(F)c(C)c3)nn2)CC1] |
| 25 | 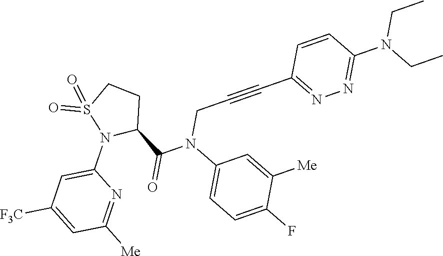 [SMILES: CCN(CC)c1ccc(C#CCN(C(=O)[C@@H]2CCS(=O)(=O)N2c2cc(C(F)(F)F)cc(C)n2)c2ccc(F)c(C)c2)nn1] |
| 26 | 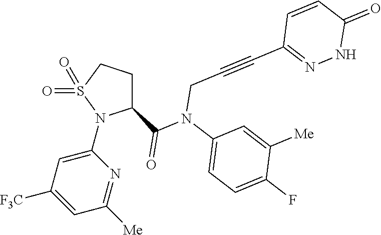 [SMILES: Cc1cc(C(F)(F)F)cc(N2[C@H](C(=O)N(CC#Cc3ccc(=O)[nH]n3)c3ccc(F)c(C)c3)CCS2(=O)=O)n1] |
| 27 | 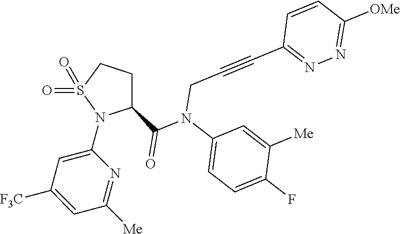 [SMILES: COc1ccc(C#CCN(C(=O)[C@@H]2CCS(=O)(=O)N2c2cc(C(F)(F)F)cc(C)n2)c2ccc(F)c(C)c2)nn1] |
| 28 | 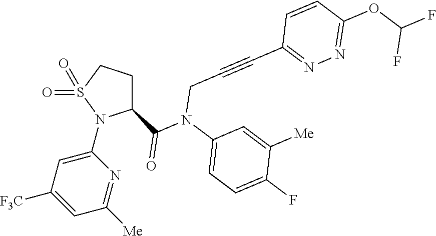 [SMILES: Cc1cc(C(F)(F)F)cc(N2[C@H](C(=O)N(CC#Cc3ccc(OC(F)F)nn3)c3ccc(F)c(C)c3)CCS2(=O)=O)n1] |
| 29 | 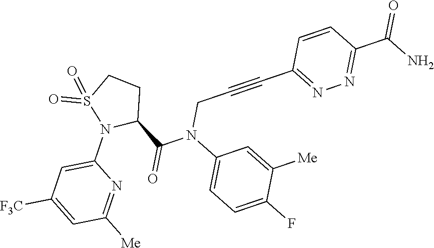 [SMILES: Cc1cc(C(F)(F)F)cc(N2[C@H](C(=O)N(CC#Cc3ccc(C(N)=O)nn3)c3ccc(F)c(C)c3)CCS2(=O)=O)n1] |
| 30 | 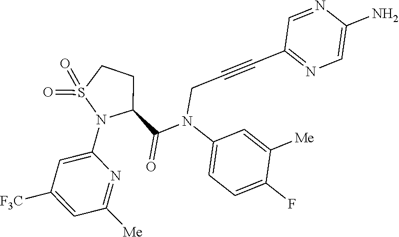 [SMILES: Cc1cc(C(F)(F)F)cc(N2[C@H](C(=O)N(CC#Cc3cnc(N)cn3)c3ccc(F)c(C)c3)CCS2(=O)=O)n1] |
| 31 | 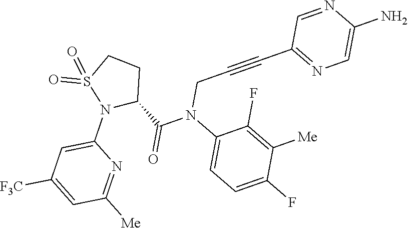 [SMILES: Cc1cc(C(F)(F)F)cc(N2[C@@H](C(=O)N(CC#Cc3cnc(N)cn3)c3ccc(F)c(C)c3F)CCS2(=O)=O)n1] |
| 32 | 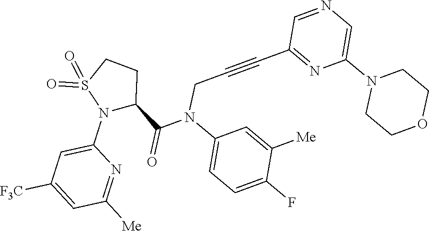 [SMILES: Cc1cc(C(F)(F)F)cc(N2[C@H](C(=O)N(CC#Cc3cncc(N4CCOCC4)n3)c3ccc(F)c(C)c3)CCS2(=O)=O)n1] |
| 33 | 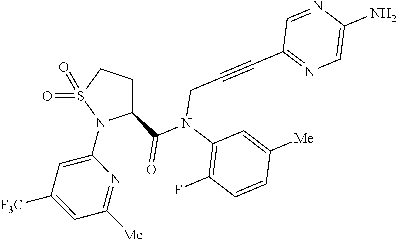 [SMILES: Cc1ccc(F)c(N(CC#Cc2cnc(N)cn2)C(=O)[C@@H]2CCS(=O)(=O)N2c2cc(C(F)(F)F)cc(C)n2)c1] |
| 34 | 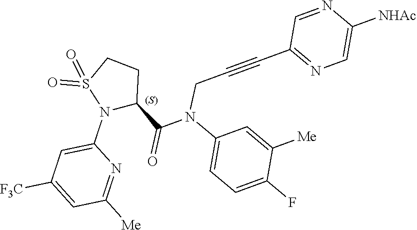 |
| 35 | 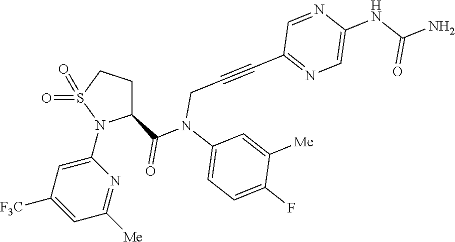 [SMILES: Cc1cc(C(F)(F)F)cc(N2[C@H](C(=O)N(CC#Cc3cnc(NC(N)=O)cn3)c3ccc(F)c(C)c3)CCS2(=O)=O)n1] |
| 36 | 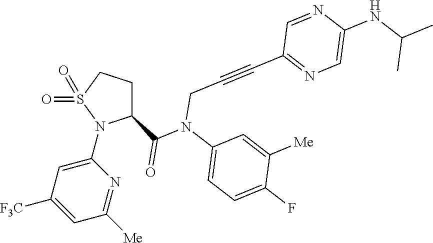 [SMILES: Cc1cc(C(F)(F)F)cc(N2[C@H](C(=O)N(CC#Cc3cnc(NC(C)C)cn3)c3ccc(F)c(C)c3)CCS2(=O)=O)n1] |
| 37 | 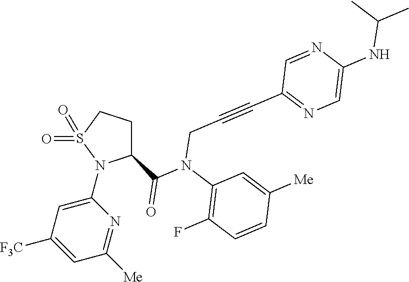 [SMILES: Cc1ccc(F)c(N(CC#Cc2cnc(NC(C)C)cn2)C(=O)[C@@H]2CCS(=O)(=O)N2c2cc(C(F)(F)F)cc(C)n2)c1] |
| 38 | 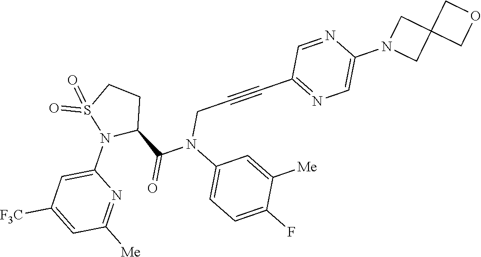 [SMILES: Cc1cc(C(F)(F)F)cc(N2[C@H](C(=O)N(CC#Cc3cnc(N4CC5(COC5)C4)cn3)c3ccc(F)c(C)c3)CCS2(=O)=O)n1] |
| 39 | 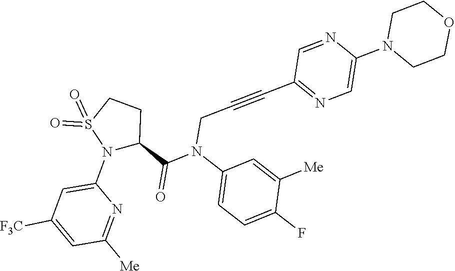 [SMILES: Cc1cc(C(F)(F)F)cc(N2[C@H](C(=O)N(CC#Cc3cnc(N4CCOCC4)cn3)c3ccc(F)c(C)c3)CCS2(=O)=O)n1] |
| 40 | 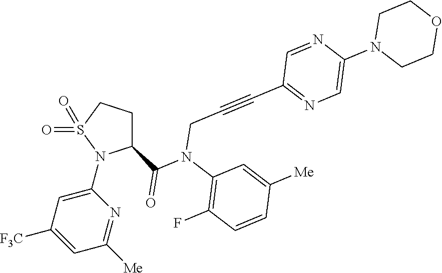 [SMILES: Cc1ccc(F)c(N(CC#Cc2cnc(N3CCOCC3)cn2)C(=O)[C@@H]2CCS(=O)(=O)N2c2cc(C(F)(F)F)cc(C)n2)c1] |
| 41 | 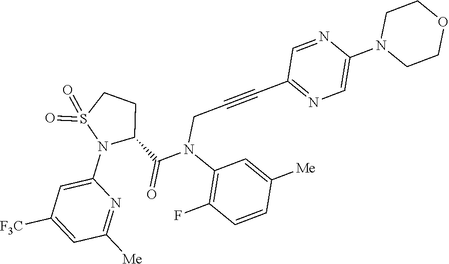 [SMILES: Cc1ccc(F)c(N(CC#Cc2cnc(N3CCOCC3)cn2)C(=O)[C@H]2CCS(=O)(=O)N2c2cc(C(F)(F)F)cc(C)n2)c1] |
| 42 | 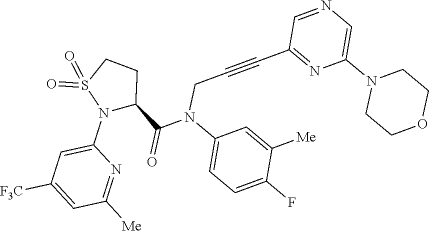 [SMILES: Cc1cc(C(F)(F)F)cc(N2[C@H](C(=O)N(CC#Cc3cncc(N4CCOCC4)n3)c3ccc(F)c(C)c3)CCS2(=O)=O)n1] |
| 43 | 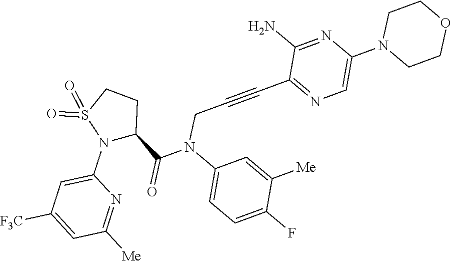 [SMILES: Cc1cc(C(F)(F)F)cc(N2[C@H](C(=O)N(CC#Cc3ncc(N4CCOCC4)nc3N)c3ccc(F)c(C)c3)CCS2(=O)=O)n1] |
| 44 | 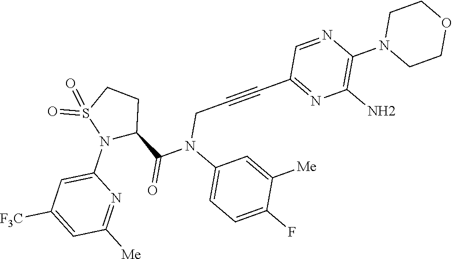 [SMILES: Cc1cc(C(F)(F)F)cc(N2[C@H](C(=O)N(CC#Cc3cnc(N4CCOCC4)c(N)n3)c3ccc(F)c(C)c3)CCS2(=O)=O)n1] |
| 45 | 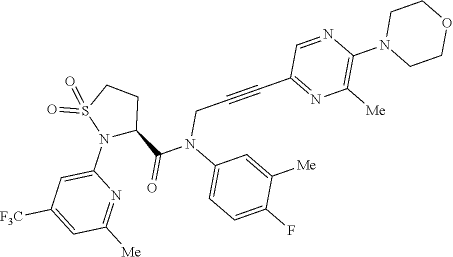 [SMILES: Cc1cc(C(F)(F)F)cc(N2[C@H](C(=O)N(CC#Cc3cnc(N4CCOCC4)c(C)n3)c3ccc(F)c(C)c3)CCS2(=O)=O)n1] |
| 46 | 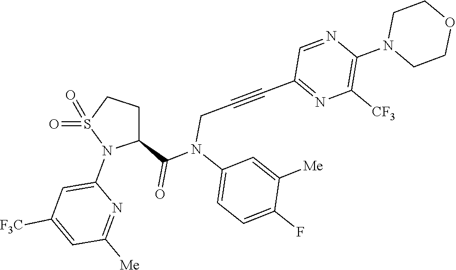 [SMILES: Cc1cc(C(F)(F)F)cc(N2[C@H](C(=O)N(CC#Cc3cnc(N4CCOCC4)c(C(F)(F)F)n3)c3ccc(F)c(C)c3)CCS2(=O)=O)n1] |
| 47 | 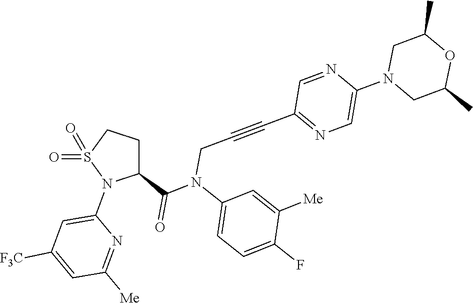 [SMILES: Cc1cc(C(F)(F)F)cc(N2[C@H](C(=O)N(CC#Cc3cnc(N4C[C@@H](C)O[C@@H](C)C4)cn3)c3ccc(F)c(C)c3)CCS2(=O)=O)n1] |
| 48 | 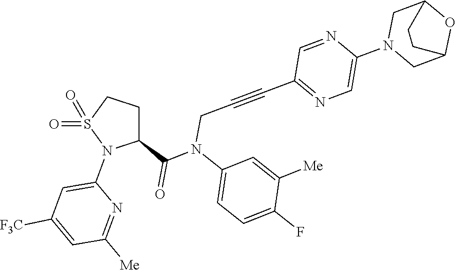 [SMILES: Cc1cc(C(F)(F)F)cc(N2[C@H](C(=O)N(CC#Cc3cnc(N4CC5CCC(C4)O5)cn3)c3ccc(F)c(C)c3)CCS2(=O)=O)n1] |
| 49 | 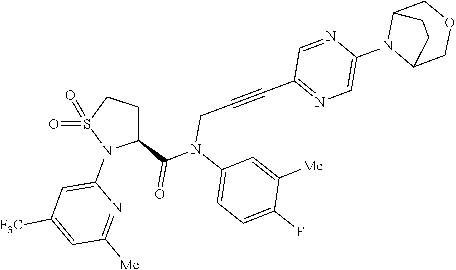 [SMILES: Cc1cc(C(F)(F)F)cc(N2[C@H](C(=O)N(CC#Cc3cnc(N4C5CCC4COC5)cn3)c3ccc(F)c(C)c3)CCS2(=O)=O)n1] |
| 50 | 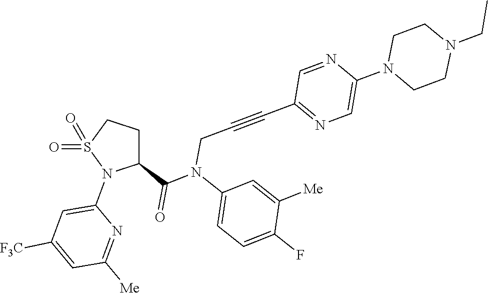 [SMILES: CCN1CCN(c2cnc(C#CCN(C(=O)[C@@H]3CCS(=O)(=O)N3c3cc(C(F)(F)F)cc(C)n3)c3ccc(F)c(C)c3)cn2)CC1] |
| 51 | 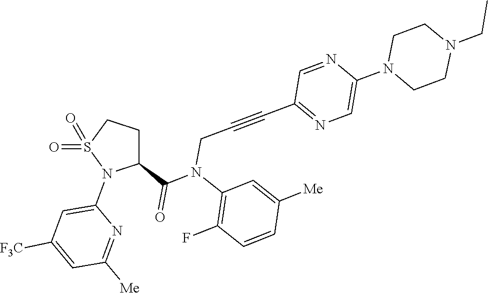 [SMILES: CCN1CCN(c2cnc(C#CCN(C(=O)[C@@H]3CCS(=O)(=O)N3c3cc(C(F)(F)F)cc(C)n3)c3cc(C)ccc3F)cn2)CC1] |
| 52 | 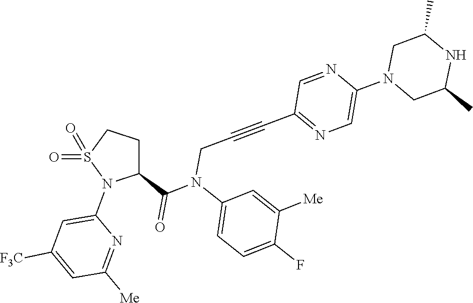 [SMILES: Cc1cc(C(F)(F)F)cc(N2[C@H](C(=O)N(CC#Cc3cnc(N4C[C@H](C)N[C@@H](C)C4)cn3)c3ccc(F)c(C)c3)CCS2(=O)=O)n1] |
| 53 | 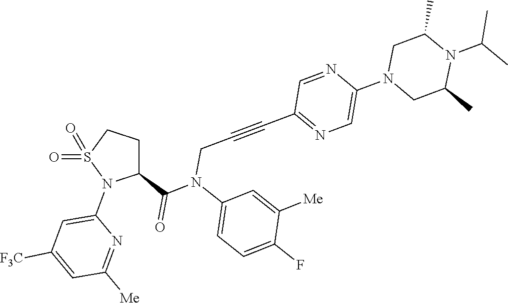 [SMILES: Cc1cc(C(F)(F)F)cc(N2[C@H](C(=O)N(CC#Cc3cnc(N4C[C@H](C)N(C(C)C)[C@@H](C)C4)cn3)c3ccc(F)c(C)c3)CCS2(=O)=O)n1] |
| 54 | 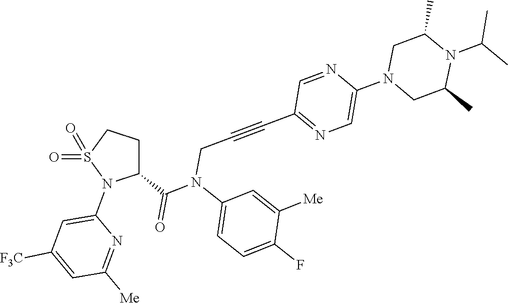 [SMILES: Cc1cc(C(F)(F)F)cc(N2[C@@H](C(=O)N(CC#Cc3cnc(N4C[C@H](C)N(C(C)C)[C@@H](C)C4)cn3)c3ccc(F)c(C)c3)CCS2(=O)=O)n1] |
| 55 | 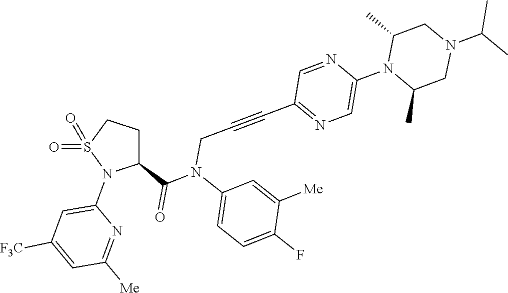 [SMILES: Cc1cc(C(F)(F)F)cc(N2[C@H](C(=O)N(CC#Cc3cnc(N4[C@H](C)CN(C(C)C)C[C@H]4C)cn3)c3ccc(F)c(C)c3)CCS2(=O)=O)n1] |
| 56 | 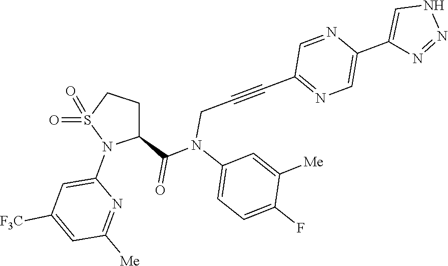 [SMILES: Cc1cc(C(F)(F)F)cc(N2[C@H](C(=O)N(CC#Cc3cnc(-c4c[nH]nn4)cn3)c3ccc(F)c(C)c3)CCS2(=O)=O)n1] |
| 57 | 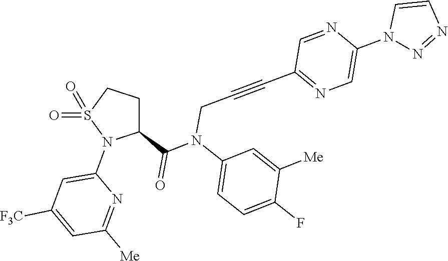 [SMILES: Cc1cc(C(F)(F)F)cc(N2[C@H](C(=O)N(CC#Cc3cnc(-n4ccnn4)cn3)c3ccc(F)c(C)c3)CCS2(=O)=O)n1] |
| 58 | 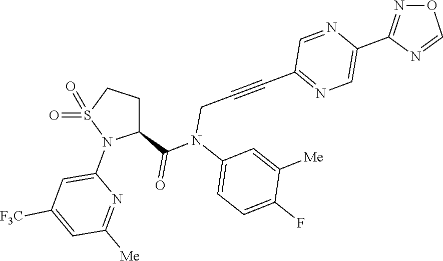 [SMILES: Cc1cc(C(F)(F)F)cc(N2[C@H](C(=O)N(CC#Cc3cnc(-c4ncon4)cn3)c3ccc(F)c(C)c3)CCS2(=O)=O)n1] |
| 59 | 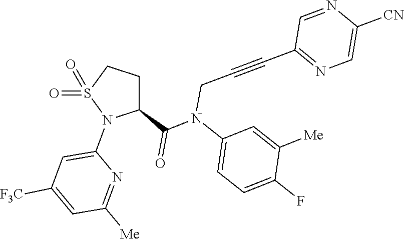 [SMILES: Cc1cc(C(F)(F)F)cc(N2[C@H](C(=O)N(CC#Cc3cnc(C#N)cn3)c3ccc(F)c(C)c3)CCS2(=O)=O)n1] |
| 60 | 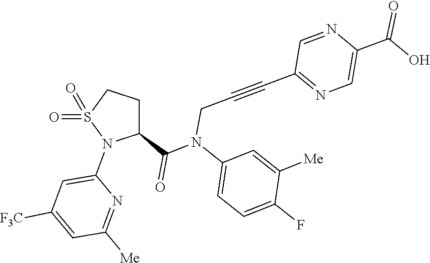 [SMILES: Cc1cc(C(F)(F)F)cc(N2[C@H](C(=O)N(CC#Cc3cnc(C(=O)O)cn3)c3ccc(F)c(C)c3)CCS2(=O)=O)n1] |
| 61 | 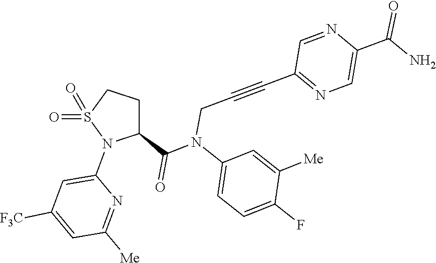 [SMILES: Cc1cc(C(F)(F)F)cc(N2[C@H](C(=O)N(CC#Cc3cnc(C(N)=O)cn3)c3ccc(F)c(C)c3)CCS2(=O)=O)n1] |
| 62 | 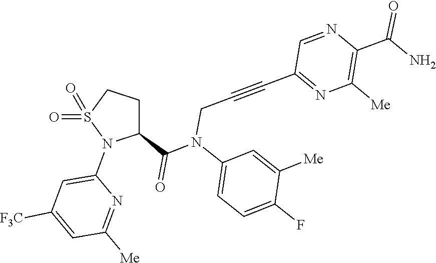 [SMILES: Cc1cc(C(F)(F)F)cc(N2[C@H](C(=O)N(CC#Cc3cnc(C(N)=O)c(C)n3)c3ccc(F)c(C)c3)CCS2(=O)=O)n1] |
| 63 | 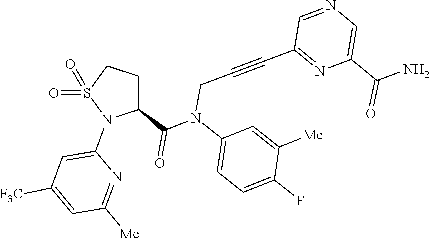 [SMILES: Cc1cc(C(F)(F)F)cc(N2[C@H](C(=O)N(CC#Cc3cncc(C(N)=O)n3)c3ccc(F)c(C)c3)CCS2(=O)=O)n1] |
| 64 | 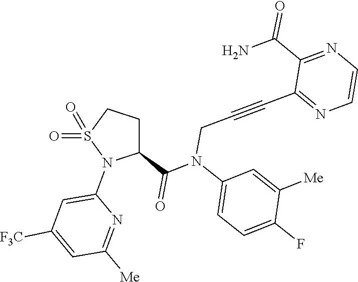 [SMILES: Cc1cc(C(F)(F)F)cc(N2[C@H](C(=O)N(CC#Cc3nccnc3C(N)=O)c3ccc(F)c(C)c3)CCS2(=O)=O)n1] |
| 65 | 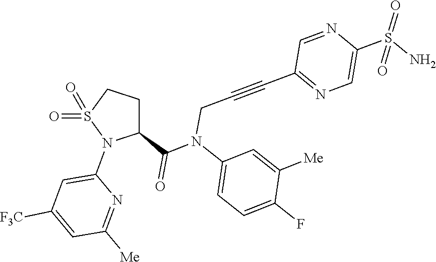 [SMILES: Cc1cc(C(F)(F)F)cc(N2[C@H](C(=O)N(CC#Cc3cnc(S(N)(=O)=O)cn3)c3ccc(F)c(C)c3)CCS2(=O)=O)n1] |
| 66 | 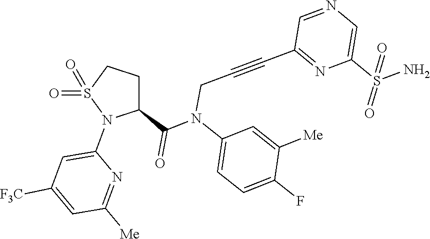 [SMILES: Cc1cc(C(F)(F)F)cc(N2[C@H](C(=O)N(CC#Cc3cncc(S(N)(=O)=O)n3)c3ccc(F)c(C)c3)CCS2(=O)=O)n1] |
| 67 | 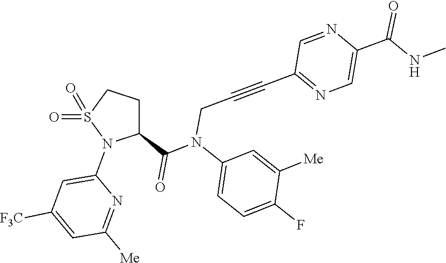 [SMILES: CNC(=O)c1cnc(C#CCN(C(=O)[C@@H]2CCS(=O)(=O)N2c2cc(C(F)(F)F)cc(C)n2)c2ccc(F)c(C)c2)cn1] |
| 68 | 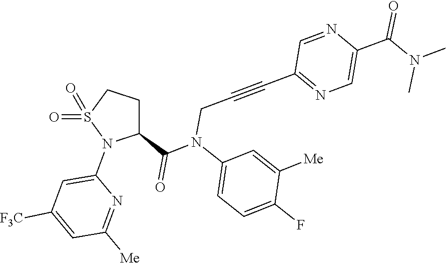 [SMILES: Cc1cc(C(F)(F)F)cc(N2[C@H](C(=O)N(CC#Cc3cnc(C(=O)N(C)C)cn3)c3ccc(F)c(C)c3)CCS2(=O)=O)n1] |
| 69 | 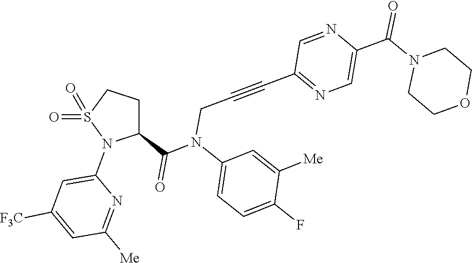 [SMILES: Cc1cc(C(F)(F)F)cc(N2[C@H](C(=O)N(CC#Cc3cnc(C(=O)N4CCOCC4)cn3)c3ccc(F)c(C)c3)CCS2(=O)=O)n1] |
| 70 | 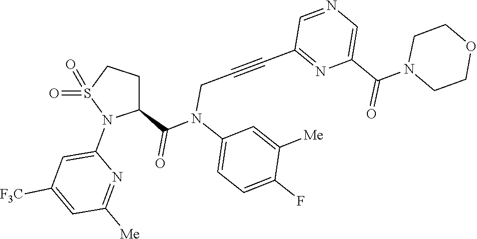 [SMILES: Cc1cc(C(F)(F)F)cc(N2[C@H](C(=O)N(CC#Cc3cncc(C(=O)N4CCOCC4)n3)c3ccc(F)c(C)c3)CCS2(=O)=O)n1] |
| 71 | 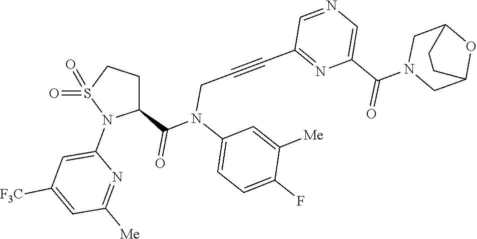 [SMILES: Cc1cc(C(F)(F)F)cc(N2[C@H](C(=O)N(CC#Cc3cncc(C(=O)N4CC5CCC(C4)O5)n3)c3ccc(F)c(C)c3)CCS2(=O)=O)n1] |
| 72 | 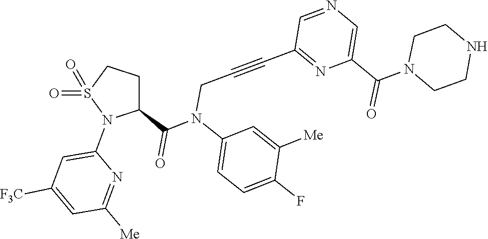 [SMILES: Cc1cc(C(F)(F)F)cc(N2[C@H](C(=O)N(CC#Cc3cncc(C(=O)N4CCNCC4)n3)c3ccc(F)c(C)c3)CCS2(=O)=O)n1] |
| 73 | 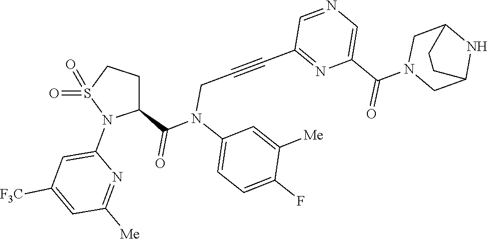 [SMILES: Cc1cc(C(F)(F)F)cc(N2[C@H](C(=O)N(CC#Cc3cncc(C(=O)N4CC5CCC(C4)N5)n3)c3ccc(F)c(C)c3)CCS2(=O)=O)n1] |
| 74 | 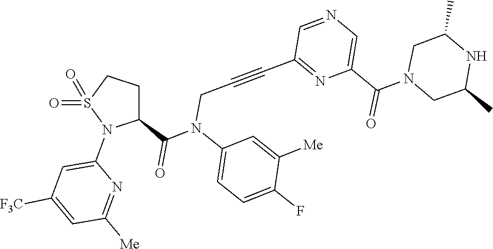 [SMILES: Cc1cc(C(F)(F)F)cc(N2[C@H](C(=O)N(CC#Cc3cncc(C(=O)N4C[C@H](C)N[C@@H](C)C4)n3)c3ccc(F)c(C)c3)CCS2(=O)=O)n1] |
| 75 | 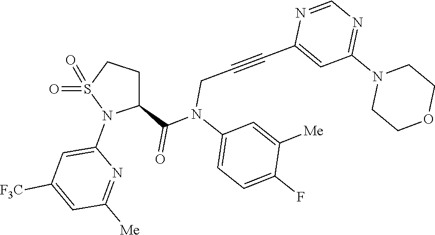 [SMILES: Cc1cc(C(F)(F)F)cc(N2[C@H](C(=O)N(CC#Cc3cc(N4CCOCC4)ncn3)c3ccc(F)c(C)c3)CCS2(=O)=O)n1] |
| 76 | 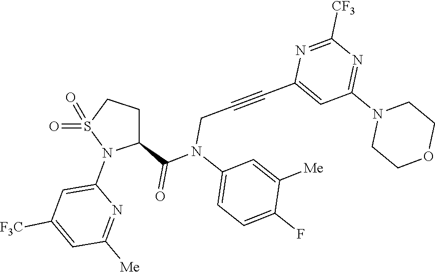 [SMILES: Cc1cc(C(F)(F)F)cc(N2[C@H](C(=O)N(CC#Cc3cc(N4CCOCC4)nc(C(F)(F)F)n3)c3ccc(F)c(C)c3)CCS2(=O)=O)n1] |
| 77 | 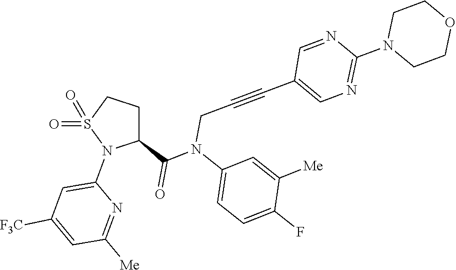 [SMILES: Cc1cc(C(F)(F)F)cc(N2[C@H](C(=O)N(CC#Cc3cnc(N4CCOCC4)nc3)c3ccc(F)c(C)c3)CCS2(=O)=O)n1] |
| 78 | 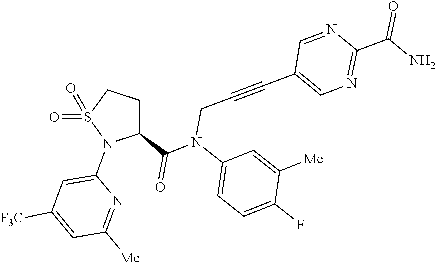 [SMILES: Cc1cc(C(F)(F)F)cc(N2[C@H](C(=O)N(CC#Cc3cnc(C(N)=O)nc3)c3ccc(F)c(C)c3)CCS2(=O)=O)n1] |
| 79 | 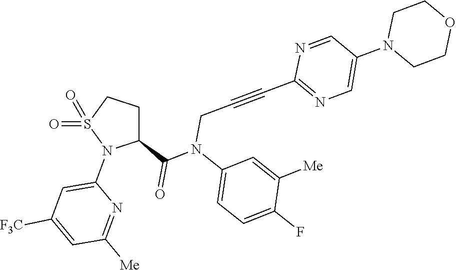 [SMILES: Cc1cc(C(F)(F)F)cc(N2[C@H](C(=O)N(CC#Cc3ncc(N4CCOCC4)cn3)c3ccc(F)c(C)c3)CCS2(=O)=O)n1] |
| 80 | 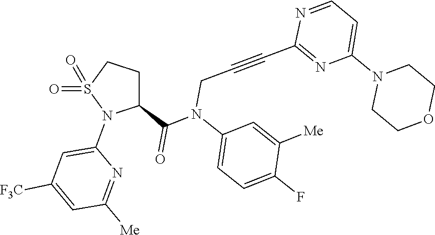 [SMILES: Cc1cc(C(F)(F)F)cc(N2[C@H](C(=O)N(CC#Cc3nccc(N4CCOCC4)n3)c3ccc(F)c(C)c3)CCS2(=O)=O)n1] |
| 81 | 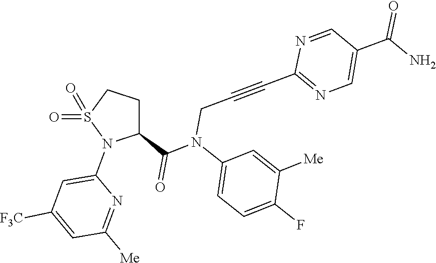 [SMILES: Cc1cc(C(F)(F)F)cc(N2[C@H](C(=O)N(CC#Cc3ncc(C(N)=O)cn3)c3ccc(F)c(C)c3)CCS2(=O)=O)n1] |
| 82 | 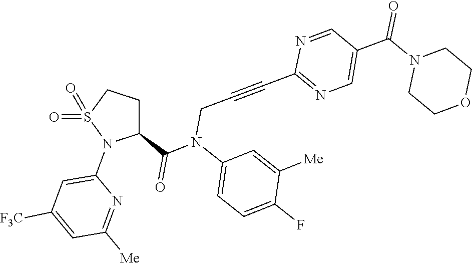 [SMILES: Cc1cc(C(F)(F)F)cc(N2[C@H](C(=O)N(CC#Cc3ncc(C(=O)N4CCOCC4)cn3)c3ccc(F)c(C)c3)CCS2(=O)=O)n1] |
| 83 | 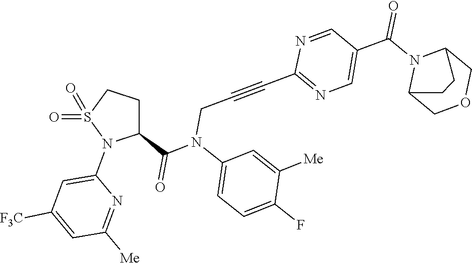 [SMILES: Cc1cc(C(F)(F)F)cc(N2[C@H](C(=O)N(CC#Cc3ncc(C(=O)N4C5CCC4COC5)cn3)c3ccc(F)c(C)c3)CCS2(=O)=O)n1] |
| 84 | 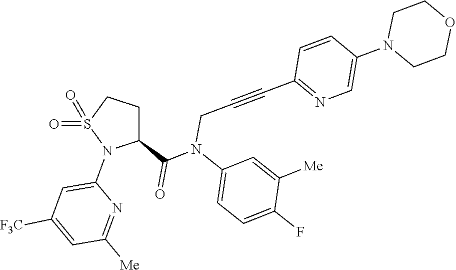 [SMILES: Cc1cc(C(F)(F)F)cc(N2[C@H](C(=O)N(CC#Cc3ccc(N4CCOCC4)cn3)c3ccc(F)c(C)c3)CCS2(=O)=O)n1] |
| 85 | 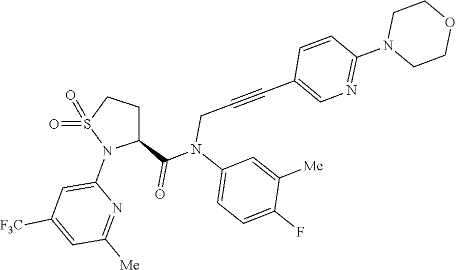 [SMILES: Cc1cc(C(F)(F)F)cc(N2[C@H](C(=O)N(CC#Cc3ccc(N4CCOCC4)nc3)c3ccc(F)c(C)c3)CCS2(=O)=O)n1] |
| 86 | 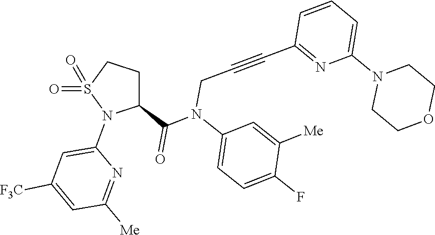 [SMILES: Cc1cc(C(F)(F)F)cc(N2[C@H](C(=O)N(CC#Cc3cccc(N4CCOCC4)n3)c3ccc(F)c(C)c3)CCS2(=O)=O)n1] |
| 87 | 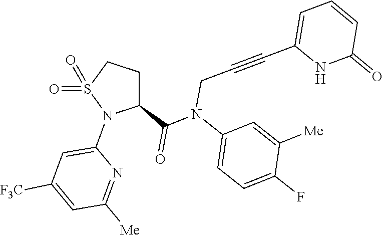 [SMILES: Cc1cc(C(F)(F)F)cc(N2[C@H](C(=O)N(CC#Cc3cccc(=O)[nH]3)c3ccc(F)c(C)c3)CCS2(=O)=O)n1] |
| 88 | 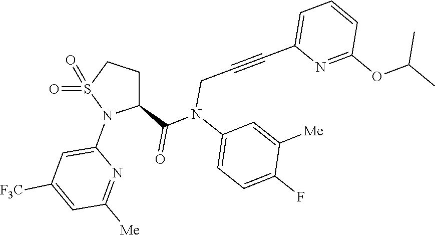 [SMILES: Cc1cc(C(F)(F)F)cc(N2[C@H](C(=O)N(CC#Cc3cccc(OC(C)C)n3)c3ccc(F)c(C)c3)CCS2(=O)=O)n1] |
| 89 | 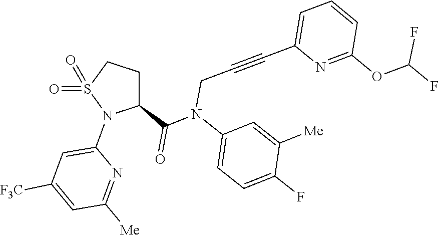 [SMILES: Cc1cc(C(F)(F)F)cc(N2[C@H](C(=O)N(CC#Cc3cccc(OC(F)F)n3)c3ccc(F)c(C)c3)CCS2(=O)=O)n1] |
| 90 | 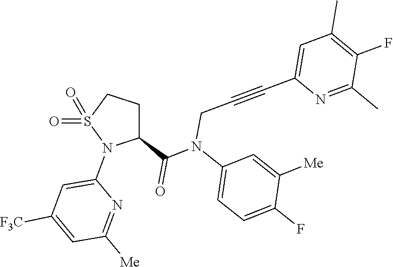 [SMILES: Cc1cc(C(F)(F)F)cc(N2[C@H](C(=O)N(CC#Cc3cc(C)c(F)c(C)n3)c3ccc(F)c(C)c3)CCS2(=O)=O)n1] |
| 91 | 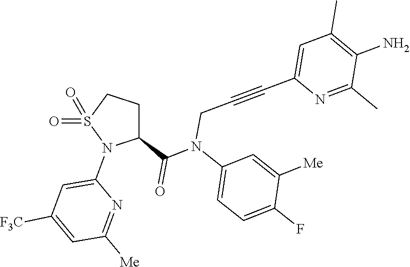 [SMILES: Cc1cc(C(F)(F)F)cc(N2[C@H](C(=O)N(CC#Cc3cc(C)c(N)c(C)n3)c3ccc(F)c(C)c3)CCS2(=O)=O)n1] |
| 92 | 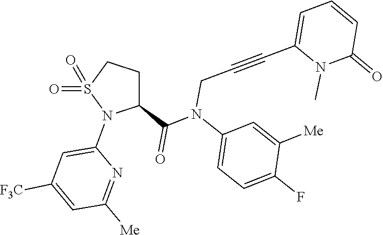 [SMILES: Cc1cc(C(F)(F)F)cc(N2[C@H](C(=O)N(CC#Cc3cccc(=O)n3C)c3ccc(F)c(C)c3)CCS2(=O)=O)n1] |
| 93 | 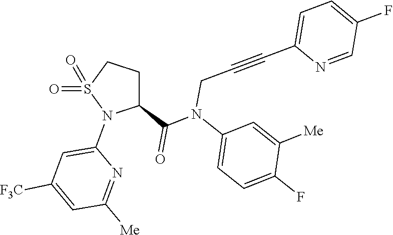 [SMILES: Cc1cc(C(F)(F)F)cc(N2[C@H](C(=O)N(CC#Cc3ccc(F)cn3)c3ccc(F)c(C)c3)CCS2(=O)=O)n1] |
| 94 | 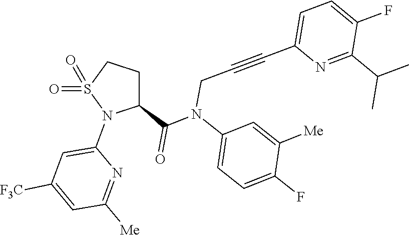 [SMILES: Cc1cc(C(F)(F)F)cc(N2[C@H](C(=O)N(CC#Cc3ccc(F)c(C(C)C)n3)c3ccc(F)c(C)c3)CCS2(=O)=O)n1] |
| 95 | 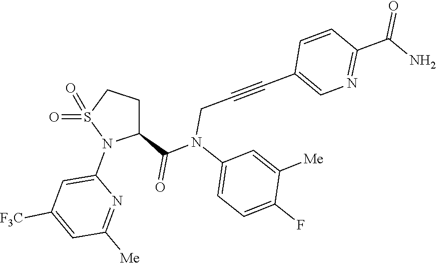 [SMILES: Cc1cc(C(F)(F)F)cc(N2[C@H](C(=O)N(CC#Cc3ccc(C(N)=O)nc3)c3ccc(F)c(C)c3)CCS2(=O)=O)n1] |
| 96 | 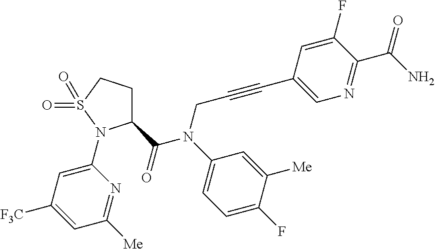 [SMILES: Cc1cc(C(F)(F)F)cc(N2[C@H](C(=O)N(CC#Cc3cnc(C(N)=O)c(F)c3)c3ccc(F)c(C)c3)CCS2(=O)=O)n1] |
| 97 | 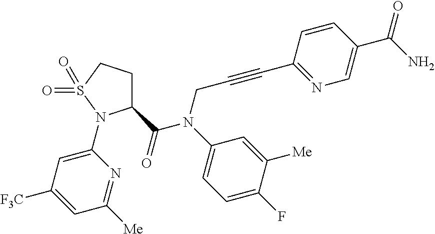 [SMILES: Cc1cc(C(F)(F)F)cc(N2[C@H](C(=O)N(CC#Cc3ccc(C(N)=O)cn3)c3ccc(F)c(C)c3)CCS2(=O)=O)n1] |
| 98 | 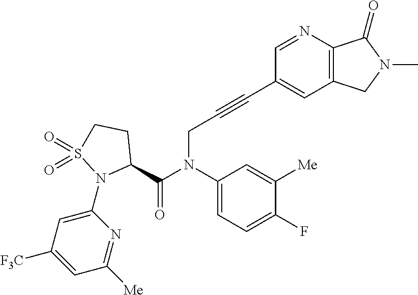 [SMILES: Cc1cc(C(F)(F)F)cc(N2[C@H](C(=O)N(CC#Cc3cnc4c(c3)CN(C)C4=O)c3ccc(F)c(C)c3)CCS2(=O)=O)n1] |
| 99 | 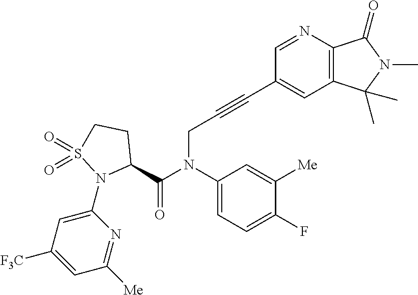 [SMILES: Cc1cc(C(F)(F)F)cc(N2[C@H](C(=O)N(CC#Cc3cnc4c(c3)C(C)(C)N(C)C4=O)c3ccc(F)c(C)c3)CCS2(=O)=O)n1] |
| 100 | 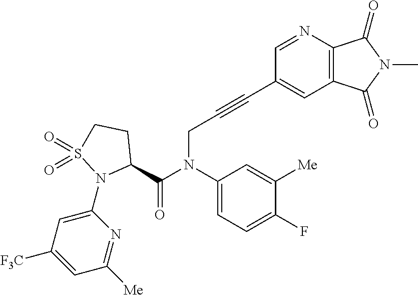 [SMILES: Cc1cc(C(F)(F)F)cc(N2[C@H](C(=O)N(CC#Cc3cnc4c(c3)C(=O)N(C)C4=O)c3ccc(F)c(C)c3)CCS2(=O)=O)n1] |
| 101 | 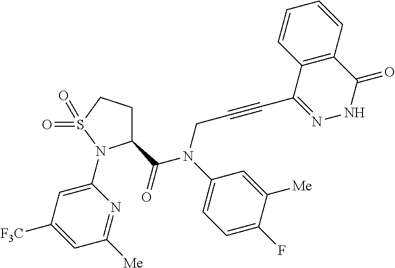 [SMILES: Cc1cc(C(F)(F)F)cc(N2[C@H](C(=O)N(CC#Cc3n[nH]c(=O)c4ccccc34)c3ccc(F)c(C)c3)CCS2(=O)=O)n1] |
| 102 | 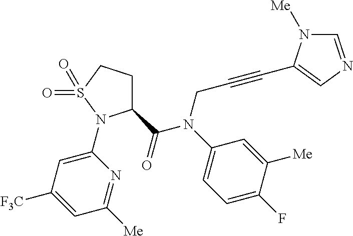 [SMILES: Cc1cc(C(F)(F)F)cc(N2[C@H](C(=O)N(CC#Cc3cncn3C)c3ccc(F)c(C)c3)CCS2(=O)=O)n1] |
| 103 | 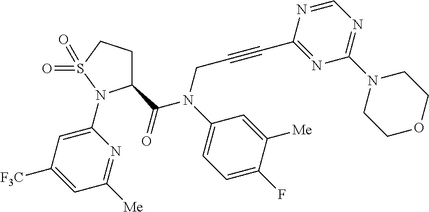 [SMILES: Cc1cc(C(F)(F)F)cc(N2[C@H](C(=O)N(CC#Cc3ncnc(N4CCOCC4)n3)c3ccc(F)c(C)c3)CCS2(=O)=O)n1] |
| 104 | 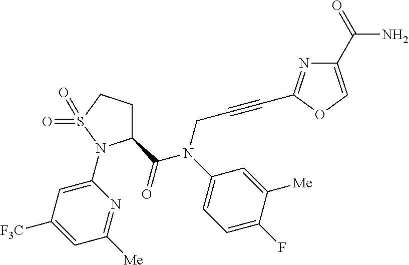 [SMILES: Cc1cc(C(F)(F)F)cc(N2[C@H](C(=O)N(CC#Cc3nc(C(N)=O)co3)c3ccc(F)c(C)c3)CCS2(=O)=O)n1] |
| 105 | 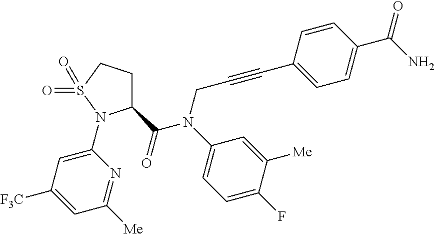 [SMILES: Cc1cc(C(F)(F)F)cc(N2[C@H](C(=O)N(CC#Cc3ccc(C(N)=O)cc3)c3ccc(F)c(C)c3)CCS2(=O)=O)n1] |
| 106 | 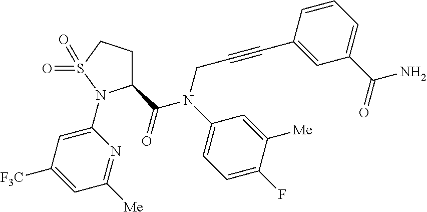 [SMILES: Cc1cc(C(F)(F)F)cc(N2[C@H](C(=O)N(CC#Cc3cccc(C(N)=O)c3)c3ccc(F)c(C)c3)CCS2(=O)=O)n1] |
| 107 | 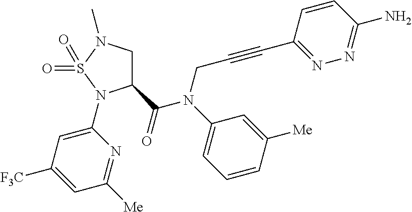 [SMILES: Cc1cccc(N(CC#Cc2ccc(N)nn2)C(=O)[C@@H]2CN(C)S(=O)(=O)N2c2cc(C(F)(F)F)cc(C)n2)c1] |
| 108 | 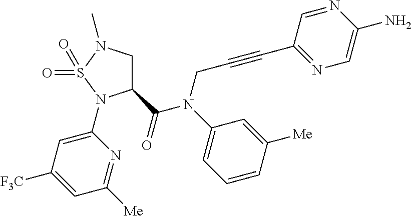 [SMILES: Cc1cccc(N(CC#Cc2cnc(N)cn2)C(=O)[C@@H]2CN(C)S(=O)(=O)N2c2cc(C(F)(F)F)cc(C)n2)c1] |
| 109 | 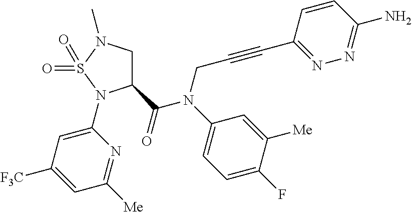 [SMILES: Cc1cc(C(F)(F)F)cc(N2[C@H](C(=O)N(CC#Cc3ccc(N)nn3)c3ccc(F)c(C)c3)CN(C)S2(=O)=O)n1] |
| 110 | 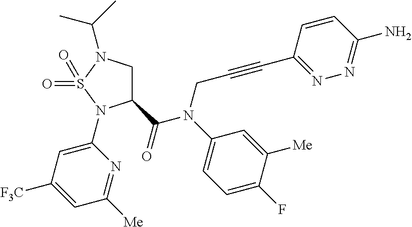 [SMILES: Cc1cc(C(F)(F)F)cc(N2[C@H](C(=O)N(CC#Cc3ccc(N)nn3)c3ccc(F)c(C)c3)CN(C(C)C)S2(=O)=O)n1] |
| 111 | 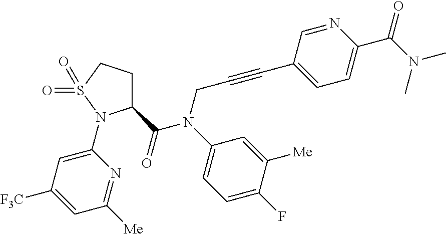 [SMILES: Cc1cc(C(F)(F)F)cc(N2[C@H](C(=O)N(CC#Cc3ccc(C(=O)N(C)C)nc3)c3ccc(F)c(C)c3)CCS2(=O)=O)n1] |
| 112 | 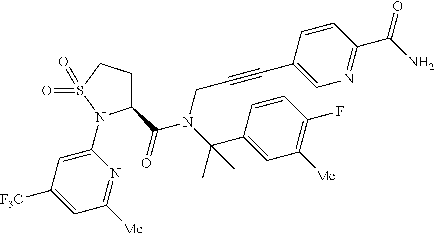 [SMILES: Cc1cc(C(F)(F)F)cc(N2[C@H](C(=O)N(CC#Cc3ccc(C(N)=O)nc3)C(C)(C)c3ccc(F)c(C)c3)CCS2(=O)=O)n1] |
| 113 | 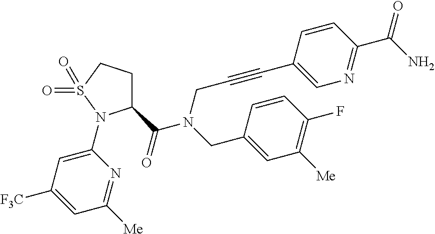 [SMILES: Cc1cc(C(F)(F)F)cc(N2[C@H](C(=O)N(CC#Cc3ccc(C(N)=O)nc3)Cc3ccc(F)c(C)c3)CCS2(=O)=O)n1] |
| 114 | 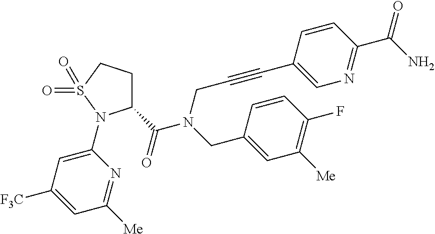 [SMILES: Cc1cc(C(F)(F)F)cc(N2[C@@H](C(=O)N(CC#Cc3ccc(C(N)=O)nc3)Cc3ccc(F)c(C)c3)CCS2(=O)=O)n1] |
| 115 | 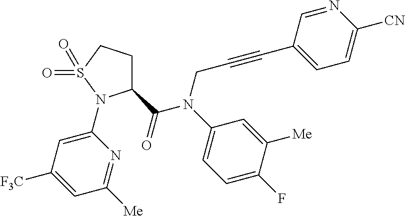 [SMILES: Cc1cc(C(F)(F)F)cc(N2[C@H](C(=O)N(CC#Cc3ccc(C#N)nc3)c3ccc(F)c(C)c3)CCS2(=O)=O)n1] |
| 116 | 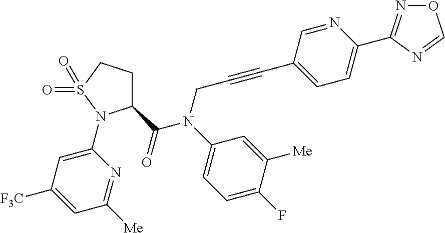 [SMILES: Cc1cc(C(F)(F)F)cc(N2[C@H](C(=O)N(CC#Cc3ccc(-c4ncon4)nc3)c3ccc(F)c(C)c3)CCS2(=O)=O)n1] |
| 117 | 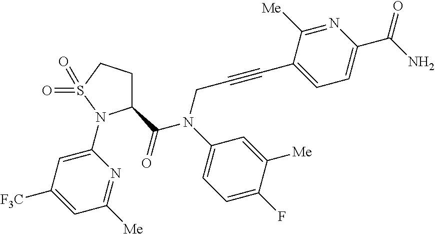 [SMILES: Cc1cc(C(F)(F)F)cc(N2[C@H](C(=O)N(CC#Cc3ccc(C(N)=O)nc3C)c3ccc(F)c(C)c3)CCS2(=O)=O)n1] |
| 118 | 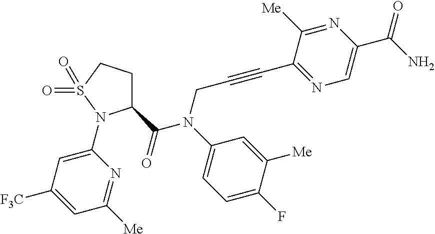 [SMILES: Cc1cc(C(F)(F)F)cc(N2[C@H](C(=O)N(CC#Cc3ncc(C(N)=O)nc3C)c3ccc(F)c(C)c3)CCS2(=O)=O)n1] |
| 119 | 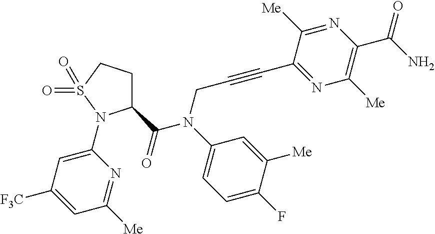 [SMILES: Cc1cc(C(F)(F)F)cc(N2[C@H](C(=O)N(CC#Cc3nc(C)c(C(N)=O)nc3C)c3ccc(F)c(C)c3)CCS2(=O)=O)n1] |
| 120 | 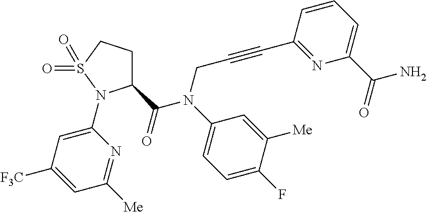 [SMILES: Cc1cc(C(F)(F)F)cc(N2[C@H](C(=O)N(CC#Cc3cccc(C(N)=O)n3)c3ccc(F)c(C)c3)CCS2(=O)=O)n1] |
| 121 | 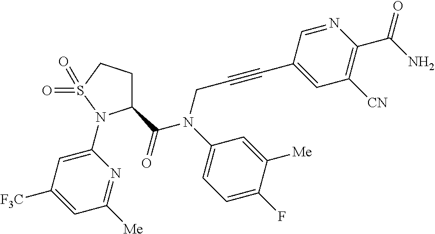 [SMILES: Cc1cc(C(F)(F)F)cc(N2[C@H](C(=O)N(CC#Cc3cnc(C(N)=O)c(C#N)c3)c3ccc(F)c(C)c3)CCS2(=O)=O)n1] |
| 122 | 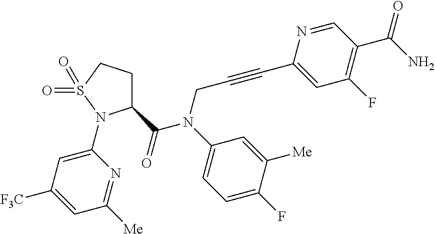 [SMILES: Cc1cc(C(F)(F)F)cc(N2[C@H](C(=O)N(CC#Cc3cc(F)c(C(N)=O)cn3)c3ccc(F)c(C)c3)CCS2(=O)=O)n1] |
| 123 | 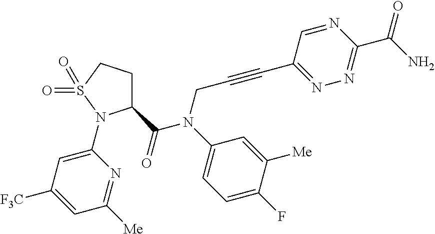 [SMILES: Cc1cc(C(F)(F)F)cc(N2[C@H](C(=O)N(CC#Cc3cnc(C(N)=O)nn3)c3ccc(F)c(C)c3)CCS2(=O)=O)n1] |
| 124 | 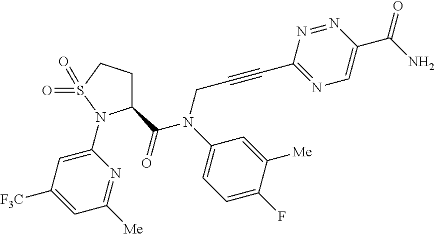 [SMILES: Cc1cc(C(F)(F)F)cc(N2[C@H](C(=O)N(CC#Cc3ncc(C(N)=O)nn3)c3ccc(F)c(C)c3)CCS2(=O)=O)n1] |
| 125 | 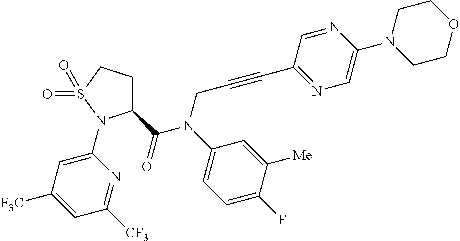 [SMILES: Cc1cc(N(CC#Cc2cnc(N3CCOCC3)cn2)C(=O)[C@@H]2CCS(=O)(=O)N2c2cc(C(F)(F)F)cc(C(F)(F)F)n2)ccc1F] |
| 126 | 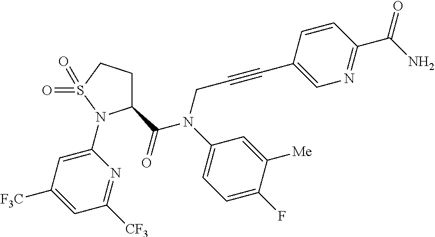 [SMILES: Cc1cc(N(CC#Cc2ccc(C(N)=O)nc2)C(=O)[C@@H]2CCS(=O)(=O)N2c2cc(C(F)(F)F)cc(C(F)(F)F)n2)ccc1F] |
| 127 | 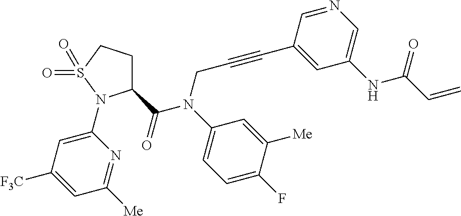 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)c1] |
| 128 | 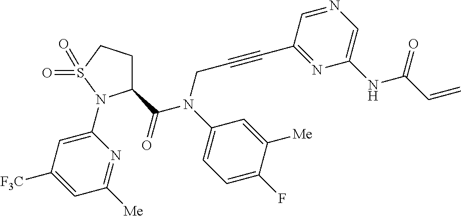 [SMILES: C=CC(=O)Nc1cncc(C#CCN(C(=O)[C@@H]2CCS(=O)(=O)N2c2cc(C(F)(F)F)cc(C)n2)c2ccc(F)c(C)c2)n1] |
| 129 | 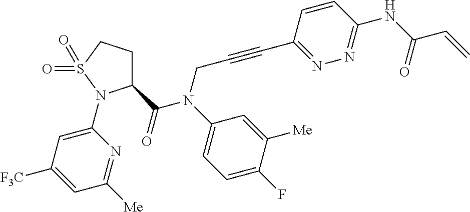 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nn1] |
| 130 | 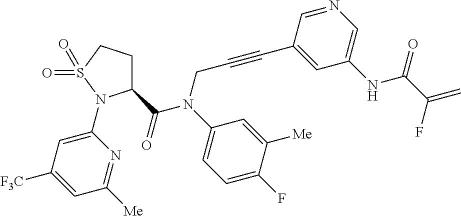 [SMILES: C=C(F)C(=O)Nc1cncc(C#CCN(C(=O)[C@@H]2CCS(=O)(=O)N2c2cc(C(F)(F)F)cc(C)n2)c2ccc(F)c(C)c2)c1] |
| 131 | 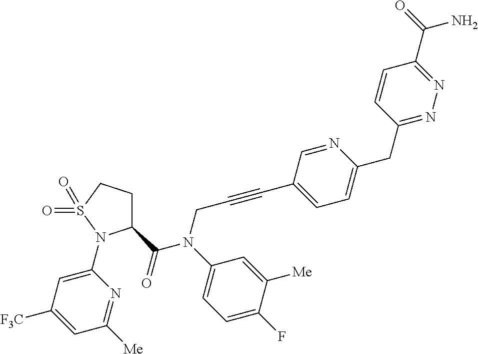 [SMILES: Cc1cc(C(F)(F)F)cc(N2[C@H](C(=O)N(CC#Cc3ccc(Cc4ccc(C(N)=O)nn4)nc3)c3ccc(F)c(C)c3)CCS2(=O)=O)n1] |
| 132 | 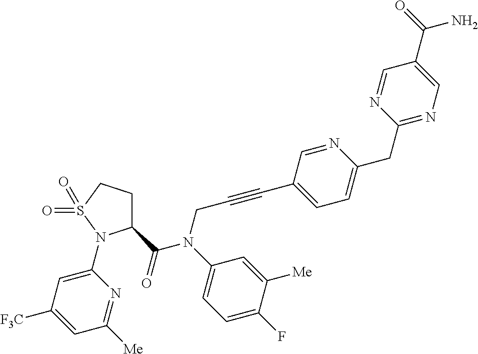 [SMILES: Cc1cc(C(F)(F)F)cc(N2[C@H](C(=O)N(CC#Cc3ccc(Cc4ncc(C(N)=O)cn4)nc3)c3ccc(F)c(C)c3)CCS2(=O)=O)n1] |
| 133 | 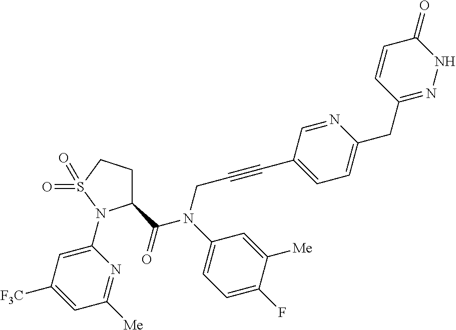 [SMILES: Cc1cc(C(F)(F)F)cc(N2[C@H](C(=O)N(CC#Cc3ccc(Cc4ccc(=O)[nH]n4)nc3)c3ccc(F)c(C)c3)CCS2(=O)=O)n1] |
| 134 | 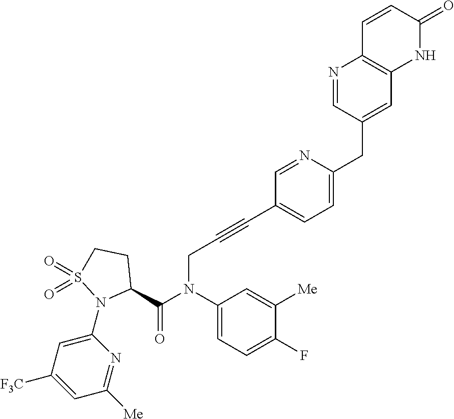 [SMILES: Cc1cc(C(F)(F)F)cc(N2[C@H](C(=O)N(CC#Cc3ccc(Cc4cnc5ccc(=O)[nH]c5c4)nc3)c3ccc(F)c(C)c3)CCS2(=O)=O)n1] |
| 135 | 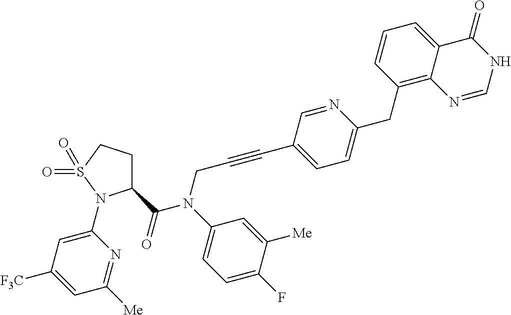 [SMILES: Cc1cc(C(F)(F)F)cc(N2[C@H](C(=O)N(CC#Cc3ccc(Cc4cccc5c(=O)[nH]cnc45)nc3)c3ccc(F)c(C)c3)CCS2(=O)=O)n1] |
| 136 | 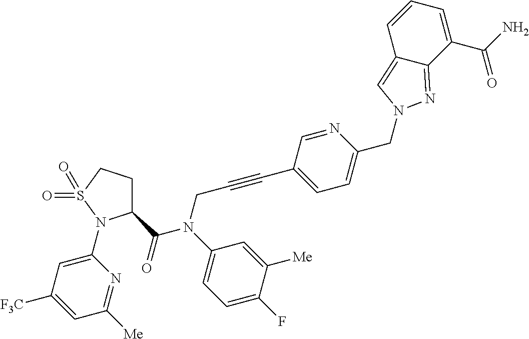 [SMILES: Cc1cc(C(F)(F)F)cc(N2[C@H](C(=O)N(CC#Cc3ccc(Cn4cc5cccc(C(N)=O)c5n4)nc3)c3ccc(F)c(C)c3)CCS2(=O)=O)n1] |
| 137 | 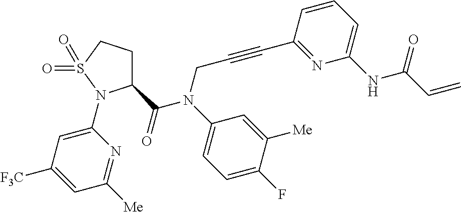 [SMILES: C=CC(=O)Nc1cccc(C#CCN(C(=O)[C@@H]2CCS(=O)(=O)N2c2cc(C(F)(F)F)cc(C)n2)c2ccc(F)c(C)c2)n1] |
| 138 | 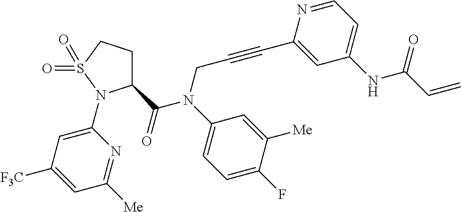 [SMILES: C=CC(=O)Nc1ccnc(C#CCN(C(=O)[C@@H]2CCS(=O)(=O)N2c2cc(C(F)(F)F)cc(C)n2)c2ccc(F)c(C)c2)c1] |
| 139 | 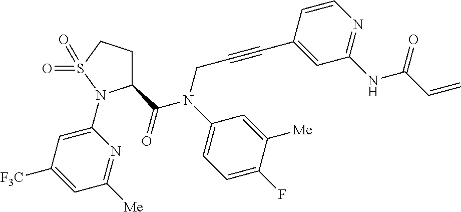 [SMILES: C=CC(=O)Nc1cc(C#CCN(C(=O)[C@@H]2CCS(=O)(=O)N2c2cc(C(F)(F)F)cc(C)n2)c2ccc(F)c(C)c2)ccn1] |
| 140 | 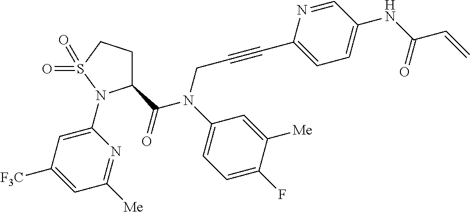 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)nc1] |
| 141 | 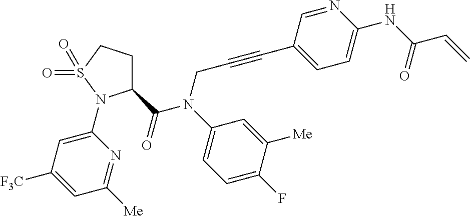 [SMILES: C=CC(=O)Nc1ccc(C#CCN(C(=O)[C@@H]2CCS(=O)(=O)N2c2cc(C(F)(F)F)cc(C)n2)c2ccc(F)c(C)c2)cn1] |
| 142 | 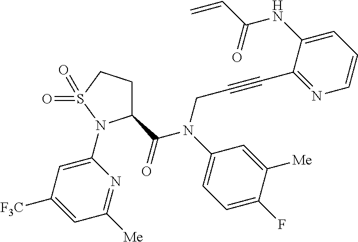 [SMILES: C=CC(=O)Nc1cccnc1C#CCN(C(=O)[C@@H]1CCS(=O)(=O)N1c1cc(C(F)(F)F)cc(C)n1)c1ccc(F)c(C)c1] |
| 143 | 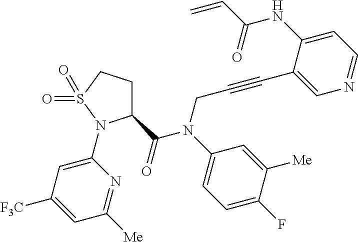 [SMILES: C=CC(=O)Nc1ccncc1C#CCN(C(=O)[C@@H]1CCS(=O)(=O)N1c1cc(C(F)(F)F)cc(C)n1)c1ccc(F)c(C)c1] |
| 144 | 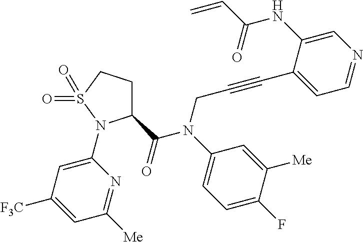 [SMILES: C=CC(=O)Nc1cnccc1C#CCN(C(=O)[C@@H]1CCS(=O)(=O)N1c1cc(C(F)(F)F)cc(C)n1)c1ccc(F)c(C)c1] |
| 145 | 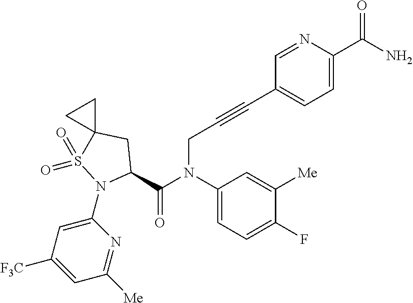 [SMILES: Cc1cc(C(F)(F)F)cc(N2[C@H](C(=O)N(CC#Cc3ccc(C(N)=O)nc3)c3ccc(F)c(C)c3)CC3(CC3)S2(=O)=O)n1] |

14. A pharmaceutical composition for preventing or treating diseases or disorders mediated by polymerase theta, preferably cancer, more preferably breast cancer, ovarian cancer, prostate cancer, pancreas cancer, or lung cancer, comprising the compound according to claim 1, or a tautomer, stereoisomer, prodrug, crystal form, isotopically labeled form, pharmaceutically acceptable salt, hydrate, or solvate thereof; and a pharmaceutically acceptable carrier(s) or excipient(s).

15. The pharmaceutical composition according to claim 14, wherein the composition is administered separately, sequentially or simultaneously with additional DNA damage response (DDR) targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).

16. A compound according to claim 1, or a tautomer, stereoisomer, prodrug, crystal form, isotopically labeled form, pharmaceutically acceptable salt, hydrate, or solvate thereof for use in the prevention or treatment of diseases or disorders mediated by polymerase theta, preferably cancer, more preferably breast cancer, ovarian cancer, prostate cancer, pancreas cancer, or lung cancer.

17. The compound according to claim 16, wherein the compound is administered separately, sequentially or simultaneously with additional DNA damage response (DDR) targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).

18. A method of treating or preventing diseases or disorders mediated by polymerase theta in a subject, comprising administering to the subject at least one compound according to claim 1, or a tautomer, stereoisomer, prodrug, crystal form, isotopically labeled form, pharmaceutically acceptable salt, hydrate, or solvate thereof.

19. The method of claim 18, wherein the diseases or disorders mediated by polymerase theta are cancer, preferably breast cancer, ovarian cancer, prostate cancer, pancreas cancer, or lung cancer.

20. The method of claim 18, further comprising administering to the subject additional DDR targeting anti-cancer agent(s), preferably PARP inhibitors (e.g., niraparib, olaparib, rucaparib, talazoparib, veliparib, E7016), ATR inhibitors (e.g., VE-821, VE-822, VX-970 (also known as M6620 or berzosertib), AZD6738 (e.g., ceralasertib), BAY 1895344, M4344), ATM inhibitors (e.g., AZD0156, AZD0156, AZD1390, M3541), DNA-PK Inhibitors (e.g., CC-115, M3814 (nedisertib or peposertib), AZD7648), CHK1/2 Inhibitors (e.g., UCN-01, AZD7762, LY2603618, MK-8776, GDC-0575, LY2606368 (e.g., prexasertib)), WEE1 Inhibitors (e.g., Adavosertib (MK-1775, or AZD1775), PLK1 Inhibitors (e.g., Volasertib (BI 6727), Onvansertib (e.g., PCM-075, NMS-1286937), APE1 inhibitors (e.g., methoxyamine), or Topoisomerase inhibitors (e.g., belotecan, CRLX101, irinotecan, LMP 400, LMP 776, NKTR-102, doxorubicin, epirubicin, etoposide, idarubicin, mitoxantrone, teniposide).


SEQUENCE LISTING (3 sequences)

SEQ ID NO: 1 - DNA, 14 bp, synthetic construct
  source 1..14: mol_type = other DNA
gcggctgtcataag

SEQ ID NO: 2 - DNA, 51 bp, synthetic construct
  source 1..51: mol_type = other DNA
gctacattgacaatggcatcaaatctcagattgcgtcttatgacagccgcg

SEQ ID NO: 3 - DNA, 51 bp, synthetic construct
  source 1..51: mol_type = other DNA
cgcggctgtcataagacgcaatctgagatttgatgccattgtcaatgtagc